(19) United States
(12) Patent Application Publication
(10) Pub. No.: US 2011/0200995 A1
(43) Pub. Date: Aug. 18, 2011

(54) OPTIMIZED PROBES AND PRIMERS AND METHODS OF USING SAME FOR THE DETECTION, SCREENING, ISOLATION AND SEQUENCING OF VANCOMYCIN RESISTANCE GENES AND VANCOMYCIN RESISTANT ENTEROCOCCI

(75) Inventors: Heinz R. Reiske, Framingham, MA (US); David L. Dolinger, Medway, MA (US); Alice A. Jacobs, Cambridge, MA (US); Juan Manuel Anzola, Waltham, MA (US)

(73) Assignee: INTELLIGENT MEDICAL DEVICES, INC., Cambridge, MA (US)

(21) Appl. No.: 12/875,849

(22) Filed: Sep. 3, 2010

Related U.S. Application Data
(60) Provisional application No. 61/239,940, filed on Sep. 4, 2009.

(51) Int. Cl. C12Q 1/68; C07H 21/00; C12P 19/34

(52) U.S. Cl. 435/6.11; 536/23.1; 435/6.1; 435/91.1; 536/24.3

(57) ABSTRACT

Described herein are primers and probes useful for detecting, screening, isolating, and sequencing of the vancomycin resistance genes and vancomycin resistant Enterococci and methods of using the described primers and probes.

### RELATED APPLICATION

This application claims the benefit of U.S. Provisional Patent Application No. 61/239,940, filed Sep. 4, 2009, the content of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Apr. 15, 2011, is named 09108055.txt.

### BACKGROUND

Enterococci are found within the normal intestinal flora and the female genital tract of humans, other mammals and birds. Enterococcus is intrinsically resistant (i.e., resistant to a low level) to β-lactam-based antibiotics (e.g., ampicillin, penicillin) and aminoglycosides (e.g., gentamicin, kanamycin, and neomycin). Enterococcus can acquire resistance to glycopeptides, such as vancomycin, and high concentrations of both β-lactam-based antibiotics and aminoglycosides, among others.

The importance of Enterococcus in vancomycin-resistant nosocomial infections or hospital acquired infections (HAIs) (vancomycin-resistant Enterococcus; VRE), has been the impetus for the development of therapeutic alternatives to vancomycin. In addition to vancomycin resistance in Enterococci, there is vancomycin resistance in Staphylococcus. Vancomycin resistant Staphylococcus aureus (VRSA) are antimicrobial-resistant Staphylococci. Patients that develop VRSA infections usually have several underlying health conditions (such as diabetes), previous infections with MRSA, and recent hospitalizations. The spread of VRSA occurs through close physical contact with infected patients or contaminated material. VRE infections can be treated with non-glycopeptide antibiotics such as cephalosporins and aminoglycosides; regardless of the phenotype, susceptibility testing is usually performed on isolates to determine the best course of treatment.

VRE is a threat to immunocompromised individuals, individuals recovering from surgical procedures and those generally in poor health. An individual can be colonized with VRE, which may or may not become a full-blown infection. Although colonized individuals can remain asymptomatic for months, even years, such persons are capable of transmitting VRE to others. VRE is rarely a concern for healthy adults, and is usually cleared from the host without intervention. Infections typically occur at sites such as wounds and urinary tract infections from in dwelling catheters. Infected patients can become septic. VRE is frequently transmitted person-to-person by healthcare workers (HCW) whose hands have become contaminated with VRE that is present in the feces, urine, or blood of an infected or colonized person. VRE can also be spread indirectly via hand contact with open wounds or contaminated environmental surfaces. Colonized individuals could also infect themselves through contact with feces, urine, blood or surfaces contaminated with their own feces, urine or blood. VRE can persist for weeks on environmental surfaces and medical instruments. Consequently, these surfaces are also potential modes of transmission and potential testing areas.

Culture-based diagnostic methods remain the definitive methods of choice for the determination of VRE. Bacterial colonies growing on Bile Esculin Azide plates supplemented with vancomycin (BEAV) could be presumed as VRE based on colonial morphology, but additional culture steps would be required for confirmation. This process can take 48 hours or longer. Culture can also have a high false negative rate. Adoption of nucleic acid-based tests (NAT) (such as polymerase chase reaction (PCR)) has led to diagnostic tests with significantly better turn-around time (2-5 hours), but many of the available tests lack sensitivity and specificity.

Detection of the vancomycin resistance genes from the genus Enterococcus, as well as other non-Enterococcal genera, would allow for improved treatments of bacterial infections. Furthermore, determination of whether the vancomycin resistance genes are from the Enterococcal genera would enable effective treatment decisions. A rapid and accurate diagnostic test panel for the detection of vancomycin-resistance genes and for detection of VRE would provide clinicians with an effective tool for diagnosis and supporting subsequent effective treatment regimens. A rapid screening panel for screening patients at risk for developing vancomycin resistance-associated and VRE-associated diseases would also provide clinicals with an efficient method to screen at-risk patients.

### SUMMARY

Described herein are nucleic acid probes and primers for detecting, isolating and sequencing all known, characterized variants of the vanA, vanB, vanC1, vanC2/3, vanD, vanE, and vanG vancomycin resistance genes (particularly the vanA and vanB genes) from the genus Enterococcus, as well as other non-Enterococcal genera, with a high degree of sensitivity and specificity. Also described herein are nucleic acid probes and primers for determining whether the vancomycin resistance genes are from the Enterococcal genera. A diagnostic test that distinguishes multiple drug resistance genes simultaneously and also determines whether the organism is VRE is necessary because such detection is critical in patient and personnel screening and surveillance of inanimate objects to eliminate the transmission of potentially deadly healthcare-associated infections (HAIs).

Patient, personnel and inanimate object screening, combined with barrier isolation and contact precautions of VRE-carriers, has been shown to be effective in controlling VRE infections; in some cases reducing to undetectable levels the VRE in clinical facilities. The assays described herein are critical components of a resistance screening program to screen patients admitted to and personnel working in clinical settings for VRE and VRSA. The assays described herein are also used to screen environmental surfaces for evidence that vancomycin-resistant organisms are or were present in a hospital setting. Additionally, the assays described herein are used to identify or confirm the identification of an isolate as containing vancomycin resistance and whether the organism is from the genera Enterococcus.

Enterococci are common commensal bacteria located in the gut microflora. Enterococcus faecium (Efm) and Enterococcus faecalis (Efs) are two of the most common Enterococcal species that have been shown to have vancomycin resistance. One marker for Efm and Efs is the sodA gene, which encodes the enzyme superoxide dismutase A (Efm sodA and Efs sodA). The sodA gene is frequently used as a bacterial species-specific marker. Other markers, identified through in silico analysis, target novel genes from Efm and Efs (Efm novel and Efs novel). An additional marker, a dual marker, identified through in silico analysis, binds to novel genes found in both E. faecium and E. faecalis (Efm/Efs dual).

Many facilities utilize culture-based methods for the determination and detection of antibiotic resistance genes, which requires days to obtain the results. The methods of detection of the resistance markers described herein occurs within a minimal number of hours, allowing clinicians to rapidly determine the appropriate contact precautions or treatment for individuals harboring vancomycin-resistant organisms, avoiding needless precautions for resistance-negative individuals and avoiding the careless use of antibiotics that have no or little treatment efficacy.

One embodiment is directed to an isolated nucleic acid sequence comprising a sequence selected from the group consisting of: SEQ ID NOS: 1-846.

One embodiment is directed to a method of hybridizing one or more isolated nucleic acid sequences comprising a sequence selected from the group consisting of: SEQ ID NOS: 1-502 to a vancomycin-resistance gene sequence, comprising contacting one or more isolated nucleic acid sequences to a sample comprising the vancomycin-resistance gene under conditions suitable for hybridization. In a particular embodiment, the vancomycin-resistance gene sequence is a genomic sequence, a naturally occurring plasmid, a naturally occurring transposable element, a template sequence or a sequence derived from an artificial construct. In a particular embodiment, the method(s) further comprise isolating and/or sequencing the hybridized vancomycin-resistance gene sequence.

One embodiment is directed to a primer set comprising at least one forward primer selected from the group consisting of SEQ ID NOS: 1, 6, 19, 22, 23, 26, 28, 29, 33, 34, 37-42, 45, 48, 53, 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); and at least one reverse primer selected from the group consisting of SEQ ID NOS: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG).

One embodiment is directed to a primer set comprising at least one forward primer selected from the group consisting of SEQ ID NOS: 517 (Efm sodA); 577, 586, 590, 598, 599, 600 (Efs sodA); and at least one reverse primer selected from the group consisting of SEQ ID NOS: 529 (Efm sodA); 617, 623, 624, 625, 637 and 640. (Efs sodA).

One embodiment is directed to a primer set comprising at least one forward primer selected from the group consisting of SEQ ID NOS: 683, 687, 692 (Efm novel); 758, 772, 773, 775 (Efs novel) and at least one reverse primer selected from the group consisting of SEQ ID NOS: 707, 720, 723 (Efm novel); 785, 791, 797, 799 and 803 (Efs novel).

One embodiment is directed to a primer set comprising at least one forward primer selected from the group consisting of SEQ ID NO: 843 (Efm/Efs dual); and at least one reverse primer selected from the group consisting of SEQ ID NOS: 845 and 846 (Efm/Efs dual).

One embodiment is directed to a primer set (at least one forward primer and at least one reverse primer) selected from the group consisting of: Groups 1-644 of Tables 5, 6, 8B, 9B, 10B, 11B, and 12.

One embodiment is directed to a method of producing a nucleic acid product, comprising contacting one or more isolated nucleic acid sequences selected from the group consisting of SEQ ID NOS: 1-846 to a sample comprising a vancomycin-resistance gene and/or an Efm and/or Efs sodA and/or Efm and/or Efs novel gene and/or dual marker genes under conditions suitable for nucleic acid polymerization. In a particular embodiment, the nucleic acid product is a vanA amplicon produced using at least one forward primer selected from the group consisting of SEQ ID NOS: 1, 6, 19, 22, 23, 26, 28, 29, 33, 34, 37-42, 45, 48, 53 and 59 and at least one reverse primer selected from the group consisting of SEQ ID NOS: 3, 5, 8, 10, 21, 31, 32, 36, 44, 47, 51, 52, 55 and 60. In a particular embodiment, the nucleic acid product is a vanB amplicon produced using at least one forward primer selected from the group consisting of SEQ ID NOS: 61, 68, 70-72, 75, 81, 83, 89, 93, 94, 103-105, 107 and 111 and at least one reverse primer selected from the group consisting of SEQ ID NOS: 63, 65, 66, 74, 77, 85-88, 90, 91, 95, and 97-102. In a particular embodiment, the nucleic acid product is a vanC1 amplicon produced using at least one forward primer selected from the group consisting of SEQ ID NOS: 123, 127, 130, 133, 138, 141, 144, 148, 151, 156, 158, 161, 162, 165, 168, 170, 171, 175, 178, 180, 183-185, 188, 191, 193, 194, 196, 198, and 200-204 and at least one reverse primer selected from the group consisting of SEQ ID NOS: 125, 129, 132, 135, 137, 140, 143, 146, 147, 150, 153, 155, 157, 160, 164, 167, 169, 173, 174, 177, 179, 187, 190 and 192. In a particular embodiment, the nucleic acid product is a vanC2/3 amplicon produced using at least one forward primer selected from the group consisting of SEQ ID NOS: 206, 210, 213, 217, 220, 221, 222, 224-226, 228-238, 240, 242 and 243 and at least one reverse primer selected from the group consisting of SEQ ID NOS: 208, 209, 212, 215, 216, 219, 223, 227, 239 and 241. In a particular embodiment, the nucleic acid product is a vanD amplicon produced using at least one forward primer selected from the group consisting of SEQ ID NOS: 388, 391, 394, 396, 399, 409, 415, 416, 425, 428, 435, 438, 440, 443, 445, 462, 465, 468, 471, 474, 477, 480, 483, 488, 491, 494-498 and 500-502 and at least one reverse primer selected from the group consisting of SEQ ID NOS: 390, 393, 395, 398, 401, 411, 417, 419, 421, 426, 430, 434, 437, 439, 442, 444, 447-455, 458-461, 464, 467, 470, 473, 476, 479, 482, 486, 490 and 493. In a particular embodiment, the nucleic acid product is a vanE amplicon produced using at least one forward primer selected from the group consisting of SEQ ID NOS: 334, 337-380 and 382-387 and at least one reverse primer selected from the group consisting of SEQ ID NOS: 336 and 381. In a particular embodiment, the nucleic acid product is a vanG amplicon produced using at least one forward primer selected from the group consisting of SEQ ID NOS: 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 and at least one reverse primer selected from the group consisting of SEQ ID NOS: 246-248, 250, 251, 254, 258, 259, 262, 266, 269, 274, 284, 286, 287, 290, 301, 304, 306, 308, 312, 320 and 324. In a particular embodiment, the nucleic acid product is a Efm sodA amplicon produced using at least one forward primer consisting of SEQ ID NOS: 517 and at least one reverse primer consisting of SEQ ID NOS: 529. In a particular embodiment, the nucleic acid product is a Efs sodA amplicon produced using at least one forward primer selected from the group consisting of SEQ ID NOS: 577, 586, 590, 598, 599, 600; and at least one reverse primer selected from the group consisting of SEQ ID NOS: 617, 623, 624, 625, 637 and 640. In a particular embodiment, the nucleic acid product is a Efm novel amplicon produced using at least one forward primer selected from the group consisting of SEQ ID NOS: 683, 687, 692; and at least one reverse primer selected from the group consisting of SEQ ID NOS: 707, 720, 723. In a particular embodiment, the nucleic acid product is a Efs novel amplicon produced using at least one forward primer selected from the group consisting of SEQ ID NOS: 758, 772, 773, 775; and at least one reverse primer selected from the group consisting of SEQ ID NOS: 785, 791, 797, 799 and 803. In a particular embodiment, the nucleic acid product is a Efm dual and Efs dual amplicon produced using at least one forward primer consisting of SEQ ID NO: 843; and at least one reverse primer consisting of SEQ ID NOS: 845 and 846.

One embodiment is directed to a probe that hybridized to an amplicon produced as described herein, e.g., using the primers described herein. In a particular embodiment, the probe comprises a sequence selected from the group consisting of SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 273, 289, 292, 294, 296, 319, 323 (vanG). In a particular embodiment, the probe comprises a sequence selected from the group consisting of SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual).

In a particular embodiment, the probe(s) is labeled with a detectable label selected from the group consisting of: a fluorescent label, a chemiluminescent label, a quencher, a radioactive label, biotin and gold.

One embodiment is directed to a set of probes that hybridize to an amplicon produced as described herein, e.g., using the primers described herein. In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), and a second probe comprises a sequence selected from the group consisting of SEQ ID NOS: 62, 64, 67, 69, 73, 76, 78-80, 82, 84, 92, 96, 108-110, 112 (vanB). In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), and a third probe comprises SEQ ID NO: 505. In a particular embodiment, a first probe comprises a sequence selected from the group consisting of SEQ ID NOS: 555, 562, 571 (Efm sodA); a second probe comprises a sequence selected from the group consisting of SEQ ID NOS: 644, 650, 654, 659, 661-665, 667, 673, 675-677 (Efs sodA). In a particular embodiment, a first probe comprises a sequence selected from the group consisting of SEQ ID NOS: 555, 562, 571 (Efm sodA); a second probe comprises a sequence selected from the group consisting of SEQ ID NOS: 644, 650, 654, 659, 661, 662, 663, 664, 665, 667, 673, 675, 676, 677 (Efs sodA); a third probe comprises SEQ ID NO: 505. In a particular embodiment, a first probe comprises a sequence selected from the group consisting of SEQ ID NOS: 728, 750 (Efm novel); a second probe comprises a sequence selected from the group consisting of SEQ ID NOS: 815, 832 (Efs novel). In a particular embodiment, a first probe comprises a sequence selected from the group consisting of SEQ ID NOS: 728, 750 (Efm novel); a second probe comprises a sequence selected from the group consisting of SEQ ID NOS: 815, 832 (Efs novel); a third probe comprises SEQ ID NO: 505. In a particular embodiment, a probe comprises a sequence consisting of SEQ ID NO: 844 (Efm/Efs dual). In a particular embodiment, a probe comprises a sequence consisting of SEQ ID NO: 844 (Efm/Efs dual) and a second probe comprises SEQ ID NO: 505.

In a particular embodiment, the first probe is labeled with a first detectable label and the second probe is labeled with a second detectable label. In a particular embodiment, the first probe and the second probe are labeled with the same detectable label. In a particular embodiment, the first probe is labeled with a first detectable label, the second probe is labeled with a second detectable label and the third probe is labeled with a third detectable label. One embodiment is directed to a probe that hybridizes directly to the genomic sequences of the target without amplification. In a particular embodiment, the probe comprises a sequence selected from the group consisting of SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 273, 289, 292, 294, 296, 319, 323 (vanG). In a particular embodiment, the probe comprises a sequence selected from the group consisting of SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel); and 844 (Efm/Efs dual).

In a particular embodiment, the probe(s) is labeled with a detectable label selected from the group consisting of: a fluorescent label, a chemiluminescent label, a quencher, a radioactive label, biotin and gold.

One embodiment is directed to a set of probes that hybridize directly to the genomic sequences of the target without amplification. In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), and a second probe comprises a sequence selected from the group consisting of SEQ ID NOS: 62, 64, 67, 69, 73, 76, 78-80, 82, 84, 92, 96, 108-110, 112 (vanB). In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), a third probe comprises SEQ ID NO: 505. In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), a third probe comprises SEQ ID NOS: 555, 562, 571 (Efm sodA); and a fourth probe comprises a sequence selected from the group consisting of SEQ ID NOS: 644, 650, 654, 659, 661, 662, 663, 664, 665, 667, 673, 675, 676, 677 (Efs sodA). In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), a third probe comprises SEQ ID NOS: 555, 562, 571 (Efm sodA); and a fourth probe comprises a sequence selected from the group consisting of SEQ ID NOS: 644, 650, 654, 659, 661, 662, 663, 664, 665, 667, 673, 675, 676, 677 (Efs sodA); a fifth probe comprises SEQ ID NO: 505. In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), a third probe comprises SEQ ID NOS: 728, 750 (Efm novel); a fourth probe comprises a sequence selected from the group consisting of SEQ ID NOS: 815, 832 (Efs novel) and a fifth probe comprises SEQ ID NO: 505. In a particular embodiment, a first probe comprises a sequence selected from the group consisting of SEQ ID NOS: 2, 4, 7, 9, 11-18, 20, 24, 25, 27, 30, 35, 43, 46, 49, 50, 54, 56-58 (vanA), a second probe comprises a sequence selected from the group consisting of SEQ ID NOS: 62, 64, 67, 69, 73, 76, 78-80, 82, 84, 92, 96, 108-110, 112 (vanB), a third probe comprises SEQ ID NOS: 844 (Efs/Efm dual) and a fourth probe comprises SEQ ID: 505.

In a particular embodiment, the first probe is labeled with a first detectable label and the second probe is labeled with a second detectable label. In a particular embodiment, the first probe and the second probe are labeled with the same detectable label. In a particular embodiment, the first probe is labeled with a first detectable label, the second probe is labeled with a second detectable label and the third probe is labeled with a third detectable label. In a particular embodiment, the first probe is labeled with a first detectable label, the second probe is labeled with a second detectable label, the third probe is labeled with a third detectable label and the fourth probe is labeled with a fourth detectable label. In a particular embodiment, the detectable labels are selected from the group consisting of: a fluorescent label, a chemiluminescent label, a quencher, a radioactive label, biotin and gold.

In one embodiment, the probe(s) is fluorescently labeled and the step of detecting the binding of the probe to the amplified product comprises measuring the fluorescence of the sample. In one embodiment, the probe comprises a fluorescent reporter moiety and a quencher of fluorescence-quenching moiety. Upon probe hybridization with the amplified product, the exonuclease activity of a DNA polymerase dissociates the probe's fluorescent reporter and the quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. In another embodiment, each probe in the multiplex reaction is labeled with a different distinguishable and detectable label.

In a particular embodiment, the probes are molecular beacons. Molecular beacons are single-stranded probes that form a stem-and-loop structure. A fluorophore is covalently linked to one end of the stem and a quencher is covalently linked to the other end of the stem forming a stem hybrid; fluorescence is quenched when the formation of the stem loop positions the fluorophore proximal to the quencher. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein may be designed and utilized as molecular beacons.

One embodiment is directed a method for detecting a vancomycin-resistance gene(s) in a sample, comprising: (a) contacting the sample with at least one forward primer comprising a sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG), and at least one reverse primer comprising a sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG) under conditions such that nucleic acid amplification occurs to yield an amplicon; and (b) contacting the amplicon with one or more probes comprising one or more sequences selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG) under conditions such that hybridization of the probe to the amplicon occurs, wherein hybridization of the probe is indicative of a vancomycin-resistance gene(s) in the sample.

One embodiment is directed a method for detecting an Enterococcal Efm sodA or Efs sodA gene(s) or novel gene or dual marker in a sample, comprising: (a) contacting the sample with at least one forward primer comprising a sequence selected from the group consisting of: SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual); and at least one reverse primer comprising a sequence selected from the group consisting of SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual) under conditions such that nucleic acid amplification occurs to yield an amplicon; and (b) contacting the amplicon with one or more probes comprising one or more sequences selected from the group consisting of: SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual).

One embodiment is directed a method for detecting a vancomycin-resistance gene(s) or an Enterococcal marker gene in a sample, comprising: (a) contacting the sample with at least one forward primer comprising a sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG), 517 (Efm sodA); 577, 586, 590, 598, 599, 600 (Efs sodA); 683, 687, 692 (Efm novel); 758, 772, 773, 775 (Efs novel); and 843 (Efm/Efs dual), and at least one reverse primer comprising a sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG), 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual) under conditions such that nucleic acid amplification occurs to yield an amplicon; and (b) contacting the amplicon with one or more probes comprising one or more sequences selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG), 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual) under conditions such that hybridization of the probe to the amplicon occurs, wherein hybridization of the probe is indicative of a vancomycin-resistance gene(s) in the sample.

In a particular embodiment, each of the one or more probes is labeled with a different detectable label. In a particular embodiment, the one or more probes are labeled with the same detectable label. In a particular embodiment, the sample is selected from the group consisting of: blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage or fibroblasts. In one embodiment, the sample is from a human, is non-human in origin, or is derived from an inanimate object or environmental surfaces. In a particular embodiment, the at least one forward primer, the at least one reverse primer and the one or more probes are selected from the group consisting of: Groups 1-212 of Table 5, Groups 213-601 of Table 6, Groups 603-605 of Table 8B, Groups 606-627 of Table 9B, Groups 628-636 of Table 10B, Groups 637-643 of Table 11B, and Group 644 of Table 12. In a particular embodiment, the method(s) further comprise isolating and/or sequencing the vancomycin-resistance gene sequence(s) and/or Enterococcal sodA or novel gene or dual marker sequence(s) in a sample.

One embodiment is directed to a primer set or collection of primer sets for amplifying DNA of a vancomycin-resistance gene (vanA gene), comprising a nucleotide sequence selected from the group consisting of: (1) SEQ ID NOS: 1 and 3; (2) SEQ ID NOS: 1 and 32; (3) SEQ ID NOS: 1 and 5; (4) SEQ ID NOS: 19 and 21; (5) SEQ ID NOS: 19 and 3; (6) SEQ ID NOS: 19 and 32; (7) SEQ ID NOS: 19 and 47; (8) SEQ ID NOS: 19 and 5; (9) SEQ ID NOS: 19 and 52; (10) SEQ ID NOS: 19 and 55; (11) SEQ ID NOS: 22 and 21; (12) SEQ ID NOS: 22 and 3; (13) SEQ ID NOS: 22 and 32; (14) SEQ ID NOS: 22 and 5; (15) SEQ ID NOS: 23 and 21; (16) SEQ ID NOS: 23 and 3; (17) SEQ ID NOS: 23 and 32; (18) SEQ ID NOS: 23 and 5; (19) SEQ ID NOS: 26 and 3; (20) SEQ ID NOS: 26 and 32; (21) SEQ ID NOS: 26 and 47; (22) SEQ ID NOS: 26 and 5; (23) SEQ ID NOS: 26 and 51; (24) SEQ ID NOS: 28 and 3; (25) SEQ ID NOS: 28 and 32; (26) SEQ ID NOS: 28 and 5; (27) SEQ ID NOS: 29 and 21; (28) SEQ ID NOS: 29 and 3; (29) SEQ ID NOS: 29 and 5; (30) SEQ ID NOS: 29 and 8; (31) SEQ ID NOS: 33 and 21; (32) SEQ ID NOS: 33 and 3; (33) SEQ ID NOS: 33 and 32; (34) SEQ ID NOS: 33 and 5; (35) SEQ ID NOS: 34 and 36; (36) SEQ ID NOS: 34 and 52; (37) SEQ ID NOS: 37 and 3; (38) SEQ ID NOS: 37 and 32; (39) SEQ ID NOS: 37 and 5; (40) SEQ ID NOS: 38 and 3; (41) SEQ ID NOS: 38 and 32; (42) SEQ ID NOS: 38 and 5; (43) SEQ ID NOS: 39 and 3; (44) SEQ ID NOS: 39 and 32; (45) SEQ ID NOS: 39 and 5; (46) SEQ ID NOS: 40 and 3; (47) SEQ ID NOS: 40 and 32; (48) SEQ ID NOS: 40 and 5; (49) SEQ ID NOS: 41 and 21; (50) SEQ ID NOS: 42 and 44; (51) SEQ ID NOS: 45 and 47; (52) SEQ ID NOS: 48 and 47; (53) SEQ ID NOS: 53 and 47; (54) SEQ ID NOS: 59 and 52; (55) SEQ ID NOS: 59 and 60; (56) SEQ ID NOS: 6 and 10; (57) SEQ ID NOS: 6 and 21; (58) SEQ ID NOS: 6 and 3; (59) SEQ ID NOS: 6 and 31; (60) SEQ ID NOS: 6 and 32; (61) SEQ ID NOS: 6 and 5; and (62) SEQ ID NOS: 6 and 8.

One embodiment is directed to a primer set or collection of primer sets for amplifying DNA of a vancomycin-resistance gene (vanB gene), comprising a nucleotide sequence selected from the group consisting of: (1) SEQ ID NOS: 103 and 65; (2) SEQ ID NOS: 103 and 66; (3) SEQ ID NOS: 103 and 86; (4) SEQ ID NOS: 103 and 87; (5) SEQ ID NOS: 103 and 88; (6) SEQ ID NOS: 104 and 66; (7) SEQ ID NOS: 105 and 66; (8) SEQ ID NOS: 107 and 66; (9) SEQ ID NOS: 111 and 63; (10) SEQ ID NOS: 111 and 66; (11) SEQ ID NOS: 111 and 88; (12) SEQ ID NOS: 61 and 63; (13) SEQ ID NOS: 61 and 65; (14) SEQ ID NOS: 61 and 66; (15) SEQ ID NOS: 61 and 74; (16) SEQ ID NOS: 61 and 77; (17) SEQ ID NOS: 61 and 97; (18) SEQ ID NOS: 68 and 63; (19) SEQ ID NOS: 68 and 65; (20) SEQ ID NOS: 68 and 66; (21) SEQ ID NOS: 68 and 74; (22) SEQ ID NOS: 68 and 77; (23) SEQ ID NOS: 70 and 63; (24) SEQ ID NOS: 70 and 74; (25) SEQ ID NOS: 70 and 77; (26) SEQ ID NOS: 71 and 63; (27) SEQ ID NOS: 71 and 74; (28) SEQ ID NOS: 71 and 77; (29) SEQ ID NOS: 72 and 74; (30) SEQ ID NOS: 75 and 74; (31) SEQ ID NOS: 81 and 65; (32) SEQ ID NOS: 81 and 66; (33) SEQ ID NOS: 81 and 77; (34) SEQ ID NOS: 81 and 87; (35) SEQ ID NOS: 81 and 88; (36) SEQ ID NOS: 81 and 95; (37) SEQ ID NOS: 83 and 101; (38) SEQ ID NOS: 83 and 65; (39) SEQ ID NOS: 83 and 66; (40) SEQ ID NOS: 83 and 85; (41) SEQ ID NOS: 83 and 86; (42) SEQ ID NOS: 83 and 87; (43) SEQ ID NOS: 83 and 88; (44) SEQ ID NOS: 83 and 95; (45) SEQ ID NOS: 89 and 100; (46) SEQ ID NOS: 89 and 101; (47) SEQ ID NOS: 89 and 102; (48) SEQ ID NOS: 89 and 65; (49) SEQ ID NOS: 89 and 66; (50) SEQ ID NOS: 89 and 85; (51) SEQ ID NOS: 89 and 86; (52) SEQ ID NOS: 89 and 87; (53) SEQ ID NOS: 89 and 88; (54) SEQ ID NOS: 89 and 90; (55) SEQ ID NOS: 89 and 91; (56) SEQ ID NOS: 89 and 95; (57) SEQ ID NOS: 89 and 98; (58) SEQ ID NOS: 89 and 99; (59) SEQ ID NOS: 93 and 101; (60) SEQ ID NOS: 93 and 65; (61) SEQ ID NOS: 93 and 66; (62) SEQ ID NOS: 93 and 85; (63) SEQ ID NOS: 93 and 86; (64) SEQ ID NOS: 93 and 87; (65) SEQ ID NOS: 93 and 88; (66) SEQ ID NOS: 93 and 90; (67) SEQ ID NOS: 93 and 95; (68) SEQ ID NOS: 93 and 98; (69) SEQ ID NOS: 94 and 65; (70) SEQ ID NOS: 94 and 66; (71) SEQ ID NOS: 94 and 87; (72) SEQ ID NOS: 94 and 88; and (73) SEQ ID NOS: 94 and 95.

One embodiment is directed to a primer set or collection of primer sets for amplifying DNA of a vancomycin-resistance gene (vanC1 gene), comprising a nucleotide sequence selected from the group consisting of: (1) SEQ ID NOS: 123 and 125; (2) SEQ ID NOS: 127 and 129; (3) SEQ ID NOS: 130 and 132; (4) SEQ ID NOS: 133 and 135; (5) SEQ ID NOS: 133 and 137; (6) SEQ ID NOS: 138 and 140; (7) SEQ ID NOS: 141 and 137; (8) SEQ ID NOS: 141 and 143; (9) SEQ ID NOS: 141 and 147; (10) SEQ ID NOS: 141 and 179; (11) SEQ ID NOS: 144 and 137; (12) SEQ ID NOS: 144 and 146; (13) SEQ ID NOS: 144 and 147; (14) SEQ ID NOS: 144 and 157; (15) SEQ ID NOS: 148 and 137; (16) SEQ ID NOS: 148 and 150; (17) SEQ ID NOS: 151 and 153; (18) SEQ ID NOS: 151 and 155; (19) SEQ ID NOS: 156 and 150; (20) SEQ ID NOS: 158 and 160; (21) SEQ ID NOS: 161 and 137; (22) SEQ ID NOS: 161 and 147; (23) SEQ ID NOS: 161 and 150; (24) SEQ ID NOS: 161 and 153; (25) SEQ ID NOS: 161 and 190; (26) SEQ ID NOS: 161 and 192; (27) SEQ ID NOS: 162 and 164; (28) SEQ ID NOS: 165 and 167; (29) SEQ ID NOS: 168 and 169; (30) SEQ ID NOS: 170 and 169; (31) SEQ ID NOS: 171 and 173; (32) SEQ ID NOS: 171 and 174; (33) SEQ ID NOS: 175 and 177; (34) SEQ ID NOS: 178 and 179; (35) SEQ ID NOS: 180 and 146; (36) SEQ ID NOS: 183 and 150; (37) SEQ ID NOS: 184 and 174; (38) SEQ ID NOS: 185 and 187; (39) SEQ ID NOS: 188 and 137; (40) SEQ ID NOS: 188 and 150; (41) SEQ ID NOS: 188 and 190; (42) SEQ ID NOS: 191 and 137; (43) SEQ ID NOS: 191 and 150; (44) SEQ ID NOS: 191 and 153; (45) SEQ ID NOS: 191 and 190; (46) SEQ ID NOS: 191 and 192; (47) SEQ ID NOS: 193 and 137; (48) SEQ ID NOS: 193 and 150; (49) SEQ ID NOS: 193 and 153; (50) SEQ ID NOS: 193 and 190; (51) SEQ ID NOS: 194 and 147; (52) SEQ ID NOS: 194 and 160; (53) SEQ ID NOS: 196 and 147; (54) SEQ ID NOS: 196 and 160; (55) SEQ ID NOS: 198 and 179; (56) SEQ ID NOS: 200 and 179; (57) SEQ ID NOS: 201 and 179; (58) SEQ ID NOS: 202 and 179; (59) SEQ ID NOS: 203 and 147; (60) SEQ ID NOS: 204 and 179; and (61) SEQ ID NOS: 204 and 187.

One embodiment is directed to a primer set or collection of primer sets for amplifying DNA of a vancomycin-resistance gene (vanC2/C3 gene), comprising a nucleotide sequence selected from the group consisting of: (1) SEQ ID NOS: 206 and 208; (2) SEQ ID NOS: 206 and 209; (3) SEQ ID NOS: 206 and 216; (4) SEQ ID NOS: 206 and 219; (5) SEQ ID NOS: 206 and 227; (6) SEQ ID NOS: 210 and 209; (7) SEQ ID NOS: 210 and 212; (8) SEQ ID NOS: 210 and 215; (9) SEQ ID NOS: 210 and 216; (10) SEQ ID NOS: 210 and 219; (11) SEQ ID NOS: 210 and 223; (12) SEQ ID NOS: 210 and 227; (13) SEQ ID NOS: 213 and 215; (14) SEQ ID NOS: 217 and 209; (15) SEQ ID NOS: 217 and 216; (16) SEQ ID NOS: 217 and 219; (17) SEQ ID NOS: 217 and 223; (18) SEQ ID NOS: 217 and 227; (19) SEQ ID NOS: 220 and 209; (20) SEQ ID NOS: 220 and 219; (21) SEQ ID NOS: 220 and 223; (22) SEQ ID NOS: 220 and 227; (23) SEQ ID NOS: 221 and 209; (24) SEQ ID NOS: 221 and 216; (25) SEQ ID NOS: 221 and 219; (26) SEQ ID NOS: 221 and 227; (27) SEQ ID NOS: 222 and 209; (28) SEQ ID NOS: 222 and 216; (29) SEQ ID NOS: 222 and 219; (30) SEQ ID NOS: 222 and 223; (31) SEQ ID NOS: 222 and 227; (32) SEQ ID NOS: 224 and 212; (33) SEQ ID NOS: 224 and 215; (34) SEQ ID NOS: 224 and 216; (35) SEQ ID NOS: 225 and 209; (36) SEQ ID NOS: 225 and 212; (37) SEQ ID NOS: 225 and 216; (38) SEQ ID NOS: 226 and 209; (39) SEQ ID NOS: 226 and 212; (40) SEQ ID NOS: 226 and 216; (41) SEQ ID NOS: 228 and 215; (42) SEQ ID NOS: 229 and 209; (43) SEQ ID NOS: 229 and 215; (44) SEQ ID NOS: 230 and 219; (45) SEQ ID NOS: 231 and 212; (46) SEQ ID NOS: 231 and 215; (47) SEQ ID NOS: 232 and 216; (48) SEQ ID NOS: 233 and 212; (49) SEQ ID NOS: 234 and 215; (50) SEQ ID NOS: 235 and 215; (51) SEQ ID NOS: 235 and 239; (52) SEQ ID NOS: 235 and 241; (53) SEQ ID NOS: 236 and 216; (54) SEQ ID NOS: 237 and 209; (55) SEQ ID NOS: 237 and 215; (56) SEQ ID NOS: 238 and 215; (57) SEQ ID NOS: 240 and 216; (58) SEQ ID NOS: 242 and 216; and (59) SEQ ID NOS: 243 and 215.

One embodiment is directed to a primer set or collection of primer sets for amplifying DNA of a vancomycin-resistance gene (vanD gene), comprising a nucleotide sequence selected from the group consisting of: (1) SEQ ID NOS: 388 and 390; (2) SEQ ID NOS: 391 and 393; (3) SEQ ID NOS: 391 and 434; (4) SEQ ID NOS: 394 and 393; (5) SEQ ID NOS: 396 and 398; (6) SEQ ID NOS: 396 and 419; (7) SEQ ID NOS: 396 and 419; (8) SEQ ID NOS: 399 and 401; (9) SEQ ID NOS: 399 and 401; (10) SEQ ID NOS: 399 and 401; (11) SEQ ID NOS: 399 and 401; (12) SEQ ID NOS: 399 and 444; (13) SEQ ID NOS: 399 and 444; (14) SEQ ID NOS: 415 and 401; (15) SEQ ID NOS: 416 and 417; (16) SEQ ID NOS: 435 and 437; (17) SEQ ID NOS: 438 and 439; (18) SEQ ID NOS: 440 and 442; (19) SEQ ID NOS: 443 and 434; (20) SEQ ID NOS: 445 and 447; (21) SEQ ID NOS: 445 and 448; (22) SEQ ID NOS: 445 and 449; (23) SEQ ID NOS: 445 and 450; (24) SEQ ID NOS: 445 and 451; (25) SEQ ID NOS: 445 and 452; (26) SEQ ID NOS: 445 and 453; (27) SEQ ID NOS: 445 and 454; (28) SEQ ID NOS: 445 and 455; (29) SEQ ID NOS: 445 and 459; (30) SEQ ID NOS: 445 and 460; (31) SEQ ID NOS: 445 and 461; (32) SEQ ID NOS: 456 and 458; (33) SEQ ID NOS: 462 and 464; (34) SEQ ID NOS: 465 and 467; (35) SEQ ID NOS: 468 and 470; (36) SEQ ID NOS: 471 and 473; (37) SEQ ID NOS: 474 and 476; (38) SEQ ID NOS: 477 and 479; (39) SEQ ID NOS: 480 and 482; (40) SEQ ID NOS: 483 and 479; (41) SEQ ID NOS: 483 and 486; (42) SEQ ID NOS: 488 and 490; (43) SEQ ID NOS: 491 and 493; (44) SEQ ID NOS: 494 and 486; (45) SEQ ID NOS: 495 and 493; (46) SEQ ID NOS: 496 and 486; (47) SEQ ID NOS: 497 and 486; (48) SEQ ID NOS: 498 and 486; (49) SEQ ID NOS: 500 and 486; (50) SEQ ID NOS: 501 and 486; and (51) SEQ ID NOS: 502 and 493.

One embodiment is directed to a primer set or collection of primer sets for amplifying DNA of a vancomycin-resistance gene (vanE gene), comprising a nucleotide sequence selected from the group consisting of: (1) SEQ ID NOS: 334 and 336; (2) SEQ ID NOS: 337 and 336; (3) SEQ ID NOS: 338 and 336; (4) SEQ ID NOS: 338 and 381; (5) SEQ ID NOS: 339 and 336; (6) SEQ ID NOS: 340 and 336; (7) SEQ ID NOS: 341 and 336; (8) SEQ ID NOS: 341 and 381; (9) SEQ ID NOS: 342 and 336; (10) SEQ ID NOS: 342 and 381; (11) SEQ ID NOS: 343 and 336; (12) SEQ ID NOS: 344 and 336; (13) SEQ ID NOS: 344 and 381; (14) SEQ ID NOS: 345 and 336; (15) SEQ ID NOS: 346 and 336; (16) SEQ ID NOS: 347 and 336; (17) SEQ ID NOS: 347 and 381; (18) SEQ ID NOS: 348 and 336; (19) SEQ ID NOS: 348 and 381; (20) SEQ ID NOS: 349 and 336; (21) SEQ ID NOS: 349 and 381; (22) SEQ ID NOS: 350 and 336; (23) SEQ ID NOS: 350 and 381; (24) SEQ ID NOS: 351 and 336; (25) SEQ ID NOS: 352 and 336; (26) SEQ ID NOS: 353 and 336; (27) SEQ ID NOS: 354 and 336; (28) SEQ ID NOS: 355 and 336; (29) SEQ ID NOS: 355 and 381; (30) SEQ ID NOS: 356 and 336; (31) SEQ ID NOS: 357 and 336; (32) SEQ ID NOS: 358 and 336; (33) SEQ ID NOS: 359 and 336; (34) SEQ ID NOS: 359 and 381; (35) SEQ ID NOS: 360 and 336; (36) SEQ ID NOS: 360 and 381; (37) SEQ ID NOS: 361 and 336; (38) SEQ ID NOS: 362 and 336; (39) SEQ ID NOS: 363 and 336; (40) SEQ ID NOS: 364 and 336; (41) SEQ ID NOS: 365 and 336; (42) SEQ ID NOS: 366 and 336; (43) SEQ ID NOS: 367 and 336; (44) SEQ ID NOS: 368 and 336; (45) SEQ ID NOS: 369 and 336; (46) SEQ ID NOS: 370 and 336; (47) SEQ ID NOS: 371 and 336; (48) SEQ ID NOS: 372 and 336; (49) SEQ ID NOS: 373 and 336; (50) SEQ ID NOS: 374 and 336; (51) SEQ ID NOS: 375 and 336; (52) SEQ ID NOS: 376 and 336; (53) SEQ ID NOS: 377 and 336; (54) SEQ ID NOS: 378 and 336; (55) SEQ ID NOS: 379 and 336; (56) SEQ ID NOS: 380 and 336; (57) SEQ ID NOS: 382 and 381; (58) SEQ ID NOS: 383 and 381; (59) SEQ ID NOS: 384 and 381; (60) SEQ ID NOS: 385 and 381; (61) SEQ ID NOS: 386 and 381; and (62) SEQ ID NOS: 387 and 381.

One embodiment is directed to a primer set or collection of primer sets for amplifying DNA of a vancomycin-resistance gene (vanG gene), comprising a nucleotide sequence selected from the group consisting of: (1) SEQ ID NOS: 244 and 246; (2) SEQ ID NOS: 244 and 247; (3) SEQ ID NOS: 244 and 248; (4) SEQ ID NOS: 244 and 250; (5) SEQ ID NOS: 244 and 251; (6) SEQ ID NOS: 244 and 254; (7) SEQ ID NOS: 244 and 258; (8) SEQ ID NOS: 244 and 259; (9) SEQ ID NOS: 244 and 284; (10) SEQ ID NOS: 244 and 286; (11) SEQ ID NOS: 244 and 287; (12) SEQ ID NOS: 249 and 246; (13) SEQ ID NOS: 249 and 248; (14) SEQ ID NOS: 249 and 286; (15) SEQ ID NOS: 249 and 301; (16) SEQ ID NOS: 249 and 306; (17) SEQ ID NOS: 249 and 308; (18) SEQ ID NOS: 249 and 312; (19) SEQ ID NOS: 252 and 246; (20) SEQ ID NOS: 252 and 262; (21) SEQ ID NOS: 253 and 246; (22) SEQ ID NOS: 255 and 246; (23) SEQ ID NOS: 256 and 246; (24) SEQ ID NOS: 260 and 258; (25) SEQ ID NOS: 263 and 258; (26) SEQ ID NOS: 264 and 266; (27) SEQ ID NOS: 267 and 269; (28) SEQ ID NOS: 270 and 266; (29) SEQ ID NOS: 270 and 269; (30) SEQ ID NOS: 271 and 266; (31) SEQ ID NOS: 272 and 274; (32) SEQ ID NOS: 275 and 269; (33) SEQ ID NOS: 276 and 269; (34) SEQ ID NOS: 277 and 269; (35) SEQ ID NOS: 278 and 266; (36) SEQ ID NOS: 279 and 269; (37) SEQ ID NOS: 280 and 266; (38) SEQ ID NOS: 280 and 269; (39) SEQ ID NOS: 281 and 269; (40) SEQ ID NOS: 282 and 266; (41) SEQ ID NOS: 282 and 269; (42) SEQ ID NOS: 283 and 269; (43) SEQ ID NOS: 285 and 259; (44) SEQ ID NOS: 288 and 290; (45) SEQ ID NOS: 288 and 304; (46) SEQ ID NOS: 291 and 290; (47) SEQ ID NOS: 293 and 290; (48) SEQ ID NOS: 293 and 304; (49) SEQ ID NOS: 295 and 258; (50) SEQ ID NOS: 297 and 290; (51) SEQ ID NOS: 298 and 290; (52) SEQ ID NOS: 298 and 304; (53) SEQ ID NOS: 299 and 290; (54) SEQ ID NOS: 300 and 290; (55) SEQ ID NOS: 302 and 274; (56) SEQ ID NOS: 303 and 290; (57) SEQ ID NOS: 303 and 304; (58) SEQ ID NOS: 305 and 274; (59) SEQ ID NOS: 305 and 290; (60) SEQ ID NOS: 307 and 274; (61) SEQ ID NOS: 307 and 290; (62) SEQ ID NOS: 309 and 290; (63) SEQ ID NOS: 310 and 290; (64) SEQ ID NOS: 311 and 258; (65) SEQ ID NOS: 313 and 290; (66) SEQ ID NOS: 314 and 290; (67) SEQ ID NOS: 314 and 320; (68) SEQ ID NOS: 315 and 290; (69) SEQ ID NOS: 316 and 290; (70) SEQ ID NOS: 317 and 290; (71) SEQ ID NOS: 318 and 290; (72) SEQ ID NOS: 321 and 258; (73) SEQ ID NOS: 322 and 324; (74) SEQ ID NOS: 325 and 266; (75) SEQ ID NOS: 325 and 274; (76) SEQ ID NOS: 326 and 274; (77) SEQ ID NOS: 327 and 266; (78) SEQ ID NOS: 328 and 274; (79) SEQ ID NOS: 329 and 274; (80) SEQ ID NOS: 330 and 274; (81) SEQ ID NOS: 331 and 274; (82) SEQ ID NOS: 332 and 266; and (83) SEQ ID NOS: 333 and 266.

One embodiment is directed to a primer set or collection of primer sets for amplifying DNA of an Efm sodA gene, comprising a nucleotide sequence SEQ ID NOS: 610 and 622.

One embodiment is directed to a primer set or collection of primer sets for amplifying DNA of an Efs sodA gene, comprising a nucleotide sequence selected from the group consisting of: 1) SEQ ID NOS: 577 and 617; (2) SEQ ID NOS: 577 and 623; (3) SEQ ID NOS: 577 and 624; (4) SEQ ID NOS: 577 and 625; (5) SEQ ID NOS: 577 and 637; (6) SEQ ID NOS: 577 and 640; (7) SEQ ID NOS: 586 and 617; (8) SEQ ID NOS: 586 and 623; (9) SEQ ID NOS: 586 and 624; (10) SEQ ID NOS: 586 and 625; (11) SEQ ID NOS: 586 and 637; (12) SEQ ID NOS: 586 and 640; (13) SEQ ID NOS: 590 and 617; (14) SEQ ID NOS: 590 and 623; (15) SEQ ID NOS: 590 and 624; (16) SEQ ID NOS: 590 and 625; (17) SEQ ID NOS: 590 and 637; (18) SEQ ID NOS: 590 and 640; (19) SEQ ID NOS: 598 and 617; (20) SEQ ID NOS: 598 and 623; (21) SEQ ID NOS: 598 and 624; (22) SEQ ID NOS: 598 and 625; (23) SEQ ID NOS: 598 and 637; (24) SEQ ID NOS: 598 and 640; (25) SEQ ID NOS: 599 and 617; (26) SEQ ID NOS: 599 and 623; (27) SEQ ID NOS: 599 and 624; (28) SEQ ID NOS: 599 and 625; (29) SEQ ID NOS: 599 and 637; (30) SEQ ID NOS: 599 and 640; (31) SEQ ID NOS: 600 and 617; (32) SEQ ID NOS: 600 and 623; (33) SEQ ID NOS: 600 and 624; (34) SEQ ID NOS: 600 and 625; (35) SEQ ID NOS: 600 and 637; (36) SEQ ID NOS: 600 and 640;

One embodiment is directed to a primer set or collection of primer sets for amplifying DNA of an Efm novel gene, comprising a nucleotide sequence selected from the from the group consisting of: SEQ ID NOS: 683 and 707; (2) SEQ ID NOS: 683 and 720; (3) SEQ ID NOS: 683 and 723; (4) SEQ ID NOS: 687 and 707; (5) SEQ ID NOS: 687 and 720; (6) SEQ ID NOS: 687 and 723; (7) SEQ ID NOS: 692 and 707; (8) SEQ ID NOS: 692 and 720; (9) SEQ ID NOS: 692 and 723.

One embodiment is directed to a primer set or collection of primer sets for amplifying DNA of an Efs novel gene, comprising a nucleotide sequence selected from the group consisting of: SEQ ID NOS: 758 and 785; (2) SEQ ID NOS: 758 and 791; (3) SEQ ID NOS: 758 and 797; (4) SEQ ID NOS: 758 and 799; (5) SEQ ID NOS: 758 and 803; (6) SEQ ID NOS: 772 and 785; (7) SEQ ID NOS: 772 and 791; (8) SEQ ID NOS: 772 and 797; (9) SEQ ID NOS: 772 and 799; (10) SEQ ID NOS: 772 and 803; (11) SEQ ID NOS: 773 and 785; (12) SEQ ID NOS: 773 and 791; (13) SEQ ID NOS: 773 and 797; (14) SEQ ID NOS: 773 and 799; (15) SEQ ID NOS: 773 and 803; (16) SEQ ID NOS: 775 and 785; (17) SEQ ID NOS: 775 and 791; (18) SEQ ID NOS: 775 and 797; (19) SEQ ID NOS: 775 and 799; (20) SEQ ID NOS: 775 and 803.

One embodiment is directed to a primer set or collection of primer sets for amplifying DNA of Efm/Efs dual genes, comprising a nucleotide sequence consisting of: SEQ ID NOS: 843, 845 and 846.

A particular embodiment is directed to oligonucleotide probes for binding to DNA of a vancomycin-resistance gene(s), comprising a nucleotide sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG).

A particular embodiment is directed to oligonucleotide probes for binding to DNA of an Efm sodA gene or Efs sodA gene or Efm novel gene or Efs novel gene or dual genes, comprising a nucleotide sequence selected from the group consisting of: SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual).

One embodiment is directed to the simultaneous detection in a multiplex format of vancomycin resistance, specifically the resistance genes vanA, vanB, vanC, vanD, vanE and vanG.

One embodiment is directed to the simultaneous detection and differentiation in a multiplex format of the vanA and vanB resistance genes.

One embodiment is directed to the simultaneous detection in a multiplex format of VRE when an isolate is tested.

One embodiment is directed to primer sets for amplifying DNA of a vancomycin-resistance gene(s) simultaneously, comprising:

(a): (1) SEQ ID NOS: 1 and 3; (2) SEQ ID NOS: 1 and 32; (3) SEQ ID NOS: 1 and 5; (4) SEQ ID NOS: 19 and 21; (5) SEQ ID NOS: 19 and 3; (6) SEQ ID NOS: 19 and 32; (7) SEQ ID NOS: 19 and 47; (8) SEQ ID NOS: 19 and 5; (9) SEQ ID NOS: 19 and 52; (10) SEQ ID NOS: 19 and 55; (11) SEQ ID NOS: 22 and 21; (12) SEQ ID NOS: 22 and 3; (13) SEQ ID NOS: 22 and 32; (14) SEQ ID NOS: 22 and 5; (15) SEQ ID NOS: 23 and 21; (16) SEQ ID NOS: 23 and 3; (17) SEQ ID NOS: 23 and 32; (18) SEQ ID NOS: 23 and 5; (19) SEQ ID NOS: 26 and 3; (20) SEQ ID NOS: 26 and 32; (21) SEQ ID NOS: 26 and 47; (22) SEQ ID NOS: 26 and 5; (23) SEQ ID NOS: 26 and 51; (24) SEQ ID NOS: 28 and 3; (25) SEQ ID NOS: 28 and 32; (26) SEQ ID NOS: 28 and 5; (27) SEQ ID NOS: 29 and 21; (28) SEQ ID NOS: 29 and 3; (29) SEQ ID NOS: 29 and 5; (30) SEQ ID NOS: 29 and 8; (31) SEQ ID NOS: 33 and 21; (32) SEQ ID NOS: 33 and 3; (33) SEQ ID NOS: 33 and 32; (34) SEQ ID NOS: 33 and 5; (35) SEQ ID NOS: 34 and 36; (36) SEQ ID NOS: 34 and 52; (37) SEQ ID NOS: 37 and 3; (38) SEQ ID NOS: 37 and 32; (39) SEQ ID NOS: 37 and 5; (40) SEQ ID NOS: 38 and 3; (41) SEQ ID NOS: 38 and 32; (42) SEQ ID NOS: 38 and 5; (43) SEQ ID NOS: 39 and 3; (44) SEQ ID NOS: 39 and 32; (45) SEQ ID NOS: 39 and 5; (46) SEQ ID NOS: 40 and 3; (47) SEQ ID NOS: 40 and 32; (48) SEQ ID NOS: 40 and 5; (49) SEQ ID NOS: 41 and 21; (50) SEQ ID NOS: 42 and 44; (51) SEQ ID NOS: 45 and 47; (52) SEQ ID NOS: 48 and 47; (53) SEQ ID NOS: 53 and 47; (54) SEQ ID NOS: 59 and 52; (55) SEQ ID NOS: 59 and 60; (56) SEQ ID NOS: 6 and 10; (57) SEQ ID NOS: 6 and 21; (58) SEQ ID NOS: 6 and 3; (59) SEQ ID NOS: 6 and 31; (60) SEQ ID NOS: 6 and 32; (61) SEQ ID NOS: 6 and 5; and (62) SEQ ID NOS: 6 and 8 (forward and reverse primers for amplifying DNA of vanA, respectively); and

(b) (1) SEQ ID NOS: 103 and 65; (2) SEQ ID NOS: 103 and 66; (3) SEQ ID NOS: 103 and 86; (4) SEQ ID NOS: 103 and 87; (5) SEQ ID NOS: 103 and 88; (6) SEQ ID NOS: 104 and 66; (7) SEQ ID NOS: 105 and 66; (8) SEQ ID NOS: 107 and 66; (9) SEQ ID NOS: 111 and 63; (10) SEQ ID NOS: 111 and 66; (11) SEQ ID NOS: 111 and 88; (12) SEQ ID NOS: 61 and 63; (13) SEQ ID NOS: 61 and 65; (14) SEQ ID NOS: 61 and 66; (15) SEQ ID NOS: 61 and 74; (16) SEQ ID NOS: 61 and 77; (17) SEQ ID NOS: 61 and 97; (18) SEQ ID NOS: 68 and 63; (19) SEQ ID NOS: 68 and 65; (20) SEQ ID NOS: 68 and 66; (21) SEQ ID NOS: 68 and 74; (22) SEQ ID NOS: 68 and 77; (23) SEQ ID NOS: 70 and 63; (24) SEQ ID NOS: 70 and 74; (25) SEQ ID NOS: 70 and 77; (26) SEQ ID NOS: 71 and 63; (27) SEQ ID NOS: 71 and 74; (28) SEQ ID NOS: 71 and 77; (29) SEQ ID NOS: 72 and 74; (30) SEQ ID NOS: 75 and 74; (31) SEQ ID NOS: 81 and 65; (32) SEQ ID NOS: 81 and 66; (33) SEQ ID NOS: 81 and 77; (34) SEQ ID NOS: 81 and 87; (35) SEQ ID NOS: 81 and 88; (36) SEQ ID NOS: 81 and 95; (37) SEQ ID NOS: 83 and 101; (38) SEQ ID NOS: 83 and 65; (39) SEQ ID NOS: 83 and 66; (40) SEQ ID NOS: 83 and 85; (41) SEQ ID NOS: 83 and 86; (42) SEQ ID NOS: 83 and 87; (43) SEQ ID NOS: 83 and 88; (44) SEQ ID NOS: 83 and 95; (45) SEQ ID NOS: 89 and 100; (46) SEQ ID NOS: 89 and 101; (47) SEQ ID NOS: 89 and 102; (48) SEQ ID NOS: 89 and 65; (49) SEQ ID NOS: 89 and 66; (50) SEQ ID NOS: 89 and 85; (51) SEQ ID NOS: 89 and 86; (52) SEQ ID NOS: 89 and 87; (53) SEQ ID NOS: 89 and 88; (54) SEQ ID NOS: 89 and 90; (55) SEQ ID NOS: 89 and 91; (56) SEQ ID NOS: 89 and 95; (57) SEQ ID NOS: 89 and 98; (58) SEQ ID NOS: 89 and 99; (59) SEQ ID NOS: 93 and 101; (60) SEQ ID NOS: 93 and 65; (61) SEQ ID NOS: 93 and 66; (62) SEQ ID NOS: 93 and 85; (63) SEQ ID NOS: 93 and 86; (64) SEQ ID NOS: 93 and 87; (65) SEQ ID NOS: 93 and 88; (66) SEQ ID NOS: 93 and 90; (67) SEQ ID NOS: 93 and 95; (68) SEQ ID NOS: 93 and 98; (69) SEQ ID NOS: 94 and 65; (70) SEQ ID NOS: 94 and 66; (71) SEQ ID NOS: 94 and 87; (72) SEQ ID NOS: 94 and 88; and (73) SEQ ID NOS: 94 and 95 (forward and reverse primers for amplifying DNA of vanB, respectively).

A particular embodiment is directed to oligonucleotide probes for binding to DNA of vancomycin-resistance gene(s), comprising a nucleotide sequence selected from the group consisting of SEQ ID NOS: 2, 4, 7, 9, 11-18, 20, 24, 25, 27, 30, 35, 43, 46, 49, 50, 54, 56-58 (vanA probes); and 62, 64, 67, 69, 73, 76, 78, 79, 80, 82, 84, 92, 96, 108-110, 112 (vanB probes).

One embodiment is directed to a kit for detecting DNA of a vancomycin-resistance gene(s) in a sample, comprising one or more probes comprising a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG) In a particular embodiment, the kit further comprises a) at least one forward primer comprising the sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); and b) at least one reverse primer comprising the sequence selected from the group consisting of: SEQ ID NOS: SEQ ID NOS: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG).

One embodiment is directed to a kit for detecting DNA of a Efm sodA or Efs sodA gene or Efm novel gene or Efs novel gene or dual genes, in a sample, comprising one or more probes comprising a sequence selected from the group consisting of: SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual). In a particular embodiment, the kit further comprises a) at least one forward primer comprising the sequence selected from the group consisting of: SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual); and at least one reverse primer comprising a sequence selected from the group consisting of SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual).

One embodiment is directed to a kit for detecting DNA of a vancomycin-resistance gene(s) or Enterococcal marker gene in a sample, comprising one or more probes comprising a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG), 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual). In a particular embodiment, the kit further comprises a) at least one forward primer comprising the sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); 517 (Efm sodA); 577, 586, 590, 598, 599, 600 (Efs sodA); 683, 687, 692 (Efm novel); 758, 772, 773, 775 (Efs novel); and 843 (Efm/Efs dual); and b) at least one reverse primer comprising the sequence selected from the group consisting of: SEQ ID NOS: SEQ ID NOS: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG); 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual).

In a particular embodiment, the kit further comprises reagents for isolating and/or sequencing the vancomycin-resistance gene(s) in the sample. In a particular embodiment, the one or more probes are labeled with different detectable labels. In a particular embodiment, the one or more probes are labeled with the same detectable labels. In a particular embodiment, the at least one forward primer, the at least one reverse primer and the one or more probes are selected from the groups consisting of: Groups 1-212 of Table 5, Groups 213-601 of Table 6, Groups 603-605 of Table 8B, Groups 606-627 of Table 9B, Groups 628-636 of Table 10B, Groups 637-643 of Table 11B, and Group 644 of Table 12.

One embodiment is directed to a method for diagnosing a condition, syndrome or disease in a human associated with a vancomycin-resistant organism, comprising: a) contacting a sample with at least one forward and reverse primer set selected from the group consisting of: Groups 1-601 of Tables 5 and 6; b) conducting an amplification reaction, thereby producing an amplicon; and c) detecting the amplicon using one or more probes selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG); wherein the generation of an amplicon is indicative of the presence of an organism resistant to vancomycin in the sample. In a particular embodiment, the sample is blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage, fibroblasts or samples derived from inanimate objects. A sample may be collected from more than one collection site, e.g., blood and an anal-rectal swab. In a particular embodiment, the complications, conditions, syndromes or diseases in humans associated with a vancomycin-resistant organism are selected from the group consisting of: infections at indwelling sites and wounds, urinary tract infections, sepsis, infections from indwelling urinary or central venous catheters, and infections from abdominal or cardiothoracic surgery.

One embodiment is directed to a method for diagnosing a condition, syndrome or disease in a human associated with an Enterococcal organism, comprising: a) contacting a sample with at least one forward and reverse primer set selected from the group consisting of: Groups 603-644 of Tables 8B, 9B, 10B, 11B, and 12; b) conducting an amplification reaction, thereby producing an amplicon; and c) detecting the amplicon using one or more probes selected from the group consisting of: SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual); wherein the generation of an amplicon is indicative of the presence of an Enterococcal organism in the sample. In a particular embodiment, the sample is blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage, fibroblasts or samples derived from inanimate objects. A sample may be collected from more than one collection site, e.g., blood and an anal-rectal swab. In a particular embodiment, the complications, conditions, syndromes or diseases in humans associated with a vancomycin-resistant organism are selected from the group consisting of: infections at indwelling sites and wounds, urinary tract infections, sepsis, infections from indwelling urinary or central venous catheters, and infections from abdominal or cardiothoracic surgery.

One embodiment is directed to a kit for amplifying and sequencing DNA of a vancomycin-resistance gene(s) in a sample, comprising: a) at least one forward primer comprising the sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); and b) at least one reverse primer comprising the sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG); and c) reagents for the sequencing of amplified DNA fragments.

One embodiment is directed to a kit for amplifying and sequencing DNA of an Enterococci specific gene in a sample, comprising: a) at least one forward primer comprising the sequence selected from the group consisting of: SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual); and at least one reverse primer comprising a sequence selected from the group consisting of SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual); and c) reagents for the sequencing of amplified DNA fragments.

In a particular embodiment, the sample is blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage, fibroblasts or samples derived from inanimate objects. In a particular embodiment, the complications, conditions, syndromes or diseases in humans associated with a vancomycin-resistant organism are selected from the group consisting of: skin infections, such as boils, impetigo, cellulitis, and scalded skin syndrome; food poisoning, leading to abdominal cramps, nausea, vomiting, and diarrhea; bacteremia, resulting in a persistent fever and other signs of blood poisoning; toxic shock syndrome, resulting in high fever, nausea, vomiting, rash on palms and soles, confusion, muscle aches, seizures, headache; and septic arthritis, resulting in joint swelling, severe pain in the affected joint, fever, and shaking chills.

One embodiment is directed to an internal control plasmid and vancomycin-resistance positive control plasmids. The non-competitive internal control plasmid is a synthetic target that does not occur naturally in clinical sample types for which this assay is intended. The synthetic target sequence incorporates an artificial, random polynucleotide sequence with a known GC content. The synthetic target sequence is:

(SEQ ID NO: 503)

5′GCGAAGTGAGAATACGCCGTGTCGCAGTTTCCTTGAGCAGTGTCTCTA
AATGCCTCAAACCGTCGCATTTTTGGTTATAGCAGTAACTATATGGAGGT
CCGTAGGCGGCGTGCGTGGGGGCACCAAACTCATCCAACGGTCGACTGC
GCCTGTAGGGTCTTAAGAAGCGGCACCTCAGACCGATAGCATAGCACTT
AAAGAGGAATTGAATAATCAAGATGGGTATCCGACCGACGCGGAGTGAC
CGAGGAAGAGGACCCTGCATGTATCCTGAGAGTATAGTTGTCAGAGCAG
CAATTGATTCACCACCAAGGGACTTAGTCT 3′.

This internal control is detected by a forward primer (SEQ ID NO: 504), a reverse primer (SEQ ID NO: 506) and a probe (SEQ ID NO: 505). A plasmid vector containing the internal control target sequence (SEQ ID NO: 503) is included in the assay. The internal control plasmid is added directly to the reaction mix to monitor the integrity of the PCR reagents and the presence of PCR inhibitors.

The vancomycin-resistance positive control plasmid contain partial sequences for one or more of the vancomycin resistance targets (i.e. vanA, vanB, vanC, etc.), respectively. The positive control plasmids comprise forward primer, probe and reverse primer sequences for the given vancomycin resistance. An artificial polynucleotide sequence is inserted within the positive control sequence corresponding to the given target to allow the amplicon generated by the target primer pairs to be differentiated from the amplicon derived by the same primer pairs from a natural target by size, by a unique restriction digest profile, and by a probe directed against the artificial sequence. The positive control plasmids are intended to be used as a control to confirm that the assay is performing within specifications.

The oligonucleotides of the present invention and their resulting amplicons do not cross react and, thus, will work together without negatively impacting each other. The primers and probes of the present invention do not cross react with other potentially contaminating species that would be present in a sample matrix.

One embodiment is directed to a method of hybridizing one or more isolated nucleic acid sequences comprising a sequence selected from the group consisting of: SEQ ID NOS: 513-846 to a Enterococcus faecium specific gene and/or Enterococcus faecalis specific gene, comprising contacting one or more isolated nucleic acid sequences to a sample comprising the Enterococcus faecium specific gene and/or Enterococcus faecalis specific gene under conditions suitable for hybridization.

One embodiment is directed to a method of hybridizing one or more isolated nucleic acid sequences comprising a sequence selected from the group consisting of: SEQ ID NOS: 1-502 and 513-846 to a vancomycin-resistance gene and/or an Enterococcus faecium specific gene and/or an Enterococcus faecalis specific gene, comprising contacting one or more isolated nucleic acid sequences to a sample comprising the vancomycin-resistance gene and/or the Enterococcus faecium specific gene and/or Enterococcus faecalis specific gene under conditions suitable for hybridization.

One embodiment is directed to a kit for detecting an Enterococcus faecium specific gene and/or an Enterococcus faecalis specific gene in a sample, comprising one or more probes comprising a sequence selected from the group consisting of: SEQ ID NOS: 513-846.

One embodiment is directed to a kit for detecting a vancomycin-resistance gene and/or an Enterococcus faecium specific gene and/or Enterococcus faecalis specific gene in a sample, comprising one or more probes comprising a sequence selected from the group consisting of: SEQ ID NOS: 1-502 and 513-846.

### BRIEF DESCRIPTION OF THE DRAWINGS

FIG. 1 is a plot diagram of the amplification of the vanA synthetic construct.

FIG. 2 is a plot diagram of the amplification of the vanB synthetic construct.

FIG. 3 is an electropherogram and legend showing the migration of vanA and vanB PCR products during gel electrophoresis.

FIG. 4 is a plot diagram of the amplification of the E. faecium sodA oligonucleotide solution, E. faecalis sodA oligonucleotide solution, and both E. faecium and E. faecalis dual oligonucleotide solution.

FIG. 5 is an electropherogram and legend showing the migration of the E. faecium sodA, E. faecalis sodA, and both E. faecium and E. faecalis dual PCR products during gel electrophoresis.

### DETAILED DESCRIPTION

A diagnostic or screening test that can detect multiple resistance genes simultaneously (the van genes), as well as determine whether a sample contains VRE, is necessary, as vancomycin resistant organisms are the major causative agents, for example, of HAIs.

Described herein are optimized probes and primers that, alone or in various combinations, allow for the amplification, detection, isolation, and sequencing of vancomycin genes that can be found in clinical isolates, including Enterococcal and Staphylococcal pathogens. Specific probes and primers, i.e., probes and primers that detect all known and characterized vancomycin have been discovered and are described herein. Nucleic acid primers and probes for detecting bacterial genetic material, especially the resistance genes vanA and vanB, and methods for designing and optimizing the respective primer and probe sequences, are described. The present invention also provides nucleic acid primers and probes for detecting the resistance genes van C, vanD, vanE and vanG. The present invention furthermore provides nucleic acid primers and probes for detecting the genus Enterococci.

The primers and probes described herein can be used, for example, to screen patients for the presence of the vanA, vanB, vanC, vanD, vanE and vanG resistance genes, Efs sodA, Efm sodA, Efs novel, Efm novel, Efm/Efs dual, e.g., in clinical isolates, including Enterococcal and Staphylococcal pathogens, in a multiplex format.

The primers and probes of the present invention can be used for the detection of the vancomycin-resistance genes in a multiplex format to allow detection of vancomycin resistant organisms (including VRE and vancomycin resistant Staphylococcus aureus (VRSA). Currently, the vancomycin-resistance genes are tested separately; however, the multiplex format option of the present invention allows relative comparisons to be made between these prevalent resistance genes.

### Vancomycin Resistance

The importance of Enterococcus in vancomycin-resistant nosocomial infections or hospital acquired infections (HAIs) (vancomycin-resistant Enterococcus; VRE), has been the impetus for the development of therapeutic alternatives to vancomycin. One such alternative that is currently in use in the United States and European Union is linezolid. Linezolid is of the oxazolidinone class and is frequently used on multi-drug resistant bacteria, such as methicillin-resistant Staphylococcus aureus (MRSA) and VRE. Vancomycin resistance is classified according to six phenotypes: VanA, VanB, VanC (C1, C2, C3), VanD, VanE and VanG. VanA and VanB are inducible and transferable, while VanC, VanD, VanE and VanG are constitutive and non-transferable. In general, the VanA and VanB phenotypes are the most clinically important and found most often in E. faecium and E. faecalis. The VanC phenotype may also be clinically important as it is frequently associated with resistance infections caused by other Enterococcus species (such as E. gallinarum, E. casseliflavus, and E. flavescens). It is less clear if the VanD, VanE and VanG phenotypes are clinically important. Vancomycin-resistant E. faecium and E. faecalis corresponding to the VanD, VanE, or VanG phenotypes have been isolated, but the prevalence of these relative to the VanA and VanB phenotypes in these species is not known and may be much lower in clinical settings. (Cetinkaya et al., Clin. Microbiol. Rev. 13:686-707 (2000); McKessar et al., Antimicrob. Agents Chemother. 44:3224-3228 (2000); Perichon et al., Antimicrob. Agents Chemother. 41:2016-2018 (1997); Boyd et al., Antimicrob. Agents Chemother. 46:1977-1979 (2002); Domingo et al., Antimicrob. Agents Chemother. 49:4784-4786 (2005)).

The presence of non-enterococcal vancomycin resistance may not alter present treatment or control measures. However, since vanB genes may be transferred from intestinal flora to enterococcal species, knowledge of potential reservoirs of glycopeptide resistance genes is critical for maintaining VRE infection control over VRE and other vancomycin-resistant species. (Ballard et al., Antimicrob. Agents Chemother. 49:77-81 (2005); Ballard et al., Antimicrob. Agents Chemother. 49:1688-1694 (2005); Domingo et al., J. Antimicrob. Chemother. 55:466-474 (2005)).

The mechanism of vancomycin resistance involves substituting the D-alanine terminating residue of cell wall precursors to which vancomycin binds, with a D-lactate residue—VanA, VanB, or VanD phenotypes, or D-serine residue—VanC and VanE phenotypes, and presumably the VanG phenotype. The modified cell wall precursors have a lower affinity for vancomycin binding, neutralizing its effect. The VanA phenotype is highly resistant to vancomycin and another glycopeptide-class antibiotic, teicoplanin. The VanB phenotype is associated with moderate to high levels of vancomycin resistance, but sensitivity to teicoplanin. The VanC, VanE and VanG phenotypes are associated with lower levels of resistance to both vancomycin and teicoplanin, while the VanD phenotype is associated with moderate levels of resistance to both vancomycin and teicoplanin (de Lalla et al., Antimicrob Agents Chemother. 36:2192-2196 (1992); McKessar et al., Antimicrob. Agents Chemother. 44: 3224-3228 (2000); Perichon et al., Antimicrob. Agents Chemother. 41:2016-2018 (1997); Leclercq et al., Clin. Infect. Dis. 24:545-556 (1997); Yean et al., BMC Microbiology 7:112 (2007); Arthur et al., J. Bacteriol. 175:117-127 (1993)). Table 1 lists the minimal inhibitory concentration (MIC) for vancomycin and teicoplanin for each phenotype. (Cetinkaya et al., Clin. Microbiol. Rev. 13:686-707 (2000); McKessar et al., Antimicrob. Agents Chemother. 44: 3224-3228 (2000)).

TABLE 1
Minimal inhibitory concentration (MIC) for vancomycin
and teicoplanin for the resistance genes vanA,
vanB, vanC, vanD, vanE and vanG

| | | MIC (μg/mL) | MIC (μg/mL) |
|---|---|---|---|
| Phenotype | Allele | vancomycin | teicoplanin |
| VanA | vanA | 64->1000 (high) | 16-512 (high) |
| VanB | vanB | 4-1024 (can be high) | ≦0.5 (sensitive) |
| VanC | vanC | 2-32 (low) | ≦0.5 (sensitive) |
| VanD | vanD | 128 (moderate) | 4 (moderate) |
| VanE | vanE | 16 (low) | 0.5 (sensitive) |
| VanG | vanG | 12-16 (low) | 0.5 (sensitive) |

VRE infections can be treated with non-glycopeptide antibiotics such as cephalosporins and aminoglycosides; regardless of the phenotype, susceptibility testing is performed on isolates to determine the best course of treatment.

VRE is a threat to immunocompromised individuals, individuals recovering from surgical procedures and those generally in poor health. An individual can be colonized with VRE, which may or may not become a full-blown infection. Although colonized individuals can remain asymptomatic for months, such persons are capable of transmitting VRE to others. VRE is rarely a concern for healthy adults, and is usually cleared from the host without intervention.

Diagnosis of VRE can be determined using bacteriological and molecular-based diagnostic tests to identify the type of vancomycin resistance (i.e. VanA, VanB, VanC phenotypes, etc.) and the infecting/colonizing Enterococcus species. Once VRE is identified, the isolate is subjected to further tests to predict its susceptibility to antibiotics (for treatment of infections) and, in some cases, is speciated to enable the infection to be tracked (for infection control).

Risk factors for VRE infection or colonization include indwelling urinary or central venous catheters; recent abdominal or cardiothoracic surgery; prolonged and/or frequent hospital stays; hospital stay on an ICU, oncology, or transplant ward; stay in a long-term care facility (LTCF); and prior treatment with vancomycin, cephalosporins, metronidazole or clindamycin, or multiple antibiotics.

A reduction or eradication of VRE can occur upon implementation of control measures. VRE incidence can be decreased in hospitals in which patient surveillance cultures are used in concert with barrier isolation of colonized patients. Active infection-control intervention, relying heavily on surveillance cultures to guide the isolation of colonized patients, is important to reducing and even eradicating VRE (Ostrowsky et al., N Engl J Med. 344:1427-1433 (2001)).

Active Surveillance Cultures (ACS), combined with contact precautions, has also been described as effective in VRE reduction and sustaining long-term control. Conversely, long-term VRE increases are observed in institutions not utilizing this approach. (Management of Multidrug-Resistant Organisms in Healthcare Settings, HIPAC/CDC (2006)).

Additional control methods include administrative controls, such as tracking and trending VRE infections/colonizations and establishing a system whereby VRE positive results trigger specific responses (i.e. administrative controls). Control methods will require screening. Testing has been shown to correlate with reduction in VRE occurrence or re-occurrence.

Culture-based methods are widely used to screen patients for the presence of VRE. Bacterial colonies growing on Bile Esculin Azide plates supplemented with vancomycin (BEAV) are preliminarily identified as VRE based on colonial morphology, but additional culture steps would be required for definitive confirmation. The Bile Esculin test differentiates enterococci and group D streptococci from non-group D viridans group streptococci. Bile Esculin positive colonies appear black and are preliminarily identified as enterococci before Gram-staining Gram-positive cocci (Enterococcus is Gram-positive) are then plated on a blood agar plate for isolation. The Gram status of the isolates is confirmed and they are subsequently checked for catalase and pyrrolinodyl peptidase activity. Catalase-negative and pyrrolinodyl peptidase positive isolates can be reported as Enterococcus spp. Positive isolates subjected to susceptibility testing can be classified as VRE if the minimal inhibitory concentration (MIC) of vancomycin is 32 μg/mL (Moellering, R., Clin Infect Dis. 14:1173-6 (1992)).

The use of automated systems allows one to speciate the Enterococcus isolate. Microscopic observation of motility can also be used to speciate Enterococcus. This entire process can take several days, with the first result suggesting Enterococcus obtained in 24-48 hours. These putative vancomycin-resistant isolates can be confirmed more quickly, perhaps within hours, by polymerase chain reaction (PCR) detection of any of the vancomycin-resistance markers. In addition, patient's clinical specimens can be screened rapidly using a PCR test designed to detect vancomycin-resistance genes. A positive result would suggest the need for barrier isolation, while a negative result may establish that barrier precautions are unnecessary.

In addition to VRE infections/colonizations, another form of vancomycin resistant bacteria has been observed. Vancomycin resistant Staphylococcus aureus (VRSA) are antimicrobial-resistant Staphylococci. Patients that develop VRSA infections usually have several underlying health conditions (such as diabetes), previous infections with MRSA, and recent hospitalizations. The spread of VRSA occurs through close physical contact with infected patients or contaminated material.

### Assays

Tables 2 and 3 demonstrate possible diagnostic outcome scenarios using the probes and primers described herein in diagnostic methods.

TABLE 2
Possible diagnostic outcome scenarios using the
probes and primers of the present invention.

| Cha. | Trg. | Results | | | |
|---|---|---|---|---|---|
| 1 | vanA | + | − | + | − |
| 2 | vanB | − | + | + | − |
| 3 | IC | + | + | + | − |

| | | | | |
|---|---|---|---|---|
| Interpretation | Type A | Type B | Type A | Invalid |
| | van. | van. | and B van. | sample |
| | resistance | resistance | resistance | result |

Cha.., Fluorescence channel; Trg., target; +, target detected; −, target not detected; vanA, target corresponding to vancomycin resistance type A; vanB, target corresponding to vancomycin resistance type B; IC, internal control

TABLE 3
Possible diagnostic outcome scenarios using the
probes and primers of the present invention.

| Chan. | Trg. | Results | |
|---|---|---|---|
| 1 | vanA and/or | + | − |
| | vanB | | |
| 2 | IC | + | − |

| | | |
|---|---|---|
| Interpretation | Type A and/or B | Invalid sample result |
| | vancomycin resistance | |

Chan., Fluorescence channel; Trg., target; +, target detected; -, target not detected; vanA, target corresponding to vancomycin resistance type A; vanB, target corresponding to vancomycin resistance type B; IC, internal control

Detection of the internal control (IC) indicates that the sample result is valid, where an absence of a signal corresponding to the IC indicates either an invalid result or that one or more of the specific targets is at a high starting concentration. A signal indicating a high starting concentration of specific target in the absence of an internal control signal is considered to be a valid sample result.

The advantages of a multiplex format are: (1) simplified and improved testing and analysis; (2) increased efficiency and cost-effectiveness; (3) decreased turnaround time (increased speed of reporting results); (4) increased productivity (less equipment time needed); and (5) coordination/standardization of results for patients for multiple organisms (reduces error from inter-assay variation).

Screening and diagnosis of the vancomycin resistance genes and VRE can lead to earlier and more effective treatment of a subject. The methods for diagnosing and detecting vancomycin resistance and VRE described herein can be coupled with effective treatment therapies (e.g., antibiotics). The antibiotic classes comprising non-glycopeptides such as cephalosporins and aminoglycosides are often prescribed for treatment of a vancomycin resistant infection. The treatments for such infections will depend upon the clinical disease state of the patient, as determinable by one of skill in the art.

The present invention therefore provides a method for specifically detecting the presence of antibiotic resistance genes in a given sample using the primers and probes provided herein. Of particular interest in this regard is the ability of the disclosed primers and probes, as well as those that can be designed according to the disclosed methods, to specifically detect all or a majority of presently characterized strains of known, characterized vancomycin-resistance genes. The optimized primers and probes are useful, therefore, for identifying and diagnosing the causative or contributing agents of disease caused by VRE, whereupon an appropriate treatment can then be administered to the individual to eradicate the bacteria.

The present invention provides one or more sets of primers that can anneal to all currently identified vancomycin-resistance genes and the genus Enterococci and thereby amplify a target from a biological sample. The present invention provides, for example, at least a first primer and at least a second primer for the vancomycin resistance genes vanA, vanB, vanC, vanD, van E and vanG, and the genus Enterococci, each of which comprises a nucleotide sequence designed according to the inventive principles disclosed herein, which are used together to amplify DNA from vancomycin-resistance genes and Enterococci in a mixed-flora sample in a multiplex assay.

Also provided herein are probes that hybridize to the vancomycin-resistance gene sequences and Enterococci sequences and/or amplified products derived from the vancomycin-resistance gene sequences and Enterococci sequences. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of DNA of vancomycin-resistance genes because it permits the detection of bacterial-derived DNA at low template concentrations that might not be conducive to visual detection as a gel-stained amplification product.

Primers and probes are sequences that anneal to a bacterial genomic or bacterial genomic derived sequence, e.g., the antibiotic resistance genes of Enterococcus and/or Staphylococcus sequences, e.g., VRE and/or VRSA sequences (the “target” sequences). The target sequence can be, for example, an antibiotic resistance gene or a bacterial genome. In one embodiment, the entire gene sequence can be “scanned” for optimized primers and probes useful for detecting the antibiotic resistance genes. In other embodiments, particular regions of the gene can be scanned, e.g., regions that are documented in the literature as being useful for detecting multiple genes, regions that are conserved, or regions where sufficient information is available in, for example, a public database, with respect to the antibiotic resistance genes.

Sets or groups of primers and probes are generated based on the target to be detected. The set of all possible primers and probes can include, for example, sequences that include the variability at every site based on the known antibiotic resistance gene, or the primers and probes can be generated based on a consensus sequence of the target. The primers and probes are generated such that the primers and probes are able to anneal to a particular sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all known and characterized vancomycin resistance genes and for the genus Enterococci. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30 C below the Tm; e.g., 5×SSPE, 0.5% SDS, at 65 C; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 bp is Tm=81.5+0.41(% (G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of antibiotic resistance genes by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native target sequence. Even if a probe were originally generated based on a consensus, the validation of the probe is in its ability to specifically anneal and detect every, or a large majority of, target sequences. The particular scoring or ranking steps performed depend upon the intended use for the primer and/or probe, the particular target nucleic acid sequence, and the number of resistance genes of that target nucleic acid sequence. The methods of the invention provide optimal primer and probe sequences because they hybridize to all or a subset of vancomycin resistance genes and the genus Enterococci. Once optimized oligonucleotides are identified that can anneal to such genes, the sequences can then further be optimized for use, for example, in conjunction with another optimized sequence as a “primer set” or for use as a probe. A “primer set” is defined as at least one forward primer and one reverse primer.

Described herein are methods for using the primers and probes for producing a nucleic acid product, for example, comprising contacting one or more nucleic acid sequences of SEQ ID NOS: 1-502 and 600-939 to a sample comprising the vancomycin-resistance genes and the Enterococci marker sequences under conditions suitable for nucleic acid polymerization. The primers and probes can additionally be used to sequence the DNA of the vancomycin-resistance genes and the Enterococci marker sequences, or used as diagnostics to, for example, detect vancomycin resistance genes in a clinical isolate sample, e.g., obtained from a subject, e.g., a mammalian subject. Particular combinations for amplifying DNA of vancomycin-resistance genes include, for example, using at least one forward primer selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG), and using at least one reverse primer selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); and 246-248, 250, 251, 254, 258, 259, 262, 266, 269, 274, 284, 286, 287, 290, 301, 304, 306, 308, 312, 320 and 324 (vanG). Particular combinations for amplifying DNA of Enterococci marker sequences include, for example, using at least one forward primer selected from the group consisting of: SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual); and at least one reverse primer comprising a sequence selected from the group consisting of SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual).

Methods are described for detecting vancomycin resistance genes in a sample, for example, comprising (1) contacting at least one forward and reverse primer set, e.g., SEQ ID NOS: SEQ ID NOS: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG) (forward primers) and SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG) (reverse primers) to a sample; (2) conducting an amplification; and (3) detecting the generation of an amplified product, wherein the generation of an amplified product indicates the presence of vancomycin genes from Enterococcus and/or Staphylococcus pathogens in a clinical isolate sample.

Methods are described for detecting the Enterococci marker sequences in a sample, for example, comprising (1) contacting at least one forward and reverse primer set, e.g., SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual) (forward primers); and SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual) (reverse primers) to a sample; (2) conducting an amplification; and (3) detecting the generation of an amplified product, wherein the generation of an amplified product indicates the presence of Enterococcus in a clinical isolate sample.

The detection of amplicons using probes described herein can be performed, for example, using a labeled probe, e.g., the probe comprising a nucleotide sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG); 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual) that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to dissociate the probe's reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

Primer or probe sequences can be ranked according to specific hybridization parameters or metrics that assign a score value indicating their ability to anneal to bacterial strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimmers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid sequences. The methods of determining the primers and probes also can comprise the step of allowing for one or more nucleotide changes when determining identity between the candidate primer and probe sequences and the target nucleic acid sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm−optimal Tm; difference between primer Tms; amplicon length−minimum amplicon length; and distance between the primer and a TaqMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid sequences are hybridized by an optimal forward primer and an optimal reverse primer, for example, by determining the number of base pair differences between target nucleic acid sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Tables 5, 6, 8A, 8B, 9A, 9B, 10A, 10B, 11A, 11B, and 12. In addition, the sequences can be incorporated into longer sequences, provided they function to specifically anneal to and identify bacterial strains. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Tables 5, 6, 8A, 8B, 9A, 9B, 10A, 10B, 11A, 11B, and 12 or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The probe and/or primer nucleic acid sequences of the invention are optimal for identifying numerous strains of a target nucleic acid, e.g., vancomycin-resistance genes and the Enterococci marker sequences. In an embodiment, the nucleic acids of the invention are primers for the synthesis (e.g., amplification) of target nucleic acid sequences and/or probes for identification, isolation, detection, or analysis of target nucleic acid sequences, e.g., an amplified target nucleic acid that is amplified using the primers of the invention.

The present oligonucleotides hybridize with more than one antibiotic resistance gene (gene as determined by differences in its sequence). The probes and primers provided herein can, for example, allow for the detection of currently identified vancomycin resistance genes or a subset thereof. In addition, the primers and probes of the present invention, depending on the vancomycin resistance gene sequence(s), can allow for the detection of previously unidentified antibiotic resistance genes and VRE. The methods of the invention provide for optimal primers and probes, and sets thereof, and combinations of sets thereof, which can hybridize with a larger number of targets than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides known or previously unknown target nucleic acid strain sequences that are identified, for example, using the methods of the invention. In an embodiment, the target nucleic acid sequence is an amplification product. In another embodiment, the target nucleic acid sequence is a native or synthetic nucleic acid. The primers, probes, and target nucleic acid sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for screening for and/or detecting the presence of vancomycin resistance and VRE in an individual and/or from a sample, are provided herein. An individual can be a human male, human female, human adult, human child, or human fetus. An individual can also be any mammal, reptile, avian, fish, or amphibian. Hence, an individual can be a primate, pig, horse, cattle, sheep, dog, rabbit, guinea pig, rodent, bird or fish. A sample includes any item, surface, material, clothing, or environment, for example, sewage or water treatment plants, in which it may be desirable to test for the presence of vancomycin resistance genes and VRE. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of vancomycin resistance genes and VRE.

A probe of the present invention can comprise a label such as, for example, a fluorescent label, a chemiluminescent label, a radioactive label, biotin, gold, dendrimers, aptamer, enzymes, proteins, quenchers and molecular motors. In an embodiment, the probe is a hydrolysis probe, such as, for example, a TaqMan® probe. In other embodiments, the probes of the invention are molecular beacons, any fluorescent probes, and probes that are replaced by any double stranded DNA binding dyes (e.g., SYBR Green®1).

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of vancomycin resistance genes and VRE can be performed by exposing such an oligonucleotide-covered surface to a sample such that the binding of a complementary strain DNA sequence to a surface-attached oligonucleotide elicits a detectable signal or reaction.

Oligonucleotides of the present invention also include primers for isolating and sequencing nucleic acid sequences derived from any identified or yet to be isolated and identified vancomycin-resistance gene and VRE.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence of vancomycin resistance genes and VRE. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of vancomycin resistance genes and VRE. Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Tables 6-8. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the inventive cell cycle gene-related oligonucleotides), normalization controls, or expression controls. In one embodiment, the negative control is a mammalian gene that is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. A probe can be labeled with an enzyme or, alternatively, the probe is labeled with a moiety that is capable of binding to another moiety that is linked to the enzyme. For example, in the biotin-streptavidin interaction, the streptavidin is conjugated to an enzyme such as horseradish peroxidase (HRP). A chromogenic substrate is added to the reaction and is processed/cleaved by the enzyme. The product of the cleavage forms a color, either in the UV or visible spectrum. In another embodiment, streptavidin alkaline phosphatase can be used in a labeled streptavidin-biotin immunoenzymatic antigen detection system.

The invention also provides methods of labeling nucleic acid molecules with cleavable mass spectrometry tags (CMST; U.S. Patent Application No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3: RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various Enterococcus and/or Staphylococcus genetic material or genetic material derived therefrom, or other genetic material derived therefrom, such as RNA, DNA, cDNA, or a PCR product.

A “sample” that is tested for the presence of vancomycin resistance genes and VRE includes, but is not limited to a tissue sample, such as, for example, blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage or fibroblasts. The tissue sample may be fresh, fixed, preserved, or frozen. A sample also includes any item, surface, material, or clothing, or environment, for example, sewage or water treatment plants, in which it may be desirable to test for the presence of vancomycin resistance genes and VRE. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of vancomycin resistance genes and VRE.

The target nucleic acid strain that is amplified may be RNA or DNA or a modification thereof. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g., fluorescent or radioactive) during an extension reaction. The detecting step comprises measuring fluorescence, mass, charge, and/or chemiluminescence.

The target nucleic acid strain may not need amplification and may be RNA or DNA or a modification thereof. If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 4 are three different 10-base candidate probe sequences that are targeted to different regions of a consensus target nucleic acid strain sequence. Each candidate probe sequence is compared to a total of 10 native sequences.

TABLE 4

| | | | | | | | | | | |
|---|---|---|---|---|---|---|---|---|---|---|
| #1. | A | A | A | C | A | C | G | T | G | C |
| | 0.7 | 1.0 | 1.0 | 1.0 | 1.0 | 1.0 | 1.0 | 1.0 | 1.0 | 1.0 |

(SEQ ID NO: 847)
→Number of target nucleic acid strain sequences that are perfectly
complimentary - 7. Three out of the ten sequences do not have an
A at position 1.

| | | | | | | | | | | |
|---|---|---|---|---|---|---|---|---|---|---|
| #2. | C | C | T | T | G | T | T | C | C | A |
| | 1.0 | 0.9 | 1.0 | 0.9 | 0.9 | 1.0 | 1.0 | 1.0 | 1.0 | 1.0 |

(SEQ ID NO: 848)
→Number of target nucleic acid strain sequences that are perfectly
complimentary - 7, 8, or 9. At least one target nucleic acid strain does
not have a C at position 2, T at position 4, or G at position 5. These
differences may all be on one target nucleic acid strain molecule or
may be on two or three separate molecules.

| | | | | | | | | | | |
|---|---|---|---|---|---|---|---|---|---|---|
| #3. | C | A | G | G | G | A | C | G | A | T |
| | 1.0 | 1.0 | 1.0 | 1.0 | 1.0 | 0.9 | 0.8 | 1.0 | 1.0 | 1.0 |

(SEQ ID NO: 849)
→Number of target nucleic acid strain sequences that are perfectly
complimentary - 7 or 8. At least one target nucleic acid strain does
not have an A at position 6 and at least two target nucleic acid strain
do not have a C at position 7. These differences may all be on one
target nucleic acid strain molecule or may be on two separate molecules.

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base—A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same 9 target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid sequences are identified with perfect complementarity to all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complementary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TaqMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan® probe. If there are two or more degenerate bases in a primer and probe set the ranking will initially select the sets where the degeneracies occur on different sequences.

### B. Coverage Score

The total number of aligned sequences is considered under a coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, bacterial strains and/or species that are linked to high frequencies of infection will have a higher score than strains that are generally regarded as benign. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted to BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting the Antibiotic Resistance Genes vanA, vanB, vanC, vanD, vanE and vanG Gene Variants

The set of primers and probes were then scored according to the methods described herein to identify the optimized primers and probes of Table 5 (vanA and vanB), and Table 6 (vanC, vanD, vanE and vanG). It should be noted that the primers, as they are sequences that anneal to a plurality of all identified or unidentified vancomycin-resistance genes, can also be used as probes either in the presence or absence of amplification of a sample.

TABLE 5
Optimized Primers and Probes for the Detection of vanA and vanB Resistance Genes.

| | | | |
|---|---|---|---|
| Group No. | Forward Primer | Probe | Reverse Primer |

vanA Sets

| | | | |
|---|---|---|---|
| 1 | SEQ ID NO: 1 | SEQ ID NO: 2 | SEQ ID NO: 3 |
| | TTGTGCGGTATTGGGAAACAGT | TGATTTGGTCCACCTCGCCAACAACTAACGC | CGACTTCCTGATGAATACGAAAGATTCC |
| 2 | SEQ ID NO: 1 | SEQ ID NO: 4 | SEQ ID NO: 3 |
| | TTGTGCGGTATTGGGAAACAGT | CCTGATTTGGTCCACCTCGCCAACAACTAACG | CGACTTCCTGATGAATACGAAAGATTCC |
| 3 | SEQ ID NO: 1 | SEQ ID NO: 2 | SEQ ID NO: 5 |
| | TTGTGCGGTATTGGGAAACAGT | TGATTTGGTCCACCTCGCCAACAACTAACGC | CTCGACTTCCTGATGAATACGAAAGATTC |
| 4 | SEQ ID NO: 1 | SEQ ID NO: 4 | SEQ ID NO: 5 |
| | TTGTGCGGTATTGGGAAACAGT | CCTGATTTGGTCCACCTCGCCAACAACTAACG | CTCGACTTCCTGATGAATACGAAAGATTC |
| 5 | SEQ ID NO: 6 | SEQ ID NO: 7 | SEQ ID NO: 8 |
| | CGCGTTCAGGCTCATCCTT | TCACAGCCCGAAACAGCCTGCTCAATTAAGAT | ACTGTTTCCCAATACCGCACAAC |
| | | TTTGC | |
| 6 | SEQ ID NO: 6 | SEQ ID NO: 9 | SEQ ID NO: 10 |
| | CGCGTTCAGGCTCATCCTT | CTCACAGCCCGAAACAGCCTGCTCAATTAAGA | ACTGTTTCCCAATACCGCACAA |
| | | TTT | |
| 7 | SEQ ID NO: 6 | SEQ ID NO: 11 | SEQ ID NO: 10 |
| | CGCGTTCAGGCTCATCCTT | CTCACAGCCCGAAACAGCCTGCTCAATTAAGA | ACTGTTTCCCAATACCGCACAA |
| | | TT | |
| 8 | SEQ ID NO: 6 | SEQ ID NO: 12 | SEQ ID NO: 10 |
| | CGCGTTCAGGCTCATCCTT | CTCACAGCCCGAAACAGCCTGCTCAATTAAGA | ACTGTTTCCCAATACCGCACAA |
| | | TTTTG | |
| 9 | SEQ ID NO: 6 | SEQ ID NO: 13 | SEQ ID NO: 10 |
| | CGCGTTCAGGCTCATCCTT | CTCACAGCCCGAAACAGCCTGCTCAATTAAGAT | ACTGTTTCCCAATACCGCACAA |
| 10 | SEQ ID NO: 6 | SEQ ID NO: 14 | SEQ ID NO: 8 |
| | CGCGTTCAGGCTCATCCTT | ACAGCCCGAAACAGCCTGCTCAATTAAGATTTT | ACTGTTTCCCAATACCGCACAAC |
| | | GCT | |
| 11 | SEQ ID NO: 6 | SEQ ID NO: 15 | SEQ ID NO: 10 |
| | CGCGTTCAGGCTCATCCTT | TCACAGCCCGAAACAGCCTGCTCAATTAAGAT | ACTGTTTCCCAATACCGCACAA |
| 12 | SEQ ID NO: 6 | SEQ ID NO: 16 | SEQ ID NO: 10 |
| | CGCGTTCAGGCTCATCCTT | TCACAGCCCGAAACAGCCTGCTCAATTAAGATT | ACTGTTTCCCAATACCGCACAA |
| 13 | SEQ ID NO: 6 | SEQ ID NO: 17 | SEQ ID NO: 10 |
| | CGCGTTCAGGCTCATCCTT | TCACAGCCCGAAACAGCCTGCTCAATTAAGAT | ACTGTTTCCCAATACCGCACAA |
| | | TTTG | |
| 14 | SEQ ID NO: 6 | SEQ ID NO: 14 | SEQ ID NO: 10 |
| | CGCGTTCAGGCTCATCCTT | ACAGCCCGAAACAGCCTGCTCAATTAAGATTTT | ACTGTTTCCCAATACCGCACAA |
| | | GCT | |
| 15 | SEQ ID NO: 6 | SEQ ID NO: 18 | SEQ ID NO: 10 |
| | CGCGTTCAGGCTCATCCTT | CAGCCCGAAACAGCCTGCTCAATTAAGATTTTG | ACTGTTTCCCAATACCGCACAA |
| | | CT | |
| 16 | SEQ ID NO: 19 | SEQ ID NO: 20 | SEQ ID NO: 21 |
| | AGCAAAATCTTAATTGAGCAGGC | CCCAATACCGCACAACCGACCTCACAG | GGTCCACCTCGCCAACA |
| | TGTTT | | |
| 17 | SEQ ID NO: 22 | SEQ ID NO: 20 | SEQ ID NO: 21 |
| | CAGCAAAATCTTAATTGAGCAGG | CCCAATACCGCACAACCGACCTCACAG | GGTCCACCTCGCCAACA |
| | CTGTTT | | |
| 18 | SEQ ID NO: 6 | SEQ ID NO: 20 | SEQ ID NO: 21 |
| | CGCGTTCAGGCTCATCCTT | CCCAATACCGCACAACCGACCTCACAG | GGTCCACCTCGCCAACA |
| 19 | SEQ ID NO: 23 | SEQ ID NO: 20 | SEQ ID NO: 21 |
| | GCAAAATCTTAATTGAGCAGGCT | CCCAATACCGCACAACCGACCTCACAG | GGTCCACCTCGCCAACA |
| | GTTT | | |
| 20 | SEQ ID NO: 6 | SEQ ID NO: 24 | SEQ ID NO: 21 |
| | CGCGTTCAGGCTCATCCTT | CAATACCGCACAACCGACCTCACAGCC | GGTCCACCTCGCCAACA |
| 21 | SEQ ID NO: 6 | SEQ ID NO: 25 | SEQ ID NO: 3 |
| | CGCGTTCAGGCTCATCCTT | CTGCAGCCTGATTTGGTCCACCTCGC | CGACTTCCTGATGAATACGAAAGATTCC |
| 22 | SEQ ID NO: 22 | SEQ ID NO: 25 | SEQ ID NO: 3 |
| | CAGCAAAATCTTAATTGAGCAGG | CTGCAGCCTGATTTGGTCCACCTCGC | CGACTTCCTGATGAATACGAAAGATTCC |
| | CTGTTT | | |
| 23 | SEQ ID NO: 19 | SEQ ID NO: 25 | SEQ ID NO: 3 |
| | AGCAAAATCTTAATTGAGCAGGC | CTGCAGCCTGATTTGGTCCACCTCGC | CGACTTCCTGATGAATACGAAAGATTCC |
| | TGTTT | | |
| 24 | SEQ ID NO: 26 | SEQ ID NO: 27 | SEQ ID NO: 3 |
| | GGCTGTGAGGTCGGTTGTG | TGCAGCCTGATTTGGTCCACCTCGC | CGACTTCCTGATGAATACGAAAGATTCC |
| 25 | SEQ ID NO: 22 | SEQ ID NO: 27 | SEQ ID NO: 3 |
| | CAGCAAAATCTTAATTGAGCAGG | TGCAGCCTGATTTGGTCCACCTCGC | CGACTTCCTGATGAATACGAAAGATTCC |
| | CTGTTT | | |
| 26 | SEQ ID NO: 19 | SEQ ID NO: 27 | SEQ ID NO: 3 |
| | AGCAAAATCTTAATTGAGCAGGC | TGCAGCCTGATTTGGTCCACCTCGC | CGACTTCCTGATGAATACGAAAGATTCC |
| | TGTTT | | |
| 27 | SEQ ID NO: 23 | SEQ ID NO: 25 | SEQ ID NO: 3 |
| | GCAAAATCTTAATTGAGCAGGCT | CTGCAGCCTGATTTGGTCCACCTCGC | CGACTTCCTGATGAATACGAAAGATTCC |
| | GTTT | | |
| 28 | SEQ ID NO: 22 | SEQ ID NO: 25 | SEQ ID NO: 5 |
| | CAGCAAAATCTTAATTGAGCAGG | CTGCAGCCTGATTTGGTCCACCTCGC | CTCGACTTCCTGATGAATACGAAAGATTC |
| | CTGTTT | | |
| 29 | SEQ ID NO: 19 | SEQ ID NO: 25 | SEQ ID NO: 5 |
| | AGCAAAATCTTAATTGAGCAGGC | CTGCAGCCTGATTTGGTCCACCTCGC | CTCGACTTCCTGATGAATACGAAAGATTC |
| | TGTTT | | |
| 30 | SEQ ID NO: 6 | SEQ ID NO: 25 | SEQ ID NO: 5 |
| | CGCGTTCAGGCTCATCCTT | CTGCAGCCTGATTTGGTCCACCTCGC | CTCGACTTCCTGATGAATACGAAAGATTC |
| 31 | SEQ ID NO: 28 | SEQ ID NO: 27 | SEQ ID NO: 3 |
| | GGCTGTGAGGTCGGTTGT | TGCAGCCTGATTTGGTCCACCTCGC | CGACTTCCTGATGAATACGAAAGATTCC |
| 32 | SEQ ID NO: 29 | SEQ ID NO: 7 | SEQ ID NO: 8 |
| | GGCGCGTTCAGGCTCATC | TCACAGCCCGAAACAGCCTGCTCAATTAAGAT | ACTGTTTCCCAATACCGCACAAC |
| | | TTTGC | |
| 33 | SEQ ID NO: 23 | SEQ ID NO: 25 | SEQ ID NO: 5 |
| | GCAAAATCTTAATTGAGCAGGCT | CTGCAGCCTGATTTGGTCCACCTCGC | CTCGACTTCCTGATGAATACGAAAGATTC |
| | GTTT | | |
| 34 | SEQ ID NO: 23 | SEQ ID NO: 27 | SEQ ID NO: 3 |
| | GCAAAATCTTAATTGAGCAGGCT | TGCAGCCTGATTTGGTCCACCTCGC | CGACTTCCTGATGAATACGAAAGATTCC |
| | GTTT | | |
| 35 | SEQ ID NO: 19 | SEQ ID NO: 27 | SEQ ID NO: 5 |
| | AGCAAAATCTTAATTGAGCAGGC | TGCAGCCTGATTTGGTCCACCTCGC | CTCGACTTCCTGATGAATACGAAAGATTC |
| | TGTTT | | |
| 36 | SEQ ID NO: 26 | SEQ ID NO: 27 | SEQ ID NO: 5 |
| | GGCTGTGAGGTCGGTTGTG | TGCAGCCTGATTTGGTCCACCTCGC | CTCGACTTCCTGATGAATACGAAAGATTC |
| 37 | SEQ ID NO: 22 | SEQ ID NO: 27 | SEQ ID NO: 5 |
| | CAGCAAAATCTTAATTGAGCAGG | TGCAGCCTGATTTGGTCCACCTCGC | CTCGACTTCCTGATGAATACGAAAGATTC |
| | CTGTTT | | |
| 38 | SEQ ID NO: 28 | SEQ ID NO: 27 | SEQ ID NO: 5 |
| | GGCTGTGAGGTCGGTTGT | TGCAGCCTGATTTGGTCCACCTCGC | CTCGACTTCCTGATGAATACGAAAGATTC |
| 39 | SEQ ID NO: 19 | SEQ ID NO: 30 | SEQ ID NO: 3 |
| | AGCAAAATCTTAATTGAGCAGGC | CAGCCTGATTTGGTCCACCTCGCCA | CGACTTCCTGATGAATACGAAAGATTCC |
| | TGTTT | | |
| 40 | SEQ ID NO: 23 | SEQ ID NO: 27 | SEQ ID NO: 5 |
| | GCAAAATCTTAATTGAGCAGGCT | TGCAGCCTGATTTGGTCCACCTCGC | CTCGACTTCCTGATGAATACGAAAGATTC |
| | GTTT | | |
| 41 | SEQ ID NO: 6 | SEQ ID NO: 14 | SEQ ID NO: 31 |
| | CGCGTTCAGGCTCATCCTT | ACAGCCCGAAACAGCCTGCTCAATTAAGATTTT | CACTGTTTCCCAATACCGCACAA |
| | | GCT | |
| 42 | SEQ ID NO: 6 | SEQ ID NO: 25 | SEQ ID NO: 32 |
| | CGCGTTCAGGCTCATCCTT | CTGCAGCCTGATTTGGTCCACCTCGC | GCTCGACTTCCTGATGAATACGAAA |
| 43 | SEQ ID NO: 19 | SEQ ID NO: 25 | SEQ ID NO: 32 |
| | AGCAAAATCTTAATTGAGCAGGC | CTGCAGCCTGATTTGGTCCACCTCGC | GCTCGACTTCCTGATGAATACGAAA |
| | TGTTT | | |
| 44 | SEQ ID NO: 22 | SEQ ID NO: 25 | SEQ ID NO: 32 |
| | CAGCAAAATCTTAATTGAGCAGG | CTGCAGCCTGATTTGGTCCACCTCGC | GCTCGACTTCCTGATGAATACGAAA |
| | CTGTTT | | |
| 45 | SEQ ID NO: 23 | SEQ ID NO: 25 | SEQ ID NO: 32 |
| | GCAAAATCTTAATTGAGCAGGCT | CTGCAGCCTGATTTGGTCCACCTCGC | GCTCGACTTCCTGATGAATACGAAA |
| | GTTT | | |
| 46 | SEQ ID NO: 22 | SEQ ID NO: 27 | SEQ ID NO: 32 |
| | CAGCAAAATCTTAATTGAGCAGG | TGCAGCCTGATTTGGTCCACCTCGC | GCTCGACTTCCTGATGAATACGAAA |
| | CTGTTT | | |
| 47 | SEQ ID NO: 26 | SEQ ID NO: 27 | SEQ ID NO: 32 |
| | GGCTGTGAGGTCGGTTGTG | TGCAGCCTGATTTGGTCCACCTCGC | GCTCGACTTCCTGATGAATACGAAA |
| 48 | SEQ ID NO: 28 | SEQ ID NO: 27 | SEQ ID NO: 32 |
| | GGCTGTGAGGTCGGTTGT | TGCAGCCTGATTTGGTCCACCTCGC | GCTCGACTTCCTGATGAATACGAAA |
| 49 | SEQ ID NO: 19 | SEQ ID NO: 27 | SEQ ID NO: 32 |
| | AGCAAAATCTTAATTGAGCAGGC | TGCAGCCTGATTTGGTCCACCTCGC | GCTCGACTTCCTGATGAATACGAAA |
| | TGTTT | | |
| 50 | SEQ ID NO: 23 | SEQ ID NO: 27 | SEQ ID NO: 32 |
| | GCAAAATCTTAATTGAGCAGGCT | TGCAGCCTGATTTGGTCCACCTCGC | GCTCGACTTCCTGATGAATACGAAA |
| | GTTT | | |
| 51 | SEQ ID NO: 33 | SEQ ID NO: 20 | SEQ ID NO: 21 |
| | GGCGCGTTCAGGCTCAT | CCCAATACCGCACAACCGACCTCACAG | GGTCCACCTCGCCAACA |
| 52 | SEQ ID NO: 29 | SEQ ID NO: 20 | SEQ ID NO: 21 |
| | GGCGCGTTCAGGCTCATC | CCCAATACCGCACAACCGACCTCACAG | GGTCCACCTCGCCAACA |
| 53 | SEQ ID NO: 33 | SEQ ID NO: 24 | SEQ ID NO: 21 |
| | GGCGCGTTCAGGCTCAT | CAATACCGCACAACCGACCTCACAGCC | GGTCCACCTCGCCAACA |
| 54 | SEQ ID NO: 34 | SEQ ID NO: 35 | SEQ ID NO: 36 |
| | GGGCTGTGAGGTCGGTTGT | CGTACTGCAGCCTGATTTGGTCCACCTCG | GCTCGACTTCCTGATGAATACGAAAGAT |
| 55 | SEQ ID NO: 37 | SEQ ID NO: 25 | SEQ ID NO: 3 |
| | GAGGTCGGTTGTGCGGTATTG | CTGCAGCCTGATTTGGTCCACCTCGC | CGACTTCCTGATGAATACGAAAGATTCC |
| 56 | SEQ ID NO: 33 | SEQ ID NO: 25 | SEQ ID NO: 3 |
| | GGCGCGTTCAGGCTCAT | CTGCAGCCTGATTTGGTCCACCTCGC | CGACTTCCTGATGAATACGAAAGATTCC |
| 57 | SEQ ID NO: 38 | SEQ ID NO: 25 | SEQ ID NO: 3 |
| | AGGTCGGTTGTGCGGTATTG | CTGCAGCCTGATTTGGTCCACCTCGC | CGACTTCCTGATGAATACGAAAGATTCC |
| 58 | SEQ ID NO: 39 | SEQ ID NO: 25 | SEQ ID NO: 3 |
| | TGAGGTCGGTTGTGCGGTATT | CTGCAGCCTGATTTGGTCCACCTCGC | CGACTTCCTGATGAATACGAAAGATTCC |
| 59 | SEQ ID NO: 40 | SEQ ID NO: 25 | SEQ ID NO: 3 |
| | GAGGTCGGTTGTGCGGTATT | CTGCAGCCTGATTTGGTCCACCTCGC | CGACTTCCTGATGAATACGAAAGATTCC |
| 60 | SEQ ID NO: 1 | SEQ ID NO: 25 | SEQ ID NO: 3 |
| | TTGTGCGGTATTGGGAAACAGT | CTGCAGCCTGATTTGGTCCACCTCGC | CGACTTCCTGATGAATACGAAAGATTCC |
| 61 | SEQ ID NO: 29 | SEQ ID NO: 25 | SEQ ID NO: 3 |
| | GGCGCGTTCAGGCTCATC | CTGCAGCCTGATTTGGTCCACCTCGC | CGACTTCCTGATGAATACGAAAGATTCC |
| 62 | SEQ ID NO: 37 | SEQ ID NO: 27 | SEQ ID NO: 3 |
| | GAGGTCGGTTGTGCGGTATTG | TGCAGCCTGATTTGGTCCACCTCGC | CGACTTCCTGATGAATACGAAAGATTCC |
| 63 | SEQ ID NO: 38 | SEQ ID NO: 27 | SEQ ID NO: 3 |
| | AGGTCGGTTGTGCGGTATTG | TGCAGCCTGATTTGGTCCACCTCGC | CGACTTCCTGATGAATACGAAAGATTCC |
| 64 | SEQ ID NO: 39 | SEQ ID NO: 27 | SEQ ID NO: 3 |
| | TGAGGTCGGTTGTGCGGTATT | TGCAGCCTGATTTGGTCCACCTCGC | CGACTTCCTGATGAATACGAAAGATTCC |
| 65 | SEQ ID NO: 40 | SEQ ID NO: 27 | SEQ ID NO: 3 |
| | GAGGTCGGTTGTGCGGTATT | TGCAGCCTGATTTGGTCCACCTCGC | CGACTTCCTGATGAATACGAAAGATTCC |
| 66 | SEQ ID NO: 33 | SEQ ID NO: 25 | SEQ ID NO: 5 |
| | GGCGCGTTCAGGCTCAT | CTGCAGCCTGATTTGGTCCACCTCGC | CTCGACTTCCTGATGAATACGAAAGATTC |
| 67 | SEQ ID NO: 40 | SEQ ID NO: 25 | SEQ ID NO: 5 |
| | GAGGTCGGTTGTGCGGTATT | CTGCAGCCTGATTTGGTCCACCTCGC | CTCGACTTCCTGATGAATACGAAAGATTC |
| 68 | SEQ ID NO: 37 | SEQ ID NO: 25 | SEQ ID NO: 5 |
| | GAGGTCGGTTGTGCGGTATTG | CTGCAGCCTGATTTGGTCCACCTCGC | CTCGACTTCCTGATGAATACGAAAGATTC |
| 69 | SEQ ID NO: 38 | SEQ ID NO: 25 | SEQ ID NO: 5 |
| | AGGTCGGTTGTGCGGTATTG | CTGCAGCCTGATTTGGTCCACCTCGC | CTCGACTTCCTGATGAATACGAAAGATTC |
| 70 | SEQ ID NO: 39 | SEQ ID NO: 25 | SEQ ID NO: 5 |
| | TGAGGTCGGTTGTGCGGTATT | CTGCAGCCTGATTTGGTCCACCTCGC | CTCGACTTCCTGATGAATACGAAAGATTC |
| 71 | SEQ ID NO: 1 | SEQ ID NO: 25 | SEQ ID NO: 5 |
| | TTGTGCGGTATTGGGAAACAGT | CTGCAGCCTGATTTGGTCCACCTCGC | CTCGACTTCCTGATGAATACGAAAGATTC |
| 72 | SEQ ID NO: 1 | SEQ ID NO: 27 | SEQ ID NO: 3 |
| | TTGTGCGGTATTGGGAAACAGT | TGCAGCCTGATTTGGTCCACCTCGC | CGACTTCCTGATGAATACGAAAGATTCC |
| 73 | SEQ ID NO: 29 | SEQ ID NO: 25 | SEQ ID NO: 5 |
| | GGCGCGTTCAGGCTCATC | CTGCAGCCTGATTTGGTCCACCTCGC | CTCGACTTCCTGATGAATACGAAAGATTC |
| 74 | SEQ ID NO: 38 | SEQ ID NO: 27 | SEQ ID NO: 5 |
| | AGGTCGGTTGTGCGGTATTG | TGCAGCCTGATTTGGTCCACCTCGC | CTCGACTTCCTGATGAATACGAAAGATTC |
| 75 | SEQ ID NO: 40 | SEQ ID NO: 27 | SEQ ID NO: 5 |
| | GAGGTCGGTTGTGCGGTATT | TGCAGCCTGATTTGGTCCACCTCGC | CTCGACTTCCTGATGAATACGAAAGATTC |
| 76 | SEQ ID NO: 37 | SEQ ID NO: 27 | SEQ ID NO: 5 |
| | GAGGTCGGTTGTGCGGTATTG | TGCAGCCTGATTTGGTCCACCTCGC | CTCGACTTCCTGATGAATACGAAAGATTC |
| 77 | SEQ ID NO: 39 | SEQ ID NO: 27 | SEQ ID NO: 5 |
| | TGAGGTCGGTTGTGCGGTATT | TGCAGCCTGATTTGGTCCACCTCGC | CTCGACTTCCTGATGAATACGAAAGATTC |
| 78 | SEQ ID NO: 1 | SEQ ID NO: 27 | SEQ ID NO: 5 |
| | TTGTGCGGTATTGGGAAACAGT | TGCAGCCTGATTTGGTCCACCTCGC | CTCGACTTCCTGATGAATACGAAAGATTC |
| 79 | SEQ ID NO: 1 | SEQ ID NO: 25 | SEQ ID NO: 32 |
| | TTGTGCGGTATTGGGAAACAGT | CTGCAGCCTGATTTGGTCCACCTCGC | GCTCGACTTCCTGATGAATACGAAA |
| 80 | SEQ ID NO: 38 | SEQ ID NO: 25 | SEQ ID NO: 32 |
| | AGGTCGGTTGTGCGGTATTG | CTGCAGCCTGATTTGGTCCACCTCGC | GCTCGACTTCCTGATGAATACGAAA |
| 81 | SEQ ID NO: 40 | SEQ ID NO: 25 | SEQ ID NO: 32 |
| | GAGGTCGGTTGTGCGGTATT | CTGCAGCCTGATTTGGTCCACCTCGC | GCTCGACTTCCTGATGAATACGAAA |
| 82 | SEQ ID NO: 33 | SEQ ID NO: 25 | SEQ ID NO: 32 |
| | GGCGCGTTCAGGCTCAT | CTGCAGCCTGATTTGGTCCACCTCGC | GCTCGACTTCCTGATGAATACGAAA |
| 83 | SEQ ID NO: 37 | SEQ ID NO: 25 | SEQ ID NO: 32 |
| | GAGGTCGGTTGTGCGGTATTG | CTGCAGCCTGATTTGGTCCACCTCGC | GCTCGACTTCCTGATGAATACGAAA |
| 84 | SEQ ID NO: 39 | SEQ ID NO: 25 | SEQ ID NO: 32 |
| | TGAGGTCGGTTGTGCGGTATT | CTGCAGCCTGATTTGGTCCACCTCGC | GCTCGACTTCCTGATGAATACGAAA |
| 85 | SEQ ID NO: 40 | SEQ ID NO: 27 | SEQ ID NO: 32 |
| | GAGGTCGGTTGTGCGGTATT | TGCAGCCTGATTTGGTCCACCTCGC | GCTCGACTTCCTGATGAATACGAAA |
| 86 | SEQ ID NO: 37 | SEQ ID NO: 27 | SEQ ID NO: 32 |
| | GAGGTCGGTTGTGCGGTATTG | TGCAGCCTGATTTGGTCCACCTCGC | GCTCGACTTCCTGATGAATACGAAA |
| 87 | SEQ ID NO: 1 | SEQ ID NO: 27 | SEQ ID NO: 32 |
| | TTGTGCGGTATTGGGAAACAGT | TGCAGCCTGATTTGGTCCACCTCGC | GCTCGACTTCCTGATGAATACGAAA |
| 88 | SEQ ID NO: 38 | SEQ ID NO: 27 | SEQ ID NO: 32 |
| | AGGTCGGTTGTGCGGTATTG | TGCAGCCTGATTTGGTCCACCTCGC | GCTCGACTTCCTGATGAATACGAAA |
| 89 | SEQ ID NO: 39 | SEQ ID NO: 27 | SEQ ID NO: 32 |
| | TGAGGTCGGTTGTGCGGTATT | TGCAGCCTGATTTGGTCCACCTCGC | GCTCGACTTCCTGATGAATACGAAA |
| 90 | SEQ ID NO: 41 | SEQ ID NO: 20 | SEQ ID NO: 21 |
| | GGCAAGACAATATGACAGCAAA | CCCAATACCGCACAACCGACCTCACAG | GGTCCACCTCGCCAACA |
| | ATCTTAATTG | | |
| 91 | SEQ ID NO: 42 | SEQ ID NO: 43 | SEQ ID NO: 44 |
| | CAGCTACGTTTACCTATCCTGTTT | CCGGCGCGTTCAGGCTCATCCTT | GCCTGCTCAATTAAGATTTTGCTGTCAT |
| | TTGTTAAG | | |
| 92 | SEQ ID NO: 45 | SEQ ID NO: 46 | SEQ ID NO: 47 |
| | TGGCGAGGTGGACCAAATCA | ACTGCGTTTTCAGAGCCTTTTTCCGGCTC | GGTCTGCGGGAACGGTTAT |
| 93 | SEQ ID NO: 48 | SEQ ID NO: 49 | SEQ ID NO: 47 |
| | GTTGGCGAGGTGGACCAAAT | AGAGCCTTTTTCCGGCTCGACTTCCTGAT | GGTCTGCGGGAACGGTTAT |
| 94 | SEQ ID NO: 26 | SEQ ID NO: 50 | SEQ ID NO: 47 |
| | GGCTGTGAGGTCGGTTGTG | ACTGCGTTTTCAGAGCCTTTTTCCGGCTCG | GGTCTGCGGGAACGGTTAT |
| 95 | SEQ ID NO: 26 | SEQ ID NO: 27 | SEQ ID NO: 51 |
| | GGCTGTGAGGTCGGTTGTG | TGCAGCCTGATTTGGTCCACCTCGC | CTGCGTTTTCAGAGCCTTTTTCC |
| 96 | SEQ ID NO: 34 | SEQ ID NO: 50 | SEQ ID NO: 52 |
| | GGGCTGTGAGGTCGGTTGT | ACTGCGTTTTCAGAGCCTTTTTCCGGCTCG | AGGTCTGCGGGAACGGTTAT |
| 97 | SEQ ID NO: 53 | SEQ ID NO: 54 | SEQ ID NO: 47 |
| | AATCAGGCTGCAGTACGGAATCT | TGCGTTTTCAGAGCCTTTTTCCGGCTCG | GGTCTGCGGGAACGGTTAT |
| | TT | | |
| 98 | SEQ ID NO: 19 | SEQ ID 50 | SEQ ID 55 |
| | AGCAAAATCTTAATTGAGCAGGC | ACTGCGTTTTCAGAGCCTTTTTCCGGCTCG | AAGGTCTGCGGGAACGGTTA |
| | TGTTT | | |
| 99 | SEQ ID NO: 19 | SEQ ID NO: 56 | SEQ ID NO: 52 |
| | AGCAAAATCTTAATTGAGCAGGC | TGCGTTTTCAGAGCCTTTTTCCGGCTCGAC | AGGTCTGCGGGAACGGTTAT |
| | TGTTT | | |
| 100 | SEQ ID NO: 19 | SEQ ID NO: 50 | SEQ ID NO: 52 |
| | AGCAAAATCTTAATTGAGCAGGC | ACTGCGTTTTCAGAGCCTTTTTCCGGCTCG | AGGTCTGCGGGAACGGTTAT |
| | TGTTT | | |
| 101 | SEQ ID NO: 19 | SEQ ID NO: 57 | SEQ ID NO: 52 |
| | AGCAAAATCTTAATTGAGCAGGC | AACTGCGTTTTCAGAGCCTTTTTCCGGCTCG | AGGTCTGCGGGAACGGTTAT |
| | TGTTT | | |
| 102 | SEQ ID NO: 19 | SEQ ID NO: 54 | SEQ ID NO: 47 |
| | AGCAAAATCTTAATTGAGCAGGC | TGCGTTTTCAGAGCCTTTTTCCGGCTCG | GGTCTGCGGGAACGGTTAT |
| | TGTTT | | |
| 103 | SEQ ID NO: 19 | SEQ ID NO: 46 | SEQ ID NO: 47 |
| | AGCAAAATCTTAATTGAGCAGGC | ACTGCGTTTTCAGAGCCTTTTTCCGGCTC | GGTCTGCGGGAACGGTTAT |
| | TGTTT | | |
| 104 | SEQ ID NO: 19 | SEQ ID NO: 58 | SEQ ID NO: 3 |
| | AGCAAAATCTTAATTGAGCAGGC | TGTTTCCCAATACCGCACAACCGACCTCAC | CGACTTCCTGATGAATACGAAAGATTCC |
| | TGTTT | | |
| 105 | SEQ ID NO: 59 | SEQ ID NO: 56 | SEQ ID NO: 52 |
| | GCAGTACGGAATCTTTCGTATTC | TGCGTTTTCAGAGCCTTTTTCCGGCTCGAC | AGGTCTGCGGGAACGGTTAT |
| | ATCAG | | |
| 106 | SEQ ID NO: 59 | SEQ ID NO: 54 | SEQ ID NO: 60 |
| | GCAGTACGGAATCTTTCGTATTC | TGCGTTTTCAGAGCCTTTTTCCGGCTCG | GGTCTGCGGGAACGGTTA |
| | ATCAG | | |

vanB Sets

| | | | |
|---|---|---|---|
| 107 | SEQ ID NO: 61 | SEQ ID NO: 62 | SEQ ID NO: 63 |
| | AAATCACTGGCCTACATTCTTACA | TTCTATCGCAGCGTTAAGTTCTTCCGTACC | CGAAATCGCTTGCTCAATTAAGAT |
| 108 | SEQ ID NO: 61 | SEQ ID NO: 64 | SEQ ID NO: 65 |
| | AAATCACTGGCCTACATTCTTACA | TATCGCAGCGTTAAGTTCTTCCGTACCGTTTA | GCTTGCTCAATTAAGATTTTTCCATCA |
| 109 | SEQ ID NO: 61 | SEQ ID NO: 64 | SEQ ID NO: 66 |
| | AAATCACTGGCCTACATTCTTACA | TATCGCAGCGTTAAGTTCTTCCGTACCGTTTA | GCTCAATTAAGATTTTTCCATCATATTGTC |
| 110 | SEQ ID NO: 61 | SEQ ID NO: 62 | SEQ ID NO: 66 |
| | AAATCACTGGCCTACATTCTTACA | TTCTATCGCAGCGTTAAGTTCTTCCGTACC | GCTCAATTAAGATTTTTCCATCATATTGTC |
| 111 | SEQ ID NO: 61 | SEQ ID NO: 67 | SEQ ID NO: 66 |
| | AAATCACTGGCCTACATTCTTACA | CTTCTATCGCAGCGTTAAGTTCTTCCGTACC | GCTCAATTAAGATTTTTCCATCATATTGTC |
| 112 | SEQ ID NO: 68 | SEQ ID NO: 64 | SEQ ID NO: 65 |
| | AAATCACTGGCCTACATTCTTAC | TATCGCAGCGTTAAGTTCTTCCGTACCGTTTA | GCTTGCTCAATTAAGATTTTTCCATCA |
| | AAAA | | |
| 113 | SEQ ID NO: 68 | SEQ ID NO: 64 | SEQ ID NO: 66 |
| | AAATCACTGGCCTACATTCTTAC | TATCGCAGCGTTAAGTTCTTCCGTACCGTTTA | GCTCAATTAAGATTTTTCCATCATATTGTC |
| | AAAA | | |
| 114 | SEQ ID NO: 68 | SEQ ID NO: 62 | SEQ ID NO: 66 |
| | AAATCACTGGCCTACATTCTTAC | TTCTATCGCAGCGTTAAGTTCTTCCGTACC | GCTCAATTAAGATTTTTCCATCATATTGTC |
| | AAAA | | |
| 115 | SEQ ID NO: 68 | SEQ ID NO: 67 | SEQ ID NO: 66 |
| | AAATCACTGGCCTACATTCTTAC | CTTCTATCGCAGCGTTAAGTTCTTCCGTACC | GCTCAATTAAGATTTTTCCATCATATTGTC |
| | AAAA | | |
| 116 | SEQ ID NO: 61 | SEQ ID NO: 69 | SEQ ID NO: 63 |
| | AAATCACTGGCCTACATTCTTACA | TTTTCCATCATATTGTCCTGCCGCTTCTATCG | CGAAATCGCTTGCTCAATTAAGAT |
| 117 | SEQ ID NO: 70 | SEQ ID NO: 69 | SEQ ID NO: 63 |
| | AAATCACTGGCCTACATTCTTAC | TTTTCCATCATATTGTCCTGCCGCTTCTATCG | CGAAATCGCTTGCTCAATTAAGAT |
| | AA | | |
| 118 | SEQ ID NO: 71 | SEQ ID NO: 69 | SEQ ID NO: 63 |
| | AAATCACTGGCCTACATTCTTAC | TTTTCCATCATATTGTCCTGCCGCTTCTATCG | CGAAATCGCTTGCTCAATTAAGAT |
| | AAA | | |
| 119 | SEQ ID NO: 68 | SEQ ID NO: 69 | SEQ ID NO: 63 |
| | AAATCACTGGCCTACATTCTTAC | TTTTCCATCATATTGTCCTGCCGCTTCTATCG | CGAAATCGCTTGCTCAATTAAGAT |
| | AAAA | | |
| 120 | SEQ ID NO: 72 | SEQ ID NO: 73 | SEQ ID NO: 74 |
| | AATCACTGGCCTACATTCTTACAA | AGATTTTTCCATCATATTGTCCTGCCGCTTCTAT | CCGAAATCGCTTGCTCAATTA |
| 121 | SEQ ID NO: 75 | SEQ ID NO: 73 | SEQ ID NO: 74 |
| | AATCACTGGCCTACATTCTTACA | AGATTTTTCCATCATATTGTCCTGCCGCTTCTAT | CCGAAATCGCTTGCTCAATTA |
| | AA | | |
| 122 | SEQ ID NO: 70 | SEQ ID NO: 76 | SEQ ID NO: 77 |
| | AAATCACTGGCCTACATTCTTAC | TAAGATTTTTCCATCATATTGTCCTGCCGCTTCT | CCGAAATCGCTTGCTCAAT |
| | AA | | |
| 123 | SEQ ID NO: 71 | SEQ ID NO: 76 | SEQ ID NO: 77 |
| | AAATCACTGGCCTACATTCTTAC | TAAGATTTTTCCATCATATTGTCCTGCCGCTTCT | CCGAAATCGCTTGCTCAAT |
| | AAA | | |
| 124 | SEQ ID NO: 61 | SEQ ID NO: 76 | SEQ ID NO: 77 |
| | AAATCACTGGCCTACATTCTTACA | TAAGATTTTTCCATCATATTGTCCTGCCGCTTCT | CCGAAATCGCTTGCTCAAT |
| 125 | SEQ ID NO: 68 | SEQ ID NO: 76 | SEQ ID NO: 77 |
| | AAATCACTGGCCTACATTCTTAC | TAAGATTTTTCCATCATATTGTCCTGCCGCTTCT | CCGAAATCGCTTGCTCAAT |
| | AAAA | | |
| 126 | SEQ ID NO: 71 | SEQ ID NO: 78 | SEQ ID NO: 77 |
| | AAATCACTGGCCTACATTCTTAC | AAGATTTTTCCATCATATTGTCCTGCCGCTTCTA | CCGAAATCGCTTGCTCAAT |
| | AAA | | |
| 127 | SEQ ID NO: 61 | SEQ ID NO: 79 | SEQ ID NO: 77 |
| | AAATCACTGGCCTACATTCTTACA | AAGATTTTTCCATCATATTGTCCTGCCGCTTCT | CCGAAATCGCTTGCTCAAT |
| 128 | SEQ ID NO: 70 | SEQ ID NO: 80 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AA | AT | |
| 129 | SEQ ID NO: 70 | SEQ ID NO: 79 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AA | | |
| 130 | SEQ ID NO: 71 | SEQ ID NO: 80 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AAA | AT | |
| 131 | SEQ ID NO: 61 | SEQ ID NO: 78 | SEQ ID NO: 77 |
| | AAATCACTGGCCTACATTCTTACA | AAGATTTTTCCATCATATTGTCCTGCCGCTTCTA | CCGAAATCGCTTGCTCAAT |
| 132 | SEQ ID NO: 71 | SEQ ID NO: 79 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AAA | | |
| 133 | SEQ ID NO: 70 | SEQ ID NO: 78 | SEQ ID NO: 77 |
| | AAATCACTGGCCTACATTCTTAC | AAGATTTTTCCATCATATTGTCCTGCCGCTTCTA | CCGAAATCGCTTGCTCAAT |
| | AA | | |
| 134 | SEQ ID NO: 61 | SEQ ID NO: 80 | SEQ ID NO: 77 |
| | AAATCACTGGCCTACATTCTTACA | AAGATTTTTCCATCATATTGTCCTGCCGCTTCT | CCGAAATCGCTTGCTCAAT |
| | | AT | |
| 135 | SEQ ID NO: 68 | SEQ ID NO: 80 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AAAA | AT | |
| 136 | SEQ ID NO: 68 | SEQ ID NO: 79 | SEQ ID NO: 77 |
| | AAATCACTGGCCTACATTCTTAC | AAGATTTTTCCATCATATTGTCCTGCCGCTTCT | CCGAAATCGCTTGCTCAAT |
| | AAAA | | |
| 137 | SEQ ID NO: 68 | SEQ ID NO: 78 | SEQ ID NO: 77 |
| | AAATCACTGGCCTACATTCTTAC | AAGATTTTTCCATCATATTGTCCTGCCGCTTCTA | CCGAAATCGCTTGCTCAAT |
| | AAAA | | |
| 138 | SEQ ID NO: 81 | SEQ ID NO: 82 | SEQ ID NO: 77 |
| | CTTACCTACCCTGTCTTTGTGA | AGATTTTTCCATCATATTGTCCTGCCGCTTCT | CCGAAATCGCTTGCTCAAT |
| 139 | SEQ ID NO: 83 | SEQ ID NO: 84 | SEQ ID NO: 85 |
| | TTACCTACCCTGTCTTTGTGAAG | CACGGTCAGGTTCGTCCTTTGGC | CGCTTGCTCAATTAAGATTTTTCCA |
| 140 | SEQ ID NO: 83 | SEQ ID NO: 84 | SEQ ID NO: 86 |
| | TTACCTACCCTGTCTTTGTGAAG | CACGGTCAGGTTCGTCCTTTGGC | CGCTTGCTCAATTAAGATTTTTCCAT |
| 141 | SEQ ID NO: 83 | SEQ ID NO: 84 | SEQ ID NO: 65 |
| | TTACCTACCCTGTCTTTGTGAAG | CACGGTCAGGTTCGTCCTTTGGC | GCTTGCTCAATTAAGATTTTTCCATCA |
| 142 | SEQ ID NO: 83 | SEQ ID NO: 84 | SEQ ID NO: 87 |
| | TTACCTACCCTGTCTTTGTGAAG | CACGGTCAGGTTCGTCCTTTGGC | GCTTGCTCAATTAAGATTTTTCCATCAT |
| 143 | SEQ ID NO: 83 | SEQ ID NO: 84 | SEQ ID NO: 88 |
| | TTACCTACCCTGTCTTTGTGAAG | CACGGTCAGGTTCGTCCTTTGGC | GCTTGCTCAATTAAGATTTTTCCATCATA |
| 144 | SEQ ID NO: 89 | SEQ ID NO: 84 | SEQ ID NO: 90 |
| | ACCTACCCTGTCTTTGTGAAG | CACGGTCAGGTTCGTCCTTTGGC | TCGCTTGCTCAATTAAGATTTTTCC |
| 145 | SEQ ID NO: 89 | SEQ ID NO: 84 | SEQ ID NO: 85 |
| | ACCTACCCTGTCTTTGTGAAG | CACGGTCAGGTTCGTCCTTTGGC | CGCTTGCTCAATTAAGATTTTTCCA |
| 146 | SEQ ID NO: 89 | SEQ ID NO: 84 | SEQ ID NO: 86 |
| | ACCTACCCTGTCTTTGTGAAG | CACGGTCAGGTTCGTCCTTTGGC | CGCTTGCTCAATTAAGATTTTTCCAT |
| 147 | SEQ ID NO: 89 | SEQ ID NO: 84 | SEQ ID NO: 65 |
| | ACCTACCCTGTCTTTGTGAAG | CACGGTCAGGTTCGTCCTTTGGC | GCTTGCTCAATTAAGATTTTTCCATCA |
| 148 | SEQ ID NO: 89 | SEQ ID NO: 84 | SEQ ID NO: 87 |
| | ACCTACCCTGTCTTTGTGAAG | CACGGTCAGGTTCGTCCTTTGGC | GCTTGCTCAATTAAGATTTTTCCATCAT |
| 149 | SEQ ID NO: 89 | SEQ ID NO: 84 | SEQ ID NO: 91 |
| | ACCTACCCTGTCTTTGTGAAG | CACGGTCAGGTTCGTCCTTTGGC | ATCGCTTGCTCAATTAAGATTTTTCC |
| 150 | SEQ ID NO: 89 | SEQ ID NO: 84 | SEQ ID NO: 88 |
| | ACCTACCCTGTCTTTGTGAAG | CACGGTCAGGTTCGTCCTTTGGC | GCTTGCTCAATTAAGATTTTTCCATCATA |
| 151 | SEQ ID NO: 61 | SEQ ID NO: 92 | SEQ ID NO: 74 |
| | AAATCACTGGCCTACATTCTTACA | TTTTTCCATCATATTGTCCTGCCGCTTCTATCG | CCGAAATCGCTTGCTCAATTA |
| 152 | SEQ ID NO: 70 | SEQ ID NO: 92 | SEQ ID NO: 74 |
| | AAATCACTGGCCTACATTCTTAC | TTTTTCCATCATATTGTCCTGCCGCTTCTATCG | CCGAAATCGCTTGCTCAATTA |
| | AA | | |
| 153 | SEQ ID NO: 71 | SEQ ID NO: 92 | SEQ ID NO: 74 |
| | AAATCACTGGCCTACATTCTTAC | TTTTTCCATCATATTGTCCTGCCGCTTCTATCG | CCGAAATCGCTTGCTCAATTA |
| | AAA | | |
| 154 | SEQ ID NO: 68 | SEQ ID NO: 92 | SEQ ID NO: 74 |
| | AAATCACTGGCCTACATTCTTAC | TTTTTCCATCATATTGTCCTGCCGCTTCTATCG | CCGAAATCGCTTGCTCAATTA |
| | AAAA | | |
| 155 | SEQ ID NO: 93 | SEQ ID NO: 84 | SEQ ID NO: 65 |
| | TACCTACCCTGTCTTTGTGAAG | CACGGTCAGGTTCGTCCTTTGGC | GCTTGCTCAATTAAGATTTTTCCATCA |
| 156 | SEQ ID NO: 93 | SEQ ID NO: 84 | SEQ ID NO: 87 |
| | TACCTACCCTGTCTTTGTGAAG | CACGGTCAGGTTCGTCCTTTGGC | GCTTGCTCAATTAAGATTTTTCCATCAT |
| 157 | SEQ ID NO: 93 | SEQ ID NO: 84 | SEQ ID NO: 88 |
| | TACCTACCCTGTCTTTGTGAAG | CACGGTCAGGTTCGTCCTTTGGC | GCTTGCTCAATTAAGATTTTTCCATCATA |
| 158 | SEQ ID NO: 93 | SEQ ID NO: 84 | SEQ ID NO: 90 |
| | TACCTACCCTGTCTTTGTGAAG | CACGGTCAGGTTCGTCCTTTGGC | TCGCTTGCTCAATTAAGATTTTTCC |
| 159 | SEQ ID NO: 93 | SEQ ID NO: 84 | SEQ ID NO: 85 |
| | TACCTACCCTGTCTTTGTGAAG | CACGGTCAGGTTCGTCCTTTGGC | CGCTTGCTCAATTAAGATTTTTCCA |
| 160 | SEQ ID NO: 93 | SEQ ID NO: 84 | SEQ ID NO: 86 |
| | TACCTACCCTGTCTTTGTGAAG | CACGGTCAGGTTCGTCCTTTGGC | CGCTTGCTCAATTAAGATTTTTCCAT |
| 161 | SEQ ID NO: 94 | SEQ ID NO: 84 | SEQ ID NO: 65 |
| | CTTACCTACCCTGTCTTTGTGAA | CACGGTCAGGTTCGTCCTTTGGC | GCTTGCTCAATTAAGATTTTTCCATCA |
| 162 | SEQ ID NO: 94 | SEQ ID NO: 84 | SEQ ID NO: 87 |
| | CTTACCTACCCTGTCTTTGTGAA | CACGGTCAGGTTCGTCCTTTGGC | GCTTGCTCAATTAAGATTTTTCCATCAT |
| 163 | SEQ ID NO: 94 | SEQ ID NO: 84 | SEQ ID NO: 88 |
| | CTTACCTACCCTGTCTTTGTGAA | CACGGTCAGGTTCGTCCTTTGGC | GCTTGCTCAATTAAGATTTTTCCATCATA |
| 164 | SEQ ID NO: 83 | SEQ ID NO: 84 | SEQ ID NO: 95 |
| | TTACCTACCCTGTCTTTGTGAAG | CACGGTCAGGTTCGTCCTTTGGC | CTTGCTCAATTAAGATTTTTCCATCATATTGTC |
| 165 | SEQ ID NO: 61 | SEQ ID NO: 96 | SEQ ID NO: 97 |
| | AAATCACTGGCCTACATTCTTACA | CGCCTCCGGCTTGTCACCTTT | ACAAAGACAGGGTAGGTAAGC |
| 166 | SEQ ID NO: 89 | SEQ ID NO: 84 | SEQ ID NO: 95 |
| | ACCTACCCTGTCTTTGTGAAG | CACGGTCAGGTTCGTCCTTTGGC | CTTGCTCAATTAAGATTTTTCCATCATATTGTC |
| 167 | SEQ ID NO: 89 | SEQ ID NO: 84 | SEQ ID NO: 66 |
| | ACCTACCCTGTCTTTGTGAAG | CACGGTCAGGTTCGTCCTTTGGC | GCTCAATTAAGATTTTTCCATCATATTGTC |
| 168 | SEQ ID NO: 83 | SEQ ID NO: 84 | SEQ ID NO: 66 |
| | TTACCTACCCTGTCTTTGTGAAG | CACGGTCAGGTTCGTCCTTTGGC | GCTCAATTAAGATTTTTCCATCATATTGTC |
| 169 | SEQ ID NO: 93 | SEQ ID NO: 84 | SEQ ID NO: 66 |
| | TACCTACCCTGTCTTTGTGAAG | CACGGTCAGGTTCGTCCTTTGGC | GCTCAATTAAGATTTTTCCATCATATTGTC |
| 170 | SEQ ID NO: 89 | SEQ ID NO: 84 | SEQ ID NO: 98 |
| | ACCTACCCTGTCTTTGTGAAG | CACGGTCAGGTTCGTCCTTTGGC | TCGCTTGCTCAATTAAGATTTTTCCA |
| 171 | SEQ ID NO: 61 | SEQ ID NO: 69 | SEQ ID NO: 74 |
| | AAATCACTGGCCTACATTCTTACA | TTTTCCATCATATTGTCCTGCCGCTTCTATCG | CCGAAATCGCTTGCTCAATTA |
| 172 | SEQ ID NO: 70 | SEQ ID NO: 69 | SEQ ID NO: 74 |
| | AAATCACTGGCCTACATTCTTAC | TTTTCCATCATATTGTCCTGCCGCTTCTATCG | CCGAAATCGCTTGCTCAATTA |
| | AA | | |
| 173 | SEQ ID NO: 71 | SEQ ID NO: 69 | SEQ ID NO: 74 |
| | AAATCACTGGCCTACATTCTTAC | TTTTCCATCATATTGTCCTGCCGCTTCTATCG | CCGAAATCGCTTGCTCAATTA |
| | AAA | | |
| 174 | SEQ ID NO: 89 | SEQ ID NO: 84 | SEQ ID NO: 99 |
| | ACCTACCCTGTCTTTGTGAAG | CACGGTCAGGTTCGTCCTTTGGC | ATCGCTTGCTCAATTAAGATTTTTCCA |
| 175 | SEQ ID NO: 68 | SEQ ID NO: 69 | SEQ ID NO: 74 |
| | AAATCACTGGCCTACATTCTTAC | TTTTCCATCATATTGTCCTGCCGCTTCTATCG | CCGAAATCGCTTGCTCAATTA |
| | AAAA | | |
| 176 | SEQ ID NO: 89 | SEQ ID NO: 84 | SEQ ID NO: 100 |
| | ACCTACCCTGTCTTTGTGAAG | CACGGTCAGGTTCGTCCTTTGGC | TCGCTTGCTCAATTAAGATTTTTCCAT |
| 177 | SEQ ID NO: 93 | SEQ ID NO: 84 | SEQ ID NO: 95 |
| | TACCTACCCTGTCTTTGTGAAG | CACGGTCAGGTTCGTCCTTTGGC | CTTGCTCAATTAAGATTTTTCCATCATATTGTC |
| 178 | SEQ ID NO: 89 | SEQ ID NO: 84 | SEQ ID NO: 101 |
| | ACCTACCCTGTCTTTGTGAAG | CACGGTCAGGTTCGTCCTTTGGC | CGCTTGCTCAATTAAGATTTTTCC |
| 179 | SEQ ID NO: 83 | SEQ ID NO: 84 | SEQ ID NO: 101 |
| | TTACCTACCCTGTCTTTGTGAAG | CACGGTCAGGTTCGTCCTTTGGC | CGCTTGCTCAATTAAGATTTTTCC |
| 180 | SEQ ID NO: 93 | SEQ ID NO: 84 | SEQ ID NO: 101 |
| | TACCTACCCTGTCTTTGTGAAG | CACGGTCAGGTTCGTCCTTTGGC | CGCTTGCTCAATTAAGATTTTTCC |
| 181 | SEQ ID NO: 94 | SEQ ID NO: 84 | SEQ ID NO: 95 |
| | CTTACCTACCCTGTCTTTGTGAA | CACGGTCAGGTTCGTCCTTTGGC | CTTGCTCAATTAAGATTTTTCCATCATATTGTC |
| 182 | SEQ ID NO: 89 | SEQ ID NO: 84 | SEQ ID NO: 102 |
| | ACCTACCCTGTCTTTGTGAAG | CACGGTCAGGTTCGTCCTTTGGC | ATCGCTTGCTCAATTAAGATTTTTCCAT |
| 183 | SEQ ID NO: 93 | SEQ ID NO: 84 | SEQ ID NO: 98 |
| | TACCTACCCTGTCTTTGTGAAG | CACGGTCAGGTTCGTCCTTTGGC | TCGCTTGCTCAATTAAGATTTTTCCA |
| 184 | SEQ ID NO: 94 | SEQ ID NO: 84 | SEQ ID NO: 66 |
| | CTTACCTACCCTGTCTTTGTGAA | CACGGTCAGGTTCGTCCTTTGGC | GCTCAATTAAGATTTTTCCATCATATTGTC |
| 185 | SEQ ID NO: 61 | SEQ ID NO: 92 | SEQ ID NO: 77 |
| | AAATCACTGGCCTACATTCTTACA | TTTTTCCATCATATTGTCCTGCCGCTTCTATCG | CCGAAATCGCTTGCTCAAT |
| 186 | SEQ ID NO: 70 | SEQ ID NO: 92 | SEQ ID NO: 77 |
| | AAATCACTGGCCTACATTCTTAC | TTTTTCCATCATATTGTCCTGCCGCTTCTATCG | CCGAAATCGCTTGCTCAAT |
| | AA | | |
| 187 | SEQ ID NO: 71 | SEQ ID NO: 92 | SEQ ID NO: 77 |
| | AAATCACTGGCCTACATTCTTAC | TTTTTCCATCATATTGTCCTGCCGCTTCTATCG | CCGAAATCGCTTGCTCAAT |
| | AAA | | |
| 188 | SEQ ID NO: 81 | SEQ ID NO: 84 | SEQ ID NO: 88 |
| | CTTACCTACCCTGTCTTTGTGA | CACGGTCAGGTTCGTCCTTTGGC | GCTTGCTCAATTAAGATTTTTCCATCATA |
| 189 | SEQ ID NO: 81 | SEQ ID NO: 84 | SEQ ID NO: 65 |
| | CTTACCTACCCTGTCTTTGTGA | CACGGTCAGGTTCGTCCTTTGGC | GCTTGCTCAATTAAGATTTTTCCATCA |
| 190 | SEQ ID NO: 81 | SEQ ID NO: 84 | SEQ ID NO: 87 |
| | CTTACCTACCCTGTCTTTGTGA | CACGGTCAGGTTCGTCCTTTGGC | GCTTGCTCAATTAAGATTTTTCCATCAT |
| 191 | SEQ ID NO: 68 | SEQ ID NO: 92 | SEQ ID NO: 77 |
| | AAATCACTGGCCTACATTCTTAC | TTTTTCCATCATATTGTCCTGCCGCTTCTATCG | CCGAAATCGCTTGCTCAAT |
| | AAAA | | |
| 192 | SEQ ID NO: 103 | SEQ ID NO: 84 | SEQ ID NO: 86 |
| | TTACCTACCCTGTCTTTGTGAA | CACGGTCAGGTTCGTCCTTTGGC | CGCTTGCTCAATTAAGATTTTTCCAT |
| 193 | SEQ ID NO: 103 | SEQ ID NO: 84 | SEQ ID NO: 66 |
| | TTACCTACCCTGTCTTTGTGAA | CACGGTCAGGTTCGTCCTTTGGC | GCTCAATTAAGATTTTTCCATCATATTGTC |
| 194 | SEQ ID NO: 103 | SEQ ID NO: 84 | SEQ ID NO: 65 |
| | TTACCTACCCTGTCTTTGTGAA | CACGGTCAGGTTCGTCCTTTGGC | GCTTGCTCAATTAAGATTTTTCCATCA |
| 195 | SEQ ID NO: 103 | SEQ ID NO: 84 | SEQ ID NO: 87 |
| | TTACCTACCCTGTCTTTGTGAA | CACGGTCAGGTTCGTCCTTTGGC | GCTTGCTCAATTAAGATTTTTCCATCAT |
| 196 | SEQ ID NO: 103 | SEQ ID NO: 84 | SEQ ID NO: 88 |
| | TTACCTACCCTGTCTTTGTGAA | CACGGTCAGGTTCGTCCTTTGGC | GCTTGCTCAATTAAGATTTTTCCATCATA |
| 197 | SEQ ID NO: 81 | SEQ ID NO: 84 | SEQ ID NO: 66 |
| | CTTACCTACCCTGTCTTTGTGA | CACGGTCAGGTTCGTCCTTTGGC | GCTCAATTAAGATTTTTCCATCATATTGTC |
| 198 | SEQ ID NO: 71 | SEQ ID NO: 69 | SEQ ID NO: 77 |
| | AAATCACTGGCCTACATTCTTAC | TTTTCCATCATATTGTCCTGCCGCTTCTATCG | CCGAAATCGCTTGCTCAAT |
| | AAA | | |
| 199 | SEQ ID NO: 61 | SEQ ID NO: 69 | SEQ ID NO: 77 |
| | AAATCACTGGCCTACATTCTTACA | TTTTCCATCATATTGTCCTGCCGCTTCTATCG | CCGAAATCGCTTGCTCAAT |
| 200 | SEQ ID NO: 70 | SEQ ID NO: 69 | SEQ ID NO: 77 |
| | AAATCACTGGCCTACATTCTTAC | TTTTCCATCATATTGTCCTGCCGCTTCTATCG | CCGAAATCGCTTGCTCAAT |
| | AA | | |
| 201 | SEQ ID NO: 81 | SEQ ID NO: 84 | SEQ ID NO: 95 |
| | CTTACCTACCCTGTCTTTGTGA | CACGGTCAGGTTCGTCCTTTGGC | CTTGCTCAATTAAGATTTTTCCATCATATTGTC |
| 202 | SEQ ID NO: 68 | SEQ ID NO: 69 | SEQ ID NO: 77 |
| | AAATCACTGGCCTACATTCTTAC | TTTTCCATCATATTGTCCTGCCGCTTCTATCG | CCGAAATCGCTTGCTCAAT |
| | AAAA | | |
| 203 | SEQ ID NO: 104 | SEQ ID NO: 84 | SEQ ID NO: 66 |
| | GCTTACCTACCCTGTCTTTGT | CACGGTCAGGTTCGTCCTTTGGC | GCTCAATTAAGATTTTTCCATCATATTGTC |
| 204 | SEQ ID NO: 105 | SEQ ID NO: 84 | SEQ ID NO: 66 |
| | CGCTTACCTACCCTGTCTTT | CACGGTCAGGTTCGTCCTTTGGC | GCTCAATTAAGATTTTTCCATCATATTGTC |
| 205 | SEQ ID NO: 89 | SEQ ID NO: 106 | SEQ ID NO: 66 |
| | ACCTACCCTGTCTTTGTGAAG | AGGTTCGTCCTTTGGCGTAACCAAAGTAAAC | GCTCAATTAAGATTTTTCCATCATATTGTC |
| 206 | SEQ ID NO: 107 | SEQ ID NO: 84 | SEQ ID NO: 66 |
| | CGCTTACCTACCCTGTCTT | CACGGTCAGGTTCGTCCTTTGGC | GCTCAATTAAGATTTTTCCATCATATTGTC |
| 207 | SEQ ID NO: 103 | SEQ ID NO: 108 | SEQ ID NO: 66 |
| | TTACCTACCCTGTCTTTGTGAA | CAGGTTCGTCCTTTGGCGTAACCA | GCTCAATTAAGATTTTTCCATCATATTGTC |
| 208 | SEQ ID NO: 93 | SEQ ID NO: 109 | SEQ ID NO: 66 |
| | TACCTACCCTGTCTTTGTGAAG | CCAAAGGACGAACCTGACCGTGC | GCTCAATTAAGATTTTTCCATCATATTGTC |
| 209 | SEQ ID NO: 89 | SEQ ID NO: 110 | SEQ ID NO: 66 |
| | ACCTACCCTGTCTTTGTGAAG | AGGACGAACCTGACCGTGCC | GCTCAATTAAGATTTTTCCATCATATTGTC |
| 210 | SEQ ID NO: 111 | SEQ ID NO: 112 | SEQ ID NO: 63 |
| | ATCACTGGCCTACATTCTTACA | CGCTTACCTACCCTGTCTTTGTGAAGC | CGAAATCGCTTGCTCAATTAAGAT |
| 211 | SEQ ID NO: 111 | SEQ ID NO: 112 | SEQ ID NO: 66 |
| | ATCACTGGCCTACATTCTTACA | CGCTTACCTACCCTGTCTTTGTGAAGC | GCTCAATTAAGATTTTTCCATCATATTGTC |
| 212 | SEQ ID NO: 111 | SEQ ID NO: 112 | SEQ ID NO: 88 |
| | ATCACTGGCCTACATTCTTACA | CGCTTACCTACCCTGTCTTTGTGAAGC | GCTTGCTCAATTAAGATTTTTCCATCATA |
| 645 | SEQ ID NO: 113 | SEQ ID NO: 114 | SEQ ID NO: 115 |
| | 0000 | 0000 | 0000 |
| 646 | SEQ ID NO: 116 | SEQ ID NO: 117 | SEQ ID NO: 118 |
| | 0000 | 0000 | 0000 |
| 647 | SEQ ID NO: 119 | SEQ ID NO: 120 | SEQ ID NO: 121 |
| | 0000 | 0000 | 0000 |
| 648 | SEQ ID NO: 122 | SEQ ID NO: 113 | SEQ ID NO: 114 |
| | 0000 | 0000 | 0000 |

A PCR primer set for amplifying a vanA gene comprises at least one of the following sets of primer sequences: (1) SEQ ID NOS: 1 and 3; (2) SEQ ID NOS: 1 and 32; (3) SEQ ID NOS: 1 and 5; (4) SEQ ID NOS: 19 and 21; (5) SEQ ID NOS: 19 and 3; (6) SEQ ID NOS: 19 and 32; (7) SEQ ID NOS: 19 and 47; (8) SEQ ID NOS: 19 and 5; (9) SEQ ID NOS: 19 and 52; (10) SEQ ID NOS: 19 and 55; (11) SEQ ID NOS: 22 and 21; (12) SEQ ID NOS: 22 and 3; (13) SEQ ID NOS: 22 and 32; (14) SEQ ID NOS: 22 and 5; (15) SEQ ID NOS: 23 and 21; (16) SEQ ID NOS: 23 and 3; (17) SEQ ID NOS: 23 and 32; (18) SEQ ID NOS: 23 and 5; (19) SEQ ID NOS: 26 and 3; (20) SEQ ID NOS: 26 and 32; (21) SEQ ID NOS: 26 and 47; (22) SEQ ID NOS: 26 and 5; (23) SEQ ID NOS: 26 and 51; (24) SEQ ID NOS: 28 and 3; (25) SEQ ID NOS: 28 and 32; (26) SEQ ID NOS: 28 and 5; (27) SEQ ID NOS: 29 and 21; (28) SEQ ID NOS: 29 and 3; (29) SEQ ID NOS: 29 and 5; (30) SEQ ID NOS: 29 and 8; (31) SEQ ID NOS: 33 and 21; (32) SEQ ID NOS: 33 and 3; (33) SEQ ID NOS: 33 and 32; (34) SEQ ID NOS: 33 and 5; (35) SEQ ID NOS: 34 and 36; (36) SEQ ID NOS: 34 and 52; (37) SEQ ID NOS: 37 and 3; (38) SEQ ID NOS: 37 and 32; (39) SEQ ID NOS: 37 and 5; (40) SEQ ID NOS: 38 and 3; (41) SEQ ID NOS: 38 and 32; (42) SEQ ID NOS: 38 and 5; (43) SEQ ID NOS: 39 and 3; (44) SEQ ID NOS: 39 and 32; (45) SEQ ID NOS: 39 and 5; (46) SEQ ID NOS: 40 and 3; (47) SEQ ID NOS: 40 and 32; (48) SEQ ID NOS: 40 and 5; (49) SEQ ID NOS: 41 and 21; (50) SEQ ID NOS: 42 and 44; (51) SEQ ID NOS: 45 and 47; (52) SEQ ID NOS: 48 and 47; (53) SEQ ID NOS: 53 and 47; (54) SEQ ID NOS: 59 and 52; (55) SEQ ID NOS: 59 and 60; (56) SEQ ID NOS: 6 and 10; (57) SEQ ID NOS: 6 and 21; (58) SEQ ID NOS: 6 and 3; (59) SEQ ID NOS: 6 and 31; (60) SEQ ID NOS: 6 and 32; (61) SEQ ID NOS: 6 and 5; and (62) SEQ ID NOS: 6 and 8.

The preceding numbering of the 62 sets of primers does not correspond exactly to the “Group” numbering scheme in Table 5 because certain groups use the same primer set, but different internal probes. For example, Groups 1 and 2 of Table 5 each employ the forward primer of SEQ ID NO: 1 and the reverse primer of SEQ ID NO: 3, but different internal probes in each instance, e.g., SEQ ID NOS: 2 and 4. Accordingly, primer set “(1)” of the preceding passage implies any one of Groups 1 or 2 of Table 5.

A probe for binding to an amplicon(s) of a vanA gene, or to a vanA gene target, comprises at least one of the following probe sequences: SEQ ID NOS: 2, 4, 7, 9, 11-18, 20, 24, 25, 27, 30, 35, 43, 46, 49, 50, 54, 56, 57, 58 (vanA probes).

A PCR primer set for amplifying a vanB gene comprises at least one of the following sets of primer sequences: (1) SEQ ID NOS: 103 and 65; (2) SEQ ID NOS: 103 and 66; (3) SEQ ID NOS: 103 and 86; (4) SEQ ID NOS: 103 and 87; (5) SEQ ID NOS: 103 and 88; (6) SEQ ID NOS: 104 and 66; (7) SEQ ID NOS: 105 and 66; (8) SEQ ID NOS: 107 and 66; (9) SEQ ID NOS: 111 and 63; (10) SEQ ID NOS: 111 and 66; (11) SEQ ID NOS: 111 and 88; (12) SEQ ID NOS: 61 and 63; (13) SEQ ID NOS: 61 and 65; (14) SEQ ID NOS: 61 and 66; (15) SEQ ID NOS: 61 and 74; (16) SEQ ID NOS: 61 and 77; (17) SEQ ID NOS: 61 and 97; (18) SEQ ID NOS: 68 and 63; (19) SEQ ID NOS: 68 and 65; (20) SEQ ID NOS: 68 and 66; (21) SEQ ID NOS: 68 and 74; (22) SEQ ID NOS: 68 and 77; (23) SEQ ID NOS: 70 and 63; (24) SEQ ID NOS: 70 and 74; (25) SEQ ID NOS: 70 and 77; (26) SEQ ID NOS: 71 and 63; (27) SEQ ID NOS: 71 and 74; (28) SEQ ID NOS: 71 and 77; (29) SEQ ID NOS: 72 and 74; (30) SEQ ID NOS: 75 and 74; (31) SEQ ID NOS: 81 and 65; (32) SEQ ID NOS: 81 and 66; (33) SEQ ID NOS: 81 and 77; (34) SEQ ID NOS: 81 and 87; (35) SEQ ID NOS: 81 and 88; (36) SEQ ID NOS: 81 and 95; (37) SEQ ID NOS: 83 and 101; (38) SEQ ID NOS: 83 and 65; (39) SEQ ID NOS: 83 and 66; (40) SEQ ID NOS: 83 and 85; (41) SEQ ID NOS: 83 and 86; (42) SEQ ID NOS: 83 and 87; (43) SEQ ID NOS: 83 and 88; (44) SEQ ID NOS: 83 and 95; (45) SEQ ID NOS: 89 and 100; (46) SEQ ID NOS: 89 and 101; (47) SEQ ID NOS: 89 and 102; (48) SEQ ID NOS: 89 and 65; (49) SEQ ID NOS: 89 and 66; (50) SEQ ID NOS: 89 and 85; (51) SEQ ID NOS: 89 and 86; (52) SEQ ID NOS: 89 and 87; (53) SEQ ID NOS: 89 and 88; (54) SEQ ID NOS: 89 and 90; (55) SEQ ID NOS: 89 and 91; (56) SEQ ID NOS: 89 and 95; (57) SEQ ID NOS: 89 and 98; (58) SEQ ID NOS: 89 and 99; (59) SEQ ID NOS: 93 and 101; (60) SEQ ID NOS: 93 and 65; (61) SEQ ID NOS: 93 and 66; (62) SEQ ID NOS: 93 and 85; (63) SEQ ID NOS: 93 and 86; (64) SEQ ID NOS: 93 and 87; (65) SEQ ID NOS: 93 and 88; (66) SEQ ID NOS: 93 and 90; (67) SEQ ID NOS: 93 and 95; (68) SEQ ID NOS: 93 and 98; (69) SEQ ID NOS: 94 and 65; (70) SEQ ID NOS: 94 and 66; (71) SEQ ID NOS: 94 and 87; (72) SEQ ID NOS: 94 and 88; and (73) SEQ ID NOS: 94 and 95.

The preceding numbering of the 73 sets of primers does not correspond exactly to the “Group” numbering scheme in Table 5 because certain groups use the same primer set, but different internal probes. For example, Groups 109-111 of Table 5 each employ the forward primer of SEQ ID NO: 61 and the reverse primer of SEQ ID NO: 66, but different internal probes in each instance, e.g., SEQ ID NOS: 64, 62, and 67, respectively. Accordingly, primer set “(14)” of the preceding passage implies any one of Groups 109-111 of Table 5.

A probe for binding to an amplicon(s) of a vanB gene, or to a vanB gene target, comprises at least one of the following probe sequences: SEQ ID NOS: 62, 64, 67, 69, 73, 76, 78, 79, 80, 82, 84, 92, 96, 108, 109, 110 and 112 (vanB probes).

Any set of primers can be used simultaneously in a multiplex reaction with one or more other primer sets, so that multiple amplicons are amplified simultaneously.

TABLE 6

Optimized Primers and Probes for the Detection of vanC, vanD, vanE and vanG
Resistance Genes.

| | | | |
|---|---|---|---|
| Group | | | |
| No. | Forward Primer | Probe | Reverse Primer |

vanC1 Sets

| | | | |
|---|---|---|---|
| 213 | SEQ ID NO: 123 | SEQ ID NO: 124 | SEQ ID NO: 125 |
| | ATGTATGAACAAATGGCTCTTGCATCA | CGCTAGTGCTCCCACTTTGCTTTTATCCCGCTA | GATCAACACAGTAGAACCGTAAGCAAAAG |
| | AC | | |
| 214 | SEQ ID NO: 123 | SEQ ID NO: 126 | SEQ ID NO: 125 |
| | ATGTATGAACAAATGGCTCTTGCATCA | CGCTAGTGCTCCCACTTTGCTTTTATCCCGC | GATCAACACAGTAGAACCGTAAGCAAAAG |
| | AC | | |
| 215 | SEQ ID NO: 127 | SEQ ID NO: 128 | SEQ ID NO: 129 |
| | AAAAGGGATCACAAAAGTAACTGACA | CGCAGCCAATTTCAATACCCGCTATCGCC | CCAATCGTCAATTGCTCATTTCCTAAGAT |
| | AAACAG | | |
| 216 | SEQ ID NO: 130 | SEQ ID NO: 131 | SEQ ID NO: 132 |
| | TCGATCGTTTTATTCAAGACCATGGAT | CCCTTTTGAAGAACCGGCTTCATTCGGCT | GATCAACACAGTAGAACCGTAAGCA |
| | TC | | |
| 217 | SEQ ID NO: 133 | SEQ ID NO: 134 | SEQ ID NO: 135 |
| | AGGGATCACAAAAGTAACTGACAAAA | TGCCGCAGCCAATTTCAATACCCGCTA | ACCAATCGTCAATTGCTCATTTCCTA |
| | CAG | | |
| 218 | SEQ ID NO: 133 | SEQ ID NO: 136 | SEQ ID NO: 137 |
| | AGGGATCACAAAAGTAACTGACAAAA | AAGATGCCGCAGCCAATTTCAATACCCGC | ACCAATCGTCAATTGCTCATTTCCT |
| | CAG | | |
| 219 | SEQ ID NO: 138 | SEQ ID NO: 139 | SEQ ID NO: 140 |
| | TCGATCGTTTTATTCAAGACCATGGAT | CCCTTTTGAAGAACCGGCTTCATTCGGCT | ACCGTAAGCAAAAGCAGTCGTTA |
| | TC | | |
| 220 | SEQ ID NO: 141 | SEQ ID NO: 142 | SEQ ID NO: 143 |
| | TTAACGACTGCTTTTGCTTACGGTTCT | ACCCGCTATCGCCTTTTGGATCAACACAGT | GTCGACAAGAGAAATCGCATCACA |
| 221 | SEQ ID NO: 144 | SEQ ID NO: 145 | SEQ ID NO: 146 |
| | CTTTTGCTTACGGTTCTACTGTGTTGA | CGCAGCCAATTTCAATACCCGCTATCGCC | ACAAGAGAAATCGCATCACAAGCA |
| 222 | SEQ ID NO: 141 | SEQ ID NO: 142 | SEQ ID NO: 147 |
| | TTAACGACTGCTTTTGCTTACGGTTCT | ACCCGCTATCGCCTTTTGGATCAACACAGT | CGCATCACAAGCACCAATCG |
| 223 | SEQ ID NO: 148 | SEQ ID NO: 136 | SEQ ID NO: 137 |
| | TCTGCATTAACGACTGCTTTTGCT | AAGATGCCGCAGCCAATTTCAATACCCGC | ACCAATCGTCAATTGCTCATTTCCT |
| 224 | SEQ ID NO: 148 | SEQ ID NO: 149 | SEQ ID NO: 150 |
| | TCTGCATTAACGACTGCTTTTGCT | TGCCGCAGCCAATTTCAATACCCGCTA | ACCAATCGTCAATTGCTCATTTCCTA |
| 225 | SEQ ID NO: 144 | SEQ ID NO: 145 | SEQ ID NO: 147 |
| | CTTTTGCTTACGGTTCTACTGTGTTGA | CGCAGCCAATTTCAATACCCGCTATCGCC | CGCATCACAAGCACCAATCG |
| 226 | SEQ ID NO: 141 | SEQ ID NO: 136 | SEQ ID NO: 137 |
| | TTAACGACTGCTTTTGCTTACGGTTCT | AAGATGCCGCAGCCAATTTCAATACCCGC | ACCAATCGTCAATTGCTCATTTCCT |
| 227 | SEQ ID NO: 151 | SEQ ID NO: 152 | SEQ ID NO: 153 |
| | GCTTTTGCTTACGGTTCTACTGTGTT | AGATGCCGCAGCCAATTTCAATACCCGC | CACCAATCGTCAATTGCTCATTTCCTA |
| 228 | SEQ ID NO: 151 | SEQ ID NO: 154 | SEQ ID NO: 155 |
| | GCTTTTGCTTACGGTTCTACTGTGTT | CGCAGCCAATTTCAATACCCGCTATCGCCTTT | CACCAATCGTCAATTGCTCATTTCCTAA |
| 229 | SEQ ID NO: 156 | SEQ ID NO: 149 | SEQ ID NO: 150 |
| | GCTTTTGCTTACGGTTCTACTGTGTTG | TGCCGCAGCCAATTTCAATACCCGCTA | ACCAATCGTCAATTGCTCATTTCCTA |
| 230 | SEQ ID NO: 144 | SEQ ID NO: 136 | SEQ ID NO: 137 |
| | CTTTTGCTTACGGTTCTACTGTGTTGA | AAGATGCCGCAGCCAATTTCAATACCCGC | ACCAATCGTCAATTGCTCATTTCCT |
| 231 | SEQ ID NO: 144 | SEQ ID NO: 145 | SEQ ID NO: 157 |
| | CTTTTGCTTACGGTTCTACTGTGTTGA | CGCAGCCAATTTCAATACCCGCTATCGCC | ACCAATCGTCAATTGCTCATTTCCTAAG |
| 232 | SEQ ID NO: 158 | SEQ ID NO: 159 | SEQ ID NO: 160 |
| | ACTGTGTTGATCCAAAAGGCGATAG | TTCCTAAGATGCCGCAGCCAATTTCAATACC | GAAATCGCATCACAAGCACCAATC |
| | CG | | |
| 233 | SEQ ID NO: 161 | SEQ ID NO: 159 | SEQ ID NO: 147 |
| | CTACTGTGTTGATCCAAAAGGCGATA | TTCCTAAGATGCCGCAGCCAATTTCAATACC | CGCATCACAAGCACCAATCG |
| | CG | | |
| 234 | SEQ ID NO: 162 | SEQ ID NO: 163 | SEQ ID NO: 164 |
| | TTTATTCAAGACCATGGATTCCCGATC | TCCCTTTTGAAGAACCGGCTTCATTCGGCT | GCGCTGTTTTGTCAGTTACTTTTGTG |
| | TTTATC | | |
| 235 | SEQ ID NO: 165 | SEQ ID NO: 166 | SEQ ID NO: 167 |
| | TTCAAGACCATGGATTCCCGATCTTTA | TGATCCCTTTTGAAGAACCGGCTTCATTCGGC | TGGAGCGCTGTTTTGTCAGTTAC |
| | TCA | | |
| 236 | SEQ ID NO: 168 | SEQ ID NO: 166 | SEQ ID NO: 169 |
| | ATTCAAGACCATGGATTCCCGATCTTT | TGATCCCTTTTGAAGAACCGGCTTCATTCGGC | GGAGCGCTGTTTTGTCAGTTACTT |
| | ATCA | | |
| 237 | SEQ ID NO: 170 | SEQ ID NO: 166 | SEQ ID NO: 169 |
| | CAAGACCATGGATTCCCGATCTTTAT | TGATCCCTTTTGAAGAACCGGCTTCATTCGGC | GGAGCGCTGTTTTGTCAGTTACTT |
| 238 | SEQ ID NO: 171 | SEQ ID NO: 172 | SEQ ID NO: 173 |
| | AAGACCATGGATTCCCGATCTTTATCA | TCCCTTTTGAAGAACCGGCTTCATTCGGC | TGGAGCGCTGTTTTGTCAGTTA |
| 239 | SEQ ID NO: 171 | SEQ ID NO: 166 | SEQ ID NO: 174 |
| | AAGACCATGGATTCCCGATCTTTATCA | TGATCCCTTTTGAAGAACCGGCTTCATTCGGC | TGGAGCGCTGTTTTGTCAGTT |
| 240 | SEQ ID NO: 175 | SEQ ID NO: 176 | SEQ ID NO: 177 |
| | ACGGTTCTACTGTGTTGATCCAAAAGG | AGATGCCGCAGCCAATTTCAATACCCGCT | CACCAATCGTCAATTGCTCATTTCCT |
| 241 | SEQ ID NO: 178 | SEQ ID NO: 142 | SEQ ID NO: 179 |
| | TCTGCATTAACGACTGCTTTTGCTTA | ACCCGCTATCGCCTTTTGGATCAACACAGT | TAAGATGCCGCAGCCAATTTCA |
| 242 | SEQ ID NO: 180 | SEQ ID NO: 181 | SEQ ID NO: 146 |
| | AAAGGCGATAGCGGGTATTGAAATTG | CCAATCGTCAATTGCTCATTTCCTAAGATGCC | ACAAGAGAAATCGCATCACAAGCA |
| | GCAG | | |
| 243 | SEQ ID NO: 175 | SEQ ID NO: 182 | SEQ ID NO: 177 |
| | ACGGTTCTACTGTGTTGATCCAAAAGG | AAGATGCCGCAGCCAATTTCAATACCCGCT | CACCAATCGTCAATTGCTCATTTCCT |
| 244 | SEQ ID NO: 183 | SEQ ID NO: 149 | SEQ ID NO: 150 |
| | TACGGTTCTACTGTGTTGATCCAAAAGG | TGCCGCAGCCAATTTCAATACCCGCTA | ACCAATCGTCAATTGCTCATTTCCTA |
| 245 | SEQ ID NO: 184 | SEQ ID NO: 166 | SEQ ID NO: 174 |
| | AGACCATGGATTCCCGATCTTTATCA | TGATCCCTTTTGAAGAACCGGCTTCATTCGGC | TGGAGCGCTGTTTTGTCAGTT |
| 246 | SEQ ID NO: 185 | SEQ ID NO: 186 | SEQ ID NO: 187 |
| | CTGCATTAACGACTGCTTTTGCTTAC | ACCCGCTATCGCCTTTTGGATCAACACAGTAG | CTAAGATGCCGCAGCCAATTTC |
| 247 | SEQ ID NO: 188 | SEQ ID NO: 189 | SEQ ID NO: 190 |
| | CGGTTCTACTGTGTTGATCCAAAAGG | TGCCGCAGCCAATTTCAATACCCGCT | CACCAATCGTCAATTGCTCATTTCC |
| 248 | SEQ ID NO: 188 | SEQ ID NO: 152 | SEQ ID NO: 137 |
| | CGGTTCTACTGTGTTGATCCAAAAGG | AGATGCCGCAGCCAATTTCAATACCCGC | ACCAATCGTCAATTGCTCATTTCCT |
| 249 | SEQ ID NO: 188 | SEQ ID NO: 149 | SEQ ID NO: 150 |
| | CGGTTCTACTGTGTTGATCCAAAAGG | TGCCGCAGCCAATTTCAATACCCGCTA | ACCAATCGTCAATTGCTCATTTCCTA |
| 250 | SEQ ID NO: 191 | SEQ ID NO: 136 | SEQ ID NO: 192 |
| | TCTACTGTGTTGATCCAAAAGGCGATA | AAGATGCCGCAGCCAATTTCAATACCCGC | GCACCAATCGTCAATTGCTCATTTC |
| 251 | SEQ ID NO: 193 | SEQ ID NO: 152 | SEQ ID NO: 153 |
| | TTCTACTGTGTTGATCCAAAAGGCGATA | AGATGCCGCAGCCAATTTCAATACCCGC | CACCAATCGTCAATTGCTCATTTCCTA |
| 252 | SEQ ID NO: 193 | SEQ ID NO: 136 | SEQ ID NO: 190 |
| | TTCTACTGTGTTGATCCAAAAGGCGATA | AAGATGCCGCAGCCAATTTCAATACCCGC | CACCAATCGTCAATTGCTCATTTCC |
| 253 | SEQ ID NO: 191 | SEQ ID NO: 152 | SEQ ID NO: 192 |
| | TCTACTGTGTTGATCCAAAAGGCGATA | AGATGCCGCAGCCAATTTCAATACCCGC | GCACCAATCGTCAATTGCTCATTTC |
| 254 | SEQ ID NO: 194 | SEQ ID NO: 195 | SEQ ID NO: 160 |
| | CAAAAGGCGATAGCGGGTATTGAAA | TCAATTGCTCATTTCCTAAGATGCCGCAGCCA | GAAATCGCATCACAAGCACCAATC |
| 255 | SEQ ID NO: 193 | SEQ ID NO: 152 | SEQ ID NO: 190 |
| | TTCTACTGTGTTGATCCAAAAGGCGATA | AGATGCCGCAGCCAATTTCAATACCCGC | CACCAATCGTCAATTGCTCATTTCC |
| 256 | SEQ ID NO: 191 | SEQ ID NO: 152 | SEQ ID NO: 190 |
| | TCTACTGTGTTGATCCAAAAGGCGATA | AGATGCCGCAGCCAATTTCAATACCCGC | CACCAATCGTCAATTGCTCATTTCC |
| 257 | SEQ ID NO: 193 | SEQ ID NO: 136 | SEQ ID NO: 137 |
| | TTCTACTGTGTTGATCCAAAAGGCGATA | AAGATGCCGCAGCCAATTTCAATACCCGC | ACCAATCGTCAATTGCTCATTTCCT |
| 258 | SEQ ID NO: 196 | SEQ ID NO: 197 | SEQ ID NO: 160 |
| | AAAAGGCGATAGCGGGTATTGAAA | TCAATTGCTCATTTCCTAAGATGCCGCAGCCAA | GAAATCGCATCACAAGCACCAATC |
| 259 | SEQ ID NO: 198 | SEQ ID NO: 142 | SEQ ID NO: 179 |
| | ATTAACGACTGCTTTTGCTTACGGTTCT | ACCCGCTATCGCCTTTTGGATCAACACAGT | TAAGATGCCGCAGCCAATTTCA |
| 260 | SEQ ID NO: 193 | SEQ ID NO: 152 | SEQ ID NO: 137 |
| | TTCTACTGTGTTGATCCAAAAGGCGATA | AGATGCCGCAGCCAATTTCAATACCCGC | ACCAATCGTCAATTGCTCATTTCCT |
| 261 | SEQ ID NO: 191 | SEQ ID NO: 152 | SEQ ID NO: 153 |
| | TCTACTGTGTTGATCCAAAAGGCGATA | AGATGCCGCAGCCAATTTCAATACCCGC | CACCAATCGTCAATTGCTCATTTCCTA |
| 262 | SEQ ID NO: 193 | SEQ ID NO: 152 | SEQ ID NO: 150 |
| | TTCTACTGTGTTGATCCAAAAGGCGATA | AGATGCCGCAGCCAATTTCAATACCCGC | ACCAATCGTCAATTGCTCATTTCCTA |
| 263 | SEQ ID NO: 191 | SEQ ID NO: 136 | SEQ ID NO: 190 |
| | TCTACTGTGTTGATCCAAAAGGCGATA | AAGATGCCGCAGCCAATTTCAATACCCGC | CACCAATCGTCAATTGCTCATTTCC |
| 264 | SEQ ID NO: 196 | SEQ ID NO: 195 | SEQ ID NO: 160 |
| | AAAAGGCGATAGCGGGTATTGAAA | TCAATTGCTCATTTCCTAAGATGCCGCAGCCA | GAAATCGCATCACAAGCACCAATC |
| 265 | SEQ ID NO: 161 | SEQ ID NO: 136 | SEQ ID NO: 192 |
| | CTACTGTGTTGATCCAAAAGGCGATA | AAGATGCCGCAGCCAATTTCAATACCCGC | GCACCAATCGTCAATTGCTCATTTC |
| 266 | SEQ ID NO: 193 | SEQ ID NO: 199 | SEQ ID NO: 150 |
| | TTCTACTGTGTTGATCCAAAAGGCGATA | TGCCGCAGCCAATTTCAATACCCGC | ACCAATCGTCAATTGCTCATTTCCTA |
| 267 | SEQ ID NO: 141 | SEQ ID NO: 142 | SEQ ID NO: 179 |
| | TTAACGACTGCTTTTGCTTACGGTTCT | ACCCGCTATCGCCTTTTGGATCAACACAGT | TAAGATGCCGCAGCCAATTTCA |
| 268 | SEQ ID NO: 161 | SEQ ID NO: 152 | SEQ ID NO: 153 |
| | CTACTGTGTTGATCCAAAAGGCGATA | AGATGCCGCAGCCAATTTCAATACCCGC | CACCAATCGTCAATTGCTCATTTCCTA |
| 269 | SEQ ID NO: 191 | SEQ ID NO: 199 | SEQ ID NO: 150 |
| | TCTACTGTGTTGATCCAAAAGGCGATA | TGCCGCAGCCAATTTCAATACCCGC | ACCAATCGTCAATTGCTCATTTCCTA |
| 270 | SEQ ID NO: 161 | SEQ ID NO: 136 | SEQ ID NO: 190 |
| | CTACTGTGTTGATCCAAAAGGCGATA | AAGATGCCGCAGCCAATTTCAATACCCGC | CACCAATCGTCAATTGCTCATTTCC |
| 271 | SEQ ID NO: 191 | SEQ ID NO: 136 | SEQ ID NO: 137 |
| | TCTACTGTGTTGATCCAAAAGGCGATA | AAGATGCCGCAGCCAATTTCAATACCCGC | ACCAATCGTCAATTGCTCATTTCCT |
| 272 | SEQ ID NO: 161 | SEQ ID NO: 152 | SEQ ID NO: 190 |
| | CTACTGTGTTGATCCAAAAGGCGATA | AGATGCCGCAGCCAATTTCAATACCCGC | CACCAATCGTCAATTGCTCATTTCC |
| 273 | SEQ ID NO: 191 | SEQ ID NO: 152 | SEQ ID NO: 137 |
| | TCTACTGTGTTGATCCAAAAGGCGATA | AGATGCCGCAGCCAATTTCAATACCCGC | ACCAATCGTCAATTGCTCATTTCCT |
| 274 | SEQ ID NO: 191 | SEQ ID NO: 152 | SEQ ID NO: 150 |
| | TCTACTGTGTTGATCCAAAAGGCGATA | AGATGCCGCAGCCAATTTCAATACCCGC | ACCAATCGTCAATTGCTCATTTCCTA |
| 275 | SEQ ID NO: 161 | SEQ ID NO: 136 | SEQ ID NO: 137 |
| | CTACTGTGTTGATCCAAAAGGCGATA | AAGATGCCGCAGCCAATTTCAATACCCGC | ACCAATCGTCAATTGCTCATTTCCT |
| 276 | SEQ ID NO: 200 | SEQ ID NO: 142 | SEQ ID NO: 179 |
| | TAACGACTGCTTTTGCTTACGGTTCT | ACCCGCTATCGCCTTTTGGATCAACACAGT | TAAGATGCCGCAGCCAATTTCA |
| 277 | SEQ ID NO: 161 | SEQ ID NO: 152 | SEQ ID NO: 137 |
| | CTACTGTGTTGATCCAAAAGGCGATA | AGATGCCGCAGCCAATTTCAATACCCGC | ACCAATCGTCAATTGCTCATTTCCT |
| 278 | SEQ ID NO: 161 | SEQ ID NO: 152 | SEQ ID NO: 150 |
| | CTACTGTGTTGATCCAAAAGGCGATA | AGATGCCGCAGCCAATTTCAATACCCGC | ACCAATCGTCAATTGCTCATTTCCTA |
| 279 | SEQ ID NO: 161 | SEQ ID NO: 199 | SEQ ID NO: 150 |
| | CTACTGTGTTGATCCAAAAGGCGATA | TGCCGCAGCCAATTTCAATACCCGC | ACCAATCGTCAATTGCTCATTTCCTA |
| 280 | SEQ ID NO: 201 | SEQ ID NO: 142 | SEQ ID NO: 179 |
| | AACGACTGCTTTTGCTTACGGTTCT | ACCCGCTATCGCCTTTTGGATCAACACAGT | TAAGATGCCGCAGCCAATTTCA |
| 281 | SEQ ID NO: 194 | SEQ ID NO: 195 | SEQ ID NO: 147 |
| | CAAAAGGCGATAGCGGGTATTGAAA | TCAATTGCTCATTTCCTAAGATGCCGCAGCCA | CGCATCACAAGCACCAATCG |
| 282 | SEQ ID NO: 202 | SEQ ID NO: 142 | SEQ ID NO: 179 |
| | ACGACTGCTTTTGCTTACGGTTCT | ACCCGCTATCGCCTTTTGGATCAACACAGT | TAAGATGCCGCAGCCAATTTCA |
| 283 | SEQ ID NO: 203 | SEQ ID NO: 195 | SEQ ID NO: 147 |
| | CAAAAGGCGATAGCGGGTATTGAA | TCAATTGCTCATTTCCTAAGATGCCGCAGCCA | CGCATCACAAGCACCAATCG |
| 284 | SEQ ID NO: 194 | SEQ ID NO: 197 | SEQ ID NO: 147 |
| | CAAAAGGCGATAGCGGGTATTGAAA | TCAATTGCTCATTTCCTAAGATGCCGCAGCCAA | CGCATCACAAGCACCAATCG |
| 285 | SEQ ID NO: 204 | SEQ ID NO: 205 | SEQ ID NO: 187 |
| | CGACTGCTTTTGCTTACGGTTCT | TACCCGCTATCGCCTTTTGGATCAACACAGT | CTAAGATGCCGCAGCCAATTTC |
| 286 | SEQ ID NO: 203 | SEQ ID NO: 197 | SEQ ID NO: 147 |
| | CAAAAGGCGATAGCGGGTATTGAA | TCAATTGCTCATTTCCTAAGATGCCGCAGCCAA | CGCATCACAAGCACCAATCG |
| 287 | SEQ ID NO: 204 | SEQ ID NO: 142 | SEQ ID NO: 187 |
| | CGACTGCTTTTGCTTACGGTTCT | ACCCGCTATCGCCTTTTGGATCAACACAGT | CTAAGATGCCGCAGCCAATTTC |
| 288 | SEQ ID NO: 204 | SEQ ID NO: 142 | SEQ ID NO: 179 |
| | CGACTGCTTTTGCTTACGGTTCT | ACCCGCTATCGCCTTTTGGATCAACACAGT | TAAGATGCCGCAGCCAATTTCA |
| 289 | SEQ ID NO: 196 | SEQ ID NO: 195 | SEQ ID NO: 147 |
| | AAAAGGCGATAGCGGGTATTGAAA | TCAATTGCTCATTTCCTAAGATGCCGCAGCCA | CGCATCACAAGCACCAATCG |
| 290 | SEQ ID NO: 196 | SEQ ID NO: 197 | SEQ ID NO: 147 |
| | AAAAGGCGATAGCGGGTATTGAAA | TCAATTGCTCATTTCCTAAGATGCCGCAGCCAA | CGCATCACAAGCACCAATCG |

vanC2/3 Sets

| | | | |
|---|---|---|---|
| 291 | SEQ ID NO: 206 | SEQ ID NO: 207 | SEQ ID NO: 208 |
| | CGCCATTGCCTGAAACGATTG | AGTCTTGGTCTTAAAGGTCTTGCTCGCATCGA | TCAAGTATAGTTCTCCTTGATCCGTGACA |
| | | CT | |
| 292 | SEQ ID NO: 206 | SEQ ID NO: 207 | SEQ ID NO: 209 |
| | CGCCATTGCCTGAAACGATTG | AGTCTTGGTCTTAAAGGTCTTGCTCGCATCGA | AGTATAGTTCTCCTTGATCCGTGACA |
| | | CT | |
| 293 | SEQ ID NO: 210 | SEQ ID NO: 211 | SEQ ID NO: 212 |
| | CCTGAAACGATTGAAACCAAGGTCAA | AGTCGATGCGAGCAAGACCTTTAAGACCAAG | AAGTATAGTTCTCCTTGATCCGTGACA |
| | | ACT | |
| 294 | SEQ ID NO: 210 | SEQ ID NO: 211 | SEQ ID NO: 209 |
| | CCTGAAACGATTGAAACCAAGGTCAA | AGTCGATGCGAGCAAGACCTTTAAGACCAAG | AGTATAGTTCTCCTTGATCCGTGACA |
| | | ACT | |
| 295 | SEQ ID NO: 213 | SEQ ID NO: 214 | SEQ ID NO: 215 |
| | GCGCCATTGCCTGAAACGAT | AAGTCGATGCGAGCAAGACCTTTAAGACCAA | AGTATAGTTCTCCTTGATCCGTGACAAA |
| | | GACT | |
| 296 | SEQ ID NO: 210 | SEQ ID NO: 211 | SEQ ID NO: 215 |
| | CCTGAAACGATTGAAACCAAGGTCAA | AGTCGATGCGAGCAAGACCTTTAAGACCAAG | AGTATAGTTCTCCTTGATCCGTGACAAA |
| | | ACT | |
| 297 | SEQ ID NO: 210 | SEQ ID NO: 211 | SEQ ID NO: 216 |
| | CCTGAAACGATTGAAACCAAGGTCAA | AGTCGATGCGAGCAAGACCTTTAAGACCAAG | GTATAGTTCTCCTTGATCCGTGACAAAAA |
| | | ACT | |
| 298 | SEQ ID NO: 217 | SEQ ID NO: 218 | SEQ ID NO: 219 |
| | CGCCATTGCCTGAAACGATTGAA | CGATGCGAGCAAGACCTTTAAGACCAAGACT | TTCTCCTTGATCCGTGACAAAAAAGT |
| | | ACG | |
| 299 | SEQ ID NO: 220 | SEQ ID NO: 218 | SEQ ID NO: 219 |
| | GCGCCATTGCCTGAAACG | CGATGCGAGCAAGACCTTTAAGACCAAGACT | TTCTCCTTGATCCGTGACAAAAAAGT |
| | | ACG | |
| 300 | SEQ ID NO: 221 | SEQ ID NO: 218 | SEQ ID NO: 219 |
| | GCCATTGCCTGAAACGATTGAAAC | CGATGCGAGCAAGACCTTTAAGACCAAGACT | TTCTCCTTGATCCGTGACAAAAAAGT |
| | | ACG | |
| 301 | SEQ ID NO: 222 | SEQ ID NO: 218 | SEQ ID NO: 219 |
| | CTGCGCCATTGCCTGAAAC | CGATGCGAGCAAGACCTTTAAGACCAAGACT | TTCTCCTTGATCCGTGACAAAAAAGT |
| | | ACG | |
| 302 | SEQ ID NO: 210 | SEQ ID NO: 218 | SEQ ID NO: 219 |
| | CCTGAAACGATTGAAACCAAGGTCAA | CGATGCGAGCAAGACCTTTAAGACCAAGACT | TTCTCCTTGATCCGTGACAAAAAAGT |
| | | ACG | |
| 303 | SEQ ID NO: 206 | SEQ ID NO: 218 | SEQ ID NO: 219 |
| | CGCCATTGCCTGAAACGATTG | CGATGCGAGCAAGACCTTTAAGACCAAGACT | TTCTCCTTGATCCGTGACAAAAAAGT |
| | | ACG | |
| 304 | SEQ ID NO: 222 | SEQ ID NO: 218 | SEQ ID NO: 216 |
| | CTGCGCCATTGCCTGAAAC | CGATGCGAGCAAGACCTTTAAGACCAAGACT | GTATAGTTCTCCTTGATCCGTGACAAAAA |
| | | ACG | |
| 305 | SEQ ID NO: 217 | SEQ ID NO: 218 | SEQ ID NO: 223 |
| | CGCCATTGCCTGAAACGATTGAA | CGATGCGAGCAAGACCTTTAAGACCAAGACT | AGTTCTCCTTGATCCGTGACAAAAAA |
| | | ACG | |
| 306 | SEQ ID NO: 221 | SEQ ID NO: 218 | SEQ ID NO: 209 |
| | GCCATTGCCTGAAACGATTGAAAC | CGATGCGAGCAAGACCTTTAAGACCAAGACT | AGTATAGTTCTCCTTGATCCGTGACA |
| | | ACG | |
| 307 | SEQ ID NO: 224 | SEQ ID NO: 211 | SEQ ID NO: 216 |
| | CCTGAAACGATTGAAACCAAGGTCAA | AGTCGATGCGAGCAAGACCTTTAAGACCAAG | GTATAGTTCTCCTTGATCCGTGACAAAAA |
| | AG | ACT | |
| 308 | SEQ ID NO: 225 | SEQ ID NO: 211 | SEQ ID NO: 216 |
| | CTGAAACGATTGAAACCAAGGTCAAAG | AGTCGATGCGAGCAAGACCTTTAAGACCAAG | GTATAGTTCTCCTTGATCCGTGACAAAAA |
| | | ACT | |
| 309 | SEQ ID NO: 226 | SEQ ID NO: 211 | SEQ ID NO: 209 |
| | CTGAAACGATTGAAACCAAGGTCAAA | AGTCGATGCGAGCAAGACCTTTAAGACCAAG | AGTATAGTTCTCCTTGATCCGTGACA |
| | GAA | ACT | |
| 310 | SEQ ID NO: 222 | SEQ ID NO: 218 | SEQ ID NO: 209 |
| | CTGCGCCATTGCCTGAAAC | CGATGCGAGCAAGACCTTTAAGACCAAGACT | AGTATAGTTCTCCTTGATCCGTGACA |
| | | ACG | |
| 311 | SEQ ID NO: 206 | SEQ ID NO: 218 | SEQ ID NO: 227 |
| | CGCCATTGCCTGAAACGATTG | CGATGCGAGCAAGACCTTTAAGACCAAGACT | TATAGTTCTCCTTGATCCGTGACAAAAAAG |
| | | ACG | |
| 312 | SEQ ID NO: 220 | SEQ ID NO: 218 | SEQ ID NO: 223 |
| | GCGCCATTGCCTGAAACG | CGATGCGAGCAAGACCTTTAAGACCAAGACT | AGTTCTCCTTGATCCGTGACAAAAAA |
| | | ACG | |
| 313 | SEQ ID NO: 228 | SEQ ID NO: 211 | SEQ ID NO: 215 |
| | CCTGAAACGATTGAAACCAAGGTCAAA | AGTCGATGCGAGCAAGACCTTTAAGACCAAG | AGTATAGTTCTCCTTGATCCGTGACAAA |
| | | ACT | |
| 314 | SEQ ID NO: 206 | SEQ ID NO: 218 | SEQ ID NO: 209 |
| | CGCCATTGCCTGAAACGATTG | CGATGCGAGCAAGACCTTTAAGACCAAGACT | AGTATAGTTCTCCTTGATCCGTGACA |
| | | ACG | |
| 315 | SEQ ID NO: 217 | SEQ ID NO: 218 | SEQ ID NO: 227 |
| | CGCCATTGCCTGAAACGATTGAA | CGATGCGAGCAAGACCTTTAAGACCAAGACT | TATAGTTCTCCTTGATCCGTGACAAAAAAG |
| | | ACG | |
| 316 | SEQ ID NO: 221 | SEQ ID NO: 218 | SEQ ID NO: 216 |
| | GCCATTGCCTGAAACGATTGAAAC | CGATGCGAGCAAGACCTTTAAGACCAAGACT | GTATAGTTCTCCTTGATCCGTGACAAAAA |
| | | ACG | |
| 317 | SEQ ID NO: 210 | SEQ ID NO: 218 | SEQ ID NO: 216 |
| | CCTGAAACGATTGAAACCAAGGTCAA | CGATGCGAGCAAGACCTTTAAGACCAAGACT | GTATAGTTCTCCTTGATCCGTGACAAAAA |
| | | ACG | |
| 318 | SEQ ID NO: 229 | SEQ ID NO: 211 | SEQ ID NO: 215 |
| | CTGAAACGATTGAAACCAAGGTCAAA | AGTCGATGCGAGCAAGACCTTTAAGACCAAG | AGTATAGTTCTCCTTGATCCGTGACAAA |
| | GA | ACT | |
| 319 | SEQ ID NO: 226 | SEQ ID NO: 211 | SEQ ID NO: 216 |
| | CTGAAACGATTGAAACCAAGGTCAAA | AGTCGATGCGAGCAAGACCTTTAAGACCAAG | GTATAGTTCTCCTTGATCCGTGACAAAAA |
| | GAA | ACT | |
| 320 | SEQ ID NO: 222 | SEQ ID NO: 218 | SEQ ID NO: 223 |
| | CTGCGCCATTGCCTGAAAC | CGATGCGAGCAAGACCTTTAAGACCAAGACT | AGTTCTCCTTGATCCGTGACAAAAAA |
| | | ACG | |
| 321 | SEQ ID NO: 230 | SEQ ID NO: 218 | SEQ ID NO: 219 |
| | CCGTCCCTGCGCCATT | CGATGCGAGCAAGACCTTTAAGACCAAGACT | TTCTCCTTGATCCGTGACAAAAAAGT |
| | | ACG | |
| 322 | SEQ ID NO: 210 | SEQ ID NO: 218 | SEQ ID NO: 223 |
| | CCTGAAACGATTGAAACCAAGGTCAA | CGATGCGAGCAAGACCTTTAAGACCAAGACT | AGTTCTCCTTGATCCGTGACAAAAAA |
| | | ACG | |
| 323 | SEQ ID NO: 224 | SEQ ID NO: 211 | SEQ ID NO: 215 |
| | CCTGAAACGATTGAAACCAAGGTCAA | AGTCGATGCGAGCAAGACCTTTAAGACCAAG | AGTATAGTTCTCCTTGATCCGTGACAAA |
| | AG | ACT | |
| 324 | SEQ ID NO: 225 | SEQ ID NO: 211 | SEQ ID NO: 212 |
| | CTGAAACGATTGAAACCAAGGTCAAAG | AGTCGATGCGAGCAAGACCTTTAAGACCAAG | AAGTATAGTTCTCCTTGATCCGTGACA |
| | | ACT | |
| 325 | SEQ ID NO: 206 | SEQ ID NO: 218 | SEQ ID NO: 216 |
| | CGCCATTGCCTGAAACGATTG | CGATGCGAGCAAGACCTTTAAGACCAAGACT | GTATAGTTCTCCTTGATCCGTGACAAAAA |
| | | ACG | |
| 326 | SEQ ID NO: 210 | SEQ ID NO: 218 | SEQ ID NO: 209 |
| | CCTGAAACGATTGAAACCAAGGTCAA | CGATGCGAGCAAGACCTTTAAGACCAAGACT | AGTATAGTTCTCCTTGATCCGTGACA |
| | | ACG | |
| 327 | SEQ ID NO: 210 | SEQ ID NO: 218 | SEQ ID NO: 227 |
| | CCTGAAACGATTGAAACCAAGGTCAA | CGATGCGAGCAAGACCTTTAAGACCAAGACT | TATAGTTCTCCTTGATCCGTGACAAAAAAG |
| | | ACG | |
| 328 | SEQ ID NO: 231 | SEQ ID NO: 211 | SEQ ID NO: 215 |
| | TGAAACGATTGAAACCAAGGTCAAAGA | AGTCGATGCGAGCAAGACCTTTAAGACCAAG | AGTATAGTTCTCCTTGATCCGTGACAAA |
| | | ACT | |
| 329 | SEQ ID NO: 222 | SEQ ID NO: 218 | SEQ ID NO: 227 |
| | CTGCGCCATTGCCTGAAAC | CGATGCGAGCAAGACCTTTAAGACCAAGACT | TATAGTTCTCCTTGATCCGTGACAAAAAAG |
| | | ACG | |
| 330 | SEQ ID NO: 217 | SEQ ID NO: 218 | SEQ ID NO: 216 |
| | CGCCATTGCCTGAAACGATTGAA | CGATGCGAGCAAGACCTTTAAGACCAAGACT | GTATAGTTCTCCTTGATCCGTGACAAAAA |
| | | ACG | |
| 331 | SEQ ID NO: 229 | SEQ ID NO: 211 | SEQ ID NO: 209 |
| | CTGAAACGATTGAAACCAAGGTCAAA | AGTCGATGCGAGCAAGACCTTTAAGACCAAG | AGTATAGTTCTCCTTGATCCGTGACA |
| | GA | ACT | |
| 332 | SEQ ID NO: 226 | SEQ ID NO: 211 | SEQ ID NO: 212 |
| | CTGAAACGATTGAAACCAAGGTCAAA | AGTCGATGCGAGCAAGACCTTTAAGACCAAG | AAGTATAGTTCTCCTTGATCCGTGACA |
| | GAA | ACT | |
| 333 | SEQ ID NO: 217 | SEQ ID NO: 218 | SEQ ID NO: 209 |
| | CGCCATTGCCTGAAACGATTGAA | CGATGCGAGCAAGACCTTTAAGACCAAGACT | AGTATAGTTCTCCTTGATCCGTGACA |
| | | ACG | |
| 334 | SEQ ID NO: 220 | SEQ ID NO: 218 | SEQ ID NO: 227 |
| | GCGCCATTGCCTGAAACG | CGATGCGAGCAAGACCTTTAAGACCAAGACT | TATAGTTCTCCTTGATCCGTGACAAAAAAG |
| | | ACG | |
| 335 | SEQ ID NO: 224 | SEQ ID NO: 211 | SEQ ID NO: 212 |
| | CCTGAAACGATTGAAACCAAGGTCAA | AGTCGATGCGAGCAAGACCTTTAAGACCAAG | AAGTATAGTTCTCCTTGATCCGTGACA |
| | AG | ACT | |
| 336 | SEQ ID NO: 225 | SEQ ID NO: 211 | SEQ ID NO: 209 |
| | CTGAAACGATTGAAACCAAGGTCAAAG | AGTCGATGCGAGCAAGACCTTTAAGACCAAG | AGTATAGTTCTCCTTGATCCGTGACA |
| | | ACT | |
| 337 | SEQ ID NO: 220 | SEQ ID NO: 218 | SEQ ID NO: 209 |
| | GCGCCATTGCCTGAAACG | CGATGCGAGCAAGACCTTTAAGACCAAGACT | AGTATAGTTCTCCTTGATCCGTGACA |
| | | ACG | |
| 338 | SEQ ID NO: 221 | SEQ ID NO: 218 | SEQ ID NO: 227 |
| | GCCATTGCCTGAAACGATTGAAAC | CGATGCGAGCAAGACCTTTAAGACCAAGACT | TATAGTTCTCCTTGATCCGTGACAAAAAAG |
| | | ACG | |
| 339 | SEQ ID NO: 231 | SEQ ID NO: 211 | SEQ ID NO: 212 |
| | TGAAACGATTGAAACCAAGGTCAAAGA | AGTCGATGCGAGCAAGACCTTTAAGACCAAG | AAGTATAGTTCTCCTTGATCCGTGACA |
| | | ACT | |
| 340 | SEQ ID NO: 232 | SEQ ID NO: 211 | SEQ ID NO: 216 |
| | ACGATTGAAACCAAGGTCAAAGAACA | AGTCGATGCGAGCAAGACCTTTAAGACCAAG | GTATAGTTCTCCTTGATCCGTGACAAAAA |
| | AG | ACT | |
| 341 | SEQ ID NO: 233 | SEQ ID NO: 211 | SEQ ID NO: 212 |
| | TGAAACGATTGAAACCAAGGTCAAAG | AGTCGATGCGAGCAAGACCTTTAAGACCAAG | AAGTATAGTTCTCCTTGATCCGTGACA |
| | AAC | ACT | |
| 342 | SEQ ID NO: 234 | SEQ ID NO: 211 | SEQ ID NO: 215 |
| | TGAAACGATTGAAACCAAGGTCAAAG | AGTCGATGCGAGCAAGACCTTTAAGACCAAG | AGTATAGTTCTCCTTGATCCGTGACAAA |
| | AACA | ACT | |
| 343 | SEQ ID NO: 235 | SEQ ID NO: 207 | SEQ ID NO: 239 |
| | AACGATTGAAACCAAGGTCAAAGAACA | AGTCTTGGTCTTAAAGGTCTTGCTCGCATCGA | AAGTATAGTTCTCCTTGATCCGTGACAAA |
| | | CT | |
| 344 | SEQ ID NO: 236 | SEQ ID NO: 211 | SEQ ID NO: 216 |
| | ACGATTGAAACCAAGGTCAAAGAACAA | AGTCGATGCGAGCAAGACCTTTAAGACCAAG | GTATAGTTCTCCTTGATCCGTGACAAAAA |
| | | ACT | |
| 345 | SEQ ID NO: 237 | SEQ ID NO: 211 | SEQ ID NO: 215 |
| | GAAACGATTGAAACCAAGGTCAAAGA | AGTCGATGCGAGCAAGACCTTTAAGACCAAG | AGTATAGTTCTCCTTGATCCGTGACAAA |
| | AC | ACT | |
| 346 | SEQ ID NO: 238 | SEQ ID NO: 211 | SEQ ID NO: 215 |
| | GAAACGATTGAAACCAAGGTCAAAGA | AGTCGATGCGAGCAAGACCTTTAAGACCAAG | AGTATAGTTCTCCTTGATCCGTGACAAA |
| | ACA | ACT | |
| 347 | SEQ ID NO: 240 | SEQ ID NO: 211 | SEQ ID NO: 216 |
| | AAACGATTGAAACCAAGGTCAAAGAA | AGTCGATGCGAGCAAGACCTTTAAGACCAAG | GTATAGTTCTCCTTGATCCGTGACAAAAA |
| | CAAG | ACT | |
| 348 | SEQ ID NO: 235 | SEQ ID NO: 211 | SEQ ID NO: 215 |
| | AACGATTGAAACCAAGGTCAAAGAACA | AGTCGATGCGAGCAAGA | AGTATAGTTCTCCTTGATCCGTGACAAA |
| | | ACT | |
| 349 | SEQ ID NO: 237 | SEQ ID NO: 211 | SEQ ID NO: 209 |
| | GAAACGATTGAAACCAAGGTCAAAGA | AGTCGATGCGAGCAAGACCTTTAAGACCAAG | AGTATAGTTCTCCTTGATCCGTGACA |
| | AC | ACT | |
| 350 | SEQ ID NO: 235 | SEQ ID NO: 207 | SEQ ID NO: 241 |
| | AACGATTGAAACCAAGGTCAAAGAACA | AGTCTTGGTCTTAAAGGTCTTGCTCGCATCGA | AGTATAGTTCTCCTTGATCCGTGACAAAAA |
| | | CT | |
| 351 | SEQ ID NO: 242 | SEQ ID NO: 211 | SEQ ID NO: 216 |
| | AAACGATTGAAACCAAGGTCAAAGAA | AGTCGATGCGAGCAAGACCTTTAAGACCAAG | GTATAGTTCTCCTTGATCCGTGACAAAAA |
| | CA | ACT | |
| 352 | SEQ ID NO: 243 | SEQ ID NO: 211 | SEQ ID NO: 215 |
| | AAACGATTGAAACCAAGGTCAAAGAA | AGTCGATGCGAGCAAGACCTTTAAGACCAAG | AGTATAGTTCTCCTTGATCCGTGACAAA |
| | CAA | ACT | |

vanG Sets

| | | | |
|---|---|---|---|
| 353 | SEQ ID NO: 244 | SEQ ID NO: 245 | SEQ ID NO: 246 |
| | TCTGGCGAAATTGTATTTAATGAGGTA | CAATACCAGGCTTTACCTCGCACAGTCGC | AGACCAATGCCTTTCATCATATTTGGA |
| | AACA | | |
| 354 | SEQ ID NO: 244 | SEQ ID NO: 245 | SEQ ID NO: 247 |
| | TCTGGCGAAATTGTATTTAATGAGGTA | CAATACCAGGCTTTACCTCGCACAGTCGC | GATAGACCAATGCCTTTCATCATATTTGGATA |
| | AACA | | |
| 355 | SEQ ID NO: 244 | SEQ ID NO: 245 | SEQ ID NO: 248 |
| | TCTGGCGAAATTGTATTTAATGAGGTA | CAATACCAGGCTTTACCTCGCACAGTCGC | GATAGACCAATGCCTTTCATCATATTTGGA |
| | AACA | | |
| 356 | SEQ ID NO: 249 | SEQ ID NO: 245 | SEQ ID NO: 246 |
| | ACCGTCTGGCGAAATTGTATTTAATGA | CAATACCAGGCTTTACCTCGCACAGTCGC | AGACCAATGCCTTTCATCATATTTGGA |
| 357 | SEQ ID NO: 244 | SEQ ID NO: 245 | SEQ ID NO: 250 |
| | TCTGGCGAAATTGTATTTAATGAGGTA | CAATACCAGGCTTTACCTCGCACAGTCGC | CGATAGACCAATGCCTTTCATCATATTTGG |
| | AACA | | |
| 358 | SEQ ID NO: 244 | SEQ ID NO: 245 | SEQ ID NO: 251 |
| | TCTGGCGAAATTGTATTTAATGAGGTA | CAATACCAGGCTTTACCTCGCACAGTCGC | CGATAGACCAATGCCTTTCATCATATTTG |
| | AACA | | GATA |
| 359 | SEQ ID NO: 252 | SEQ ID NO: 245 | SEQ ID NO: 246 |
| | CACCGTCTGGCGAAATTGTATTTAATG | CAATACCAGGCTTTACCTCGCACAGTCGC | AGACCAATGCCTTTCATCATATTTGGA |
| 360 | SEQ ID NO: 253 | SEQ ID NO: 245 | SEQ ID NO: 246 |
| | ACACCGTCTGGCGAAATTGTAT | CAATACCAGGCTTTACCTCGCACAGTCGC | AGACCAATGCCTTTCATCATATTTGGA |
| 361 | SEQ ID NO: 244 | SEQ ID NO: 245 | SEQ ID NO: 254 |
| | TCTGGCGAAATTGTATTTAATGAGGTA | CAATACCAGGCTTTACCTCGCACAGTCGC | AACGATAGACCAATGCCTTTCATCATATTT |
| | AACA | | |
| 362 | SEQ ID NO: 255 | SEQ ID NO: 245 | SEQ ID NO: 246 |
| | ACACCGTCTGGCGAAATTGTATTTA | CAATACCAGGCTTTACCTCGCACAGTCGC | AGACCAATGCCTTTCATCATATTTGGA |
| 363 | SEQ ID NO: 256 | SEQ ID NO: 245 | SEQ ID NO: 246 |
| | ACACCGTCTGGCGAAATTGTATT | CAATACCAGGCTTTACCTCGCACAGTCGC | AGACCAATGCCTTTCATCATATTTGGA |
| 364 | SEQ ID NO: 249 | SEQ ID NO: 245 | SEQ ID NO: 248 |
| | ACCGTCTGGCGAAATTGTATTTAATGA | CAATACCAGGCTTTACCTCGCACAGTCGC | GATAGACCAATGCCTTTCATCATATTTGGA |
| 365 | SEQ ID NO: 244 | SEQ ID NO: 257 | SEQ ID NO: 258 |
| | TCTGGCGAAATTGTATTTAATGAGGTA | AGGCTTTACCTCGCACAGTCGCTATCCAA | GAACGATAGACCAATGCCTTTCATCA |
| | AACA | | |
| 366 | SEQ ID NO: 244 | SEQ ID NO: 257 | SEQ ID NO: 259 |
| | TCTGGCGAAATTGTATTTAATGAGGTA | AGGCTTTACCTCGCACAGTCGCTATCCAA | GAACGATAGACCAATGCCTTTCATCATA |
| | AACA | | |
| 367 | SEQ ID NO: 260 | SEQ ID NO: 261 | SEQ ID NO: 258 |
| | ACCGTCTGGCGAAATTGTATTTAATGAG | CCAGGCTTTACCTCGCACAGTCGCTATCC | GAACGATAGACCAATGCCTTTCATCA |
| 368 | SEQ ID NO: 252 | SEQ ID NO: 257 | SEQ ID NO: 262 |
| | CACCGTCTGGCGAAATTGTATTTAATG | AGGCTTTACCTCGCACAGTCGCTATCCAA | GAACGATAGACCAATGCCTTTCATCAT |
| 369 | SEQ ID NO: 263 | SEQ ID NO: 257 | SEQ ID NO: 258 |
| | TTTATACACCGTCTGGCGAAATTGT | AGGCTTTACCTCGCACAGTCGCTATCCAA | GAACGATAGACCAATGCCTTTCATCA |
| 370 | SEQ ID NO: 264 | SEQ ID NO: 265 | SEQ ID NO: 266 |
| | GCCGAAGCAGAAAAACGGATACA | CCACTCTGGAAAAACCCGAACAGCCCAGA | CAATTTCGCCAGACGGTGTATAAAACATA |
| 371 | SEQ ID NO: 267 | SEQ ID NO: 268 | SEQ ID NO: 269 |
| | GCCGAAGCAGAAAAACGGATACAA | TATCCACTCTGGAAAAACCCGAACAGCCCA | ACAATTTCGCCAGACGGTGTATAAAA |
| 372 | SEQ ID NO: 270 | SEQ ID NO: 265 | SEQ ID NO: 266 |
| | TGCCGAAGCAGAAAAACGGATAC | CCACTCTGGAAAAACCCGAACAGCCCAGA | CAATTTCGCCAGACGGTGTATAAAACATA |
| 373 | SEQ ID NO: 270 | SEQ ID NO: 268 | SEQ ID NO: 269 |
| | TGCCGAAGCAGAAAAACGGATAC | TATCCACTCTGGAAAAACCCGAACAGCCCA | ACAATTTCGCCAGACGGTGTATAAAA |
| 374 | SEQ ID NO: 271 | SEQ ID NO: 265 | SEQ ID NO: 266 |
| | ATGCCGAAGCAGAAAAACGGATAC | CCACTCTGGAAAAACCCGAACAGCCCAGA | CAATTTCGCCAGACGGTGTATAAAACATA |
| 375 | SEQ ID NO: 272 | SEQ ID NO: 273 | SEQ ID NO: 274 |
| | AAGGATTGATGCCGAAGCAGAA | CTGGAAAAACCCGAACAGCCCAGAGCTT | GCCAGACGGTGTATAAAACATATCCA |
| 376 | SEQ ID NO: 275 | SEQ ID NO: 268 | SEQ ID NO: 269 |
| | ATGCCGAAGCAGAAAAACGGATA | TATCCACTCTGGAAAAACCCGAACAGCCCA | ACAATTTCGCCAGACGGTGTATAAAA |
| 377 | SEQ ID NO: 276 | SEQ ID NO: 268 | SEQ ID NO: 269 |
| | GATGCCGAAGCAGAAAAACGGATA | TATCCACTCTGGAAAAACCCGAACAGCCCA | ACAATTTCGCCAGACGGTGTATAAAA |
| 378 | SEQ ID NO: 277 | SEQ ID NO: 268 | SEQ ID NO: 269 |
| | AGGATTGATGCCGAAGCAGAAAA | TATCCACTCTGGAAAAACCCGAACAGCCCA | ACAATTTCGCCAGACGGTGTATAAAA |
| 379 | SEQ ID NO: 278 | SEQ ID NO: 265 | SEQ ID NO: 266 |
| | AAGGATTGATGCCGAAGCAGAAAAAC | CCACTCTGGAAAAACCCGAACAGCCCAGA | CAATTTCGCCAGACGGTGTATAAAACATA |
| 380 | SEQ ID NO: 279 | SEQ ID NO: 268 | SEQ ID NO: 269 |
| | AAGGATTGATGCCGAAGCAGAAA | TATCCACTCTGGAAAAACCCGAACAGCCCA | ACAATTTCGCCAGACGGTGTATAAAA |
| 381 | SEQ ID NO: 280 | SEQ ID NO: 265 | SEQ ID NO: 266 |
| | CAAGGATTGATGCCGAAGCAGAA | CCACTCTGGAAAAACCCGAACAGCCCAGA | CAATTTCGCCAGACGGTGTATAAAACATA |
| 382 | SEQ ID NO: 281 | SEQ ID NO: 268 | SEQ ID NO: 269 |
| | AAGGATTGATGCCGAAGCAGAAAA | TATCCACTCTGGAAAAACCCGAACAGCCCA | ACAATTTCGCCAGACGGTGTATAAAA |
| 383 | SEQ ID NO: 280 | SEQ ID NO: 268 | SEQ ID NO: 269 |
| | CAAGGATTGATGCCGAAGCAGAA | TATCCACTCTGGAAAAACCCGAACAGCCCA | ACAATTTCGCCAGACGGTGTATAAAA |
| 384 | SEQ ID NO: 282 | SEQ ID NO: 265 | SEQ ID NO: 266 |
| | GCAAGGATTGATGCCGAAGCA | CCACTCTGGAAAAACCCGAACAGCCCAGA | CAATTTCGCCAGACGGTGTATAAAACATA |
| 385 | SEQ ID NO: 283 | SEQ ID NO: 268 | SEQ ID NO: 269 |
| | CAAGGATTGATGCCGAAGCAGAAA | TATCCACTCTGGAAAAACCCGAACAGCCCA | ACAATTTCGCCAGACGGTGTATAAAA |
| 386 | SEQ ID NO: 282 | SEQ ID NO: 268 | SEQ ID NO: 269 |
| | GCAAGGATTGATGCCGAAGCA | TATCCACTCTGGAAAAACCCGAACAGCCCA | ACAATTTCGCCAGACGGTGTATAAAA |
| 387 | SEQ ID NO: 244 | SEQ ID NO: 257 | SEQ ID NO: 284 |
| | TCTGGCGAAATTGTATTTAATGAGGTA | AGGCTTTACCTCGCACAGTCGCTATCCAA | ACATACAGACCTATCAGCTTATCCAACA |
| | AACA | | |
| 388 | SEQ ID NO: 285 | SEQ ID NO: 257 | SEQ ID NO: 259 |
| | CGGGTTTTTCCAGAGTGGATATGT | AGGCTTTACCTCGCACAGTCGCTATCCAA | GAACGATAGACCAATGCCTTTCATCATA |
| 389 | SEQ ID NO: 244 | SEQ ID NO: 257 | SEQ ID NO: 286 |
| | TCTGGCGAAATTGTATTTAATGAGGTA | AGGCTTTACCTCGCACAGTCGCTATCCAA | ACATACAGACCTATCAGCTTATCCAACATT |
| | AACA | | |
| 390 | SEQ ID NO: 244 | SEQ ID NO: 257 | SEQ ID NO: 287 |
| | TCTGGCGAAATTGTATTTAATGAGGTA | AGGCTTTACCTCGCACAGTCGCTATCCAA | ACATACAGACCTATCAGCTTATCCAACAT |
| | AACA | | |
| 391 | SEQ ID NO: 288 | SEQ ID NO: 289 | SEQ ID NO: 290 |
| | TCAAGCGGCTTTTTTGATTATACAGAG | TTTCTGCTTCGGCATCAATCCTTGCAGGC | CAGCCCAGAGCTTTATATATGGTTACAG |
| | AAAT | | |
| 392 | SEQ ID NO: 291 | SEQ ID NO: 292 | SEQ ID NO: 290 |
| | TCAAGCGGCTTTTTTGATTATACAGAG | TTCTGCTTCGGCATCAATCCTTGCAGGC | CAGCCCAGAGCTTTATATATGGTTACAG |
| | AAATA | | |
| 393 | SEQ ID NO: 293 | SEQ ID NO: 294 | SEQ ID NO: 290 |
| | TCAAGCGGCTTTTTTGATTATACAGAGA | TTTTCTGCTTCGGCATCAATCCTTGCAGGC | CAGCCCAGAGCTTTATATATGGTTACAG |
| 394 | SEQ ID NO: 295 | SEQ ID NO: 296 | SEQ ID NO: 258 |
| | GTTCGGGTTTTTCCAGAGTGGAT | ACCAGGCTTTACCTCGCACAGTCGC | GAACGATAGACCAATGCCTTTCATCA |
| 395 | SEQ ID NO: 293 | SEQ ID NO: 289 | SEQ ID NO: 290 |
| | TCAAGCGGCTTTTTTGATTATACAGAGA | TTTCTGCTTCGGCATCAATCCTTGCAGGC | CAGCCCAGAGCTTTATATATGGTTACAG |
| 396 | SEQ ID NO: 288 | SEQ ID NO: 294 | SEQ ID NO: 290 |
| | TCAAGCGGCTTTTTTGATTATACAGAG | TTTTCTGCTTCGGCATCAATCCTTGCAGGC | CAGCCCAGAGCTTTATATATGGTTACAG |
| | AAAT | | |
| 397 | SEQ ID NO: 297 | SEQ ID NO: 294 | SEQ ID NO: 290 |
| | TCAAGCGGCTTTTTTGATTATACAGAG | TTTTCTGCTTCGGCATCAATCCTTGCAGGC | CAGCCCAGAGCTTTATATATGGTTACAG |
| | AAATATAC | | |
| 398 | SEQ ID NO: 291 | SEQ ID NO: 289 | SEQ ID NO: 290 |
| | TCAAGCGGCTTTTTTGATTATACAGAG | TTTCTGCTTCGGCATCAATCCTTGCAGGC | CAGCCCAGAGCTTTATATATGGTTACAG |
| | AAATA | | |
| 399 | SEQ ID NO: 249 | SEQ ID NO: 257 | SEQ ID NO: 286 |
| | ACCGTCTGGCGAAATTGTATTTAATGA | AGGCTTTACCTCGCACAGTCGCTATCCAA | ACATACAGACCTATCAGCTTATCCAACATT |
| 400 | SEQ ID NO: 298 | SEQ ID NO: 292 | SEQ ID NO: 290 |
| | GTCAAGCGGCTTTTTTGATTATACAGA | TTCTGCTTCGGCATCAATCCTTGCAGGC | CAGCCCAGAGCTTTATATATGGTTACAG |
| | GA | | |
| 401 | SEQ ID NO: 298 | SEQ ID NO: 294 | SEQ ID NO: 290 |
| | GTCAAGCGGCTTTTTTGATTATACAGA | TTTTCTGCTTCGGCATCAATCCTTGCAGGC | CAGCCCAGAGCTTTATATATGGTTACAG |
| | GA | | |
| 402 | SEQ ID NO: 298 | SEQ ID NO: 289 | SEQ ID NO: 290 |
| | GTCAAGCGGCTTTTTTGATTATACAGA | TTTCTGCTTCGGCATCAATCCTTGCAGGC | CAGCCCAGAGCTTTATATATGGTTACAG |
| | GA | | |
| 403 | SEQ ID NO: 299 | SEQ ID NO: 294 | SEQ ID NO: 290 |
| | TGTCAAGCGGCTTTTTTGATTATACAGA | TTTTCTGCTTCGGCATCAATCCTTGCAGGC | CAGCCCAGAGCTTTATATATGGTTACAG |
| 404 | SEQ ID NO: 300 | SEQ ID NO: 289 | SEQ ID NO: 290 |
| | TGTCAAGCGGCTTTTTTGATTATACAG | TTTCTGCTTCGGCATCAATCCTTGCAGGC | CAGCCCAGAGCTTTATATATGGTTACAG |
| | AGA | | |
| 405 | SEQ ID NO: 249 | SEQ ID NO: 257 | SEQ ID NO: 301 |
| | ACCGTCTGGCGAAATTGTATTTAATGA | AGGCTTTACCTCGCACAGTCGCTATCCAA | CACATACAGACCTATCAGCTTATCCAACATT |
| 406 | SEQ ID NO: 299 | SEQ ID NO: 289 | SEQ ID NO: 290 |
| | TGTCAAGCGGCTTTTTTGATTATACAGA | TTTCTGCTTCGGCATCAATCCTTGCAGGC | CAGCCCAGAGCTTTATATATGGTTACAG |
| 407 | SEQ ID NO: 300 | SEQ ID NO: 292 | SEQ ID NO: 290 |
| | TGTCAAGCGGCTTTTTTGATTATACAG | TTCTGCTTCGGCATCAATCCTTGCAGGC | CAGCCCAGAGCTTTATATATGGTTACAG |
| | AGA | | |
| 408 | SEQ ID NO: 302 | SEQ ID NO: 273 | SEQ ID NO: 274 |
| | ATCTTCAAAGATATATATGCCTGCAAG | CTGGAAAAACCCGAACAGCCCAGAGCTT | GCCAGACGGTGTATAAAACATATCCA |
| | GATTG | | |
| 409 | SEQ ID NO: 299 | SEQ ID NO: 292 | SEQ ID NO: 290 |
| | TGTCAAGCGGCTTTTTTGATTATACAGA | TTCTGCTTCGGCATCAATCCTTGCAGGC | CAGCCCAGAGCTTTATATATGGTTACAG |
| 410 | SEQ ID NO: 300 | SEQ ID NO: 294 | SEQ ID NO: 290 |
| | TGTCAAGCGGCTTTTTTGATTATACAG | TTTTCTGCTTCGGCATCAATCCTTGCAGGC | CAGCCCAGAGCTTTATATATGGTTACAG |
| | AGA | | |
| 411 | SEQ ID NO: 303 | SEQ ID NO: 292 | SEQ ID NO: 290 |
| | CTGTCAAGCGGCTTTTTTGATTATACAG | TTCTGCTTCGGCATCAATCCTTGCAGGC | CAGCCCAGAGCTTTATATATGGTTACAG |
| 412 | SEQ ID NO: 293 | SEQ ID NO: 294 | SEQ ID NO: 304 |
| | TCAAGCGGCTTTTTTGATTATACAGAGA | TTTTCTGCTTCGGCATCAATCCTTGCAGGC | GAACAGCCCAGAGCTTTATATATGGTTAC |
| 413 | SEQ ID NO: 305 | SEQ ID NO: 294 | SEQ ID NO: 290 |
| | CTGTCAAGCGGCTTTTTTGATTATACA | TTTTCTGCTTCGGCATCAATCCTTGCAGGC | CAGCCCAGAGCTTTATATATGGTTACAG |
| | GA | | |
| 414 | SEQ ID NO: 288 | SEQ ID NO: 289 | SEQ ID NO: 304 |
| | TCAAGCGGCTTTTTTGATTATACAGAG | TTTCTGCTTCGGCATCAATCCTTGCAGGC | GAACAGCCCAGAGCTTTATATATGGTTAC |
| | AAAT | | |
| 415 | SEQ ID NO: 305 | SEQ ID NO: 289 | SEQ ID NO: 290 |
| | CTGTCAAGCGGCTTTTTTGATTATACA | TTTCTGCTTCGGCATCAATCCTTGCAGGC | CAGCCCAGAGCTTTATATATGGTTACAG |
| | GA | | |
| 416 | SEQ ID NO: 303 | SEQ ID NO: 294 | SEQ ID NO: 290 |
| | CTGTCAAGCGGCTTTTTTGATTATACAG | TTTTCTGCTTCGGCATCAATCCTTGCAGGC | CAGCCCAGAGCTTTATATATGGTTACAG |
| 417 | SEQ ID NO: 249 | SEQ ID NO: 257 | SEQ ID NO: 306 |
| | ACCGTCTGGCGAAATTGTATTTAATGA | AGGCTTTACCTCGCACAGTCGCTATCCAA | CCACATACAGACCTATCAGCTTATCCA |
| 418 | SEQ ID NO: 298 | SEQ ID NO: 289 | SEQ ID NO: 304 |
| | GTCAAGCGGCTTTTTTGATTATACAGA | TTTCTGCTTCGGCATCAATCCTTGCAGGC | GAACAGCCCAGAGCTTTATATATGGTTAC |
| | GA | | |
| 419 | SEQ ID NO: 307 | SEQ ID NO: 292 | SEQ ID NO: 290 |
| | ACTGTCAAGCGGCTTTTTTGATTATACA | TTCTGCTTCGGCATCAATCCTTGCAGGC | CAGCCCAGAGCTTTATATATGGTTACAG |
| 420 | SEQ ID NO: 249 | SEQ ID NO: 257 | SEQ ID NO: 308 |
| | ACCGTCTGGCGAAATTGTATTTAATGA | AGGCTTTACCTCGCACAGTCGCTATCCAA | TCCACATACAGACCTATCAGCTTATCC |
| 421 | SEQ ID NO: 309 | SEQ ID NO: 294 | SEQ ID NO: 290 |
| | ACTGTCAAGCGGCTTTTTTGATTATAC | TTTTCTGCTTCGGCATCAATCCTTGCAGGC | CAGCCCAGAGCTTTATATATGGTTACAG |
| | AG | | |
| 422 | SEQ ID NO: 310 | SEQ ID NO: 289 | SEQ ID NO: 290 |
| | ACTGTCAAGCGGCTTTTTTGATTATAC | TTTCTGCTTCGGCATCAATCCTTGCAGGC | CAGCCCAGAGCTTTATATATGGTTACAG |
| | AGA | | |
| 423 | SEQ ID NO: 309 | SEQ ID NO: 292 | SEQ ID NO: 290 |
| | ACTGTCAAGCGGCTTTTTTGATTATAC | TTCTGCTTCGGCATCAATCCTTGCAGGC | CAGCCCAGAGCTTTATATATGGTTACAG |
| | AG | | |
| 424 | SEQ ID NO: 311 | SEQ ID NO: 261 | SEQ ID NO: 258 |
| | GGGCTGTTCGGGTTTTTCCA | CCAGGCTTTACCTCGCACAGTCGCTATCC | GAACGATAGACCAATGCCTTTCATCA |
| 425 | SEQ ID NO: 249 | SEQ ID NO: 257 | SEQ ID NO: 312 |
| | ACCGTCTGGCGAAATTGTATTTAATGA | AGGCTTTACCTCGCACAGTCGCTATCCAA | TTCCACATACAGACCTATCAGCTTATCC |
| 426 | SEQ ID NO: 313 | SEQ ID NO: 292 | SEQ ID NO: 290 |
| | AACTGTCAAGCGGCTTTTTTGATTATA | TTCTGCTTCGGCATCAATCCTTGCAGGC | CAGCCCAGAGCTTTATATATGGTTACAG |
| | CA | | |
| 427 | SEQ ID NO: 314 | SEQ ID NO: 292 | SEQ ID NO: 290 |
| | AACTGTCAAGCGGCTTTTTTGATTATA | TTCTGCTTCGGCATCAATCCTTGCAGGC | CAGCCCAGAGCTTTATATATGGTTACAG |
| | CAG | | |
| 428 | SEQ ID NO: 303 | SEQ ID NO: 292 | SEQ ID NO: 304 |
| | CTGTCAAGCGGCTTTTTTGATTATACAG | TTCTGCTTCGGCATCAATCCTTGCAGGC | GAACAGCCCAGAGCTTTATATATGGTTAC |
| 429 | SEQ ID NO: 315 | SEQ ID NO: 292 | SEQ ID NO: 290 |
| | GAACTGTCAAGCGGCTTTTTTGATTAT | TTCTGCTTCGGCATCAATCCTTGCAGGC | CAGCCCAGAGCTTTATATATGGTTACAG |
| | AC | | |
| 430 | SEQ ID NO: 316 | SEQ ID NO: 292 | SEQ ID NO: 290 |
| | CGAACTGTCAAGCGGCTTTTTTGAT | TTCTGCTTCGGCATCAATCCTTGCAGGC | CAGCCCAGAGCTTTATATATGGTTACAG |
| 431 | SEQ ID NO: 317 | SEQ ID NO: 292 | SEQ ID NO: 290 |
| | TCGAACTGTCAAGCGGCTTTTTT | TTCTGCTTCGGCATCAATCCTTGCAGGC | CAGCCCAGAGCTTTATATATGGTTACAG |
| 432 | SEQ ID NO: 318 | SEQ ID NO: 292 | SEQ ID NO: 290 |
| | ATCGAACTGTCAAGCGGCTTTTT | TTCTGCTTCGGCATCAATCCTTGCAGGC | CAGCCCAGAGCTTTATATATGGTTACAG |
| 433 | SEQ ID NO: 314 | SEQ ID NO: 319 | SEQ ID NO: 320 |
| | AACTGTCAAGCGGCTTTTTTGATTATA | TCTGCTTCGGCATCAATCCTTGCAGGC | CCGAACAGCCCAGAGCTTTA |
| | CAG | | |
| 434 | SEQ ID NO: 321 | SEQ ID NO: 261 | SEQ ID NO: 258 |
| | AACCATATATAAAGCTCTGGGCTGTTC | CCAGGCTTTACCTCGCACAGTCGCTATCC | GAACGATAGACCAATGCCTTTCATCA |
| 435 | SEQ ID NO: 322 | SEQ ID NO: 323 | SEQ ID NO: 324 |
| | CTGCAAGGATTGATGCCGAAGCA | ATCCACTCTGGAAAAACCCGAACAGCCCAGA | GGATAGCGACTGTGCGAGGTA |
| 436 | SEQ ID NO: 325 | SEQ ID NO: 273 | SEQ ID NO: 274 |
| | GTCAAGCGGCTTTTTTGATTATACAGA | CTGGAAAAACCCGAACAGCCCAGAGCTT | GCCAGACGGTGTATAAAACATATCCA |
| | GAAATA | | |
| 437 | SEQ ID NO: 305 | SEQ ID NO: 273 | SEQ ID NO: 274 |
| | CTGTCAAGCGGCTTTTTTGATTATACA | CTGGAAAAACCCGAACAGCCCAGAGCTT | GCCAGACGGTGTATAAAACATATCCA |
| | GA | | |
| 438 | SEQ ID NO: 307 | SEQ ID NO: 273 | SEQ ID NO: 274 |
| | ACTGTCAAGCGGCTTTTTTGATTATACA | CTGGAAAAACCCGAACAGCCCAGAGCTT | GCCAGACGGTGTATAAAACATATCCA |
| 439 | SEQ ID NO: 326 | SEQ ID NO: 273 | SEQ ID NO: 274 |
| | CGAACTGTCAAGCGGCTTTTTTGATTA | CTGGAAAAACCCGAACAGCCCAGAGCTT | GCCAGACGGTGTATAAAACATATCCA |
| 440 | SEQ ID NO: 327 | SEQ ID NO: 265 | SEQ ID NO: 266 |
| | GTCAAGCGGCTTTTTTGATTATACAGA | CCACTCTGGAAAAACCCGAACAGCCCAGA | CAATTTCGCCAGACGGTGTATAAAACATA |
| | GAAAT | | |
| 441 | SEQ ID NO: 325 | SEQ ID NO: 265 | SEQ ID NO: 266 |
| | GTCAAGCGGCTTTTTTGATTATACAGA | CCACTCTGGAAAAACCCGAACAGCCCAGA | CAATTTCGCCAGACGGTGTATAAAACATA |
| | GAAATA | | |
| 442 | SEQ ID NO: 328 | SEQ ID NO: 273 | SEQ ID NO: 274 |
| | AATCGAACTGTCAAGCGGCTTTTTT | CTGGAAAAACCCGAACAGCCCAGAGCTT | GCCAGACGGTGTATAAAACATATCCA |
| 443 | SEQ ID NO: 329 | SEQ ID NO: 273 | SEQ ID NO: 274 |
| | AAATCGAACTGTCAAGCGGCTTTTT | CTGGAAAAACCCGAACAGCCCAGAGCTT | GCCAGACGGTGTATAAAACATATCCA |
| 444 | SEQ ID NO: 330 | SEQ ID NO: 273 | SEQ ID NO: 274 |
| | GAAATCGAACTGTCAAGCGGCTTTT | CTGGAAAAACCCGAACAGCCCAGAGCTT | GCCAGACGGTGTATAAAACATATCCA |
| 445 | SEQ ID NO: 331 | SEQ ID NO: 273 | SEQ ID NO: 274 |
| | GAAATCGAACTGTCAAGCGGCTTTTT | CTGGAAAAACCCGAACAGCCCAGAGCTT | GCCAGACGGTGTATAAAACATATCCA |
| 446 | SEQ ID NO: 332 | SEQ ID NO: 265 | SEQ ID NO: 266 |
| | ATCGAACTGTCAAGCGGCTTTTTT | CCACTCTGGAAAAACCCGAACAGCCCAGA | CAATTTCGCCAGACGGTGTATAAAACATA |
| 447 | SEQ ID NO: 333 | SEQ ID NO: 265 | SEQ ID NO: 266 |
| | AATCGAACTGTCAAGCGGCTTTT | CCACTCTGGAAAAACCCGAACAGCCCAGA | CAATTTCGCCAGACGGTGTATAAAACATA |

vanE Sets

| | | | |
|---|---|---|---|
| 448 | SEQ ID NO: 334 | SEQ ID NO: 335 | SEQ ID NO: 336 |
| | GGTATCGGAGCTGCAGCAAT | TCCCACAGCCAATTTCTACCCCTTTCACTTCCT | CTGATTTGGTCACATTCTCCAACGA |
| 449 | SEQ ID NO: 337 | SEQ ID NO: 335 | SEQ ID NO: 336 |
| | TGGTGTAAAAAGCACCCCTAGTATGA | TCCCACAGCCAATTTCTACCCCTTTCACTTCCT | CTGATTTGGTCACATTCTCCAACGA |
| 450 | SEQ ID NO: 338 | SEQ ID NO: 335 | SEQ ID NO: 336 |
| | AGACGAAGCTTCAAAATATGATAGCC | TCCCACAGCCAATTTCTACCCCTTTCACTTCCT | CTGATTTGGTCACATTCTCCAACGA |
| | GTAT | | |
| 451 | SEQ ID NO: 339 | SEQ ID NO: 335 | SEQ ID NO: 336 |
| | CAGCAATCTCCATGAATAAAATAATG | TCCCACAGCCAATTTCTACCCCTTTCACTTCCT | CTGATTTGGTCACATTCTCCAACGA |
| | CTCCAT | | |
| 452 | SEQ ID NO: 340 | SEQ ID NO: 335 | SEQ ID NO: 336 |
| | TGGTGTAAAAAGCACCCCTAGTATGAT | TCCCACAGCCAATTTCTACCCCTTTCACTTCCT | CTGATTTGGTCACATTCTCCAACGA |
| 453 | SEQ ID NO: 341 | SEQ ID NO: 335 | SEQ ID NO: 336 |
| | TTGGTGTAAAAAGCACCCCTAGTATGA | TCCCACAGCCAATTTCTACCCCTTTCACTTCCT | CTGATTTGGTCACATTCTCCAACGA |
| 454 | SEQ ID NO: 342 | SEQ ID NO: 335 | SEQ ID NO: 336 |
| | TTGGTGTAAAAAGCACCCCTAGTATGAT | TCCCACAGCCAATTTCTACCCCTTTCACTTCCT | CTGATTTGGTCACATTCTCCAACGA |
| 455 | SEQ ID NO: 343 | SEQ ID NO: 335 | SEQ ID NO: 336 |
| | TGGTGTAAAAAGCACCCCTAGTATGAT | TCCCACAGCCAATTTCTACCCCTTTCACTTCCT | CTGATTTGGTCACATTCTCCAACGA |
| | TA | | |
| 456 | SEQ ID NO: 344 | SEQ ID NO: 335 | SEQ ID NO: 336 |
| | TTGGTGTAAAAAGCACCCCTAGTATGA | TCCCACAGCCAATTTCTACCCCTTTCACTTCCT | CTGATTTGGTCACATTCTCCAACGA |
| | TTA | | |
| 457 | SEQ ID NO: 345 | SEQ ID NO: 335 | SEQ ID NO: 336 |
| | ACCCCTAGTATGATTATAGAAAAGGG | TCCCACAGCCAATTTCTACCCCTTTCACTTCCT | CTGATTTGGTCACATTCTCCAACGA |
| | ACAAGA | | |
| 458 | SEQ ID NO: 346 | SEQ ID NO: 335 | SEQ ID NO: 336 |
| | AGGGACAAGACCTACAAAAAGTCGAT | TCCCACAGCCAATTTCTACCCCTTTCACTTCCT | CTGATTTGGTCACATTCTCCAACGA |
| 459 | SEQ ID NO: 347 | SEQ ID NO: 335 | SEQ ID NO: 336 |
| | AAAGGGACAAGACCTACAAAAAGTCG | TCCCACAGCCAATTTCTACCCCTTTCACTTCCT | CTGATTTGGTCACATTCTCCAACGA |
| | AT | | |
| 460 | SEQ ID NO: 348 | SEQ ID NO: 335 | SEQ ID NO: 336 |
| | TTGGTGTAAAAAGCACCCCTAGTATGA | TCCCACAGCCAATTTCTACCCCTTTCACTTCCT | CTGATTTGGTCACATTCTCCAACGA |
| | TT | | |
| 461 | SEQ ID NO: 349 | SEQ ID NO: 335 | SEQ ID NO: 336 |
| | TGCAGCAATCTCCATGAATAAAATAAT | TCCCACAGCCAATTTCTACCCCTTTCACTTCCT | CTGATTTGGTCACATTCTCCAACGA |
| | GCT | | |
| 462 | SEQ ID NO: 350 | SEQ ID NO: 335 | SEQ ID NO: 336 |
| | TAGACGAAGCTTCAAAATATGATAGC | TCCCACAGCCAATTTCTACCCCTTTCACTTCCT | CTGATTTGGTCACATTCTCCAACGA |
| | CGTAT | | |
| 463 | SEQ ID NO: 351 | SEQ ID NO: 335 | SEQ ID NO: 336 |
| | GAGCTGCAGCAATCTCCATGAATAAA | TCCCACAGCCAATTTCTACCCCTTTCACTTCCT | CTGATTTGGTCACATTCTCCAACGA |
| | AT | | |
| 464 | SEQ ID NO: 352 | SEQ ID NO: 335 | SEQ ID NO: 336 |
| | GAGCTGCAGCAATCTCCATGAATAAA | TCCCACAGCCAATTTCTACCCCTTTCACTTCCT | CTGATTTGGTCACATTCTCCAACGA |
| | ATAA | | |
| 465 | SEQ ID NO: 353 | SEQ ID NO: 335 | SEQ ID NO: 336 |
| | TGAGGCAGGCTCATCAAAAGG | TCCCACAGCCAATTTCTACCCCTTTCACTTCCT | CTGATTTGGTCACATTCTCCAACGA |
| 466 | SEQ ID NO: 354 | SEQ ID NO: 335 | SEQ ID NO: 336 |
| | TGGTGTAAAAAGCACCCCTAGTATGATT | TCCCACAGCCAATTTCTACCCCTTTCACTTCCT | CTGATTTGGTCACATTCTCCAACGA |
| 467 | SEQ ID NO: 355 | SEQ ID NO: 335 | SEQ ID NO: 336 |
| | GCAATCTCCATGAATAAAATAATGCTC | TCCCACAGCCAATTTCTACCCCTTTCACTTCCT | CTGATTTGGTCACATTCTCCAACGA |
| | CATCA | | |
| 468 | SEQ ID NO: 356 | SEQ ID NO: 335 | SEQ ID NO: 336 |
| | AGCTGCAGCAATCTCCATGAATAAAAT | TCCCACAGCCAATTTCTACCCCTTTCACTTCCT | CTGATTTGGTCACATTCTCCAACGA |
| 469 | SEQ ID NO: 357 | SEQ ID NO: 335 | SEQ ID NO: 336 |
| | AAAAGGGACAAGACCTACAAAAAGTC | TCCCACAGCCAATTTCTACCCCTTTCACTTCCT | CTGATTTGGTCACATTCTCCAACGA |
| | GAT | | |
| 470 | SEQ ID NO: 358 | SEQ ID NO: 335 | SEQ ID NO: 336 |
| | CACCCCTAGTATGATTATAGAAAAGG | TCCCACAGCCAATTTCTACCCCTTTCACTTCCT | CTGATTTGGTCACATTCTCCAACGA |
| | GACAA | | |
| 471 | SEQ ID NO: 359 | SEQ ID NO: 335 | SEQ ID NO: 336 |
| | CCCCTAGTATGATTATAGAAAAGGGA | TCCCACAGCCAATTTCTACCCCTTTCACTTCCT | CTGATTTGGTCACATTCTCCAACGA |
| | CAAGAC | | |
| 472 | SEQ ID NO: 360 | SEQ ID NO: 335 | SEQ ID NO: 336 |
| | GAGCTGCAGCAATCTCCATGAATAAA | TCCCACAGCCAATTTCTACCCCTTTCACTTCCT | CTGATTTGGTCACATTCTCCAACGA |
| | ATA | | |
| 473 | SEQ ID NO: 361 | SEQ ID NO: 335 | SEQ ID NO: 336 |
| | CCCCTAGTATGATTATAGAAAAGGGA | TCCCACAGCCAATTTCTACCCCTTTCACTTCCT | CTGATTTGGTCACATTCTCCAACGA |
| | CAAGA | | |
| 474 | SEQ ID NO: 362 | SEQ ID NO: 335 | SEQ ID NO: 336 |
| | GGCAGGCTCATCAAAAGGAATTAGC | TCCCACAGCCAATTTCTACCCCTTTCACTTCCT | CTGATTTGGTCACATTCTCCAACGA |
| 475 | SEQ ID NO: 363 | SEQ ID NO: 335 | SEQ ID NO: 336 |
| | AAGGAATTAGCAAGGTAGAACAAAAA | TCCCACAGCCAATTTCTACCCCTTTCACTTCCT | CTGATTTGGTCACATTCTCCAACGA |
| | AGTGATT | | |
| 476 | SEQ ID NO: 364 | SEQ ID NO: 335 | SEQ ID NO: 336 |
| | AGCTGCAGCAATCTCCATGAATAAAA | TCCCACAGCCAATTTCTACCCCTTTCACTTCCT | CTGATTTGGTCACATTCTCCAACGA |
| | TAAT | | |
| 477 | SEQ ID NO: 365 | SEQ ID NO: 335 | SEQ ID NO: 336 |
| | CCATCAATTTGCTGAAACAATTGGTGT | TCCCACAGCCAATTTCTACCCCTTTCACTTCCT | CTGATTTGGTCACATTCTCCAACGA |
| | AAAA | | |
| 478 | SEQ ID NO: 366 | SEQ ID NO: 335 | SEQ ID NO: 336 |
| | AGCTGCAGCAATCTCCATGAATAAAA | TCCCACAGCCAATTTCTACCCCTTTCACTTCCT | CTGATTTGGTCACATTCTCCAACGA |
| | TAA | | |
| 479 | SEQ ID NO: 367 | SEQ ID NO: 335 | SEQ ID NO: 336 |
| | CTGCAGCAATCTCCATGAATAAAATA | TCCCACAGCCAATTTCTACCCCTTTCACTTCCT | CTGATTTGGTCACATTCTCCAACGA |
| | ATGC | | |
| 480 | SEQ ID NO: 368 | SEQ ID NO: 335 | SEQ ID NO: 336 |
| | GACGAAGCTTCAAAATATGATAGCCG | TCCCACAGCCAATTTCTACCCCTTTCACTTCCT | CTGATTTGGTCACATTCTCCAACGA |
| | TAT | | |
| 481 | SEQ ID NO: 369 | SEQ ID NO: 335 | SEQ ID NO: 336 |
| | CCATCAATTTGCTGAAACAATTGGTGT | TCCCACAGCCAATTTCTACCCCTTTCACTTCCT | CTGATTTGGTCACATTCTCCAACGA |
| | AAA | | |
| 482 | SEQ ID NO: 370 | SEQ ID NO: 335 | SEQ ID NO: 336 |
| | AGCTGCAGCAATCTCCATGAATAAA | TCCCACAGCCAATTTCTACCCCTTTCACTTCCT | CTGATTTGGTCACATTCTCCAACGA |
| 483 | SEQ ID NO: 371 | SEQ ID NO: 335 | SEQ ID NO: 336 |
| | TTGGTGTAAAAAGCACCCCTAGTATG | TCCCACAGCCAATTTCTACCCCTTTCACTTCCT | CTGATTTGGTCACATTCTCCAACGA |
| 484 | SEQ ID NO: 372 | SEQ ID NO: 335 | SEQ ID NO: 336 |
| | GAAAAGGGACAAGACCTACAAAAAGT | TCCCACAGCCAATTTCTACCCCTTTCACTTCCT | CTGATTTGGTCACATTCTCCAACGA |
| | CGAT | | |
| 485 | SEQ ID NO: 373 | SEQ ID NO: 335 | SEQ ID NO: 336 |
| | AGCTGCAGCAATCTCCATGAATAAAA | TCCCACAGCCAATTTCTACCCCTTTCACTTCCT | CTGATTTGGTCACATTCTCCAACGA |
| | TA | | |
| 486 | SEQ ID NO: 374 | SEQ ID NO: 335 | SEQ ID NO: 336 |
| | GGTGTAAAAAGCACCCCTAGTATGATT | TCCCACAGCCAATTTCTACCCCTTTCACTTCCT | CTGATTTGGTCACATTCTCCAACGA |
| 487 | SEQ ID NO: 375 | SEQ ID NO: 335 | SEQ ID NO: 336 |
| | GCTGCAGCAATCTCCATGAATAAAAT | TCCCACAGCCAATTTCTACCCCTTTCACTTCCT | CTGATTTGGTCACATTCTCCAACGA |
| | AATGC | | |
| 488 | SEQ ID NO: 376 | SEQ ID NO: 335 | SEQ ID NO: 336 |
| | ACCCCTAGTATGATTATAGAAAAGGG | TCCCACAGCCAATTTCTACCCCTTTCACTTCCT | CTGATTTGGTCACATTCTCCAACGA |
| | ACAA | | |
| 489 | SEQ ID NO: 377 | SEQ ID NO: 335 | SEQ ID NO: 336 |
| | AGTATGATTATAGAAAAGGGACAAGA | TCCCACAGCCAATTTCTACCCCTTTCACTTCCT | CTGATTTGGTCACATTCTCCAACGA |
| | CCTACA | | |
| 490 | SEQ ID NO: 378 | SEQ ID NO: 335 | SEQ ID NO: 336 |
| | TGGTGTAAAAAGCACCCCTAGTATG | TCCCACAGCCAATTTCTACCCCTTTCACTTCCT | CTGATTTGGTCACATTCTCCAACGA |
| 491 | SEQ ID NO: 379 | SEQ ID NO: 335 | SEQ ID NO: 336 |
| | AGCTGCAGCAATCTCCATGAATAA | TCCCACAGCCAATTTCTACCCCTTTCACTTCCT | CTGATTTGGTCACATTCTCCAACGA |
| 492 | SEQ ID NO: 380 | SEQ ID NO: 335 | SEQ ID NO: 336 |
| | ATTTGCTGAAACAATTGGTGTAAAAA | TCCCACAGCCAATTTCTACCCCTTTCACTTCCT | CTGATTTGGTCACATTCTCCAACGA |
| | GCA | | |
| 493 | SEQ ID NO: 347 | SEQ ID NO: 335 | SEQ ID NO: 381 |
| | AAAGGGACAAGACCTACAAAAAGTCG | TCCCACAGCCAATTTCTACCCCTTTCACTTCCT | CCACAAGACTGATTTGGTCACATTCT |
| | AT | | |
| 494 | SEQ ID NO: 348 | SEQ ID NO: 335 | SEQ ID NO: 381 |
| | TTGGTGTAAAAAGCACCCCTAGTATGA | TCCCACAGCCAATTTCTACCCCTTTCACTTCCT | CCACAAGACTGATTTGGTCACATTCT |
| | TT | | |
| 495 | SEQ ID NO: 349 | SEQ ID NO: 335 | SEQ ID NO: 381 |
| | TGCAGCAATCTCCATGAATAAAATAAT | TCCCACAGCCAATTTCTACCCCTTTCACTTCCT | CCACAAGACTGATTTGGTCACATTCT |
| | GCT | | |
| 496 | SEQ ID NO: 350 | SEQ ID NO: 335 | SEQ ID NO: 381 |
| | TAGACGAAGCTTCAAAATATGATAGC | TCCCACAGCCAATTTCTACCCCTTTCACTTCCT | CCACAAGACTGATTTGGTCACATTCT |
| | CGTAT | | |
| 497 | SEQ ID NO: 382 | SEQ ID NO: 335 | SEQ ID NO: 381 |
| | TCCATCAATTTGCTGAAACAATTGGTG | TCCCACAGCCAATTTCTACCCCTTTCACTTCCT | CCACAAGACTGATTTGGTCACATTCT |
| | TAAAA | | |
| 498 | SEQ ID NO: 383 | SEQ ID NO: 335 | SEQ ID NO: 381 |
| | GAGCTGCAGCAATCTCCATGA | TCCCACAGCCAATTTCTACCCCTTTCACTTCCT | CCACAAGACTGATTTGGTCACATTCT |
| 499 | SEQ ID NO: 355 | SEQ ID NO: 335 | SEQ ID NO: 381 |
| | GCAATCTCCATGAATAAAATAATGCTC | TCCCACAGCCAATTTCTACCCCTTTCACTTCCT | CCACAAGACTGATTTGGTCACATTCT |
| | CATCA | | |
| 500 | SEQ ID NO: 359 | SEQ ID NO: 335 | SEQ ID NO: 381 |
| | CCCCTAGTATGATTATAGAAAAGGGA | TCCCACAGCCAATTTCTACCCCTTTCACTTCCT | CCACAAGACTGATTTGGTCACATTCT |
| | CAAGAC | | |
| 501 | SEQ ID NO: 360 | SEQ ID NO: 335 | SEQ ID NO: 381 |
| | GAGCTGCAGCAATCTCCATGAATAAA | TCCCACAGCCAATTTCTACCCCTTTCACTTCCT | CCACAAGACTGATTTGGTCACATTCT |
| | ATA | | |
| 502 | SEQ ID NO: 338 | SEQ ID NO: 335 | SEQ ID NO: 381 |
| | AGACGAAGCTTCAAAATATGATAGCC | TCCCACAGCCAATTTCTACCCCTTTCACTTCCT | CCACAAGACTGATTTGGTCACATTCT |
| | GTAT | | |
| 503 | SEQ ID NO: 384 | SEQ ID NO: 335 | SEQ ID NO: 381 |
| | TCAATTTGCTGAAACAATTGGTGTAAA | TCCCACAGCCAATTTCTACCCCTTTCACTTCCT | CCACAAGACTGATTTGGTCACATTCT |
| | AAGC | | |
| 504 | SEQ ID NO: 385 | SEQ ID NO: 335 | SEQ ID NO: 381 |
| | GACAAGACCTACAAAAAGTCGATGAA | TCCCACAGCCAATTTCTACCCCTTTCACTTCCT | CCACAAGACTGATTTGGTCACATTCT |
| | TTTGC | | |
| 505 | SEQ ID NO: 386 | SEQ ID NO: 335 | SEQ ID NO: 381 |
| | GAGCTGCAGCAATCTCCATGAAT | TCCCACAGCCAATTTCTACCCCTTTCACTTCCT | CCACAAGACTGATTTGGTCACATTCT |
| 506 | SEQ ID NO: 341 | SEQ ID NO: 335 | SEQ ID NO: 381 |
| | TTGGTGTAAAAAGCACCCCTAGTATGA | TCCCACAGCCAATTTCTACCCCTTTCACTTCCT | CCACAAGACTGATTTGGTCACATTCT |
| 507 | SEQ ID NO: 342 | SEQ ID NO: 335 | SEQ ID NO: 381 |
| | TTGGTGTAAAAAGCACCCCTAGTATGAT | TCCCACAGCCAATTTCTACCCCTTTCACTTCCT | CCACAAGACTGATTTGGTCACATTCT |
| 508 | SEQ ID NO: 344 | SEQ ID NO: 335 | SEQ ID NO: 381 |
| | TTGGTGTAAAAAGCACCCCTAGTATGA | TCCCACAGCCAATTTCTACCCCTTTCACTTCCT | CCACAAGACTGATTTGGTCACATTCT |
| | TTA | | |
| 509 | SEQ ID NO: 387 | SEQ ID NO: 335 | SEQ ID NO: 381 |
| | ATCAATTTGCTGAAACAATTGGTGTAA | TCCCACAGCCAATTTCTACCCCTTTCACTTCCT | CCACAAGACTGATTTGGTCACATTCT |
| | AAAGC | | |

vanD Sets

| | | | |
|---|---|---|---|
| 510 | SEQ ID NO: 388 | SEQ ID NO: 389 | SEQ ID NO: 390 |
| | TGCGCCATACTGGGAAACG | TGATCCACCTCGCCAGCCATGAGATCATTT | AGCCGTGTCTCAGCTCAATC |
| 511 | SEQ ID NO: 391 | SEQ ID NO: 392 | SEQ ID NO: 393 |
| | CTGCGCCATACTGGGAAACG | TCTGATCCACCTCGCCAGCCATGAGA | TTAAAAAAGCCGTGTCTCAGCTCAA |
| 512 | SEQ ID NO: 394 | SEQ ID NO: 392 | SEQ ID NO: 395 |
| | GGTAGGCTGCGCCATACTG | TCTGATCCACCTCGCCAGCCATGAGA | AAAAGCCGTGTCTCAGCTCAA |
| 513 | SEQ ID NO: 396 | SEQ ID NO: 397 | SEQ ID NO: 398 |
| | GCTGCGCCATACTGGGAAAC | TCTGATCCACCTCGCCAGCCATGAGATCATTT | CTTAAAAAAGCCGTGTCTCAGCTCAA |
| 514 | SEQ ID NO: 399 | SEQ ID NO: 400 | SEQ ID NO: 401 |
| | AGATTTTGATTGAAGAGGCCGTTACC | TTCCCAGTATGGCGCAGCCTACCTCAC | CTCGCCAGCCATGAGATCATTT |
| 515 | SEQ ID NO: 399 | SEQ ID NO: 402 | SEQ ID NO: 401 |
| | AGATTTTGATTGAAGAGGCCGTTACC | CCCAGTATGGCGCAGCCTACCTCACT | CTCGCCAGCCATGAGATCATTT |
| 516 | SEQ ID NO: 399 | SEQ ID NO: 403 | SEQ ID NO: 401 |
| | AGATTTTGATTGAAGAGGCCGTTACC | TGGCGCAGCCTACCTCACTCCC | CTCGCCAGCCATGAGATCATTT |
| 517 | SEQ ID NO: 399 | SEQ ID NO: 404 | SEQ ID NO: 401 |
| | AGATTTTGATTGAAGAGGCCGTTACC | TCCCAGTATGGCGCAGCCTACCTCA | CTCGCCAGCCATGAGATCATTT |
| 518 | SEQ ID NO: 399 | SEQ ID NO: 405 | SEQ ID NO: 401 |
| | AGATTTTGATTGAAGAGGCCGTTACC | TCCCAGTATGGCGCAGCCTACCTCAC | CTCGCCAGCCATGAGATCATTT |
| 519 | SEQ ID NO: 399 | SEQ ID NO: 406 | SEQ ID NO: 401 |
| | AGATTTTGATTGAAGAGGCCGTTACC | AGTATGGCGCAGCCTACCTCACTCCC | CTCGCCAGCCATGAGATCATTT |
| 520 | SEQ ID NO: 399 | SEQ ID NO: 407 | SEQ ID NO: 401 |
| | AGATTTTGATTGAAGAGGCCGTTACC | TTTCCCAGTATGGCGCAGCCTACCTCA | CTCGCCAGCCATGAGATCATTT |
| 521 | SEQ ID NO: 399 | SEQ ID NO: 408 | SEQ ID NO: 401 |
| | AGATTTTGATTGAAGAGGCCGTTACC | TTTCCCAGTATGGCGCAGCCTACCTCAC | CTCGCCAGCCATGAGATCATTT |
| 522 | SEQ ID NO: 409 | SEQ ID NO: 410 | SEQ ID NO: 411 |
| | AGGTAGGCTGCGCCATACTG | CTGATCCACCTCGCCAGCCATGAGATCATTT | AAAAAAGCCGTGTCTCAGCTCAAT |
| 523 | SEQ ID NO: 399 | SEQ ID NO: 412 | SEQ ID NO: 401 |
| | AGATTTTGATTGAAGAGGCCGTTACC | CCCAGTATGGCGCAGCCTACCTCAC | CTCGCCAGCCATGAGATCATTT |
| 524 | SEQ ID NO: 399 | SEQ ID NO: 413 | SEQ ID NO: 401 |
| | AGATTTTGATTGAAGAGGCCGTTACC | ATGGCGCAGCCTACCTCACTCCC | CTCGCCAGCCATGAGATCATTT |
| 525 | SEQ ID NO: 399 | SEQ ID NO: 414 | SEQ ID NO: 401 |
| | AGATTTTGATTGAAGAGGCCGTTACC | TTCCCAGTATGGCGCAGCCTACCTCA | CTCGCCAGCCATGAGATCATTT |
| 526 | SEQ ID NO: 415 | SEQ ID NO: 406 | SEQ ID NO: 401 |
| | AAGATTTTGATTGAAGAGGCCGTTACC | AGTATGGCGCAGCCTACCTCACTCCC | CTCGCCAGCCATGAGATCATTT |
| 527 | SEQ ID NO: 415 | SEQ ID NO: 407 | SEQ ID NO: 401 |
| | AAGATTTTGATTGAAGAGGCCGTTACC | TTTCCCAGTATGGCGCAGCCTACCTCA | CTCGCCAGCCATGAGATCATTT |
| 528 | SEQ ID NO: 415 | SEQ ID NO: 408 | SEQ ID NO: 401 |
| | AAGATTTTGATTGAAGAGGCCGTTACC | TTTCCCAGTATGGCGCAGCCTACCTCAC | CTCGCCAGCCATGAGATCATTT |
| 529 | SEQ ID NO: 394 | SEQ ID NO: 410 | SEQ ID NO: 393 |
| | GGTAGGCTGCGCCATACTG | CTGATCCACCTCGCCAGCCATGAGATCATTT | TTAAAAAAGCCGTGTCTCAGCTCAA |
| 530 | SEQ ID NO: 415 | SEQ ID NO: 412 | SEQ ID NO: 401 |
| | AAGATTTTGATTGAAGAGGCCGTTACC | CCCAGTATGGCGCAGCCTACCTCAC | CTCGCCAGCCATGAGATCATTT |
| 531 | SEQ ID NO: 415 | SEQ ID NO: 413 | SEQ ID NO: 401 |
| | AAGATTTTGATTGAAGAGGCCGTTACC | ATGGCGCAGCCTACCTCACTCCC | CTCGCCAGCCATGAGATCATTT |
| 532 | SEQ ID NO: 415 | SEQ ID NO: 414 | SEQ ID NO: 401 |
| | AAGATTTTGATTGAAGAGGCCGTTACC | TTCCCAGTATGGCGCAGCCTACCTCA | CTCGCCAGCCATGAGATCATTT |
| 533 | SEQ ID NO: 415 | SEQ ID NO: 400 | SEQ ID NO: 401 |
| | AAGATTTTGATTGAAGAGGCCGTTACC | TTCCCAGTATGGCGCAGCCTACCTCAC | CTCGCCAGCCATGAGATCATTT |
| 534 | SEQ ID NO: 394 | SEQ ID NO: 389 | SEQ ID NO: 393 |
| | GGTAGGCTGCGCCATACTG | TGATCCACCTCGCCAGCCATGAGATCATTT | TTAAAAAAGCCGTGTCTCAGCTCAA |
| 535 | SEQ ID NO: 415 | SEQ ID NO: 402 | SEQ ID NO: 401 |
| | AAGATTTTGATTGAAGAGGCCGTTACC | CCCAGTATGGCGCAGCCTACCTCACT | CTCGCCAGCCATGAGATCATTT |
| 536 | SEQ ID NO: 415 | SEQ ID NO: 403 | SEQ ID NO: 401 |
| | AAGATTTTGATTGAAGAGGCCGTTACC | TGGCGCAGCCTACCTCACTCCC | CTCGCCAGCCATGAGATCATTT |
| 537 | SEQ ID NO: 415 | SEQ ID NO: 404 | SEQ ID NO: 401 |
| | AAGATTTTGATTGAAGAGGCCGTTACC | TCCCAGTATGGCGCAGCCTACCTCA | CTCGCCAGCCATGAGATCATTT |
| 538 | SEQ ID NO: 394 | SEQ ID NO: 397 | SEQ ID NO: 393 |
| | GGTAGGCTGCGCCATACTG | TCTGATCCACCTCGCCAGCCATGAGATCATTT | TTAAAAAAGCCGTGTCTCAGCTCAA |
| 539 | SEQ ID NO: 415 | SEQ ID NO: 405 | SEQ ID NO: 401 |
| | AAGATTTTGATTGAAGAGGCCGTTACC | TCCCAGTATGGCGCAGCCTACCTCAC | CTCGCCAGCCATGAGATCATTT |
| 540 | SEQ ID NO: 416 | SEQ ID NO: 400 | SEQ ID NO: 417 |
| | CAAGATTTTGATTGAAGAGGCCGTTACC | TTCCCAGTATGGCGCAGCCTACCTCAC | CCTCGCCAGCCATGAGAT |
| 541 | SEQ ID NO: 396 | SEQ ID NO: 418 | SEQ ID NO: 419 |
| | GCTGCGCCATACTGGGAAAC | CAATCTGATCCACCTCGCCAGCCATGAGA | CCTGATGAATCTTAAAAAAGCCGTGTCT |
| 542 | SEQ ID NO: 396 | SEQ ID NO: 397 | SEQ ID NO: 419 |
| | GCTGCGCCATACTGGGAAAC | TCTGATCCACCTCGCCAGCCATGAGATCATTT | CCTGATGAATCTTAAAAAAGCCGTGTCT |
| 543 | SEQ ID NO: 391 | SEQ ID NO: 420 | SEQ ID NO: 421 |
| | CTGCGCCATACTGGGAAACG | CTCAATCTGATCCACCTCGCCAGCCATGAGA | TCCTGATGAATCTTAAAAAAGCCGTGTCT |
| 544 | SEQ ID NO: 396 | SEQ ID NO: 420 | SEQ ID NO: 419 |
| | GCTGCGCCATACTGGGAAAC | CTCAATCTGATCCACCTCGCCAGCCATGAGA | CCTGATGAATCTTAAAAAAGCCGTGTCT |
| 545 | SEQ ID NO: 396 | SEQ ID NO: 422 | SEQ ID NO: 419 |
| | GCTGCGCCATACTGGGAAAC | CTCAATCTGATCCACCTCGCCAGCCATGAGAT | CCTGATGAATCTTAAAAAAGCCGTGTCT |
| 546 | SEQ ID NO: 396 | SEQ ID NO: 423 | SEQ ID NO: 419 |
| | GCTGCGCCATACTGGGAAAC | CTCAATCTGATCCACCTCGCCAGCCATGAG | CCTGATGAATCTTAAAAAAGCCGTGTCT |
| 547 | SEQ ID NO: 396 | SEQ ID NO: 424 | SEQ ID NO: 419 |
| | GCTGCGCCATACTGGGAAAC | TCAATCTGATCCACCTCGCCAGCCATGAGA | CCTGATGAATCTTAAAAAAGCCGTGTCT |
| 548 | SEQ ID NO: 425 | SEQ ID NO: 406 | SEQ ID NO: 426 |
| | AGCAAGATTTTGATTGAAGAGGCCGTTA | AGTATGGCGCAGCCTACCTCACTCCC | CACCTCGCCAGCCATGA |
| 549 | SEQ ID NO: 396 | SEQ ID NO: 427 | SEQ ID NO: 419 |
| | GCTGCGCCATACTGGGAAAC | TCAATCTGATCCACCTCGCCAGCCATGAGAT | CCTGATGAATCTTAAAAAAGCCGTGTCT |
| 550 | SEQ ID NO: 428 | SEQ ID NO: 429 | SEQ ID NO: 430 |
| | GGCTTGCTTGAATTGTCAGGCATT | CACGGAGCTTTGAATATCGCATCCCACATAC | AACGGTATATGCAAGCGCCTTATC |
| | | GG | |
| 551 | SEQ ID NO: 391 | SEQ ID NO: 431 | SEQ ID NO: 421 |
| | CTGCGCCATACTGGGAAACG | AGCTCAATCTGATCCACCTCGCCAGCC | TCCTGATGAATCTTAAAAAAGCCGTGTCT |
| 552 | SEQ ID NO: 396 | SEQ ID NO: 432 | SEQ ID NO: 419 |
| | GCTGCGCCATACTGGGAAAC | TCAATCTGATCCACCTCGCCAGCCATGAG | CCTGATGAATCTTAAAAAAGCCGTGTCT |
| 553 | SEQ ID NO: 391 | SEQ ID NO: 433 | SEQ ID NO: 434 |
| | CTGCGCCATACTGGGAAACG | TCTGATCCACCTCGCCAGCCATGAGAT | CGGCTGTGCTTCCTGATGA |
| 554 | SEQ ID NO: 435 | SEQ ID NO: 436 | SEQ ID NO: 437 |
| | CCATACAAGGCTTGCTTGAATTGTCA | ACGGAGCTTTGAATATCGCATCCCACATACG | ATACCCGCATTTTTCACAACGGTAT |
| | GAA | | |
| 555 | SEQ ID NO: 438 | SEQ ID NO: 420 | SEQ ID NO: 439 |
| | GGCCGTTACCGGGAGTGA | CTCAATCTGATCCACCTCGCCAGCCATGAGA | TTCCTGATGAATCTTAAAAAAGCCGTGTCT |
| 556 | SEQ ID NO: 440 | SEQ ID NO: 441 | SEQ ID NO: 442 |
| | AGGAAGCACAGCCGGAGAA | CCTCATCCGGTAAGGCGGCTGGAACT | AGCCAAGTATCCGGTAAATCTTCATTG |
| 557 | SEQ ID NO: 443 | SEQ ID NO: 392 | SEQ ID NO: 434 |
| | CCGTTACCGGGAGTGAGGTA | TCTGATCCACCTCGCCAGCCATGAGA | CGGCTGTGCTTCCTGATGA |
| 558 | SEQ ID NO: 399 | SEQ ID NO: 397 | SEQ ID NO: 444 |
| | AGATTTTGATTGAAGAGGCCGTTACC | TCTGATCCACCTCGCCAGCCATGAGATCATTT | TGATGAATCTTAAAAAAGCCGTGTCTCA |
| 559 | SEQ ID NO: 399 | SEQ ID NO: 410 | SEQ ID NO: 444 |
| | AGATTTTGATTGAAGAGGCCGTTACC | CTGATCCACCTCGCCAGCCATGAGATCATTT | TGATGAATCTTAAAAAAGCCGTGTCTCA |
| 560 | SEQ ID NO: 445 | SEQ ID NO: 446 | SEQ ID NO: 447 |
| | CGCTCTCGTATCCGGTCTTTG | CCGTTCCGGCTCCTCTTTTGGCGTGAATAA | TTCCGGCTGTGCTTCCTGAT |
| 561 | SEQ ID NO: 445 | SEQ ID NO: 446 | SEQ ID NO: 448 |
| | CGCTCTCGTATCCGGTCTTTG | CCGTTCCGGCTCCTCTTTTGGCGTGAATAA | CCGGCTGTGCTTCCTGATG |
| 562 | SEQ ID NO: 445 | SEQ ID NO: 446 | SEQ ID NO: 449 |
| | CGCTCTCGTATCCGGTCTTTG | CCGTTCCGGCTCCTCTTTTGGCGTGAATAA | TCCGGCTGTGCTTCCTGAT |
| 563 | SEQ ID NO: 445 | SEQ ID NO: 446 | SEQ ID NO: 450 |
| | CGCTCTCGTATCCGGTCTTTG | CCGTTCCGGCTCCTCTTTTGGCGTGAATAA | TCCTACCTCACTCCCGGAAAC |
| 564 | SEQ ID NO: 445 | SEQ ID NO: 446 | SEQ ID NO: 451 |
| | CGCTCTCGTATCCGGTCTTTG | CCGTTCCGGCTCCTCTTTTGGCGTGAATAA | CCTACCTCACTCCCGGAAAC |
| 565 | SEQ ID NO: 445 | SEQ ID NO: 446 | SEQ ID NO: 452 |
| | CGCTCTCGTATCCGGTCTTTG | CCGTTCCGGCTCCTCTTTTGGCGTGAATAA | GACCGCTGCCTGCAGTTC |
| 566 | SEQ ID NO: 445 | SEQ ID NO: 446 | SEQ ID NO: 453 |
| | CGCTCTCGTATCCGGTCTTTG | CCGTTCCGGCTCCTCTTTTGGCGTGAATAA | ATCCTACCTCACTCCCGGAAAC |
| 567 | SEQ ID NO: 445 | SEQ ID NO: 446 | SEQ ID NO: 454 |
| | CGCTCTCGTATCCGGTCTTTG | CCGTTCCGGCTCCTCTTTTGGCGTGAATAA | CATCCTACCTCACTCCCGGAAA |
| 568 | SEQ ID NO: 445 | SEQ ID NO: 446 | SEQ ID NO: 455 |
| | CGCTCTCGTATCCGGTCTTTG | CCGTTCCGGCTCCTCTTTTGGCGTGAATAA | CCGGCTGTGCTTCCTGAT |
| 569 | SEQ ID NO: 456 | SEQ ID NO: 457 | SEQ ID NO: 458 |
| | GGTTGTGAAAAATGCGGGAATTGAG | CTGCCCGTTCCGGCTCCTCTTTTGG | TCTGCCCGGCATACCTTATTCAC |
| 570 | SEQ ID NO: 445 | SEQ ID NO: 446 | SEQ ID NO: 459 |
| | CGCTCTCGTATCCGGTCTTTG | CCGTTCCGGCTCCTCTTTTGGCGTGAATAA | CAGTATGGCACATCCTACCTCACT |
| 571 | SEQ ID NO: 445 | SEQ ID NO: 446 | SEQ ID NO: 460 |
| | CGCTCTCGTATCCGGTCTTTG | CCGTTCCGGCTCCTCTTTTGGCGTGAATAA | CAGCCAAGTACCCGGTAAATCTTC |
| 572 | SEQ ID NO: 445 | SEQ ID NO: 446 | SEQ ID NO: 461 |
| | CGCTCTCGTATCCGGTCTTTG | CCGTTCCGGCTCCTCTTTTGGCGTGAATAA | GCTGTGCTTCCTGATGGATCTTAAAAA |
| 573 | SEQ ID NO: 462 | SEQ ID NO: 463 | SEQ ID NO: 464 |
| | CGTTCCGGCTCCTCTTTTGG | ACCGCTGCCTGCAGTTCCTCTGC | CGGAAACGGCCTCCTCAAC |
| 574 | SEQ ID NO: 465 | SEQ ID NO: 466 | SEQ ID NO: 467 |
| | GGCTCCTCTTTTGGCGTGAA | TGCAGTTCCTCTGCCCGGCATACCT | CTACCTCACTCCCGGAAACG |
| 575 | SEQ ID NO: 468 | SEQ ID NO: 469 | SEQ ID NO: 470 |
| | CCGTTCCGGCTCCTCTTTT | CTCTGCCCGGCATACCTTATTCACGCC | ACCGCTGCCTGCAGTT |
| 576 | SEQ ID NO: 471 | SEQ ID NO: 472 | SEQ ID NO: 473 |
| | CTGCTTGAGCTGTCCGGCATT | AGAACTCGAAACCCAGGTACCTCAATTCCCGC | TCCAGGCTGTCCCCCTTTT |
| 577 | SEQ ID NO: 474 | SEQ ID NO: 475 | SEQ ID NO: 476 |
| | TAAGGTATGCCGGGCAGAGGAA | TCCCGGAAACGGCCTCCTCAACCAAT | CCCAGTATGGCACATCCTACCT |
| 578 | SEQ ID NO: 477 | SEQ ID NO: 478 | SEQ ID NO: 479 |
| | GTCGCTCGCTTATATGGTTGTGAA | ACTCGAAACCCAGGTACCTCAATTCCCGCAT | AGGCTGTCCCCCTTTTGTAGA |
| 579 | SEQ ID NO: 480 | SEQ ID NO: 481 | SEQ ID NO: 482 |
| | CTCCTCTTTTGGCGTGAATAAGGT | TCTGTGACCGCTGCCTGCAGTTCC | AAACGGCCTCCTCAACCAAT |
| 580 | SEQ ID NO: 483 | SEQ ID NO: 484 | SEQ ID NO: 479 |
| | TGGATAAGTCGCTCGCTTATATGGT | ACTCGAAACCCAGGTACCTCAATTCCCGCA | AGGCTGTCCCCCTTTTGTAGA |
| 581 | SEQ ID NO: 483 | SEQ ID NO: 485 | SEQ ID NO: 486 |
| | TGGATAAGTCGCTCGCTTATATGGT | AACCCAGGTACCTCAATTCCCGCATTTTTCACA | CAGGCTGTCCCCCTTTTGTA |
| 582 | SEQ ID NO: 483 | SEQ ID NO: 487 | SEQ ID NO: 486 |
| | TGGATAAGTCGCTCGCTTATATGGT | ACCCAGGTACCTCAATTCCCGCATTTTTCACA | CAGGCTGTCCCCCTTTTGTA |
| 583 | SEQ ID NO: 488 | SEQ ID NO: 489 | SEQ ID NO: 490 |
| | CCTCTTTTGGCGTGAATAAGGTATGC | CTCTGTGACCGCTGCCTGCAGTTCC | GAAACGGCCTCCTCAACCA |
| 584 | SEQ ID NO: 491 | SEQ ID NO: 492 | SEQ ID NO: 493 |
| | TCGCTCGCTTATATGGTTGTGAAAA | TCGAAACCCAGGTACCTCAATTCCCGCA | CAGGCTGTCCCCCTTTTGT |
| 585 | SEQ ID NO: 494 | SEQ ID NO: 485 | SEQ ID NO: 486 |
| | ATGGATAAGTCGCTCGCTTATATGGT | ACCCAGGTACCTCAATTCCCGCATTTTTCACA | CAGGCTGTCCCCCTTTTGTA |
| 586 | SEQ ID NO: 494 | SEQ ID NO: 487 | SEQ ID NO: 486 |
| | ATGGATAAGTCGCTCGCTTATATGGT | AACCCAGGTACCTCAATTCCCGCATTTTTCACA | CAGGCTGTCCCCCTTTTGTA |
| 587 | SEQ ID NO: 495 | SEQ ID NO: 492 | SEQ ID NO: 493 |
| | GATAAGTCGCTCGCTTATATGGTTGT | TCGAAACCCAGGTACCTCAATTCCCGCA | CAGGCTGTCCCCCTTTTGT |
| 588 | SEQ ID NO: 496 | SEQ ID NO: 485 | SEQ ID NO: 486 |
| | TATGGATAAGTCGCTCGCTTATATGGT | ACCCAGGTACCTCAATTCCCGCATTTTTCACA | CAGGCTGTCCCCCTTTTGTA |
| 589 | SEQ ID NO: 496 | SEQ ID NO: 487 | SEQ ID NO: 486 |
| | TATGGATAAGTCGCTCGCTTATATGGT | AACCCAGGTACCTCAATTCCCGCATTTTTCACA | CAGGCTGTCCCCCTTTTGTA |
| 590 | SEQ ID NO: 497 | SEQ ID NO: 485 | SEQ ID NO: 486 |
| | GTATGGATAAGTCGCTCGCTTATATGGT | ACCCAGGTACCTCAATTCCCGCATTTTTCACA | CAGGCTGTCCCCCTTTTGTA |
| 591 | SEQ ID NO: 497 | SEQ ID NO: 487 | SEQ ID NO: 486 |
| | GTATGGATAAGTCGCTCGCTTATATGGT | AACCCAGGTACCTCAATTCCCGCATTTTTCACA | CAGGCTGTCCCCCTTTTGTA |
| 592 | SEQ ID NO: 498 | SEQ ID NO: 499 | SEQ ID NO: 486 |
| | TGTATGGATAAGTCGCTCGCTTATATGG | ACCCAGGTACCTCAATTCCCGCATTTTTCACAA | CAGGCTGTCCCCCTTTTGTA |
| 593 | SEQ ID NO: 498 | SEQ ID NO: 485 | SEQ ID NO: 486 |
| | TGTATGGATAAGTCGCTCGCTTATATGG | ACCCAGGTACCTCAATTCCCGCATTTTTCACA | CAGGCTGTCCCCCTTTTGTA |
| 594 | SEQ ID NO: 498 | SEQ ID NO: 487 | SEQ ID NO: 486 |
| | TGTATGGATAAGTCGCTCGCTTATATGG | AACCCAGGTACCTCAATTCCCGCATTTTTCACA | CAGGCTGTCCCCCTTTTGTA |
| 595 | SEQ ID NO: 500 | SEQ ID NO: 499 | SEQ ID NO: 486 |
| | GTATGGATAAGTCGCTCGCTTATATGG | ACCCAGGTACCTCAATTCCCGCATTTTTCACAA | CAGGCTGTCCCCCTTTTGTA |
| 596 | SEQ ID NO: 500 | SEQ ID NO: 485 | SEQ ID NO: 486 |
| | GTATGGATAAGTCGCTCGCTTATATGG | ACCCAGGTACCTCAATTCCCGCATTTTTCACA | CAGGCTGTCCCCCTTTTGTA |
| 597 | SEQ ID NO: 500 | SEQ ID NO: 487 | SEQ ID NO: 486 |
| | GTATGGATAAGTCGCTCGCTTATATGG | AACCCAGGTACCTCAATTCCCGCATTTTTCACA | CAGGCTGTCCCCCTTTTGTA |
| 598 | SEQ ID NO: 501 | SEQ ID NO: 499 | SEQ ID NO: 486 |
| | CTGTATGGATAAGTCGCTCGCTTATAT | ACCCAGGTACCTCAATTCCCGCATTTTTCACAA | CAGGCTGTCCCCCTTTTGTA |
| | GG | | |
| 599 | SEQ ID NO: 501 | SEQ ID NO: 485 | SEQ ID NO: 486 |
| | CTGTATGGATAAGTCGCTCGCTTATAT | ACCCAGGTACCTCAATTCCCGCATTTTTCACA | CAGGCTGTCCCCCTTTTGTA |
| | GG | | |
| 600 | SEQ ID NO: 501 | SEQ ID NO: 487 | SEQ ID NO: 486 |
| | CTGTATGGATAAGTCGCTCGCTTATAT | AACCCAGGTACCTCAATTCCCGCATTTTTCACA | CAGGCTGTCCCCCTTTTGTA |
| | GG | | |
| 601 | SEQ ID NO: 502 | SEQ ID NO: 492 | SEQ ID NO: 493 |
| | TGTATGGATAAGTCGCTCGCTTATATG | TCGAAACCCAGGTACCTCAATTCCCGCA | CAGGCTGTCCCCCTTTTGT |
| | GTT | | |

A PCR primer set for amplifying a vanC1 gene comprises at least one of the following sets of primer sequences: (1) SEQ ID NOS: 123 and 125; (2) SEQ ID NOS: 127 and 129; (3) SEQ ID NOS: 130 and 132; (4) SEQ ID NOS: 133 and 135; (5) SEQ ID NOS: 133 and 137; (6) SEQ ID NOS: 138 and 140; (7) SEQ ID NOS: 141 and 137; (8) SEQ ID NOS: 141 and 143; (9) SEQ ID NOS: 141 and 147; (10) SEQ ID NOS: 141 and 179; (11) SEQ ID NOS: 144 and 137; (12) SEQ ID NOS: 144 and 146; (13) SEQ ID NOS: 144 and 147; (14) SEQ ID NOS: 144 and 157; (15) SEQ ID NOS: 148 and 137; (16) SEQ ID NOS: 148 and 150; (17) SEQ ID NOS: 151 and 153; (18) SEQ ID NOS: 151 and 155; (19) SEQ ID NOS: 156 and 150; (20) SEQ ID NOS: 158 and 160; (21) SEQ ID NOS: 161 and 137; (22) SEQ ID NOS: 161 and 147; (23) SEQ ID NOS: 161 and 150; (24) SEQ ID NOS: 161 and 153; (25) SEQ ID NOS: 161 and 190; (26) SEQ ID NOS: 161 and 192; (27) SEQ ID NOS: 162 and 164; (28) SEQ ID NOS: 165 and 167; (29) SEQ ID NOS: 168 and 169; (30) SEQ ID NOS: 170 and 169; (31) SEQ ID NOS: 171 and 173; (32) SEQ ID NOS: 171 and 174; (33) SEQ ID NOS: 175 and 177; (34) SEQ ID NOS: 178 and 179; (35) SEQ ID NOS: 180 and 146; (36) SEQ ID NOS: 183 and 150; (37) SEQ ID NOS: 184 and 174; (38) SEQ ID NOS: 185 and 187; (39) SEQ ID NOS: 188 and 137; (40) SEQ ID NOS: 188 and 150; (41) SEQ ID NOS: 188 and 190; (42) SEQ ID NOS: 191 and 137; (43) SEQ ID NOS: 191 and 150; (44) SEQ ID NOS: 191 and 153; (45) SEQ ID NOS: 191 and 190; (46) SEQ ID NOS: 191 and 192; (47) SEQ ID NOS: 193 and 137; (48) SEQ ID NOS: 193 and 150; (49) SEQ ID NOS: 193 and 153; (50) SEQ ID NOS: 193 and 190; (51) SEQ ID NOS: 194 and 147; (52) SEQ ID NOS: 194 and 160; (53) SEQ ID NOS: 196 and 147; (54) SEQ ID NOS: 196 and 160; (55) SEQ ID NOS: 198 and 179; (56) SEQ ID NOS: 200 and 179; (57) SEQ ID NOS: 201 and 179; (58) SEQ ID NOS: 202 and 179; (59) SEQ ID NOS: 203 and 147; (60) SEQ ID NOS: 204 and 179; and (61) SEQ ID NOS: 204 and 187.

A PCR primer set for amplifying a vanC2/3 gene comprises at least one of the following sets of primer sequences: (1) SEQ ID NOS: 206 and 208; (2) SEQ ID NOS: 206 and 209; (3) SEQ ID NOS: 206 and 216; (4) SEQ ID NOS: 206 and 219; (5) SEQ ID NOS: 206 and 227; (6) SEQ ID NOS: 210 and 209; (7) SEQ ID NOS: 210 and 212; (8) SEQ ID NOS: 210 and 215; (9) SEQ ID NOS: 210 and 216; (10) SEQ ID NOS: 210 and 219; (11) SEQ ID NOS: 210 and 223; (12) SEQ ID NOS: 210 and 227; (13) SEQ ID NOS: 213 and 215; (14) SEQ ID NOS: 217 and 209; (15) SEQ ID NOS: 217 and 216; (16) SEQ ID NOS: 217 and 219; (17) SEQ ID NOS: 217 and 223; (18) SEQ ID NOS: 217 and 227; (19) SEQ ID NOS: 220 and 209; (20) SEQ ID NOS: 220 and 219; (21) SEQ ID NOS: 220 and 223; (22) SEQ ID NOS: 220 and 227; (23) SEQ ID NOS: 221 and 209; (24) SEQ ID NOS: 221 and 216; (25) SEQ ID NOS: 221 and 219; (26) SEQ ID NOS: 221 and 227; (27) SEQ ID NOS: 222 and 209; (28) SEQ ID NOS: 222 and 216; (29) SEQ ID NOS: 222 and 219; (30) SEQ ID NOS: 222 and 223; (31) SEQ ID NOS: 222 and 227; (32) SEQ ID NOS: 224 and 212; (33) SEQ ID NOS: 224 and 215; (34) SEQ ID NOS: 224 and 216; (35) SEQ ID NOS: 225 and 209; (36) SEQ ID NOS: 225 and 212; (37) SEQ ID NOS: 225 and 216; (38) SEQ ID NOS: 226 and 209; (39) SEQ ID NOS: 226 and 212; (40) SEQ ID NOS: 226 and 216; (41) SEQ ID NOS: 228 and 215; (42) SEQ ID NOS: 229 and 209; (43) SEQ ID NOS: 229 and 215; (44) SEQ ID NOS: 230 and 219; (45) SEQ ID NOS: 231 and 212; (46) SEQ ID NOS: 231 and 215; (47) SEQ ID NOS: 232 and 216; (48) SEQ ID NOS: 233 and 212; (49) SEQ ID NOS: 234 and 215; (50) SEQ ID NOS: 235 and 215; (51) SEQ ID NOS: 235 and 239; (52) SEQ ID NOS: 235 and 241; (53) SEQ ID NOS: 236 and 216; (54) SEQ ID NOS: 237 and 209; (55) SEQ ID NOS: 237 and 215; (56) SEQ ID NOS: 238 and 215; (57) SEQ ID NOS: 240 and 216; (58) SEQ ID NOS: 242 and 216; and (59) SEQ ID NOS: 243 and 215.

A PCR primer set for amplifying a vanD gene comprises at least one of the following sets of primer sequences: (1) SEQ ID NOS: 388 and 390; (2) SEQ ID NOS: 391 and 393; (3) SEQ ID NOS: 391 and 434; (4) SEQ ID NOS: 394 and 393; (5) SEQ ID NOS: 396 and 398; (6) SEQ ID NOS: 396 and 419; (7) SEQ ID NOS: 396 and 419; (8) SEQ ID NOS: 399 and 401; (9) SEQ ID NOS: 399 and 401; (10) SEQ ID NOS: 399 and 401; (11) SEQ ID NOS: 399 and 401; (12) SEQ ID NOS: 399 and 444; (13) SEQ ID NOS: 399 and 444; (14) SEQ ID NOS: 415 and 401; (15) SEQ ID NOS: 416 and 417; (16) SEQ ID NOS: 435 and 437; (17) SEQ ID NOS: 438 and 439; (18) SEQ ID NOS: 440 and 442; (19) SEQ ID NOS: 443 and 434; (20) SEQ ID NOS: 445 and 447; (21) SEQ ID NOS: 445 and 448; (22) SEQ ID NOS: 445 and 449; (23) SEQ ID NOS: 445 and 450; (24) SEQ ID NOS: 445 and 451; (25) SEQ ID NOS: 445 and 452; (26) SEQ ID NOS: 445 and 453; (27) SEQ ID NOS: 445 and 454; (28) SEQ ID NOS: 445 and 455; (29) SEQ ID NOS: 445 and 459; (30) SEQ ID NOS: 445 and 460; (31) SEQ ID NOS: 445 and 461; (32) SEQ ID NOS: 456 and 458; (33) SEQ ID NOS: 462 and 464; (34) SEQ ID NOS: 465 and 467; (35) SEQ ID NOS: 468 and 470; (36) SEQ ID NOS: 471 and 473; (37) SEQ ID NOS: 474 and 476; (38) SEQ ID NOS: 477 and 479; (39) SEQ ID NOS: 480 and 482; (40) SEQ ID NOS: 483 and 479; (41) SEQ ID NOS: 483 and 486; (42) SEQ ID NOS: 488 and 490; (43) SEQ ID NOS: 491 and 493; (44) SEQ ID NOS: 494 and 486; (45) SEQ ID NOS: 495 and 493; (46) SEQ ID NOS: 496 and 486; (47) SEQ ID NOS: 497 and 486; (48) SEQ ID NOS: 498 and 486; (49) SEQ ID NOS: 500 and 486; (50) SEQ ID NOS: 501 and 486; and (51) SEQ ID NOS: 502 and 493.

A PCR primer set for amplifying a vanE gene comprises at least one of the following sets of primer sequences: (1) SEQ ID NOS: 334 and 336; (2) SEQ ID NOS: 337 and 336; (3) SEQ ID NOS: 338 and 336; (4) SEQ ID NOS: 338 and 381; (5) SEQ ID NOS: 339 and 336; (6) SEQ ID NOS: 340 and 336; (7) SEQ ID NOS: 341 and 336; (8) SEQ ID NOS: 341 and 381; (9) SEQ ID NOS: 342 and 336; (10) SEQ ID NOS: 342 and 381; (11) SEQ ID NOS: 343 and 336; (12) SEQ ID NOS: 344 and 336; (13) SEQ ID NOS: 344 and 381; (14) SEQ ID NOS: 345 and 336; (15) SEQ ID NOS: 346 and 336; (16) SEQ ID NOS: 347 and 336; (17) SEQ ID NOS: 347 and 381; (18) SEQ ID NOS: 348 and 336; (19) SEQ ID NOS: 348 and 381; (20) SEQ ID NOS: 349 and 336; (21) SEQ ID NOS: 349 and 381; (22) SEQ ID NOS: 350 and 336; (23) SEQ ID NOS: 350 and 381; (24) SEQ ID NOS: 351 and 336; (25) SEQ ID NOS: 352 and 336; (26) SEQ ID NOS: 353 and 336; (27) SEQ ID NOS: 354 and 336; (28) SEQ ID NOS: 355 and 336; (29) SEQ ID NOS: 355 and 381; (30) SEQ ID NOS: 356 and 336; (31) SEQ ID NOS: 357 and 336; (32) SEQ ID NOS: 358 and 336; (33) SEQ ID NOS: 359 and 336; (34) SEQ ID NOS: 359 and 381; (35) SEQ ID NOS: 360 and 336; (36) SEQ ID NOS: 360 and 381; (37) SEQ ID NOS: 361 and 336; (38) SEQ ID NOS: 362 and 336; (39) SEQ ID NOS: 363 and 336; (40) SEQ ID NOS: 364 and 336; (41) SEQ ID NOS: 365 and 336; (42) SEQ ID NOS: 366 and 336; (43) SEQ ID NOS: 367 and 336; (44) SEQ ID NOS: 368 and 336; (45) SEQ ID NOS: 369 and 336; (46) SEQ ID NOS: 370 and 336; (47) SEQ ID NOS: 371 and 336; (48) SEQ ID NOS: 372 and 336; (49) SEQ ID NOS: 373 and 336; (50) SEQ ID NOS: 374 and 336; (51) SEQ ID NOS: 375 and 336; (52) SEQ ID NOS: 376 and 336; (53) SEQ ID NOS: 377 and 336; (54) SEQ ID NOS: 378 and 336; (55) SEQ ID NOS: 379 and 336; (56) SEQ ID NOS: 380 and 336; (57) SEQ ID NOS: 382 and 381; (58) SEQ ID NOS: 383 and 381; (59) SEQ ID NOS: 384 and 381; (60) SEQ ID NOS: 385 and 381; (61) SEQ ID NOS: 386 and 381; and (62) SEQ ID NOS: 387 and 381.

A PCR primer set for amplifying a vanG gene comprises at least one of the following sets of primer sequences: (1) SEQ ID NOS: 244 and 246; (2) SEQ ID NOS: 244 and 247; (3) SEQ ID NOS: 244 and 248; (4) SEQ ID NOS: 244 and 250; (5) SEQ ID NOS: 244 and 251; (6) SEQ ID NOS: 244 and 254; (7) SEQ ID NOS: 244 and 258; (8) SEQ ID NOS: 244 and 259; (9) SEQ ID NOS: 244 and 284; (10) SEQ ID NOS: 244 and 286; (11) SEQ ID NOS: 244 and 287; (12) SEQ ID NOS: 249 and 246; (13) SEQ ID NOS: 249 and 248; (14) SEQ ID NOS: 249 and 286; (15) SEQ ID NOS: 249 and 301; (16) SEQ ID NOS: 249 and 306; (17) SEQ ID NOS: 249 and 308; (18) SEQ ID NOS: 249 and 312; (19) SEQ ID NOS: 252 and 246; (20) SEQ ID NOS: 252 and 262; (21) SEQ ID NOS: 253 and 246; (22) SEQ ID NOS: 255 and 246; (23) SEQ ID NOS: 256 and 246; (24) SEQ ID NOS: 260 and 258; (25) SEQ ID NOS: 263 and 258; (26) SEQ ID NOS: 264 and 266; (27) SEQ ID NOS: 267 and 269; (28) SEQ ID NOS: 270 and 266; (29) SEQ ID NOS: 270 and 269; (30) SEQ ID NOS: 271 and 266; (31) SEQ ID NOS: 272 and 274; (32) SEQ ID NOS: 275 and 269; (33) SEQ ID NOS: 276 and 269; (34) SEQ ID NOS: 277 and 269; (35) SEQ ID NOS: 278 and 266; (36) SEQ ID NOS: 279 and 269; (37) SEQ ID NOS: 280 and 266; (38) SEQ ID NOS: 280 and 269; (39) SEQ ID NOS: 281 and 269; (40) SEQ ID NOS: 282 and 266; (41) SEQ ID NOS: 282 and 269; (42) SEQ ID NOS: 283 and 269; (43) SEQ ID NOS: 285 and 259; (44) SEQ ID NOS: 288 and 290; (45) SEQ ID NOS: 288 and 304; (46) SEQ ID NOS: 291 and 290; (47) SEQ ID NOS: 293 and 290; (48) SEQ ID NOS: 293 and 304; (49) SEQ ID NOS: 295 and 258; (50) SEQ ID NOS: 297 and 290; (51) SEQ ID NOS: 298 and 290; (52) SEQ ID NOS: 298 and 304; (53) SEQ ID NOS: 299 and 290; (54) SEQ ID NOS: 300 and 290; (55) SEQ ID NOS: 302 and 274; (56) SEQ ID NOS: 303 and 290; (57) SEQ ID NOS: 303 and 304; (58) SEQ ID NOS: 305 and 274; (59) SEQ ID NOS: 305 and 290; (60) SEQ ID NOS: 307 and 274; (61) SEQ ID NOS: 307 and 290; (62) SEQ ID NOS: 309 and 290; (63) SEQ ID NOS: 310 and 290; (64) SEQ ID NOS: 311 and 258; (65) SEQ ID NOS: 313 and 290; (66) SEQ ID NOS: 314 and 290; (67) SEQ ID NOS: 314 and 320; (68) SEQ ID NOS: 315 and 290; (69) SEQ ID NOS: 316 and 290; (70) SEQ ID NOS: 317 and 290; (71) SEQ ID NOS: 318 and 290; (72) SEQ ID NOS: 321 and 258; (73) SEQ ID NOS: 322 and 324; (74) SEQ ID NOS: 325 and 266; (75) SEQ ID NOS: 325 and 274; (76) SEQ ID NOS: 326 and 274; (77) SEQ ID NOS: 327 and 266; (78) SEQ ID NOS: 328 and 274; (79) SEQ ID NOS: 329 and 274; (80) SEQ ID NOS: 330 and 274; (81) SEQ ID NOS: 331 and 274; (82) SEQ ID NOS: 332 and 266; and (83) SEQ ID NOS: 333 and 266.

The preceding numbering of the sets of primers does not correspond exactly to the “Group” numbering scheme in Table 6 because certain groups use the same primer set, but different internal probes. For example, Groups 213 and 214 of Table 6 each employ the forward primer of SEQ ID NO: 123 and the reverse primer of SEQ ID NO: 125, but different internal probes in each instance, e.g., SEQ ID NOS: 124 and 126. Accordingly, primer set “(1)” of the preceding passage relating to the vanC1 primers implies any one of Groups 213 or 214 of Table 6.

Any set of primers can be used simultaneously in a multiplex reaction with one or more other primer sets, so that multiple amplicons are amplified simultaneously.

A probe for binding to an amplicon(s) of a vanC, vanD, vanE and/or vanG gene, or to a vanC, vanD, vanE and/or vanG gene target, comprises at least one of the following probe sequences: SEQ ID NOS: 124, 126, 128, 131, 134, 136, 139, 142, 145, 149, 152, 154, 159, 163, 166, 172, 176, 181, 182, 186, 189, 195, 197, 199, 205, 207, 211 (vanC probes); SEQ ID NOS: 389, 392, 397, 400, 402-408, 410, 412-414, 418, 420, 422-424, 427, 429, 431-433, 436, 441, 446, 457, 463, 466, 469, 472, 478, 481, 484, 485, 487, 489, 492, 499 (vanD probes); SEQ ID NOS: 335 (vanE probe) and SEQ ID NOS: 245, 257, 261, 265, 268, 273, 292, 294, 296, 319, 323 (vanG probes).

Any set of primers can be used simultaneously in a multiplex reaction with one or more other primer sets, so that multiple amplicons are amplified simultaneously.

Primer sets for simultaneously amplifying the vanA and/or vanB and/or vanC and/or vanD and/or vanE and/or vanG comprises a nucleotide sequence selected from the primer sets consisting of: Groups 1-601 of Tables 5 and 6. Oligonucleotide probes for binding to the vanA and/or vanB and/or vanC and/or vanD and/or vanE and/or vanG genes comprises a nucleotide sequence selected from the group consisting of: SEQ ID NOS: 2, 4, 7, 9, 11-18, 20, 24, 25, 27, 30, 35, 43, 46, 49, 50, 54, 56, 57, 58 (vanA probes); 63, 65, 66, 74, 77, 85-88, 90, 91, 95, 97-102 (vanB probes); 124, 126, 128, 131, 134, 136, 139, 142, 145, 149, 152, 154, 159, 163, 166, 172, 176, 181, 182, 186, 189, 195, 197, 199, 205, 207, 211 (vanC probes); SEQ ID NOS: 389, 392, 397, 400, 402-408, 410, 412-414, 418, 420, 422-424, 427, 429, 431-433, 436, 441, 446, 457, 463, 466, 469, 472, 478, 481, 484, 485, 487, 489, 492, 499 (vanD probes); SEQ ID NOS: 335 (vanE probe); and SEQ ID NOS: 245, 257, 261, 265, 268, 273, 292, 294, 296, 319, 323 (vanG probes).

The internal control is detected by a forward primer (SEQ ID NO: 504), a reverse primer (SEQ ID NO: 506) and a probe (SEQ ID NO: 505). A plasmid vector containing the internal control target sequence (SEQ ID NO: 503): GCGAAGTGAGAATACGCCGTGTCGCAGTTTCCTTGAGCAGTGTCTCTAAATGCCTCA AACCGTCGCATTTTTGGTTATAGCAGTAACTATATGGAGGTCCGTAGGCGGCGTGCG TGGGGGCACCAAACTCATCCAACGGTCGACTGCGCCTGTAGGGTCTTAAGAAGCGG CACCTCAGACCGATAGCATAGCACTTAAAGAGGAATTGAATAATCAAGATGGGTAT CCGACCGACGCGGAGTGACCGAGGAAGAGGACCCTGCATGTATCCTGAGAGTATAG TTGTCAGAGCAGCAATTGATTCACCACCAAGGGACTTAGTCT is included in the assay. The internal control plasmid is added directly to the reaction mix to monitor the integrity of the PCR reagents and the presence of PCR inhibitors.

TABLE 7
Internal Control.

| Group | | | |
|---|---|---|---|
| No. | Forward primer | Probe | Reverse primer |
| 602 | SEQ ID NO: 504 | SEQ ID NO: 505 | SEQ ID NO: 506 |
| | CAGACCGATAGCA | TGCTGCTCTGACA | TCCCTTGGTGGTG |
| | TAGCACTTAAA | ACTATACTCTCAG | AATCAAT |
| | | GATACA | |

### Example 5

### Enterococcus Species-Specific Markers

The vanA and vanB markers, which are carried on a transferable element, are indicative of the presence of VRE. vanA is almost always associated with VRE, while vanB is usually associated with VRE. vanB can also occur in species other than Enterococcus (e.g. Clostridium). In either case, a direct link cannot be made between Enterococcus and the detection of vanA or vanB in a mixed flora population. In some cases, detection of vanA or vanB harboring organisms is followed by an attempt to isolate the vancomycin resistant organism and conclusively identify it as Enterococcus.

Thus, in one respect, a species-specific marker is useful for identifying vancomycin-resistant clinical isolates as Enterococcus faecium (E. faecium or Efm) and/or Enterococcus faecalis (E. faecalis or Efs), which are the two most common Type A and Type B Enterococcus species. These two species are also the most important with regard to VRE.

One embodiment is directed to species-specific markers for the detection of Efm, Efs or both Efm and Efs (“Efm/Efs dual”). Two approaches were utilized within this embodiment. One approach targeted the sodA gene, which encodes the enzyme superoxide dismutase A. The sodA gene is frequently used as a bacterial species-specific marker. A second approach targeted novel genes from Efm, Efs that were identified through in silico analyses. Below are the sodA markers for Efm and Efs, the novel marker for Efm and Efs and a dual marker (dual Efm/Efs dual). The dual marker detects both Efm and Efs. Table 8A-12 describe the nucleic acid primers and probes used for detection and screening of Efm and/or Efs based on the target. Below are the sequences of the sodA for Efm and Efs, novel genes for Efm and Efs, and dual genes for Efm and Efs.

Efm_sodA
(SEQ ID NO: 507)
TAGAAAGATTATTATCTGATATGGACGCTATTCCAACAGATATCAAGACAGCTGTAC
GTAACAATGGTGGCGGACATGCTAACCATTCATTTTTCTGGGAAATCATGGCACCAA
ATGCTGGTGGCGAACCTACAGGAGAAATAAAAGAAGCGATTAATGAAGCTTTTGGT
GATTTTTCTTCTTTTAAAGAAGAATTCAAAAAAGCAGCCGCTGGACGATTTGGTTCT
GGATGGGCTTGGCATGTAATGGAAATTGGAAAATTAGCTATTACCTCTACTGCAAAT
CAAGATTCTCCATT
Efs_sodA
(SEQ ID NO: 508)
TCTGTAGAAAACCTAATTTCAGATATGAATGCTATTCCTGAAGATATCCGCACAGCT
GTTCGTAACAATGGTGGCGGTCACGCAAACCATACATTCTTCTGGGAAATTATGGCA
CCAAATGCTGGTGGACAACCAACTGGCGCTATTAAAGAAGCAATCGATGAAACATT
TGGTAGCTTTGATGAAATGAAAGCTGCTTTCAAAACAGCTGCAACTGGCCGCTTTGG
TTCAGGTTGGGCTTGGTTAGTTGTGAATAACGGTAAATTAGAAATCACTTCAACACC
AA
Efm_novel_marker
(SEQ ID NO: 509)
ACGCGTTCGTTTTCGTTCTCTTCTAGCAAAAATACTCCCCGTATCCCGCTTCAGAGGT
TTCGCCTTCTTATGGATGTCCTTATAGCGATCCATCCGACTCATTGTGGTTCCTCCTC
GTTTCTTACTGATAAATAAAGGATACGATAACCAATCGACAAAAGCCTATCATTTTT
TGATAATTTAATAAAAAATAACGAAATAAATGCTTCGATACATAAAG
Efs_novel_marker
(SEQ ID NO: 510)
CTCGTTCCTTTTAGCAAAAGTAATTGGGACAAGCGTCGCACATCTTCAGGAGCTAAA
TCAATTTTTTTAATCGTATGATACAGCGTTTCTAATTGTTTCACTGTCTCTGGATTAG
GTACTCCGTCAAGTACTCGTACTTGAAAATCATCTAAAATATTTTCACCATTTTCAAT
ATACGCATCTAAAAAAGAAGTATCCAATGACTGTTGCAATAACTGGATTGCTTGTTC
CATTGGCTCGAAAGCCTCTTCAATTTTTTCTGGGAACAAATTAGCACCTCTATTCTAT
TCAAAATGTAACAACTTCCATACCTGTTTTATTTTAACGAAAAAAGAAAGAATAATC
AATGCCTTTACTGTCTTTCTTCTAAAATCTAATAATTTGTAGAAAAACAACGCTATTC
CAGTATTGTTTCCGCTTTTTTTTGATGAGAAACTCTCTCTTGATATTGGTATTTTCGAC
CAGATTTGTTTAATAAAACAGTTATTCTTAATTGTTCATTTTTGTATGTATAATCGAG
GGTCACTTGCGCGTATACGAGCCTTTCTTCTGTTTTTAACTTTCGTCTATTTTGTTTAA
CATCTGCAAGAAATAAATGAAAGATACTTTTCCCTGCATAGTATTCCTGGGTAGCTT
GCGTAAATTTTTGTGTTAACTGACGTTCTTCTAGTAACTGAAGGGCGAGAAAACTCA
TCAAATAAACAATGGCCATGGCCGTGAACAATAAGTTTCCTGAATATTTTTGCCTCA
TTAAAATTCTCCCTTTATCGGAAAAAAACTATCAAATGTTTTTCCAGAAGTAAAAGT
AACTTCCAAAGTAAACGATTGTCCTTCATTTTTAAACTGTCCATTTTTGATACCAATT
AACAGCGGTTGATAGCCTTTACCATTGACTCGCTTAATAATTTTATCTTCTTTGATTT
GAATTGAAATAGTCTTGTTTGTTTCTGAGTCGAGGAAAGAAATCTCTTGAGCAGAGC
CTGTTTGGAAAACGAGCTTTTGACATTCTTTTTCTAATTGAATTAAAAAAATATGCCA
GGATTTCGAATCATTGTTTTTCAAATATTGATTTCCGATAAAACTTTGTTGAATCATC
AACTGAAACAATTGACACATACAACTTAAAACGACTAGTGCTACTAAACACTCTAA
CATCGTAAAACCCGCATATTTTTTATTGACCAATCAAAATCGCTCCTCTTGAATCTGT
GACTTTTAGTATCCTTCTTCCATCGTTCTCCATCAAAGAAAATCGGTAAGTTTCTCCT
TGATATATCCGCTCTTTGAAACGTAAATCACCCGTCTGCTCTAAAAATAAAATTGCT
TCGTATCCTAGACGAGTGCGCGTTAATTCTTGCTCCCGTTGATAATTTTGCTGAATTA
ATTGTGTAATAGTCAGGGAGAATATCCCTGCTATCACACATACAATACTAAAACTAA
CGAGACTCTCTAATAAAATAAAACCGCTATAAATAGTTAATTTTCTTCGTATATTTGC
CACTCCCCATCT
Efm_Efs_dual_novel_marker (from Efm)
(SEQ ID NO: 511)
CGAGCGTTCTCGAGAATACTCGACGCTGAAATTTTTGCACCATTTATTGCTTTATCGT
GACGTGACTTTGCAATAACAAATCCTAAACCAAGACCGACAATCAAACCGACGATA
GCGAGGAGAATATTTAAAACCATATTTCCACCTCCATACTATCTTTTT
Efm_Efs_dual_novel_marker (from Efs)
(SEQ ID NO: 512)
GCTGATGATTGTGCACCAGCTATTTCTTTCTCGTGACGTGACTTTGCGACCATAAACC
CTAAACCAAGACCGACAATTAAACCGATGATAGCGAGGAGAATGTTGAATACCATA
AAATCCACCTCCATACTATCTTTTT

TABLE 8A
Efm sodA gene nucleic acid primers and probes

Primer
SEQ ID NO: 513
GGACATGCTAACCATTCATTT
SEQ ID NO: 514
CGCTGGACGATTTGGTTCTG
SEQ ID NO: 515
AGCGATTAATGAAGCTTTTGGTGA
SEQ ID NO: 516
GGAAATCATGGCACCGAATG
SEQ ID NO: 517
TGGTGGACATGCTAACCATTCA
SEQ ID NO: 518
AGCGATTAATGAAGCTTTTGGTGAT
SEQ ID NO: 519
CAGTAGAGGTAATAGCTAATTTTCC
SEQ ID NO: 520
ATTTCTCCTGTAGGTTCGC
SEQ ID NO: 521
GCCAAGCCCATCCAGA
SEQ ID NO: 522
CCATTACAAGCCAAGCC
SEQ ID NO: 523
AGCTTCATTAATCGCTTCTTTT
SEQ ID NO: 524
GCCAAGCCCATCCAGAAC
SEQ ID NO: 525
GCCAAGCCCAACCAGA
SEQ ID NO: 526
GCTTCATTAATTGCTTCTTTTATTG
SEQ ID NO: 527
GGCAGTAGAGGTAATAGCTAA
SEQ ID NO: 528
GGCAGTAGAGGTAATAGCTAAT
SEQ ID NO: 529
CCAAAAGCTTCATTAATCGCTTCTT
SEQ ID NO: 530
CATCCAGAACCAAATCGTCCAG
SEQ ID NO: 531
TCACCAAAAGCTTCATTAATCGCT
SEQ ID NO: 532
TCTTTTATTTCTCCTGTAGGTTCGC
SEQ ID NO: 533
CATTAATCGCTTCTTTTATTTCTCC
SEQ ID NO: 534
TAATCGCTTCTTTTATTTCTCCTGT
SEQ ID NO: 535
CATTTTCCATTACAAGCCAAGCC
SEQ ID NO: 536
CAAGCCCATCCAGAACCAAATC
SEQ ID NO: 537
GTCCAGCGGCTGCTTTT
SEQ ID NO: 538
CATCCAGAACCAAATCGTCCA
SEQ ID NO: 539
TGCCAAGCCCATCCAGAAC
SEQ ID NO: 540
CAACCAGAACCAAAACGTCCA
SEQ ID NO: 541
TCGCCAAAAGCTTCATTAATTGCTT
SEQ ID NO: 542
AAAATCGCCAAAAGCTTCATT
SEQ ID NO: 543
GCCAAGCCCAACCAGAAC
SEQ ID NO: 544
ATTACAAGCCAAGCCCAACCA
SEQ ID NO: 545
ATTCTCCATTACAAGCCAAGCC
SEQ ID NO: 546
CGTCCAGCTGCTGCTTTT
SEQ ID NO: 547
TAATTGCTTCTTTTATTGCCCCAGT
SEQ ID NO: 548
TTTCCCATTCTCCATTACAAGCCA
SEQ ID NO: 549
AGCTAATTTCCCATTCTCCATTACA
SEQ ID NO: 550
TGGAGAATCTTGATTGGCAGTAGAG
SEQ ID NO: 551
TTGATTGGCAGTAGAGGTAATAGC
SEQ ID NO: 552
GATTGGCAGTAGAGGTAATAGCTAA
SEQ ID NO: 553
TATTACAAGCCAAGCCCATCCAG
Probe
SEQ ID NO: 554
CATGGCACCAAATGCTGGT
SEQ ID NO: 555
CGGTGCCATGATTTCCCAGAAAA
SEQ ID NO: 556
CCAAAACGTCCAGCTGCTGC
SEQ ID NO: 557
ATGGCACCGAATGCGGG
SEQ ID NO: 558
AGAAGCAATTAATGAAGCTTTTGGCGA
SEQ ID NO: 559
ATTGCTTCTTTTATTGCCCCAGTAGG
SEQ ID NO: 560
AGCTAATTTTCCATTTTCCATTACAAGCCAAGCCC
SEQ ID NO: 561
TGGGCTTGGCTTGTAATGGAAAATGGAAAATTAGC
SEQ ID NO: 562
CCAGCATTTGGTGCCATGATTTCCCAGAAAA
SEQ ID NO: 563
TTTTCCATTTTCCATTACAAGCCAAGCCCATCCA
SEQ ID NO: 564
TTTTCCATTACAAGCCAAGCCCATCCAGAACC
SEQ ID NO: 565
ATTACAAGCCAAGCCCATCCAGAACCAAAT
SEQ ID NO: 566
ACCAAATGCTGGTGGCGAACCTACA
SEQ ID NO: 567
ATTTGGTTCTGGATGGGCTTGGCTTGTA
SEQ ID NO: 568
CAACCAGAACCAAAACGTCCAGCTGC
SEQ ID NO: 569
CGCCAAAAGCTTCATTAATTGCTTCTTTTATTGCC
SEQ ID NO: 570
CGCATTCGGTGCCATGATTTCCCAGAA
SEQ ID NO: 571
TTTTCTGGGAAATCATGGCACCGAATGC
SEQ ID NO: 572
TTCTGGGAAATCATGGCACCGAATGCG
SEQ ID NO: 573
CAATAAAAGAAGCAATTAATGAAGCTTTTGGCGAT
SEQ ID NO: 574
AGCTTCATTAATTGCTTCTTTTATTGCCCCAGTAG

TABLE 8B
Efm sodA gene solutions

| Group | | | |
|---|---|---|---|
| No. | Forward Primer | Probe | Reverse Primer |
| 603 | SEQ ID NO: 517 | SEQ ID NO: 571 | SEQ ID NO: 529 |
| | TGGTGGACATGC | TTTTCTGGGAAATC | CCAAAAGCTTCATT |
| | TAACCATTCA | ATGGCACCGAATGC | AATCGCTTCTT |
| 604 | SEQ ID NO: 517 | SEQ ID NO: 555 | SEQ ID NO: 529 |
| | TGGTGGACATG | CGGTGCCATGATTT | CCAAAAGCTTCATT |
| | CTAACCATTCA | CCCAGAAAA | AATCGCTTCTT |
| 605 | SEQ ID NO: 517 | SEQ ID NO: 562 | SEQ ID NO: 529 |
| | TGGTGGACATGC | CCAGCATTTGGTG | CCAAAAGCTTCATT |
| | TAACCATTCA | CCATGATTTCCCA | AATCGCTTCTT |
| | | GAAAA | |

TABLE 9A
Efs sodA gene nucleic acid primers and probes

Primer
SEQ ID NO: 575
CTGGCCGCTTTGGTT
SEQ ID NO: 576
ACATTCTTCTGGGAAATTATGG
SEQ ID NO: 577
TGAATGCTATTCCTGAAGATATCCG
SEQ ID NO: 578
AAAGAAGCAATCGATGAAACATTTG
SEQ ID NO: 579
GGGAAATTATGGCACCAAAT
SEQ ID NO: 580
TTCTGGGAAATTATGGCACCAAATG
SEQ ID NO: 581
TGGCGCTATTAAAGAAGCAATCGA
SEQ ID NO: 582
TTCTTCTGGGAAATTATGGCACCAA
SEQ ID NO: 583
GCAATCGATGAAACATTTGGTAGC
SEQ ID NO: 584
CAACTGGCGCTATTAAAGAAGCA
SEQ ID NO: 585
ACAGCTGTTCGTAACAATGGTG
SEQ ID NO: 586
TCAGATATGAATGCTATTCCTGAAG
SEQ ID NO: 587
GGTAGCTTTGATGAAATGAAAGCTG
SEQ ID NO: 588
TGCTGGTGGACAACCAACTG
SEQ ID NO: 589
ACCAAATGCTGGTGGACAAC
SEQ ID NO: 590
GGTCACGCAAACCATACATTCTTC
SEQ ID NO: 591
TGAAAGCTGCTTTCAAAACAGCTG
SEQ ID NO: 592
TTGATGAAATGAAAGCTGCTTTCAA
SEQ ID NO: 593
GAAATGAAAGCTGCTTTCAAAACAG
SEQ ID NO: 594
CTATTCCTGAAGATATCCGTACTGC
SEQ ID NO: 595
AACATTCTTCTGGGAAATTATGGCA
SEQ ID NO: 596
GCAAACCAAACATTCTTCTGGGAAA
SEQ ID NO: 597
CAAACATTCTTCTGGGAAATTATGG
SEQ ID NO: 598
GGTCACGCAAACCAAACATTCT
SEQ ID NO: 599
GATGAAACATTTGGCAGCTTTGATG
SEQ ID NO: 600
ACATTTGGCAGCTTTGATGAAATG
SEQ ID NO: 601
TGGCGGGCACGCAA
SEQ ID NO: 602
CAACCAACTGGCGCTATTAAAGA
SEQ ID NO: 603
GGACAACCAACTGGCGCTA
SEQ ID NO: 604
TGCGACTGGCCGCTTT
SEQ ID NO: 605
TTTCAAAACAGCTGCGACTGG
SEQ ID NO: 606
GTTTCATCGATTGCTTCTTTAATAG
SEQ ID NO: 607
CCAAAGCGGCCAGT
SEQ ID NO: 608
AGAAGAATGTATGGTTTGCG
SEQ ID NO: 609
GCCATAATTTCCCAGAAGAATG
SEQ ID NO: 610
TTCTAATTTACCGTTATTCACAACT
SEQ ID NO: 611
GAAAGCAGCTTTCATTTCATC
SEQ ID NO: 612
GGTGATTTCTAATTTACCGTTATTC
SEQ ID NO: 613
AGCGCCAGTTGGTTG
SEQ ID NO: 614
TTCTTTAATAGCGCCAGTTGGTTG
SEQ ID NO: 615
CCAAATGTTTCATCGATTGCTTCTT
SEQ ID NO: 616
TTTCATCGATTGCTTCTTTAATAGC
SEQ ID NO: 617
TTTGGTGCCATAATTTCCCAGAAGA
SEQ ID NO: 618
GATTGCTTCTTTAATAGCGCCAGTT
SEQ ID NO: 619
TGAACCAAAGCGGCCAGT
SEQ ID NO: 620
CAAAGCGGCCAGTTGCA
SEQ ID NO: 621
AGCGGCCAGTTGCAG
SEQ ID NO: 622
TACCGTTATTCACAACTAACCAAGC
SEQ ID NO: 623
ATTTACCGTTATTCACAACTAACCA
SEQ ID NO: 624
AATTTACCGTTATTCACAACTAACC
SEQ ID NO: 625
CCGTTATTCACAACTAACCAAGCC
SEQ ID NO: 626
AAGCAGCTTTCATTTCATCAAAGC
SEQ ID NO: 627
CCAGTTGGTTGTCCACCAG
SEQ ID NO: 628
ACAACTAACCAAGCCCAACC
SEQ ID NO: 629
CAGTTGCAGCTGTTTTGAAAGCA
SEQ ID NO: 630
TAACCAAGCCCAACCTGAACC
SEQ ID NO: 631
GCTGTTTTGAAAGCAGCTTTCATT
SEQ ID NO: 632
GCCCAACCTGAACCAAAGC
SEQ ID NO: 633
TCAAAGCTACCAAATGTTTCATCGA
SEQ ID NO: 634
ATTTCCCAGAAGAATGTTTGGTTTG
SEQ ID NO: 635
TTTCCCAGAAGAATGTTTGGTTTGC
SEQ ID NO: 636
GCTGCCAAATGTTTCATCGATTG
SEQ ID NO: 637
TGCCAAATGTTTCATCGATTGCTT
SEQ ID NO: 638
TTTCATCAAAGCTGCCAAATGTTTC
SEQ ID NO: 639
TTCATCAAAGCTGCCAAATGTTTCA
SEQ ID NO: 640
CCAGCATTTGGTGCCATAATTTC
Probe
SEQ ID NO: 641
CTTCTTTAATAGCGCCAGTTGGTTG
SEQ ID NO: 642
CGCTATTAAAGAAGCAATCGATGAAACATTTG
SEQ ID NO: 643
CAGGTTGGGCTTGGTTAGTTGT
SEQ ID NO: 644
CTGGGAAATTATGGCACCAAATGCT
SEQ ID NO: 645
AGAAGCAATCGATGAAACATTTGGTAGCTTTGATG
SEQ ID NO: 646
CCAACTGGCGCTATTAAAGAAGCAATCGATGAAAC
SEQ ID NO: 647
AATGTTTCATCGATTGCTTCTTTAATAGCGCCAGT
SEQ ID NO: 648
ATCGATGAAACATTTGGTAGCTTTGATGAAATGAA
SEQ ID NO: 649
ATCGATTGCTTCTTTAATAGCGCCAGTTGGTTG
SEQ ID NO: 650
TGGCCGCTTTGGTTCAGGTTGGG
SEQ ID NO: 651
TGCCATAATTTCCCAGAAGAATGTATGGTTTGCG
SEQ ID NO: 652
CAGCATTTGGTGCCATAATTTCCCAGAAGAATGT
SEQ ID NO: 653
CCAACCTGAACCAAAGCGGCCAG
SEQ ID NO: 654
CAGCTGTTCGTAACAATGGTGGCGG
SEQ ID NO: 655
TTCTTTAATAGCGCCAGTTGGTTGTCCACCAG
SEQ ID NO: 656
TAACCAAGCCCAACCTGAACCAAAGCG
SEQ ID NO: 657
TGGTGGACAACCAACTGGCGCTATTAAAGAAG
SEQ ID NO: 658
AGCAGCTTTCATTTCATCAAAGCTACCAAATGTTT
SEQ ID NO: 659
CAAATGCTGGTGGACAACCAACTGGC
SEQ ID NO: 660
AAACATTTGGTAGCTTTGATGAAATGAAAGCTGCT
SEQ ID NO: 661
CGTGACCGCCACCATTGTTACGAACA
SEQ ID NO: 662
CGCCAGTTGGTTGTCCACCAGC
SEQ ID NO: 663
CTGCTTTCAAAACAGCTGCAACTGGCC
SEQ ID NO: 664
AGAAGAATGTATGGTTTGCGTGACCGCC
SEQ ID NO: 665
AAAGCGGCCAGTTGCAGCTGT
SEQ ID NO: 666
AACCAAACATTCTTCTGGGAAATTATGGCACCAAA
SEQ ID NO: 667
CGCCACCATTGTTACGAACGGCTGT
SEQ ID NO: 668
CACGCAAACCATACATTCTTCTGGGAAATTATGGC
SEQ ID NO: 669
TTCATTTCATCAAAGCTGCCAAATGTTTCATCGAT
SEQ ID NO: 670
CGCTATTAAAGAAGCAATCGATGAAACATTTGGCA
SEQ ID NO: 671
AAGCTGCCAAATGTTTCATCGATTGCTTCTTTAAT
SEQ ID NO: 672
TCATCAAAGCTGCCAAATGTTTCATCGATTGCTTC
SEQ ID NO: 673
CCAGTTGCAGCTGTTTTGAAAGCAGC
SEQ ID NO: 674
CATTCTTCTGGGAAATTATGGCACCAAATGCTGG
SEQ ID NO: 675
TGGGAAATTATGGCACCAAATGCTGGCG
SEQ ID NO: 676
CTGCGACTGGCCGCTTTGGTTCA
SEQ ID NO: 677
TGAACCAAAGCGGCCAGTCGCAG

TABLE 9B
Efs sodA gene solutions

| Group | | | |
|---|---|---|---|
| No. | Forward | Probe | Reverse |
| 606 | SEQ ID NO: 577 | SEQ ID NO: 661 | SEQ ID NO: 640 |
| | TGAATGCTATTCCTGAAGATATCCG | CGTGACCGCCACCATTGTTACGAACA | CCAGCATTTGGTGCCATAATTTC |
| 607 | SEQ ID NO: 577 | SEQ ID NO: 654 | SEQ ID NO: 640 |
| | TGAATGCTATTCCTGAAGATATCCG | CAGCTGTTCGTAACAATGGTGGCGG | CCAGCATTTGGTGCCATAATTTC |
| 608 | SEQ ID NO: 600 | SEQ ID NO: 650 | SEQ ID NO: 623 |
| | ACATTTGGCAGCTTTGATGAAATG | TGGCCGCTTTGGTTCAGGTTGGG | ATTTACCGTTATTCACAACTAACCA |
| 609 | SEQ ID NO: 590 | SEQ ID NO: 675 | SEQ ID NO: 637 |
| | GGTCACGCAAACCATACATTCTTC | TGGGAAATTATGGCACCAAATGCTGGCG | TGCCAAATGTTTCATCGATTGCTT |
| 610 | SEQ ID NO: 598 | SEQ ID NO: 644 | SEQ ID NO: 637 |
| | GGTCACGCAAACCAAACATTCT | CTGGGAAATTATGGCACCAAATGCT | TGCCAAATGTTTCATCGATTGCTT |
| 611 | SEQ ID NO: 577 | SEQ ID NO: 664 | SEQ ID NO: 640 |
| | TGAATGCTATTCCTGAAGATATCCG | AGAAGAATGTATGGTTTGCGTGACCGCC | CCAGCATTTGGTGCCATAATTTC |
| 612 | SEQ ID NO: 577 | SEQ ID NO: 667 | SEQ ID NO: 640 |
| | TGAATGCTATTCCTGAAGATATCCG | CGCCACCATTGTTACGAACGGCTGT | CCAGCATTTGGTGCCATAATTTC |
| 613 | SEQ ID NO: 590 | SEQ ID NO: 659 | SEQ ID NO: 637 |
| | GGTCACGCAAACCATACATTCTTC | CAAATGCTGGTGGACAACCAACTGGC | TGCCAAATGTTTCATCGATTGCTT |
| 614 | SEQ ID NO: 590 | SEQ ID NO: 662 | SEQ ID NO: 637 |
| | GGTCACGCAAACCATACATTCTTC | CGCCAGTTGGTTGTCCACCAGC | TGCCAAATGTTTCATCGATTGCTT |
| 615 | SEQ ID NO: 600 | SEQ ID NO: 676 | SEQ ID NO: 624 |
| | ACATTTGGCAGCTTTGATGAAATG | CTGCGACTGGCCGCTTTGGTTCA | AATTTACCGTTATTCACAACTAACC |
| 616 | SEQ ID NO: 599 | SEQ ID NO: 673 | SEQ ID NO: 625 |
| | GATGAAACATTTGGCAGCTTTGATG | CCAGTTGCAGCTGTTTTGAAAGCAGC | CCGTTATTCACAACTAACCAAGCC |
| 617 | SEQ ID NO: 600 | SEQ ID NO: 673 | SEQ ID NO: 624 |
| | ACATTTGGCAGCTTTGATGAAATG | CCAGTTGCAGCTGTTTTGAAAGCAGC | AATTTACCGTTATTCACAACTAACC |
| 618 | SEQ ID NO: 600 | SEQ ID NO: 677 | SEQ ID NO: 624 |
| | ACATTTGGCAGCTTTGATGAAATG | TGAACCAAAGCGGCCAGTCGCAG | AATTTACCGTTATTCACAACTAACC |
| 619 | SEQ ID NO: 599 | SEQ ID NO: 676 | SEQ ID NO: 625 |
| | GATGAAACATTTGGCAGCTTTGATG | CTGCGACTGGCCGCTTTGGTTCA | CCGTTATTCACAACTAACCAAGCC |
| 620 | SEQ ID NO: 599 | SEQ ID NO: 677 | SEQ ID NO: 625 |
| | GATGAAACATTTGGCAGCTTTGATG | TGAACCAAAGCGGCCAGTCGCAG | CCGTTATTCACAACTAACCAAGCC |
| 621 | SEQ ID NO: 586 | SEQ ID NO: 667 | SEQ ID NO: 617 |
| | TCAGATATGAATGCTATTCCTGAAG | CGCCACCATTGTTACGAACGGCTGT | TTTGGTGCCATAATTTCCCAGAAGA |
| 622 | SEQ ID NO: 599 | SEQ ID NO: 665 | SEQ ID NO: 625 |
| | GATGAAACATTTGGCAGCTTTGATG | AAAGCGGCCAGTTGCAGCTGT | CCGTTATTCACAACTAACCAAGCC |
| 623 | SEQ ID NO: 599 | SEQ ID NO: 663 | SEQ ID NO: 625 |
| | GATGAAACATTTGGCAGCTTTGATG | CTGCTTTCAAAACAGCTGCAACTGGCC | CCGTTATTCACAACTAACCAAGCC |
| 624 | SEQ ID NO: 600 | SEQ ID NO: 665 | SEQ ID NO: 624 |
| | ACATTTGGCAGCTTTGATGAAATG | AAAGCGGCCAGTTGCAGCTGT | AATTTACCGTTATTCACAACTAACC |
| 625 | SEQ ID NO: 586 | SEQ ID NO: 661 | SEQ ID NO: 617 |
| | TCAGATATGAATGCTATTCCTGAAG | CGTGACCGCCACCATTGTTACGAACA | TTTGGTGCCATAATTTCCCAGAAGA |
| 626 | SEQ ID NO: 586 | SEQ ID NO: 654 | SEQ ID NO: 617 |
| | TCAGATATGAATGCTATTCCTGAAG | CAGCTGTTCGTAACAATGGTGGCGG | TTTGGTGCCATAATTTCCCAGAAGA |
| 627 | SEQ ID NO: 600 | SEQ ID NO: 663 | SEQ ID NO: 624 |
| | ACATTTGGCAGCTTTGATGAAATG | CTGCTTTCAAAACAGCTGCAACTGGCC | AATTTACCGTTATTCACAACTAACC |

TABLE 10A
Efm novel gene nucleic acid primers and probes

Primer
SEQ ID NO: 678
CCTCCTCGTTTCTTACTGAT
SEQ ID NO: 679
GGTTCCTCCTCGTTTCTT
SEQ ID NO: 680
GTTTTCGTTCTCTTCTAGCAAAA
SEQ ID NO: 681
CGATCCATCCGACTCATTG
SEQ ID NO: 682
CGATCCATCCGACTCATT
SEQ ID NO: 683
TTTCGCCTTCTTATGGATGTCCTTA
SEQ ID NO: 684
GATCCATCCGACTCATTGTGGT
SEQ ID NO: 685
TTATAGCGATCCATCCGACTCATTG
SEQ ID NO: 686
TTATAGCGATCCATCCGACTCATT
SEQ ID NO: 687
TCTTATGGATGTCCTTATAGCGATC
SEQ ID NO: 688
TGGATGTCCTTATAGCGATCCATC
SEQ ID NO: 689
TTGTGGTTCCTCCTCGTTTCTTAC
SEQ ID NO: 690
TCATTGTGGTTCCTCCTCGTTTC
SEQ ID NO: 691
TCCGACTCATTGTGGTTCCTC
SEQ ID NO: 692
TTCAGAGGTTTCGCCTTCTTATGG
SEQ ID NO: 693
GCTTCAGAGGTTTCGCCTTCTTA
SEQ ID NO: 694
GCTTCAGAGGTTTCGCCTTCTT
SEQ ID NO: 695
GTATCCCGCTTCAGAGGTTTC
SEQ ID NO: 696
TCCCGCTTCAGAGGTTTCG
SEQ ID NO: 697
CGTATCCCGCTTCAGAGG
SEQ ID NO: 698
GGTTTCGCCTTCTTATGGATGTC
SEQ ID NO: 699
TACTCCCCGTATCCCGCTTCA
SEQ ID NO: 700
CCCGTATCCCGCTTCAGA
SEQ ID NO: 701
AAGAAACGAGGAGGAACC
SEQ ID NO: 702
CGCTATAAGGACATCCATAAGA
SEQ ID NO: 703
CGAGGAGGAACCACAATG
SEQ ID NO: 704
CTTTTGTCGATTGGTTATCGTA
SEQ ID NO: 705
CTTTTGTCGATTGGTTATCGTAT
SEQ ID NO: 706
TATCAGTAAGAAACGAGGAGGAACC
SEQ ID NO: 707
ATGATAGGCTTTTGTCGATTGGTTA
SEQ ID NO: 708
CACAATGAGTCGGATGGATCG
SEQ ID NO: 709
ACCACAATGAGTCGGATGGA
SEQ ID NO: 710
CAATGAGTCGGATGGATCGCTAT
SEQ ID NO: 711
CAATGAGTCGGATGGATCGCTA
SEQ ID NO: 712
GTCGGATGGATCGCTATAAGG
SEQ ID NO: 713
ATTTATCAGTAAGAAACGAGGAGGA
SEQ ID NO: 714
GGATGGATCGCTATAAGGACATCC
SEQ ID NO: 715
TAAGAAACGAGGAGGAACCACAAT
SEQ ID NO: 716
GAGGAGGAACCACAATGAGTCG
SEQ ID NO: 717
ATAAGGACATCCATAAGAAGGCGAA
SEQ ID NO: 718
ATCGCTATAAGGACATCCATAAGAA
SEQ ID NO: 719
TGTATCGAAGCATTTATTTCGTTAT
SEQ ID NO: 720
GGCTTTTGTCGATTGGTTATCGTAT
SEQ ID NO: 721
GGCTTTTGTCGATTGGTTATCGTA
SEQ ID NO: 722
TTTGTCGATTGGTTATCGTATCCTT
SEQ ID NO: 723
TGTCGATTGGTTATCGTATCCTTTA
SEQ ID NO: 724
CGAGGAGGAACCACAATGAG
SEQ ID NO: 725
AAATGATAAGCTTTTGTCGATTGGT
Probe
SEQ ID NO: 726
CGGATGGATCGCTATAAGGACATCCAT
SEQ ID NO: 727
AGGATACGATAACCAATCGACAAAAGC
SEQ ID NO: 728
CGATCCATCCGACTCATTGTGGT
SEQ ID NO: 729
ACTGATAAATAAAGGATACGATAACCAATCGACAA
SEQ ID NO: 730
CCCGTATCCCGCTTCAGAGG
SEQ ID NO: 731
AGGCGAAACCTCTGAAGCG
SEQ ID NO: 732
TTCTTATGGATGTCCTTATAGCGATCCATCCGACT
SEQ ID NO: 733
TGGATGTCCTTATAGCGATCCATCCGACTCATTG
SEQ ID NO: 734
TGGTTCCTCCTCGTTTCTTACTGATAAATAAAGGA
SEQ ID NO: 735
TTCGCCTTCTTATGGATGTCCTTATAGCGATCCA
SEQ ID NO: 736
AATGATAGGCTTTTGTCGATTGGTTATCGTATCCT
SEQ ID NO: 737
CAATGAGTCGGATGGATCGCTATAAGGACATCCAT
SEQ ID NO: 738
CCTTTATTTATCAGTAAGAAACGAGGAGGAACCAC
SEQ ID NO: 739
TTATAGCGATCCATCCGACTCATTGTGGTTCCTC
SEQ ID NO: 740
TCAGTAAGAAACGAGGAGGAACCACAATGAGTCG
SEQ ID NO: 741
TCGCTATAAGGACATCCATAAGAAGGCGAAACCTC
SEQ ID NO: 742
CTCCTCGTTTCTTACTGATAAATAAAGGATACGAT
SEQ ID NO: 743
TCGTATCCTTTATTTATCAGTAAGAAACGAGGAGG
SEQ ID NO: 744
AAATAAAGGATACGATAACCAATCGACAAAAGCCT
SEQ ID NO: 745
ATAAATAAAGGATACGATAACCAATCGACAAAAGC
SEQ ID NO: 746
CTTCAGAGGTTTCGCCTTCTTATGGATGTCCTTAT
SEQ ID NO: 747
TAAGGACATCCATAAGAAGGCGAAACCTCTGAAGC
SEQ ID NO: 748
TTGTCGATTGGTTATCGTATCCTTTATTTATCAGT
SEQ ID NO: 749
CATCCATAAGAAGGCGAAACCTCTGAAGCG
SEQ ID NO: 750
CGAGGAGGAACCACAATGAGTCGGATG
SEQ ID NO: 751
CGACTCATTGTGGTTCCTCCTCGTTTCTTACTG
SEQ ID NO: 752
AGGATACGATAACCAATCGACAAAAGCTTATCATT
SEQ ID NO: 753
AAGCTTTTGTCGATTGGTTATCGTATCCTTTATTT

TABLE 10B
Efm novel gene solutions

| Group | | | |
|---|---|---|---|
| No. | Forward | Probe | Reverse |
| 628 | SEQ ID NO: 687 | SEQ ID NO: 750 | SEQ ID NO: 723 |
| | TCTTATGGATGTCCTTATAGCGATC | CGAGGAGGAACCACAATGAGTCGGATG | TGTCGATTGGTTATCGTATCCTTTA |
| 629 | SEQ ID NO: 687 | SEQ ID NO: 750 | SEQ ID NO: 720 |
| | TCTTATGGATGTCCTTATAGCGATC | CGAGGAGGAACCACAATGAGTCGGATG | GGCTTTTGTCGATTGGTTATCGTAT |
| 630 | SEQ ID NO: 683 | SEQ ID NO: 728 | SEQ ID NO: 723 |
| | TTTCGCCTTCTTATGGATGTCCTTA | CGATCCATCCGACTCATTGTGGT | TGTCGATTGGTTATCGTATCCTTTA |
| 631 | SEQ ID NO: 683 | SEQ ID NO: 750 | SEQ ID NO: 723 |
| | TTTCGCCTTCTTATGGATGTCCTTA | CGAGGAGGAACCACAATGAGTCGGATG | TGTCGATTGGTTATCGTATCCTTTA |
| 632 | SEQ ID NO: 687 | SEQ ID NO: 750 | SEQ ID NO: 707 |
| | TCTTATGGATGTCCTTATAGCGATC | CGAGGAGGAACCACAATGAGTCGGATG | ATGATAGGCTTTTGTCGATTGGTTA |
| 633 | SEQ ID NO: 683 | SEQ ID NO: 728 | SEQ ID NO: 720 |
| | TTTCGCCTTCTTATGGATGTCCTTA | CGATCCATCCGACTCATTGTGGT | GGCTTTTGTCGATTGGTTATCGTAT |
| 634 | SEQ ID NO: 683 | SEQ ID NO: 750 | SEQ ID NO: 720 |
| | TTTCGCCTTCTTATGGATGTCCTTA | CGAGGAGGAACCACAATGAGTCGGATG | GGCTTTTGTCGATTGGTTATCGTAT |
| 635 | SEQ ID NO: 692 | SEQ ID NO: 728 | SEQ ID NO: 723 |
| | TTCAGAGGTTTCGCCTTCTTATGG | CGATCCATCCGACTCATTGTGGT | TGTCGATTGGTTATCGTATCCTTTA |
| 636 | SEQ ID NO: 692 | SEQ ID NO: 750 | SEQ ID NO: 723 |
| | TTCAGAGGTTTCGCCTTCTTATGG | CGAGGAGGAACCACAATGAGTCGGATG | TGTCGATTGGTTATCGTATCCTTTA |

TABLE 11A
Efs novel gene nucleic acid primers and probes

Primer
SEQ ID NO: 754
CATCTTCAGGAGCTAAATCAAT
SEQ ID NO: 755
AAGTACTCGTACTTGAAAATCATCT
SEQ ID NO: 756
GTCGCACATCTTCAGGAGCTAAA
SEQ ID NO: 757
CTAATTGTTTCACTGTCTCTGGATT
SEQ ID NO: 758
AAGAAGTATCCAATGACTGTTGCAA
SEQ ID NO: 759
TCCAATGACTGTTGCAATAACTGGA
SEQ ID NO: 760
TTCACTGTCTCTGGATTAGGTACTC
SEQ ID NO: 761
AATTGTTTCACTGTCTCTGGATTAG
SEQ ID NO: 762
TCACCATTTTCAATATACGCATCTA
SEQ ID NO: 763
TTCACCATTTTCAATATACGCATCT
SEQ ID NO: 764
CGTATGATACAGCGTTTCTAATTGT
SEQ ID NO: 765
AGCGTTTCTAATTGTTTCACTGTCT
SEQ ID NO: 766
CAGCGTTTCTAATTGTTTCACTGTC
SEQ ID NO: 767
TTAGGTACTCCGTCAAGTACTCGT
SEQ ID NO: 768
GACAAGCGTCGCACATCTTC
SEQ ID NO: 769
AAAAGAAGTATCCAATGACTGTTGC
SEQ ID NO: 770
TTTTAATCGTATGATACAGCGTTTC
SEQ ID NO: 771
GGAACAAATTAGCACCTCTATTCTA
SEQ ID NO: 772
GATTGCTTGTTCCATTGGCT
SEQ ID NO: 773
TGTTGCAATAACTGGATTGCTTGTT
SEQ ID NO: 774
TCTGGATTAGGTACTCCGTCAAGT
SEQ ID NO: 775
TAACTGGATTGCTTGTTCCATTGG
SEQ ID NO: 776
TAATTGGGACAAGCGTCGCA
SEQ ID NO: 777
TTTCTGGGAACAAATTAGCACCTCT
SEQ ID NO: 778
TCCGTCAAGTACTCGTACTTGAAAA
SEQ ID NO: 779
ATTGTTTCACTGTCTCTGGATTAGG
SEQ ID NO: 780
GCTTTCGAGCCAATGGAACA
SEQ ID NO: 781
CGGAGTACCTAATCCAGAG
SEQ ID NO: 782
TTCGTTAAAATAAAACAGGTATGGA
SEQ ID NO: 783
AGTAAAGGCATTGATTATTCTTTCT
SEQ ID NO: 784
ACAGTAAAGGCATTGATTATTCTTT
SEQ ID NO: 785
ATGGAAGTTGTTACATTTTGAATAG
SEQ ID NO: 786
TTGCAACAGTCATTGGATACTTCTT
SEQ ID NO: 787
TTAGATGATTTTCAAGTACGAGTAC
SEQ ID NO: 788
GAGCCAATGGAACAAGCAATCCA
SEQ ID NO: 789
GAGGTGCTAATTTGTTCCCAGAAAA
SEQ ID NO: 790
GTACGAGTACTTGACGGAGTACCTA
SEQ ID NO: 791
AAAATAAAACAGGTATGGAAGTTG
SEQ ID NO: 792
TCCAGTTATTGCAACAGTCATTGGA
SEQ ID NO: 793
AGAAGAAAGACAGTAAAGGCATTGA
SEQ ID NO: 794
TAAAACAGGTATGGAAGTTGTTACA
SEQ ID NO: 795
TTTCGAGCCAATGGAACAAGCA
SEQ ID NO: 796
TGGAACAAGCAATCCAGTTATTGCA
SEQ ID NO: 797
TGAATAGAATAGAGGTGCTAATTTG
SEQ ID NO: 798
GAGCCAATGGAACAAGCAAT
SEQ ID NO: 799
AACAGGTATGGAAGTTGTTACATTT
SEQ ID NO: 800
CAAGCAATCCAGTTATTGCAACAG
SEQ ID NO: 801
AGACAGTAAAGGCATTGATTATTCT
SEQ ID NO: 802
GCAACAGTCATTGGATACTTCTTTT
SEQ ID NO: 803
TAGAGGTGCTAATTTGTTCCCAGAA
SEQ ID NO: 804
AAAGGCATTGATTATTCTTTCTTTT
SEQ ID NO: 805
GAATAGAATAGAGGTGCTAATTTGT
SEQ ID NO: 806
ATAGAATAGAGGTGCTAATTTGTTC
SEQ ID NO: 807
GTACTTGACGGAGTACCTAATCCAG
SEQ ID NO: 808
TTTTCAAGTACGAGTACTTGACGGA
SEQ ID NO: 809
AGATGCGTATATTGAAAATGGTGAA
Probe
SEQ ID NO: 810
CCAGTTATTGCAACAGTCATTGGATACTTC
SEQ ID NO: 811
CGTTAAAATAAAACAGGTATGGAAGTTGTTACATT
SEQ ID NO: 812
CCGTCAAGTACTCGTACTTGAAAATCATCTAAAAT
SEQ ID NO: 813
ATGTAACAACTTCCATACCTGTTTTATTTTAACGA
SEQ ID NO: 814
ACATTTTGAATAGAATAGAGGTGCTAATTTGTTCC
SEQ ID NO: 815
AATAGAGGTGCTAATTTGTTCCCAGAAAA
SEQ ID NO: 816
CCAGAGACAGTGAAACAATTAGAAACGCTGTATCA
SEQ ID NO: 817
CGTATGATACAGCGTTTCTAATTGTTTCACTGTCT
SEQ ID NO: 818
AGAAGTATCCAATGACTGTTGCAATAACTGGATTG
SEQ ID NO: 819
ACAGTGAAACAATTAGAAACGCTGTATCATACGAT
SEQ ID NO: 820
CTGTCTCTGGATTAGGTACTCCGTCAAGTACTCGT
SEQ ID NO: 821
CTGGATTAGGTACTCCGTCAAGTACTCGTACTTGA
SEQ ID NO: 822
AGGTACTCCGTCAAGTACTCGTACTTGAAAATCAT
SEQ ID NO: 823
AGCGTTTCTAATTGTTTCACTGTCTCTGGATTAGG
SEQ ID NO: 824
ATCCAATGACTGTTGCAATAACTGGATTGCTTGTT
SEQ ID NO: 825
AAACAGGTATGGAAGTTGTTACATTTTGAATAGAA
SEQ ID NO: 826
AATGGAACAAGCAATCCAGTTATTGCAACAGTCAT
SEQ ID NO: 827
CTTTCGAGCCAATGGAACAAGCAATCCAGTTATT
SEQ ID NO: 828
ACCTAATCCAGAGACAGTGAAACAATTAGAAACGC
SEQ ID NO: 829
AGCACCTCTATTCTATTCAAAATGTAACAACTTCC
SEQ ID NO: 830
ATTGTTTCACTGTCTCTGGATTAGGTACTCCGTCA
SEQ ID NO: 831
TGTTGCAATAACTGGATTGCTTGTTCCATTGGCTC
SEQ ID NO: 832
TAACTGGATTGCTTGTTCCATTGGCTCGAAAGC
SEQ ID NO: 833
ATTCTATTCAAAATGTAACAACTTCCATACCTGTT
SEQ ID NO: 834
ATGATTTTCAAGTACGAGTACTTGACGGAGTACCT
SEQ ID NO: 835
CTGGGAACAAATTAGCACCTCTATTCTATTCAAAA
SEQ ID NO: 836
CAAGTACGAGTACTTGACGGAGTACCTAATCCAGA
SEQ ID NO: 837
TGACGGAGTACCTAATCCAGAGACAGTGAAACAAT
SEQ ID NO: 838
TGGAAGTTGTTACATTTTGAATAGAATAGAGGTGC
SEQ ID NO: 839
TGAATAGAATAGAGGTGCTAATTTGTTCCCAGAAA
SEQ ID NO: 840
CGAGTACTTGACGGAGTACCTAATCCAGAGACAG
SEQ ID NO: 841
ATTTTAGATGATTTTCAAGTACGAGTACTTGACGG
SEQ ID NO: 842
AGCAATCCAGTTATTGCAACAGTCATTGGATACTT

TABLE 11B
Efs novel gene solutions

| Group | | | |
|---|---|---|---|
| No. | Forward | Probe | Reverse |
| 637 | SEQ ID NO: 758 | SEQ ID NO: 832 | SEQ ID NO: 803 |
| | AAGAAGTATCCAATGACTGTTGCAA | TAACTGGATTGCTTGTTCCATTGGCTCGAAAGC | TAGAGGTGCTAATTTGTTCCCAGAA |
| 638 | SEQ ID NO: 775 | SEQ ID NO: 815 | SEQ ID NO: 785 |
| | TAACTGGATTGCTTGTTCCATTGG | AATAGAGGTGCTAATTTGTTCCCAGAAAA | ATGGAAGTTGTTACATTTTGAATAG |
| 639 | SEQ ID NO: 772 | SEQ ID NO: 815 | SEQ ID NO: 799 |
| | GATTGCTTGTTCCATTGGCT | AATAGAGGTGCTAATTTGTTCCCAGAAAA | AACAGGTATGGAAGTTGTTACATTT |
| 640 | SEQ ID NO: 758 | SEQ ID NO: 832 | SEQ ID NO: 797 |
| | AAGAAGTATCCAATGACTGTTGCAA | TAACTGGATTGCTTGTTCCATTGGCTCGAAAGC | TGAATAGAATAGAGGTGCTAATTTG |
| 641 | SEQ ID NO: 775 | SEQ ID NO: 815 | SEQ ID NO: 799 |
| | TAACTGGATTGCTTGTTCCATTGG | AATAGAGGTGCTAATTTGTTCCCAGAAAA | AACAGGTATGGAAGTTGTTACATTT |
| 642 | SEQ ID NO: 772 | SEQ ID NO: 815 | SEQ ID NO: 791 |
| | GATTGCTTGTTCCATTGGCT | AATAGAGGTGCTAATTTGTTCCCAGAAAA | AAAATAAAACAGGTATGGAAGTTG |
| 643 | SEQ ID NO: 773 | SEQ ID NO: 815 | SEQ ID NO: 785 |
| | TGTTGCAATAACTGGATTGCTTGTT | AATAGAGGTGCTAATTTGTTCCCAGAAAA | ATGGAAGTTGTTACATTTTGAATAG |

TABLE 12
Efm/Efs dual nucleic acid primers and probes

| Group | | | |
|---|---|---|---|
| No. | Forward | Probe | Reverse |
| 644 | SEQ ID NO: 843 | SEQ ID NO: 844 | SEQ ID NO: 845 |
| | TTGCTTTATCGT | CATTCTCCTCGCT | GATAGTATGGAGGT |
| | GACGTGACTTTG | ATCATCGGTTTAA | GGAAATATGGT |
| | | TTGTCGG | SEQ ID NO: 846 |
| | | | TATGGAGGTGGAT |
| | | | TTTATGGTATTC |

### Example 6

### Testing vanA and vanB Primer and Probe Sequences for their Ability to Amplify their Intended Target Sequences

The oligonucleotide sequences listed in Table 5 were tested for their ability to amplify their intended target sequences. About 25 μA PCRs were formulated using iQTM Supermix for qPCR (BioRad) and oligos at a final concentration of 400 nM each.

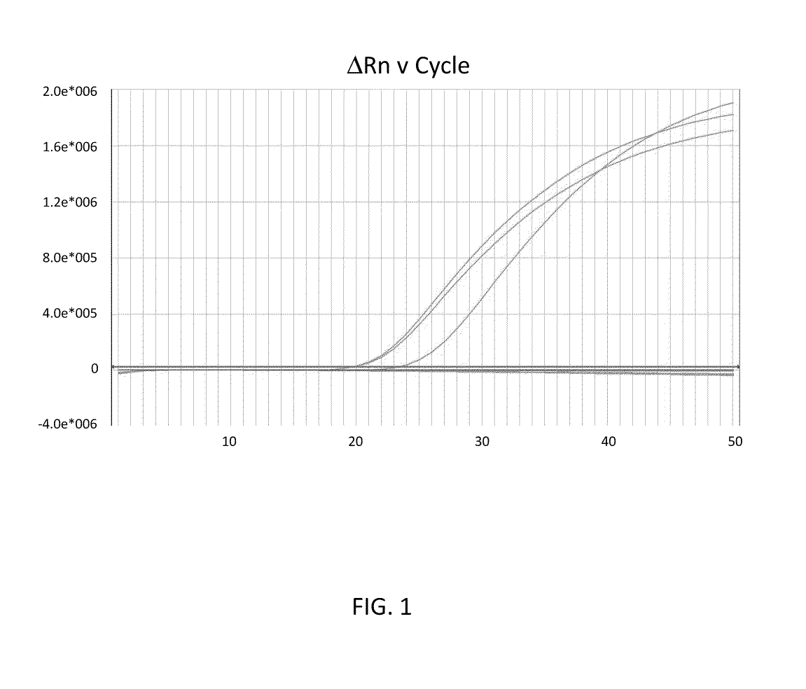

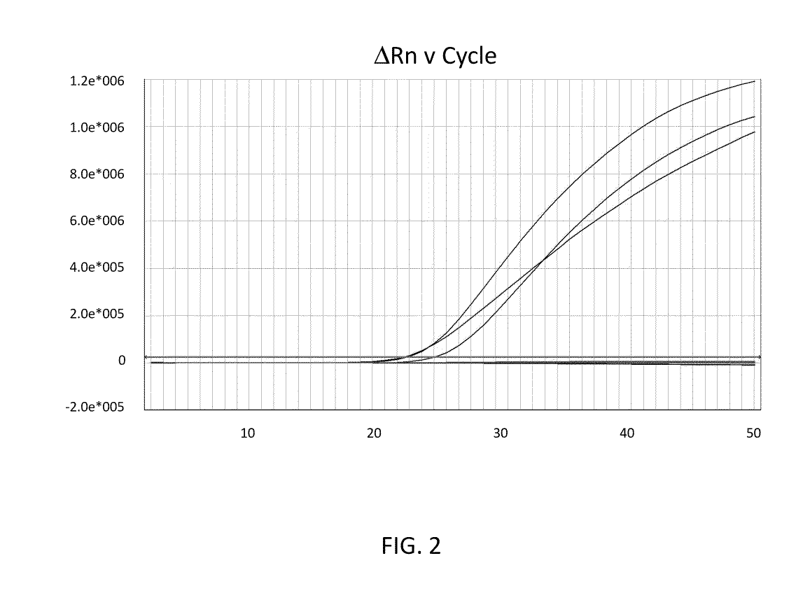

Genomic DNA isolated from E. faecium (ATCC No. 5159, strain MMC4, vanA) and E. faecalis (ATCC No. 700802, strain V583, vanB) were loaded into real-time PCRs using oligonucleotide solutions specific for vanA and vanB. The specificity of the oligonucleotide solutions was assessed by attempting to amplify E. faecalis (vanB) gDNA with vanA oligos and E. faecium (vanA) gDNA with vanB oligos. Amplification plots are illustrated in FIGS. 1 and 2, which show detection of vanA and vanB. Synthetic constructs encoding the vanA and vanB targets were amplified and compared to duplicate reactions where E. faecium (ATCC No. 5159, strain MMC4, vanA) and E. faecalis (ATCC No. 700802, strain V583, vanB) gDNA was amplified.

PCRs were run on the ABI 7500 with the following Thermal protocol:
95° C. 5 minute, initial denaturation
50 cycles of:
   95° C. 15 sec, denaturation, and
   60° C. 1 min, annealing/extension

In FIG. 1, the vanA solution (SEQ ID NO: 1, 2, and 5) included amplification of E. faecium gDNA (*) and the E. faecium vanA synthetic construct (§), while the vanB solution (SEQ ID NOS: 103, 108, and 66) in FIG. 2 included amplification of the E. faecalis vanB synthetic construct (¥) and E. faecalis gDNA (+). PCR products were not evident in any of the No template controls (NTC). Ct values corresponding to the presence of PCR product are shown in Table 13 below.

TABLE 13
Ct values showing presence of vanA and vanB.

| | Oligo solution | |
|---|---|---|
| Input | vanA Ct | vanB Ct |
| Synthetic construct | 23.36 | 24.04 |
| NTC | UND | UND |
| E. faecalis (ATCC No. 700802, strain V583, vanB) | UND | 21.85 |
| | UND | 21.53 |
| E. faecium (ATCC No. 5159, strain MMC4 vanA) | 19.67 | UND |
| | 19.80 | UND |

### Example 7

### Gel Electrophoresis Analysis for vanA and vanB

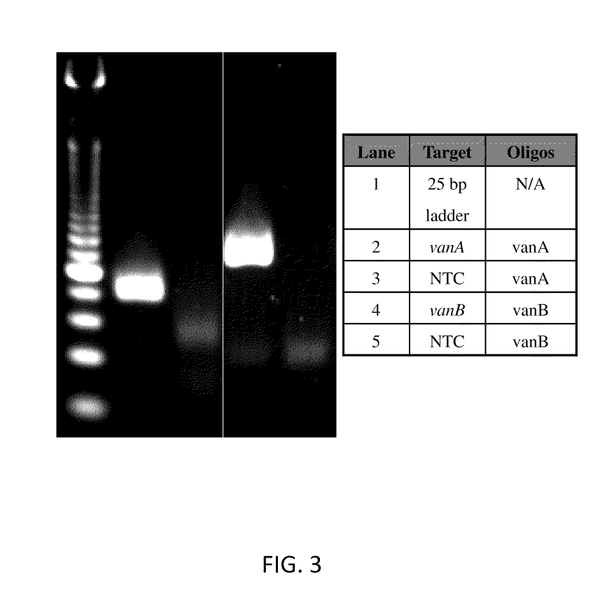

A gel electrophoresis analysis was performed using 20 μL aliquots of post-amplification vanA and vanB PCR products on an agarose gel plate. The gel electrophoresis of FIG. 3 illustrates that the molecular weight marker was a 25 bp ladder (Invitrogen); gel, 4% agarose e-gel (Invitrogen), EtBr stained; inputs are shown in the table of FIG. 3. The arrows on the gel point to the 100 bp and 150 bp markers. The gel illustrates that the vanA and vanB PCR products migrate according to their predicted sizes.

### Example 8

### Testing sodA and Efm/EFs Dual Sequences for Ability to Amplify their Intended Target Sequences

The nucleic acid primers and probes listed in Tables 8B, 9B and 12 were tested for their ability to amplify their intended target sequences. About 25 μL PCRs were formulated using iQTM Supermix for qPCR (BioRad) and oligonucleotides at a final concentration of 400 nM each.

In this example, 1×10⁴ copies of genomic DNA from E. faecium (ATCC No. 51559), E. faecalis (ATCC. No 700802) and C. difficile (ATCC No. 43598) were loaded into real-time PCRs using oligonucleotide solutions directed against E. faecium sodA, E. faecalis sodA novel markers for both E. faecalis and E. faecium (Efm/Efs dual). PCRs were run on the ABI 7500 with the following Thermal protocol:
95° C. 5 minute, initial denaturation
50 cycles of:
   95° C. 15 sec, denaturation
   60° C. 1 min, annealing/extension

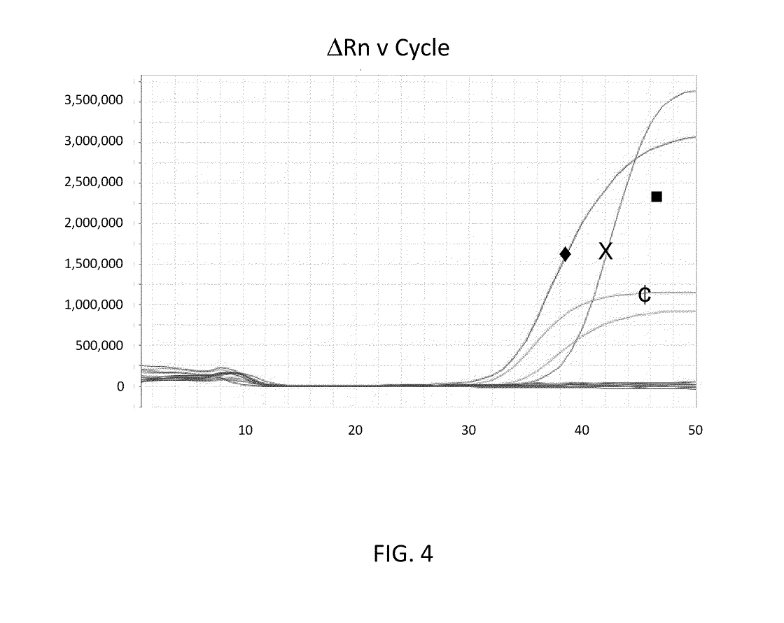

In FIG. 4, amplification plots show detection of E. faecium and not E. faecalis or C. difficile using the E. faecium sodA oligonucleotide solution (♦—SEQ ID NO: 517, 571, and 529); detection of E. faecalis and not E. faecium or C. difficile using the E. faecalis sodA oligonulceotide solution (X—SEQ ID NO: 599, 663, and 625); and detection of both E. faecium and E. faecalis but not C. difficile using the Efm/Efs dual oligonucleotide solution (·—SEQ ID NO: 843, 844, 845 and 846). Ct values corresponding to the presence of PCR product are shown in Table 14 below. PCR products were not evident in any of the No template controls (NTC).

TABLE 14
Ct values showing presence of sodA for E. faecium,
E. faecalis, and Efs/Efm dual.

| Target Input | Oligos | Ct |
|---|---|---|
| E. faecium | E. faecium | 31.45 |
| E. faecalis | | UND |
| C. difficile | | UND |
| NTC | | UND |
| E. faecium | E. faecalis | UND |
| E. faecalis | | 36.52 |
| C. difficile | | UND |
| NTC | | UND |
| E. faecium | Dual | 32.45 |
| E. faecalis | | 34.93 |
| C. difficile | | UND |
| NTC | | UND |

### Example 9

### Gel Electrophoresis Analysis for Soda of E. faecium, E. faecalis, and Efm/Efs Dual

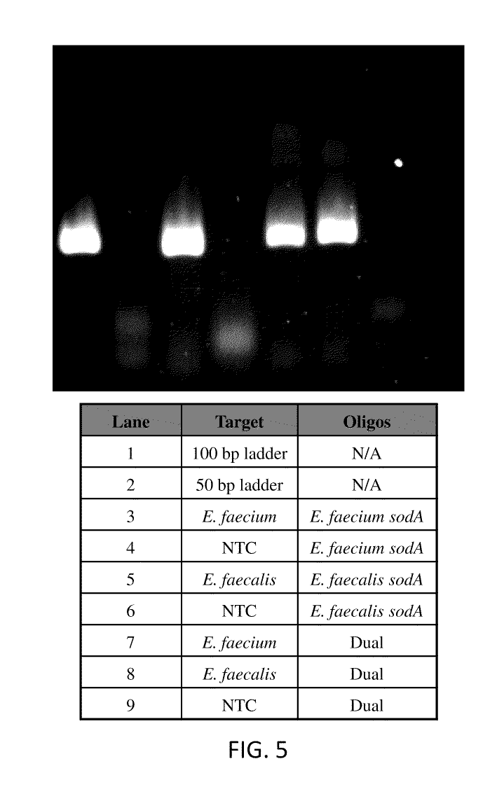

A gel electrophoresis analysis was performed using 20 μL aliquots of post-amplification PCR sodA and dual Efs/Efm products on an agarose gel plate. The gel electrophoresis was shown in FIG. 5 where molecular weight markers were the 100 bp ladder and 50 bp ladder (Invitrogen); gel, 4% agarose e-gel (Invitrogen), EtBr stained; inputs as shown in the table of FIG. 5. The arrow on the gel of FIG. 5 points to the 100 bp marker. The gel illustrates that the PCR products migrate according to their predicted sizes.

TABLE 15
Additional Sequences
SEQ ID NO: 113
0000
SEQ ID NO: 114
0000
SEQ ID NO: 115
0000
SEQ ID NO: 116
0000
SEQ ID NO: 117
0000
SEQ ID NO: 118
0000
SEQ ID NO: 119
0000
SEQ ID NO: 120
0000
SEQ ID NO: 121
0000
SEQ ID NO: 122
0000

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

1. An isolated nucleic acid sequence comprising a sequence selected from the group consisting of: SEQ ID NOS: 1-846.

2. A method of hybridizing one or more isolated nucleic acid sequences comprising a sequence selected from the group consisting of: SEQ ID NOS: 1-502 to a vancomycin-resistance gene, comprising contacting one or more isolated nucleic acid sequences to a sample comprising the vancomycin-resistance gene under conditions suitable for hybridization.

3. The method of claim 2, further comprising isolating the hybridized vancomycin-resistance gene.

4. The method of claim 2, further comprising sequencing the hybridized vancomycin-resistance gene.

5. A primer set comprising at least one forward primer selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG), and at least one reverse primer selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG).

6. The primer set of claim 5, wherein the primer set is selected from the
group consisting of: Groups 1-601 of Tables 5 and 6.

7. A method of producing a nucleic acid product, comprising contacting one or more isolated nucleic acid sequences selected from the group consisting of: SEQ ID NOS: 1, 3, 5, 6, 8, 10, 19, 21, 22, 23, 26, 28, 29, 31, 32, 33, 34, 36, 37-42, 44, 45, 47, 48, 51-53, 55, 59-61, 63, 65, 66, 68, 70-72, 74, 75, 77, 81, 83, 85-91, 93, 94, 95, 97-105, 107, 111, 123, 125, 127, 129, 130, 132, 133, 135, 137, 138, 140, 141, 143, 144, 146-148, 150, 151, 153, 155-158, 160-162, 164, 165, 167-171, 173-175, 177-180, 183-185, 187, 188, 190-194, 196, 198, 200-204, 206, 208-210, 212, 213, 215-217, 219-243, 246-248, 250, 251, 254, 259, 262, 266, 269, 274, 284, 286, 287, 290, 301, 304, 306, 308, 312, 320, 324, 336, 381, 388, 390, 391, 393-396, 398, 399, 401, 409, 411, 415-417, 419, 421, 425, 426, 428, 430, 434, 435, 437-440, 442-445, 447-455, 458-462, 464, 465, 467, 468, 470, 471, 473, 474, 476, 477, 479, 480, 482, 483, 486, 488, 490, 491, 493-498 and 500-502, to a sample comprising a vancomycin-resistance gene under conditions suitable for nucleic acid polymerization.

8. The method of claim 7, wherein the nucleic acid product is an amplicon produced using at least one forward primer selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG), and at least one reverse primer selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG).

9. The method of claim 8, wherein the vancomycin-resistance gene is selected from the group consisting of: vanA, vanB, vanC1, vanC2, vanC3, vanD, vanE and vanG.

10. A probe that hybridizes to the nucleic acid product of claim 7.

11. The probe of claim 10, wherein the probe comprises a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG).

12. The probe of claim 10, wherein the probe is labeled with a detectable label selected from the group consisting of: a fluorescent label, a chemiluminescent label, a quencher, a radioactive label, biotin and gold.

13. A set of probes that hybridize to the amplicon of claim 8, wherein a first probe comprises a sequence selected from the group consisting of: SEQ ID NOS: 2, 4, 7, 9, 11-18, 20, 24, 25, 27, 30, 35, 43, 46, 49, 50, 54, 56-58 (vanA probes), and a second probe comprises a sequence selected from the group consisting of: SEQ ID NOS: 62, 64, 67, 69, 73, 76, 78-80, 82, 84, 92, 96, 108-110, 112 (vanB probes).

14. The set of probes of claim 13, wherein the first probe is labeled with a first detectable label and the second probe is labeled with a second detectable label.

15. The set of probes of claim 13, wherein the first probe and the second probe are labeled with the same detectable label.

16. The set of probes of claim 14, wherein the detectable labels are selected from the group consisting of: a fluorescent label, a chemiluminescent label, a quencher, a radioactive label, biotin and gold.

17. A method for detecting a vancomycin-resistance gene in a sample, comprising:
a) contacting the sample with at least one forward primer comprising a sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 263, 264, 267, 270-272, 275-283, 285, 288, 291, 293, 295, 297-300, 302, 303, 305, 307, 309-311, 313-318, 321, 322, 325-333 (vanG), and at least one reverse primer comprising a sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG) under conditions such that nucleic acid amplification occurs to yield an amplicon; and
b) contacting the amplicon with one or more probes comprising one or more sequences selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG),
under conditions such that hybridization of the probe to the amplicon occurs, wherein hybridization of the probe is indicative of a vancomycin-resistance gene in the sample.

18. The method of claim 17, wherein each of the one or more probes is labeled with a different detectable label.

19. The method of claim 17, wherein the one or more probes are labeled with the same detectable label.

20. The method of claim 17, wherein the at least one forward primer, the at least one reverse primer and the one or more probes are selected from the group consisting of: Groups 1-601 of Tables 5 and 6.

21. A kit for detecting a vancomycin-resistance gene in a sample, comprising one or more probes comprising a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG).

22. The kit of claim 21, further comprising:
a) at least one forward primer comprising the sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); and
b) at least one reverse primer comprising the sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG).

23. The kit of claim 21, further comprising reagents for sequencing a vancomycin-resistance gene in the sample.

24. The kit of claim 21, wherein the one or more probes are labeled with different detectable labels.

25. The kit of claim 21, wherein the one or more probes are labeled with the same detectable label.

26. The kit of claim 22, wherein the at least one forward primer and the at least one reverse primer are selected from the group consisting of: Groups 1-601 of Tables 5 and 6.

27. A method of diagnosing a condition, syndrome or disease in a human associated with a vancomycin-resistance organism, comprising:
a) contacting a sample with at least one forward and reverse primer set selected from the group consisting of: Groups 1-601 of Tables 5 and 6;
b) conducting an amplification reaction, thereby producing an amplicon; and
c) detecting the amplicon using one or more probes selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG);
wherein the detection of an amplicon is indicative of the presence of a vancomycin-resistance organism in the sample.

28. A method of diagnosing a condition, syndrome or disease in a human associated with a vancomycin-resistant organism, comprising contacting a denatured target from a sample with one or more probes comprising a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG) under conditions for hybridization to occur; wherein hybridization of the one or more probes to a denatured target is indicative of the presence of a vancomycin-resistant organism in the sample.

29. A probe that hybridizes to a vancomycin-resistance gene target.

30. The probe of claim 29, wherein the probe comprises a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); and 245, 257, 261, 265, 268, 273, 289, 292, 294, 296, 319, 323 (vanG).

31. The probe of claim 29, wherein the probe is labeled with a detectable label selected from the group consisting of: a fluorescent label, a chemiluminescent label, a quencher, a radioactive label, biotin and gold.

32. A method of hybridizing one or more isolated nucleic acid sequences comprising a sequence selected from the group consisting of: SEQ ID NOS: 513-846 to a Enterococcus faecium specific gene and/or an Enterococcus faecalis specific gene, comprising contacting one or more isolated nucleic acid sequences to a sample comprising the Enterococcus faecium specific gene and/or Enterococcus faecalis specific gene under conditions suitable for hybridization.

33. The method of claim 32, further comprising isolating the hybridized Enterococcus faecium specific gene and/or Enterococcus faecalis specific gene.

33. (canceled)

34. A method of hybridizing one or more isolated nucleic acid sequences comprising a sequence selected from the group consisting of: SEQ ID NOS: 1-502 and 507-846 to a vancomycin-resistance gene and/or an Enterococcus faecium specific gene and/or an Enterococcus faecalis specific gene, comprising contacting one or more isolated nucleic acid sequences to a sample comprising the vancomycin-resistance gene and/or the Enterococcus faecium specific gene and/or the Enterococcus faecalis specific gene under conditions suitable for hybridization.

35. A kit for detecting an Enterococcus faecium specific gene and/or an Enterococcus faecalis specific gene in a sample, comprising one or more probes comprising a sequence selected from the group consisting of: SEQ ID NOS: 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual).

36. The kit of claim 35, further comprising:
a) at least one forward primer comprising the sequence selected from the group consisting of: SEQ ID NOS: 517 (Efm sodA); 577, 586, 590, 598, 599, 600 (Efs sodA); 683, 687, 692 (Efm novel); 758, 772, 773, 775 (Efs novel); and 843 (Efm/Efs dual); and
b) at least one reverse primer comprising the sequence selected from the group consisting of: SEQ ID NOS: 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual).

37. The kit of claim 35, further comprising reagents for sequencing an Enterococcus faecium specific gene and/or an Enterococcus faecalis specific gene.

38. The kit of claim 35, wherein the one or more probes are labeled with different detectable labels.

39. The kit of claim 35, wherein the one or more probes are labeled with the same detectable label.

40. The kit of claim 36, wherein the at least one forward primer and the at least one reverse primer are selected from the group consisting of: Groups 603-605 of Table 8B, Groups 606-627 of Table 9B, Groups 628-636 of Table 10B, Groups 637-643 of Table 11B, and Group 644 of Table 12.

41. A kit for detecting a vancomycin-resistance gene and/or an Enterococcus faecium specific gene and/or Enterococcus faecalis specific gene in a sample, comprising one or more probes comprising a sequence selected from the group consisting of: SEQ ID NOS: 2, 4, 7, 9, 11-18, 20, 24, 25, 27, 30, 35, 43, 46, 49, 50, 54, 56-58 (vanA); 62, 64, 67, 69, 73, 76, 78-80, 82, 84, 92, 96, 108-110, 112 (vanB); 124, 126, 128, 131, 134, 136, 139, 142, 145, 149, 152, 154, 159, 163, 166, 172, 176, 181, 182, 186, 189, 195, 197, 199, 205 (vanC1); 207, 211 (vanC2/3); 389, 392, 397, 400, 402-408, 410, 412-414, 418, 420, 422-424, 427, 429, 431-433, 436, 441, 446, 457, 463, 466, 469, 472, 475, 478, 481, 484, 485, 487, 489, 492, 499 (vanD); 335 (vanE); 245, 257, 261, 265, 268, 273, 289, 292, 294, 296, 319, 323 (vanG); 555, 562, 571 (Efm sodA); 644, 650, 654, 659, 661, 662, 663, 664, 665, 667, 673, 675, 676, 677 (Efs sodA); 728, 750 (Efm novel); 815, 832 (Efs novel), and 844 (Efm/Efs dual).

42. The kit of claim 41, further comprising:
a) at least one forward primer comprising the sequence selected from the group consisting of: SEQ ID NOS: 1, 6, 19, 22, 23, 26, 28, 29, 33, 34, 37-42, 45, 48, 53 and 59 (vanA); 61, 68, 70-72, 75, 81, 83, 89, 93, 94, 103-105, 107 and 111 (vanB); 123, 127, 130, 133, 138, 141, 144, 148, 151, 156, 158, 161, 162, 165, 168, 170, 171, 175, 178, 180, 183-185, 188, 191, 193, 194, 196, 198, and 200-204 (vanC1); 206, 210, 213, 217, 220, 221, 222, 224-226, 228-238, 240, 242 and 243 (vanC2/3); 388, 391, 394, 396, 399, 409, 415, 416, 425, 428, 435, 438, 440, 443, 445, 462, 465, 468, 471, 474, 477, 480, 483, 488, 491, 494-498 and 500-502 (vanD); 334, 337-380 and 382-387 (vanE); 244, 249, 252, 253, 255, 256, 260, 263, 264, 267, 270-272, 275-283, 285, 288, 291, 293, 295, 297-300, 302, 303, 305, 307, 309-311, 313-318, 321, 322, 325-333 (vanG); 517 (Efm sodA); 577, 586, 590, 598, 599, 600 (Efs sodA); 683, 687, 692 (Efm novel); 758, 772, 773, 775 (Efs novel); and 843 (Efm/Efs dual); and
b) at least one reverse primer comprising the sequence selected from the group consisting of: SEQ ID NOS: 3, 5, 8, 10, 21, 31, 32, 36, 44, 47, 51, 52, 55 and 60 (vanA); 63, 65, 66, 74, 77, 85-88, 90, 91, 95, and 97-102 (vanB); 125, 129, 132, 135, 137, 140, 143, 146, 147, 150, 153, 155, 157, 160, 164, 167, 169, 173, 174, 177, 179, 187, 190 and 192 (vanC1); 208, 209, 212, 215, 216, 219, 223, 227, 239 and 241 (vanC2/3); 390, 393, 395, 398, 401, 411, 417, 419, 421, 426, 430, 434, 437, 439, 442, 444, 447-455, 458-461, 464, 467, 470, 473, 476, 479, 482, 486, 490 and 493 (vanD); 336 and 381 (vanE); 246-248, 250, 251, 254, 258, 259, 262, 266, 269, 274, 284, 286, 287, 290, 301, 304, 306, 308, 312, 320 and 324 (vanG); 529 (Efm sodA); 617, 623, 624, 625, 637, 640 (Efs sodA); 707, 720, 723 (Efm novel); 785, 791, 797, 799, 803 (Efs novel); 845 and 846 (Efm/Efs dual).

43. The kit of claim 41, further comprising reagents for sequencing a vancomycin-resistance gene and/or an Enterococcus faecium specific gene and/or an Enterococcus faecalis specific gene in the sample.

44. The kit of claim 41, wherein the one or more probes are labeled with different detectable labels.

45. The kit of claim 41, wherein the one or more probes are labeled with the same detectable label.

46. The kit of claim 42, wherein the at least one forward primer and the at least one reverse primer are selected from the group consisting of: Groups 1-601 and 603-644 of Tables 5, 6, 8B, 9B, 10B, 11B, and 12.

47. A probe that hybridizes to a Enterococcus faecium novel marker gene corresponding to SEQ ID: 509.

48. A probe that hybridizes to a Enterococcus faecalis novel marker gene corresponding to SEQ ID: 510.

49. A probe that hybridizes to a novel marker gene corresponding to SEQ ID: 511 or SEQ ID: 512 that binds to both Enterococcus faecalis and Enterococcus faecium genes.

50. The method of claim 32, further comprising sequencing the hybridized Enterococcus faecium specific gene and/or Enterococcus faecalis specific gene.


SEQUENCE LISTING (849 sequences)

SEQ ID NO: 1 - DNA, 22 bp, Artificial Sequence
  Description of Artificial Sequence: Synthetic primer
ttgtgcggtattgggaaacagt

SEQ ID NO: 2 - DNA, 31 bp, Artificial Sequence
  Description of Artificial Sequence: Synthetic probe
tgatttggtccacctcgccaacaactaacgc

SEQ ID NO: 3 - DNA, 28 bp, Artificial Sequence
  Description of Artificial Sequence: Synthetic primer
cgacttcctgatgaatacgaaagattcc

SEQ ID NO: 4 - DNA, 32 bp, Artificial Sequence
  Description of Artificial Sequence: Synthetic probe
cctgatttggtccacctcgccaacaactaacg

SEQ ID NO: 5 - DNA, 29 bp, Artificial Sequence
  Description of Artificial Sequence: Synthetic primer
ctcgacttcctgatgaatacgaaagattc

SEQ ID NO: 6 - DNA, 19 bp, Artificial Sequence
  Description of Artificial Sequence: Synthetic primer
cgcgttcaggctcatcctt

SEQ ID NO: 7 - DNA, 37 bp, Artificial Sequence
  Description of Artificial Sequence: Synthetic probe
tcacagcccgaaacagcctgctcaattaagattttgc

SEQ ID NO: 8 - DNA, 23 bp, Artificial Sequence
  Description of Artificial Sequence: Synthetic primer
actgtttcccaataccgcacaac

SEQ ID NO: 9 - DNA, 35 bp, Artificial Sequence
  Description of Artificial Sequence: Synthetic probe
ctcacagcccgaaacagcctgctcaattaagattt

SEQ ID NO: 10 - DNA, 22 bp, Artificial Sequence
  Description of Artificial Sequence: Synthetic primer
actgtttcccaataccgcacaa

SEQ ID NO: 11 - DNA, 34 bp, Artificial Sequence
  Description of Artificial Sequence: Synthetic probe
ctcacagcccgaaacagcctgctcaattaagatt

SEQ ID NO: 12 - DNA, 37 bp, Artificial Sequence
  Description of Artificial Sequence: Synthetic probe
ctcacagcccgaaacagcctgctcaattaagattttg

SEQ ID NO: 13 - DNA, 33 bp, Artificial Sequence
  Description of Artificial Sequence: Synthetic probe
ctcacagcccgaaacagcctgctcaattaagat

SEQ ID NO: 14 - DNA, 36 bp, Artificial Sequence
  Description of Artificial Sequence: Synthetic probe
acagcccgaaacagcctgctcaattaagattttgct

SEQ ID NO: 15 - DNA, 32 bp, Artificial Sequence
  Description of Artificial Sequence: Synthetic probe
tcacagcccgaaacagcctgctcaattaagat

SEQ ID NO: 16 - DNA, 33 bp, Artificial Sequence
  Description of Artificial Sequence: Synthetic probe
tcacagcccgaaacagcctgctcaattaagatt

SEQ ID NO: 17 - DNA, 36 bp, Artificial Sequence
  Description of Artificial Sequence: Synthetic probe
tcacagcccgaaacagcctgctcaattaagattttg

SEQ ID NO: 18 - DNA, 35 bp, Artificial Sequence
  Description of Artificial Sequence: Synthetic probe
cagcccgaaacagcctgctcaattaagattttgct

SEQ ID NO: 19 - DNA, 28 bp, Artificial Sequence
  Description of Artificial Sequence: Synthetic primer
agcaaaatcttaattgagcaggctgttt

SEQ ID NO: 20 - DNA, 27 bp, Artificial Sequence
  Description of Artificial Sequence: Synthetic probe
cccaataccgcacaaccgacctcacag

SEQ ID NO: 21 - DNA, 17 bp, Artificial Sequence
  Description of Artificial Sequence: Synthetic primer
ggtccacctcgccaaca

SEQ ID NO: 22 - DNA, 29 bp, Artificial Sequence
  Description of Artificial Sequence: Synthetic primer
cagcaaaatcttaattgagcaggctgttt

SEQ ID NO: 23 - DNA, 27 bp, Artificial Sequence
  Description of Artificial Sequence: Synthetic primer
gcaaaatcttaattgagcaggctgttt

SEQ ID NO: 24 - DNA, 27 bp, Artificial Sequence
  Description of Artificial Sequence: Synthetic probe
caataccgcacaaccgacctcacagcc

SEQ ID NO: 25 - DNA, 26 bp, Artificial Sequence
  Description of Artificial Sequence: Synthetic probe
ctgcagcctgatttggtccacctcgc

SEQ ID NO: 26 - DNA, 19 bp, Artificial Sequence
  Description of Artificial Sequence: Synthetic primer
ggctgtgaggtcggttgtg

SEQ ID NO: 27 - DNA, 25 bp, Artificial Sequence
  Description of Artificial Sequence: Synthetic probe
tgcagcctgatttggtccacctcgc

SEQ ID NO: 28 - DNA, 18 bp, Artificial Sequence
  Description of Artificial Sequence: Synthetic primer
ggctgtgaggtcggttgt

SEQ ID NO: 29 - DNA, 18 bp, Artificial Sequence
  Description of Artificial Sequence: Synthetic primer
ggcgcgttcaggctcatc

SEQ ID NO: 30 - DNA, 25 bp, Artificial Sequence
  Description of Artificial Sequence: Synthetic probe
cagcctgatttggtccacctcgcca

SEQ ID NO: 31 - DNA, 23 bp, Artificial Sequence
  Description of Artificial Sequence: Synthetic primer
cactgtttcccaataccgcacaa

SEQ ID NO: 32 - DNA, 25 bp, Artificial Sequence
  Description of Artificial Sequence: Synthetic primer
gctcgacttcctgatgaatacgaaa

SEQ ID NO: 33 - DNA, 17 bp, Artificial Sequence
  Description of Artificial Sequence: Synthetic primer
ggcgcgttcaggctcat

SEQ ID NO: 34 - DNA, 19 bp, Artificial Sequence
  Description of Artificial Sequence: Synthetic primer
gggctgtgaggtcggttgt

SEQ ID NO: 35 - DNA, 29 bp, Artificial Sequence
  Description of Artificial Sequence: Synthetic probe
cgtactgcagcctgatttggtccacctcg

SEQ ID NO: 36 - DNA, 28 bp, Artificial Sequence
  Description of Artificial Sequence: Synthetic primer
gctcgacttcctgatgaatacgaaagat

SEQ ID NO: 37 - DNA, 21 bp, Artificial Sequence
  Description of Artificial Sequence: Synthetic primer
gaggtcggttgtgcggtattg

SEQ ID NO: 38 - DNA, 20 bp, Artificial Sequence
  Description of Artificial Sequence: Synthetic primer
aggtcggttgtgcggtattg

SEQ ID NO: 39 - DNA, 21 bp, Artificial Sequence
  Description of Artificial Sequence: Synthetic primer
tgaggtcggttgtgcggtatt

SEQ ID NO: 40 - DNA, 20 bp, Artificial Sequence
  Description of Artificial Sequence: Synthetic primer
gaggtcggttgtgcggtatt

SEQ ID NO: 41 - DNA, 32 bp, Artificial Sequence
  Description of Artificial Sequence: Synthetic primer
ggcaagacaatatgacagcaaaatcttaattg

SEQ ID NO: 42 - DNA, 32 bp, Artificial Sequence
  Description of Artificial Sequence: Synthetic primer
cagctacgtttacctatcctgtttttgttaag

SEQ ID NO: 43 - DNA, 23 bp, Artificial Sequence
  Description of Artificial Sequence: Synthetic probe
ccggcgcgttcaggctcatcctt

SEQ ID NO: 44 - DNA, 28 bp, Artificial Sequence
  Description of Artificial Sequence: Synthetic primer
gcctgctcaattaagattttgctgtcat

SEQ ID NO: 45 - DNA, 20 bp, Artificial Sequence
  Description of Artificial Sequence: Synthetic primer
tggcgaggtggaccaaatca

SEQ ID NO: 46 - DNA, 29 bp, Artificial Sequence
  Description of Artificial Sequence: Synthetic probe
actgcgttttcagagcctttttccggctc

SEQ ID NO: 47 - DNA, 19 bp, Artificial Sequence
  Description of Artificial Sequence: Synthetic primer
ggtctgcgggaacggttat

SEQ ID NO: 48 - DNA, 20 bp, Artificial Sequence
  Description of Artificial Sequence: Synthetic primer
gttggcgaggtggaccaaat

SEQ ID NO: 49 - DNA, 29 bp, Artificial Sequence
  Description of Artificial Sequence: Synthetic probe
agagcctttttccggctcgacttcctgat

SEQ ID NO: 50 - DNA, 30 bp, Artificial Sequence
  Description of Artificial Sequence: Synthetic probe
actgcgttttcagagcctttttccggctcg

SEQ ID NO: 51 - DNA, 23 bp, Artificial Sequence
  Description of Artificial Sequence: Synthetic primer
ctgcgttttcagagcctttttcc

SEQ ID NO: 52 - DNA, 20 bp, Artificial Sequence
  Description of Artificial Sequence: Synthetic primer
aggtctgcgggaacggttat

SEQ ID NO: 53 - DNA, 25 bp, Artificial Sequence
  Description of Artificial Sequence: Synthetic primer
aatcaggctgcagtacggaatcttt

SEQ ID NO: 54 - DNA, 28 bp, Artificial Sequence
  Description of Artificial Sequence: Synthetic probe
tgcgttttcagagcctttttccggctcg

SEQ ID NO: 55 - DNA, 20 bp, Artificial Sequence
  Description of Artificial Sequence: Synthetic primer
aaggtctgcgggaacggtta

SEQ ID NO: 56 - DNA, 30 bp, Artificial Sequence
  Description of Artificial Sequence: Synthetic probe
tgcgttttcagagcctttttccggctcgac

SEQ ID NO: 57 - DNA, 31 bp, Artificial Sequence
  Description of Artificial Sequence: Synthetic probe
aactgcgttttcagagcctttttccggctcg

SEQ ID NO: 58 - DNA, 30 bp, Artificial Sequence
  Description of Artificial Sequence: Synthetic probe
tgtttcccaataccgcacaaccgacctcac

SEQ ID NO: 59 - DNA, 28 bp, Artificial Sequence
  Description of Artificial Sequence: Synthetic primer
gcagtacggaatctttcgtattcatcag

SEQ ID NO: 60 - DNA, 18 bp, Artificial Sequence
  Description of Artificial Sequence: Synthetic primer
ggtctgcgggaacggtta

SEQ ID NO: 61 - DNA, 24 bp, Artificial Sequence
  Description of Artificial Sequence: Synthetic primer
aaatcactggcctacattcttaca

SEQ ID NO: 62 - DNA, 30 bp, Artificial Sequence
  Description of Artificial Sequence: Synthetic probe
ttctatcgcagcgttaagttcttccgtacc

SEQ ID NO: 63 - DNA, 24 bp, Artificial Sequence
  Description of Artificial Sequence: Synthetic primer
cgaaatcgcttgctcaattaagat

SEQ ID NO: 64 - DNA, 32 bp, Artificial Sequence
  Description of Artificial Sequence: Synthetic probe
tatcgcagcgttaagttcttccgtaccgttta

SEQ ID NO: 65 - DNA, 27 bp, Artificial Sequence
  Description of Artificial Sequence: Synthetic primer
gcttgctcaattaagatttttccatca

SEQ ID NO: 66 - DNA, 30 bp, Artificial Sequence
  Description of Artificial Sequence: Synthetic primer
gctcaattaagatttttccatcatattgtc

SEQ ID NO: 67 - DNA, 31 bp, Artificial Sequence
  Description of Artificial Sequence: Synthetic probe
cttctatcgcagcgttaagttcttccgtacc

SEQ ID NO: 68 - DNA, 27 bp, Artificial Sequence
  Description of Artificial Sequence: Synthetic primer
aaatcactggcctacattcttacaaaa

SEQ ID NO: 69 - DNA, 32 bp, Artificial Sequence
  Description of Artificial Sequence: Synthetic probe
ttttccatcatattgtcctgccgcttctatcg

SEQ ID NO: 70 - DNA, 25 bp, Artificial Sequence
  Description of Artificial Sequence: Synthetic primer
aaatcactggcctacattcttacaa

SEQ ID NO: 71 - DNA, 26 bp, Artificial Sequence
  Description of Artificial Sequence: Synthetic primer
aaatcactggcctacattcttacaaa

SEQ ID NO: 72 - DNA, 24 bp, Artificial Sequence
  Description of Artificial Sequence: Synthetic primer
aatcactggcctacattcttacaa

SEQ ID NO: 73 - DNA, 34 bp, Artificial Sequence
  Description of Artificial Sequence: Synthetic probe
agatttttccatcatattgtcctgccgcttctat

SEQ ID NO: 74 - DNA, 21 bp, Artificial Sequence
  Description of Artificial Sequence: Synthetic primer
ccgaaatcgcttgctcaatta

SEQ ID NO: 75 - DNA, 25 bp, Artificial Sequence
  Description of Artificial Sequence: Synthetic primer
aatcactggcctacattcttacaaa

SEQ ID NO: 76 - DNA, 34 bp, Artificial Sequence
  Description of Artificial Sequence: Synthetic probe
taagatttttccatcatattgtcctgccgcttct

SEQ ID NO: 77 - DNA, 19 bp, Artificial Sequence
  Description of Artificial Sequence: Synthetic primer
ccgaaatcgcttgctcaat

SEQ ID NO: 78 - DNA, 34 bp, Artificial Sequence
  Description of Artificial Sequence: Synthetic probe
aagatttttccatcatattgtcctgccgcttcta

SEQ ID NO: 79 - DNA, 33 bp, Artificial Sequence
  Description of Artificial Sequence: Synthetic probe
aagatttttccatcatattgtcctgccgcttct

SEQ ID NO: 80 - DNA, 35 bp, Artificial Sequence
  Description of Artificial Sequence: Synthetic probe
aagatttttccatcatattgtcctgccgcttctat

SEQ ID NO: 81 - DNA, 22 bp, Artificial Sequence
  Description of Artificial Sequence: Synthetic primer
cttacctaccctgtctttgtga

SEQ ID NO: 82 - DNA, 32 bp, Artificial Sequence
  Description of Artificial Sequence: Synthetic probe
agatttttccatcatattgtcctgccgcttct

SEQ ID NO: 83 - DNA, 23 bp, Artificial Sequence
  Description of Artificial Sequence: Synthetic primer
ttacctaccctgtctttgtgaag

SEQ ID NO: 84 - DNA, 23 bp, Artificial Sequence
  Description of Artificial Sequence: Synthetic probe
cacggtcaggttcgtcctttggc

SEQ ID NO: 85 - DNA, 25 bp, Artificial Sequence
  Description of Artificial Sequence: Synthetic primer
cgcttgctcaattaagatttttcca

SEQ ID NO: 86 - DNA, 26 bp, Artificial Sequence
  Description of Artificial Sequence: Synthetic primer
cgcttgctcaattaagatttttccat

SEQ ID NO: 87 - DNA, 28 bp, Artificial Sequence
  Description of Artificial Sequence: Synthetic primer
gcttgctcaattaagatttttccatcat

SEQ ID NO: 88 - DNA, 29 bp, Artificial Sequence
  Description of Artificial Sequence: Synthetic primer
gcttgctcaattaagatttttccatcata

SEQ ID NO: 89 - DNA, 21 bp, Artificial Sequence
  Description of Artificial Sequence: Synthetic primer
acctaccctgtctttgtgaag

SEQ ID NO: 90 - DNA, 25 bp, Artificial Sequence
  Description of Artificial Sequence: Synthetic primer
tcgcttgctcaattaagatttttcc

SEQ ID NO: 91 - DNA, 26 bp, Artificial Sequence
  Description of Artificial Sequence: Synthetic primer
atcgcttgctcaattaagatttttcc

SEQ ID NO: 92 - DNA, 33 bp, Artificial Sequence
  Description of Artificial Sequence: Synthetic probe
tttttccatcatattgtcctgccgcttctatcg

SEQ ID NO: 93 - DNA, 22 bp, Artificial Sequence
  Description of Artificial Sequence: Synthetic primer
tacctaccctgtctttgtgaag

SEQ ID NO: 94 - DNA, 23 bp, Artificial Sequence
  Description of Artificial Sequence: Synthetic primer
cttacctaccctgtctttgtgaa

SEQ ID NO: 95 - DNA, 33 bp, Artificial Sequence
  Description of Artificial Sequence: Synthetic primer
cttgctcaattaagatttttccatcatattgtc

SEQ ID NO: 96 - DNA, 21 bp, Artificial Sequence
  Description of Artificial Sequence: Synthetic probe
cgcctccggcttgtcaccttt

SEQ ID NO: 97 - DNA, 21 bp, Artificial Sequence
  Description of Artificial Sequence: Synthetic primer
acaaagacagggtaggtaagc

SEQ ID NO: 98 - DNA, 26 bp, Artificial Sequence
  Description of Artificial Sequence: Synthetic primer
tcgcttgctcaattaagatttttcca

SEQ ID NO: 99 - DNA, 27 bp, Artificial Sequence
  Description of Artificial Sequence: Synthetic primer
atcgcttgctcaattaagatttttcca

SEQ ID NO: 100 - DNA, 27 bp, Artificial Sequence
  Description of Artificial Sequence: Synthetic primer
tcgcttgctcaattaagatttttccat

SEQ ID NO: 101 - DNA, 24 bp, Artificial Sequence
  Description of Artificial Sequence: Synthetic primer
cgcttgctcaattaagatttttcc

SEQ ID NO: 102 - DNA, 28 bp, Artificial Sequence
  Description of Artificial Sequence: Synthetic primer
atcgcttgctcaattaagatttttccat

SEQ ID NO: 103 - DNA, 22 bp, Artificial Sequence
  Description of Artificial Sequence: Synthetic primer
ttacctaccctgtctttgtgaa

SEQ ID NO: 104 - DNA, 21 bp, Artificial Sequence
  Description of Artificial Sequence: Synthetic primer
gcttacctaccctgtctttgt

SEQ ID NO: 105 - DNA, 20 bp, Artificial Sequence
  Description of Artificial Sequence: Synthetic primer
cgcttacctaccctgtcttt

SEQ ID NO: 106 - DNA, 31 bp, Artificial Sequence
  Description of Artificial Sequence: Synthetic probe
aggttcgtcctttggcgtaaccaaagtaaac

SEQ ID NO: 107 - DNA, 19 bp, Artificial Sequence
  Description of Artificial Sequence: Synthetic primer
cgcttacctaccctgtctt

SEQ ID NO: 108 - DNA, 24 bp, Artificial Sequence
  Description of Artificial Sequence: Synthetic probe
caggttcgtcctttggcgtaacca

SEQ ID NO: 109 - DNA, 23 bp, Artificial Sequence
  Description of Artificial Sequence: Synthetic probe
ccaaaggacgaacctgaccgtgc

SEQ ID NO: 110 - DNA, 20 bp, Artificial Sequence
  Description of Artificial Sequence: Synthetic probe
aggacgaacctgaccgtgcc

SEQ ID NO: 111 - DNA, 22 bp, Artificial Sequence
  Description of Artificial Sequence: Synthetic primer
atcactggcctacattcttaca

SEQ ID NO: 112 - DNA, 27 bp, Artificial Sequence
  Description of Artificial Sequence: Synthetic probe
cgcttacctaccctgtctttgtgaagc

SEQ ID NO: 113-122 - skipped (10 entries)

SEQ ID NO: 123 - DNA, 29 bp, Artificial Sequence
  Description of Artificial Sequence: Synthetic primer
atgtatgaacaaatggctcttgcatcaac

SEQ ID NO: 124 - DNA, 33 bp, Artificial Sequence
  Description of Artificial Sequence: Synthetic probe
cgctagtgctcccactttgcttttatcccgcta

SEQ ID NO: 125 - DNA, 29 bp, Artificial Sequence
  Description of Artificial Sequence: Synthetic primer
gatcaacacagtagaaccgtaagcaaaag

SEQ ID NO: 126 - DNA, 31 bp, Artificial Sequence
  Description of Artificial Sequence: Synthetic probe
cgctagtgctcccactttgcttttatcccgc

SEQ ID NO: 127 - DNA, 32 bp, Artificial Sequence
  Description of Artificial Sequence: Synthetic primer
aaaagggatcacaaaagtaactgacaaaacag

SEQ ID NO: 128 - DNA, 29 bp, Artificial Sequence
  Description of Artificial Sequence: Synthetic probe
cgcagccaatttcaatacccgctatcgcc

SEQ ID NO: 129 - DNA, 29 bp, Artificial Sequence
  Description of Artificial Sequence: Synthetic primer
ccaatcgtcaattgctcatttcctaagat

SEQ ID NO: 130 - DNA, 29 bp, Artificial Sequence
  Description of Artificial Sequence: Synthetic primer
tcgatcgttttattcaagaccatggattc

SEQ ID NO: 131 - DNA, 29 bp, Artificial Sequence
  Description of Artificial Sequence: Synthetic probe
cccttttgaagaaccggcttcattcggct

SEQ ID NO: 132 - DNA, 25 bp, Artificial Sequence
  Description of Artificial Sequence: Synthetic primer
gatcaacacagtagaaccgtaagca

SEQ ID NO: 133 - DNA, 29 bp, Artificial Sequence
  Description of Artificial Sequence: Synthetic primer
agggatcacaaaagtaactgacaaaacag

SEQ ID NO: 134 - DNA, 27 bp, Artificial Sequence
  Description of Artificial Sequence: Synthetic probe
tgccgcagccaatttcaatacccgcta

SEQ ID NO: 135 - DNA, 26 bp, Artificial Sequence
  Description of Artificial Sequence: Synthetic primer
accaatcgtcaattgctcatttccta

SEQ ID NO: 136 - DNA, 29 bp, Artificial Sequence
  Description of Artificial Sequence: Synthetic probe
aagatgccgcagccaatttcaatacccgc

SEQ ID NO: 137 - DNA, 25 bp, Artificial Sequence
  Description of Artificial Sequence: Synthetic primer
accaatcgtcaattgctcatttcct

SEQ ID NO: 138 - DNA, 29 bp, Artificial Sequence
  Description of Artificial Sequence: Synthetic primer
tcgatcgttttattcaagaccatggattc

SEQ ID NO: 139 - DNA, 29 bp, Artificial Sequence
  Description of Artificial Sequence: Synthetic probe
cccttttgaagaaccggcttcattcggct

SEQ ID NO: 140 - DNA, 23 bp, Artificial Sequence
  Description of Artificial Sequence: Synthetic primer
accgtaagcaaaagcagtcgtta

SEQ ID NO: 141 - DNA, 27 bp, Artificial Sequence
  Description of Artificial Sequence: Synthetic primer
ttaacgactgcttttgcttacggttct

SEQ ID NO: 142 - DNA, 30 bp, Artificial Sequence
  Description of Artificial Sequence: Synthetic probe
acccgctatcgccttttggatcaacacagt

SEQ ID NO: 143 - DNA, 24 bp, Artificial Sequence
  Description of Artificial Sequence: Synthetic primer
gtcgacaagagaaatcgcatcaca

SEQ ID NO: 144 - DNA, 27 bp, Artificial Sequence
  Description of Artificial Sequence: Synthetic primer
cttttgcttacggttctactgtgttga

SEQ ID NO: 145 - DNA, 29 bp, Artificial Sequence
  Description of Artificial Sequence: Synthetic probe
cgcagccaatttcaatacccgctatcgcc

SEQ ID NO: 146 - DNA, 24 bp, Artificial Sequence
  Description of Artificial Sequence: Synthetic primer
acaagagaaatcgcatcacaagca

SEQ ID NO: 147 - DNA, 20 bp, Artificial Sequence
  Description of Artificial Sequence: Synthetic primer
cgcatcacaagcaccaatcg

SEQ ID NO: 148 - DNA, 24 bp, Artificial Sequence
  Description of Artificial Sequence: Synthetic primer
tctgcattaacgactgcttttgct

SEQ ID NO: 149 - DNA, 27 bp, Artificial Sequence
  Description of Artificial Sequence: Synthetic probe
tgccgcagccaatttcaatacccgcta

SEQ ID NO: 150 - DNA, 26 bp, Artificial Sequence
  Description of Artificial Sequence: Synthetic primer
accaatcgtcaattgctcatttccta

SEQ ID NO: 151 - DNA, 26 bp, Artificial Sequence
  Description of Artificial Sequence: Synthetic primer
gcttttgcttacggttctactgtgtt

SEQ ID NO: 152 - DNA, 28 bp, Artificial Sequence
  Description of Artificial Sequence: Synthetic probe
agatgccgcagccaatttcaatacccgc

SEQ ID NO: 153 - DNA, 27 bp, Artificial Sequence
  Description of Artificial Sequence: Synthetic primer
caccaatcgtcaattgctcatttccta

SEQ ID NO: 154 - DNA, 32 bp, Artificial Sequence
  Description of Artificial Sequence: Synthetic probe
cgcagccaatttcaatacccgctatcgccttt

SEQ ID NO: 155 - DNA, 28 bp, Artificial Sequence
  Description of Artificial Sequence: Synthetic primer
caccaatcgtcaattgctcatttcctaa

SEQ ID NO: 156 - DNA, 27 bp, Artificial Sequence
  Description of Artificial Sequence: Synthetic primer
gcttttgcttacggttctactgtgttg

SEQ ID NO: 157 - DNA, 28 bp, Artificial Sequence
  Description of Artificial Sequence: Synthetic primer
accaatcgtcaattgctcatttcctaag

SEQ ID NO: 158 - DNA, 25 bp, Artificial Sequence
  Description of Artificial Sequence: Synthetic primer
actgtgttgatccaaaaggcgatag

SEQ ID NO: 159 - DNA, 33 bp, Artificial Sequence
  Description of Artificial Sequence: Synthetic probe
ttcctaagatgccgcagccaatttcaatacccg

SEQ ID NO: 160 - DNA, 24 bp, Artificial Sequence
  Description of Artificial Sequence: Synthetic primer
gaaatcgcatcacaagcaccaatc

SEQ ID NO: 161 - DNA, 26 bp, Artificial Sequence
  Description of Artificial Sequence: Synthetic primer
ctactgtgttgatccaaaaggcgata

SEQ ID NO: 162 - DNA, 33 bp, Artificial Sequence
  Description of Artificial Sequence: Synthetic primer
tttattcaagaccatggattcccgatctttatc

SEQ ID NO: 163 - DNA, 30 bp, Artificial Sequence
  Description of Artificial Sequence: Synthetic probe
tcccttttgaagaaccggcttcattcggct

SEQ ID NO: 164 - DNA, 26 bp, Artificial Sequence
  Description of Artificial Sequence: Synthetic primer
gcgctgttttgtcagttacttttgtg

SEQ ID NO: 165 - DNA, 30 bp, Artificial Sequence
  Description of Artificial Sequence: Synthetic primer
ttcaagaccatggattcccgatctttatca

SEQ ID NO: 166 - DNA, 32 bp, Artificial Sequence
  Description of Artificial Sequence: Synthetic probe
tgatcccttttgaagaaccggcttcattcggc

SEQ ID NO: 167 - DNA, 23 bp, Artificial Sequence
  Description of Artificial Sequence: Synthetic primer
tggagcgctgttttgtcagttac

SEQ ID NO: 168 - DNA, 31 bp, Artificial Sequence
  Description of Artificial Sequence: Synthetic primer
attcaagaccatggattcccgatctttatca

SEQ ID NO: 169 - DNA, 24 bp, Artificial Sequence
  Description of Artificial Sequence: Synthetic primer
ggagcgctgttttgtcagttactt

SEQ ID NO: 170 - DNA, 26 bp, Artificial Sequence
  Description of Artificial Sequence: Synthetic primer
caagaccatggattcccgatctttat

SEQ ID NO: 171 - DNA, 27 bp, Artificial Sequence
  Description of Artificial Sequence: Synthetic primer
aagaccatggattcccgatctttatca

SEQ ID NO: 172 - DNA, 29 bp, Artificial Sequence
  Description of Artificial Sequence: Synthetic probe
tcccttttgaagaaccggcttcattcggc

SEQ ID NO: 173 - DNA, 22 bp, Artificial Sequence
  Description of Artificial Sequence: Synthetic primer
tggagcgctgttttgtcagtta

SEQ ID NO: 174 - DNA, 21 bp, Artificial Sequence
  Description of Artificial Sequence: Synthetic primer
tggagcgctgttttgtcagtt

SEQ ID NO: 175 - DNA, 27 bp, Artificial Sequence
  Description of Artificial Sequence: Synthetic primer
acggttctactgtgttgatccaaaagg

SEQ ID NO: 176 - DNA, 29 bp, Artificial Sequence
  Description of Artificial Sequence: Synthetic probe
agatgccgcagccaatttcaatacccgct

SEQ ID NO: 177 - DNA, 26 bp, Artificial Sequence
  Description of Artificial Sequence: Synthetic primer
caccaatcgtcaattgctcatttcct

SEQ ID NO: 178 - DNA, 26 bp, Artificial Sequence
  Description of Artificial Sequence: Synthetic primer
tctgcattaacgactgcttttgctta

SEQ ID NO: 179 - DNA, 22 bp, Artificial Sequence
  Description of Artificial Sequence: Synthetic primer
taagatgccgcagccaatttca

SEQ ID NO: 180 - DNA, 26 bp, Artificial Sequence
  Description of Artificial Sequence: Synthetic primer
aaaggcgatagcgggtattgaaattg

SEQ ID NO: 181 - DNA, 36 bp, Artificial Sequence
  Description of Artificial Sequence: Synthetic probe
ccaatcgtcaattgctcatttcctaagatgccgcag

SEQ ID NO: 182 - DNA, 30 bp, Artificial Sequence
  Description of Artificial Sequence: Synthetic probe
aagatgccgcagccaatttcaatacccgct

SEQ ID NO: 183 - DNA, 28 bp, Artificial Sequence
  Description of Artificial Sequence: Synthetic primer
tacggttctactgtgttgatccaaaagg

SEQ ID NO: 184 - DNA, 26 bp, Artificial Sequence
  Description of Artificial Sequence: Synthetic primer
agaccatggattcccgatctttatca

SEQ ID NO: 185 - DNA, 26 bp, Artificial Sequence
  Description of Artificial Sequence: Synthetic primer
ctgcattaacgactgcttttgcttac

SEQ ID NO: 186 - DNA, 32 bp, Artificial Sequence
  Description of Artificial Sequence: Synthetic probe
acccgctatcgccttttggatcaacacagtag

SEQ ID NO: 187 - DNA, 22 bp, Artificial Sequence
  Description of Artificial Sequence: Synthetic primer
ctaagatgccgcagccaatttc

SEQ ID NO: 188 - DNA, 26 bp, Artificial Sequence
  Description of Artificial Sequence: Synthetic primer
cggttctactgtgttgatccaaaagg

SEQ ID NO: 189 - DNA, 26 bp, Artificial Sequence
  Description of Artificial Sequence: Synthetic probe
tgccgcagccaatttcaatacccgct

SEQ ID NO: 190 - DNA, 25 bp, Artificial Sequence
  Description of Artificial Sequence: Synthetic primer
caccaatcgtcaattgctcatttcc

SEQ ID NO: 191 - DNA, 27 bp, Artificial Sequence
  Description of Artificial Sequence: Synthetic primer
tctactgtgttgatccaaaaggcgata

SEQ ID NO: 192 - DNA, 25 bp, Artificial Sequence
  Description of Artificial Sequence: Synthetic primer
gcaccaatcgtcaattgctcatttc

SEQ ID NO: 193 - DNA, 28 bp, Artificial Sequence
  Description of Artificial Sequence: Synthetic primer
ttctactgtgttgatccaaaaggcgata

SEQ ID NO: 194 - DNA, 25 bp, Artificial Sequence
  Description of Artificial Sequence: Synthetic primer
caaaaggcgatagcgggtattgaaa

SEQ ID NO: 195 - DNA, 32 bp, Artificial Sequence
  Description of Artificial Sequence: Synthetic probe
tcaattgctcatttcctaagatgccgcagcca

SEQ ID NO: 196 - DNA, 24 bp, Artificial Sequence
  Description of Artificial Sequence: Synthetic primer
aaaaggcgatagcgggtattgaaa

SEQ ID NO: 197 - DNA, 33 bp, Artificial Sequence
  Description of Artificial Sequence: Synthetic probe
tcaattgctcatttcctaagatgccgcagccaa

SEQ ID NO: 198 - DNA, 28 bp, Artificial Sequence
  Description of Artificial Sequence: Synthetic primer
attaacgactgcttttgcttacggttct

SEQ ID NO: 199 - DNA, 25 bp, Artificial Sequence
  Description of Artificial Sequence: Synthetic probe
tgccgcagccaatttcaatacccgc

SEQ ID NO: 200 - DNA, 26 bp, Artificial Sequence
  Description of Artificial Sequence: Synthetic primer
taacgactgcttttgcttacggttct

SEQ ID NO: 201 - DNA, 25 bp, Artificial Sequence
  Description of Artificial Sequence: Synthetic primer
aacgactgcttttgcttacggttct

SEQ ID NO: 202 - DNA, 24 bp, Artificial Sequence
  Description of Artificial Sequence: Synthetic primer
acgactgcttttgcttacggttct

SEQ ID NO: 203 - DNA, 24 bp, Artificial Sequence
  Description of Artificial Sequence: Synthetic primer
caaaaggcgatagcgggtattgaa

SEQ ID NO: 204 - DNA, 23 bp, Artificial Sequence
  Description of Artificial Sequence: Synthetic primer
cgactgcttttgcttacggttct

SEQ ID NO: 205 - DNA, 31 bp, Artificial Sequence
  Description of Artificial Sequence: Synthetic probe
tacccgctatcgccttttggatcaacacagt

SEQ ID NO: 206 - DNA, 21 bp, Artificial Sequence
  Description of Artificial Sequence: Synthetic primer
cgccattgcctgaaacgattg

SEQ ID NO: 207 - DNA, 34 bp, Artificial Sequence
  Description of Artificial Sequence: Synthetic probe
agtcttggtcttaaaggtcttgctcgcatcgact

SEQ ID NO: 208 - DNA, 29 bp, Artificial Sequence
  Description of Artificial Sequence: Synthetic primer
tcaagtatagttctccttgatccgtgaca

SEQ ID NO: 209 - DNA, 26 bp, Artificial Sequence
  Description of Artificial Sequence: Synthetic primer
agtatagttctccttgatccgtgaca

SEQ ID NO: 210 - DNA, 26 bp, Artificial Sequence
  Description of Artificial Sequence: Synthetic primer
cctgaaacgattgaaaccaaggtcaa

SEQ ID NO: 211 - DNA, 34 bp, Artificial Sequence
  Description of Artificial Sequence: Synthetic probe
agtcgatgcgagcaagacctttaagaccaagact

SEQ ID NO: 212 - DNA, 27 bp, Artificial Sequence
  Description of Artificial Sequence: Synthetic primer
aagtatagttctccttgatccgtgaca

SEQ ID NO: 213 - DNA, 20 bp, Artificial Sequence
  Description of Artificial Sequence: Synthetic primer
gcgccattgcctgaaacgat

SEQ ID NO: 214 - DNA, 35 bp, Artificial Sequence
  Description of Artificial Sequence: Synthetic probe
aagtcgatgcgagcaagacctttaagaccaagact

SEQ ID NO: 215 - DNA, 28 bp, Artificial Sequence
  Description of Artificial Sequence: Synthetic primer
agtatagttctccttgatccgtgacaaa

SEQ ID NO: 216 - DNA, 29 bp, Artificial Sequence
  Description of Artificial Sequence: Synthetic primer
gtatagttctccttgatccgtgacaaaaa

SEQ ID NO: 217 - DNA, 23 bp, Artificial Sequence
  Description of Artificial Sequence: Synthetic primer
cgccattgcctgaaacgattgaa

SEQ ID NO: 218 - DNA, 34 bp, Artificial Sequence
  Description of Artificial Sequence: Synthetic probe
cgatgcgagcaagacctttaagaccaagactacg

SEQ ID NO: 219 - DNA, 26 bp, Artificial Sequence
  Description of Artificial Sequence: Synthetic primer
ttctccttgatccgtgacaaaaaagt

SEQ ID NO: 220 - DNA, 18 bp, Artificial Sequence
  Description of Artificial Sequence: Synthetic primer
gcgccattgcctgaaacg

SEQ ID NO: 221 - DNA, 24 bp, Artificial Sequence
  Description of Artificial Sequence: Synthetic primer
gccattgcctgaaacgattgaaac

SEQ ID NO: 222 - DNA, 19 bp, Artificial Sequence
  Description of Artificial Sequence: Synthetic primer
ctgcgccattgcctgaaac

SEQ ID NO: 223 - DNA, 26 bp, Artificial Sequence
  Description of Artificial Sequence: Synthetic primer
agttctccttgatccgtgacaaaaaa

SEQ ID NO: 224 - DNA, 28 bp, Artificial Sequence
  Description of Artificial Sequence: Synthetic primer
cctgaaacgattgaaaccaaggtcaaag

SEQ ID NO: 225 - DNA, 27 bp, Artificial Sequence
  Description of Artificial Sequence: Synthetic primer
ctgaaacgattgaaaccaaggtcaaag

SEQ ID NO: 226 - DNA, 29 bp, Artificial Sequence
  Description of Artificial Sequence: Synthetic primer
ctgaaacgattgaaaccaaggtcaaagaa

SEQ ID NO: 227 - DNA, 30 bp, Artificial Sequence
  Description of Artificial Sequence: Synthetic primer
tatagttctccttgatccgtgacaaaaaag

SEQ ID NO: 228 - DNA, 27 bp, Artificial Sequence
  Description of Artificial Sequence: Synthetic primer
cctgaaacgattgaaaccaaggtcaaa

SEQ ID NO: 229 - DNA, 28 bp, Artificial Sequence
  Description of Artificial Sequence: Synthetic primer
ctgaaacgattgaaaccaaggtcaaaga

SEQ ID NO: 230 - DNA, 16 bp, Artificial Sequence
  Description of Artificial Sequence: Synthetic primer
ccgtccctgcgccatt

SEQ ID NO: 231 - DNA, 27 bp, Artificial Sequence
  Description of Artificial Sequence: Synthetic primer
tgaaacgattgaaaccaaggtcaaaga

SEQ ID NO: 232 - DNA, 28 bp, Artificial Sequence
  Description of Artificial Sequence: Synthetic primer
acgattgaaaccaaggtcaaagaacaag

SEQ ID NO: 233 - DNA, 29 bp, Artificial Sequence
  Description of Artificial Sequence: Synthetic primer
tgaaacgattgaaaccaaggtcaaagaac

SEQ ID NO: 234 - DNA, 30 bp, Artificial Sequence
  Description of Artificial Sequence: Synthetic primer
tgaaacgattgaaaccaaggtcaaagaaca

SEQ ID NO: 235 - DNA, 27 bp, Artificial Sequence
  Description of Artificial Sequence: Synthetic primer
aacgattgaaaccaaggtcaaagaaca

SEQ ID NO: 236 - DNA, 27 bp, Artificial Sequence
  Description of Artificial Sequence: Synthetic primer
acgattgaaaccaaggtcaaagaacaa

SEQ ID NO: 237 - DNA, 28 bp, Artificial Sequence
  Description of Artificial Sequence: Synthetic primer
gaaacgattgaaaccaaggtcaaagaac

SEQ ID NO: 238 - DNA, 29 bp, Artificial Sequence
  Description of Artificial Sequence: Synthetic primer
gaaacgattgaaaccaaggtcaaagaaca

SEQ ID NO: 239 - DNA, 29 bp, Artificial Sequence
  Description of Artificial Sequence: Synthetic primer
aagtatagttctccttgatccgtgacaaa

SEQ ID NO: 240 - DNA, 30 bp, Artificial Sequence
  Description of Artificial Sequence: Synthetic primer
aaacgattgaaaccaaggtcaaagaacaag

SEQ ID NO: 241 - DNA, 30 bp, Artificial Sequence
  Description of Artificial Sequence: Synthetic primer
agtatagttctccttgatccgtgacaaaaa

SEQ ID NO: 242 - DNA, 28 bp, Artificial Sequence
  Description of Artificial Sequence: Synthetic primer
aaacgattgaaaccaaggtcaaagaaca

SEQ ID NO: 243 - DNA, 29 bp, Artificial Sequence
  Description of Artificial Sequence: Synthetic primer
aaacgattgaaaccaaggtcaaagaacaa

SEQ ID NO: 244 - DNA, 31 bp, Artificial Sequence
  Description of Artificial Sequence: Synthetic primer
tctggcgaaattgtatttaatgaggtaaaca

SEQ ID NO: 245 - DNA, 29 bp, Artificial Sequence
  Description of Artificial Sequence: Synthetic probe
caataccaggctttacctcgcacagtcgc

SEQ ID NO: 246 - DNA, 27 bp, Artificial Sequence
  Description of Artificial Sequence: Synthetic primer
agaccaatgcctttcatcatatttgga

SEQ ID NO: 247 - DNA, 32 bp, Artificial Sequence
  Description of Artificial Sequence: Synthetic primer
gatagaccaatgcctttcatcatatttggata

SEQ ID NO: 248 - DNA, 30 bp, Artificial Sequence
  Description of Artificial Sequence: Synthetic primer
gatagaccaatgcctttcatcatatttgga

SEQ ID NO: 249 - DNA, 27 bp, Artificial Sequence
  Description of Artificial Sequence: Synthetic primer
accgtctggcgaaattgtatttaatga

SEQ ID NO: 250 - DNA, 30 bp, Artificial Sequence
  Description of Artificial Sequence: Synthetic primer
cgatagaccaatgcctttcatcatatttgg

SEQ ID NO: 251 - DNA, 33 bp, Artificial Sequence
  Description of Artificial Sequence: Synthetic primer
cgatagaccaatgcctttcatcatatttggata

SEQ ID NO: 252 - DNA, 27 bp, Artificial Sequence
  Description of Artificial Sequence: Synthetic primer
caccgtctggcgaaattgtatttaatg

SEQ ID NO: 253 - DNA, 22 bp, Artificial Sequence
  Description of Artificial Sequence: Synthetic primer
acaccgtctggcgaaattgtat

SEQ ID NO: 254 - DNA, 30 bp, Artificial Sequence
  Description of Artificial Sequence: Synthetic primer
aacgatagaccaatgcctttcatcatattt

SEQ ID NO: 255 - DNA, 25 bp, Artificial Sequence
  Description of Artificial Sequence: Synthetic primer
acaccgtctggcgaaattgtattta

SEQ ID NO: 256 - DNA, 23 bp, Artificial Sequence
  Description of Artificial Sequence: Synthetic primer
acaccgtctggcgaaattgtatt

SEQ ID NO: 257 - DNA, 29 bp, Artificial Sequence
  Description of Artificial Sequence: Synthetic probe
aggctttacctcgcacagtcgctatccaa

SEQ ID NO: 258 - DNA, 26 bp, Artificial Sequence
  Description of Artificial Sequence: Synthetic primer
gaacgatagaccaatgcctttcatca

SEQ ID NO: 259 - DNA, 28 bp, Artificial Sequence
  Description of Artificial Sequence: Synthetic primer
gaacgatagaccaatgcctttcatcata

SEQ ID NO: 260 - DNA, 28 bp, Artificial Sequence
  Description of Artificial Sequence: Synthetic primer
accgtctggcgaaattgtatttaatgag

SEQ ID NO: 261 - DNA, 29 bp, Artificial Sequence
  Description of Artificial Sequence: Synthetic probe
ccaggctttacctcgcacagtcgctatcc

SEQ ID NO: 262 - DNA, 27 bp, Artificial Sequence
  Description of Artificial Sequence: Synthetic primer
gaacgatagaccaatgcctttcatcat

SEQ ID NO: 263 - DNA, 25 bp, Artificial Sequence
  Description of Artificial Sequence: Synthetic primer
tttatacaccgtctggcgaaattgt

SEQ ID NO: 264 - DNA, 23 bp, Artificial Sequence
  Description of Artificial Sequence: Synthetic primer
gccgaagcagaaaaacggataca

SEQ ID NO: 265 - DNA, 29 bp, Artificial Sequence
  Description of Artificial Sequence: Synthetic probe
ccactctggaaaaacccgaacagcccaga

SEQ ID NO: 266 - DNA, 29 bp, Artificial Sequence
  Description of Artificial Sequence: Synthetic primer
caatttcgccagacggtgtataaaacata

SEQ ID NO: 267 - DNA, 24 bp, Artificial Sequence
  Description of Artificial Sequence: Synthetic primer
gccgaagcagaaaaacggatacaa

SEQ ID NO: 268 - DNA, 30 bp, Artificial Sequence
  Description of Artificial Sequence: Synthetic probe
tatccactctggaaaaacccgaacagccca

SEQ ID NO: 269 - DNA, 26 bp, Artificial Sequence
  Description of Artificial Sequence: Synthetic primer
acaatttcgccagacggtgtataaaa

SEQ ID NO: 270 - DNA, 23 bp, Artificial Sequence
  Description of Artificial Sequence: Synthetic primer
tgccgaagcagaaaaacggatac

SEQ ID NO: 271 - DNA, 24 bp, Artificial Sequence
  Description of Artificial Sequence: Synthetic primer
atgccgaagcagaaaaacggatac

SEQ ID NO: 272 - DNA, 22 bp, Artificial Sequence
  Description of Artificial Sequence: Synthetic primer
aaggattgatgccgaagcagaa

SEQ ID NO: 273 - DNA, 28 bp, Artificial Sequence
  Description of Artificial Sequence: Synthetic probe
ctggaaaaacccgaacagcccagagctt

SEQ ID NO: 274 - DNA, 26 bp, Artificial Sequence
  Description of Artificial Sequence: Synthetic primer
gccagacggtgtataaaacatatcca

SEQ ID NO: 275 - DNA, 23 bp, Artificial Sequence
  Description of Artificial Sequence: Synthetic primer
atgccgaagcagaaaaacggata

SEQ ID NO: 276 - DNA, 24 bp, Artificial Sequence
  Description of Artificial Sequence: Synthetic primer
gatgccgaagcagaaaaacggata

SEQ ID NO: 277 - DNA, 23 bp, Artificial Sequence
  Description of Artificial Sequence: Synthetic primer
aggattgatgccgaagcagaaaa

SEQ ID NO: 278 - DNA, 26 bp, Artificial Sequence
  Description of Artificial Sequence: Synthetic primer
aaggattgatgccgaagcagaaaaac

SEQ ID NO: 279 - DNA, 23 bp, Artificial Sequence
  Description of Artificial Sequence: Synthetic primer
aaggattgatgccgaagcagaaa

SEQ ID NO: 280 - DNA, 23 bp, Artificial Sequence
  Description of Artificial Sequence: Synthetic primer
caaggattgatgccgaagcagaa

SEQ ID NO: 281 - DNA, 24 bp, Artificial Sequence
  Description of Artificial Sequence: Synthetic primer
aaggattgatgccgaagcagaaaa

SEQ ID NO: 282 - DNA, 21 bp, Artificial Sequence
  Description of Artificial Sequence: Synthetic primer
gcaaggattgatgccgaagca

SEQ ID NO: 283 - DNA, 24 bp, Artificial Sequence
  Description of Artificial Sequence: Synthetic primer
caaggattgatgccgaagcagaaa

SEQ ID NO: 284 - DNA, 28 bp, Artificial Sequence
  Description of Artificial Sequence: Synthetic primer
acatacagacctatcagcttatccaaca

SEQ ID NO: 285 - DNA, 24 bp, Artificial Sequence
  Description of Artificial Sequence: Synthetic primer
cgggtttttccagagtggatatgt

SEQ ID NO: 286 - DNA, 30 bp, Artificial Sequence
  Description of Artificial Sequence: Synthetic primer
acatacagacctatcagcttatccaacatt

SEQ ID NO: 287 - DNA, 29 bp, Artificial Sequence
  Description of Artificial Sequence: Synthetic primer
acatacagacctatcagcttatccaacat

SEQ ID NO: 288 - DNA, 31 bp, Artificial Sequence
  Description of Artificial Sequence: Synthetic primer
tcaagcggcttttttgattatacagagaaat

SEQ ID NO: 289 - DNA, 29 bp, Artificial Sequence
  Description of Artificial Sequence: Synthetic probe
tttctgcttcggcatcaatccttgcaggc

SEQ ID NO: 290 - DNA, 28 bp, Artificial Sequence
  Description of Artificial Sequence: Synthetic primer
cagcccagagctttatatatggttacag

SEQ ID NO: 291 - DNA, 32 bp, Artificial Sequence
  Description of Artificial Sequence: Synthetic primer
tcaagcggcttttttgattatacagagaaata

SEQ ID NO: 292 - DNA, 28 bp, Artificial Sequence
  Description of Artificial Sequence: Synthetic probe
ttctgcttcggcatcaatccttgcaggc

SEQ ID NO: 293 - DNA, 28 bp, Artificial Sequence
  Description of Artificial Sequence: Synthetic primer
tcaagcggcttttttgattatacagaga

SEQ ID NO: 294 - DNA, 30 bp, Artificial Sequence
  Description of Artificial Sequence: Synthetic probe
ttttctgcttcggcatcaatccttgcaggc

SEQ ID NO: 295 - DNA, 23 bp, Artificial Sequence
  Description of Artificial Sequence: Synthetic primer
gttcgggtttttccagagtggat

SEQ ID NO: 296 - DNA, 25 bp, Artificial Sequence
  Description of Artificial Sequence: Synthetic probe
accaggctttacctcgcacagtcgc

SEQ ID NO: 297 - DNA, 35 bp, Artificial Sequence
  Description of Artificial Sequence: Synthetic primer
tcaagcggcttttttgattatacagagaaatatac

SEQ ID NO: 298 - DNA, 29 bp, Artificial Sequence
  Description of Artificial Sequence: Synthetic primer
gtcaagcggcttttttgattatacagaga

SEQ ID NO: 299 - DNA, 28 bp, Artificial Sequence
  Description of Artificial Sequence: Synthetic primer
tgtcaagcggcttttttgattatacaga

SEQ ID NO: 300 - DNA, 30 bp, Artificial Sequence
  Description of Artificial Sequence: Synthetic primer
tgtcaagcggcttttttgattatacagaga

SEQ ID NO: 301 - DNA, 31 bp, Artificial Sequence
  Description of Artificial Sequence: Synthetic primer
cacatacagacctatcagcttatccaacatt

SEQ ID NO: 302 - DNA, 32 bp, Artificial Sequence
  Description of Artificial Sequence: Synthetic primer
atcttcaaagatatatatgcctgcaaggattg

SEQ ID NO: 303 - DNA, 28 bp, Artificial Sequence
  Description of Artificial Sequence: Synthetic primer
ctgtcaagcggcttttttgattatacag

SEQ ID NO: 304 - DNA, 29 bp, Artificial Sequence
  Description of Artificial Sequence: Synthetic primer
gaacagcccagagctttatatatggttac

SEQ ID NO: 305 - DNA, 29 bp, Artificial Sequence
  Description of Artificial Sequence: Synthetic primer
ctgtcaagcggcttttttgattatacaga

SEQ ID NO: 306 - DNA, 27 bp, Artificial Sequence
  Description of Artificial Sequence: Synthetic primer
ccacatacagacctatcagcttatcca

SEQ ID NO: 307 - DNA, 28 bp, Artificial Sequence
  Description of Artificial Sequence: Synthetic primer
actgtcaagcggcttttttgattataca

SEQ ID NO: 308 - DNA, 27 bp, Artificial Sequence
  Description of Artificial Sequence: Synthetic primer
tccacatacagacctatcagcttatcc

SEQ ID NO: 309 - DNA, 29 bp, Artificial Sequence
  Description of Artificial Sequence: Synthetic primer
actgtcaagcggcttttttgattatacag

SEQ ID NO: 310 - DNA, 30 bp, Artificial Sequence
  Description of Artificial Sequence: Synthetic primer
actgtcaagcggcttttttgattatacaga

SEQ ID NO: 311 - DNA, 20 bp, Artificial Sequence
  Description of Artificial Sequence: Synthetic primer
gggctgttcgggtttttcca

SEQ ID NO: 312 - DNA, 28 bp, Artificial Sequence
  Description of Artificial Sequence: Synthetic primer
ttccacatacagacctatcagcttatcc

SEQ ID NO: 313 - DNA, 29 bp, Artificial Sequence
  Description of Artificial Sequence: Synthetic primer
aactgtcaagcggcttttttgattataca

SEQ ID NO: 314 - DNA, 30 bp, Artificial Sequence
  Description of Artificial Sequence: Synthetic primer
aactgtcaagcggcttttttgattatacag

SEQ ID NO: 315 - DNA, 29 bp, Artificial Sequence
  Description of Artificial Sequence: Synthetic primer
gaactgtcaagcggcttttttgattatac

SEQ ID NO: 316 - DNA, 25 bp, Artificial Sequence
  Description of Artificial Sequence: Synthetic primer
cgaactgtcaagcggcttttttgat

SEQ ID NO: 317 - DNA, 23 bp, Artificial Sequence
  Description of Artificial Sequence: Synthetic primer
tcgaactgtcaagcggctttttt

SEQ ID NO: 318 - DNA, 23 bp, Artificial Sequence
  Description of Artificial Sequence: Synthetic primer
atcgaactgtcaagcggcttttt

SEQ ID NO: 319 - DNA, 27 bp, Artificial Sequence
  Description of Artificial Sequence: Synthetic probe
tctgcttcggcatcaatccttgcaggc

SEQ ID NO: 320 - DNA, 20 bp, Artificial Sequence
  Description of Artificial Sequence: Synthetic primer
ccgaacagcccagagcttta

SEQ ID NO: 321 - DNA, 27 bp, Artificial Sequence
  Description of Artificial Sequence: Synthetic primer
aaccatatataaagctctgggctgttc

SEQ ID NO: 322 - DNA, 23 bp, Artificial Sequence
  Description of Artificial Sequence: Synthetic primer
ctgcaaggattgatgccgaagca

SEQ ID NO: 323 - DNA, 31 bp, Artificial Sequence
  Description of Artificial Sequence: Synthetic probe
atccactctggaaaaacccgaacagcccaga

SEQ ID NO: 324 - DNA, 21 bp, Artificial Sequence
  Description of Artificial Sequence: Synthetic primer
ggatagcgactgtgcgaggta

SEQ ID NO: 325 - DNA, 33 bp, Artificial Sequence
  Description of Artificial Sequence: Synthetic primer
gtcaagcggcttttttgattatacagagaaata

SEQ ID NO: 326 - DNA, 27 bp, Artificial Sequence
  Description of Artificial Sequence: Synthetic primer
cgaactgtcaagcggcttttttgatta

SEQ ID NO: 327 - DNA, 32 bp, Artificial Sequence
  Description of Artificial Sequence: Synthetic primer
gtcaagcggcttttttgattatacagagaaat

SEQ ID NO: 328 - DNA, 25 bp, Artificial Sequence
  Description of Artificial Sequence: Synthetic primer
aatcgaactgtcaagcggctttttt

SEQ ID NO: 329 - DNA, 25 bp, Artificial Sequence
  Description of Artificial Sequence: Synthetic primer
aaatcgaactgtcaagcggcttttt

SEQ ID NO: 330 - DNA, 25 bp, Artificial Sequence
  Description of Artificial Sequence: Synthetic primer
gaaatcgaactgtcaagcggctttt

SEQ ID NO: 331 - DNA, 26 bp, Artificial Sequence
  Description of Artificial Sequence: Synthetic primer
gaaatcgaactgtcaagcggcttttt

SEQ ID NO: 332 - DNA, 24 bp, Artificial Sequence
  Description of Artificial Sequence: Synthetic primer
atcgaactgtcaagcggctttttt

SEQ ID NO: 333 - DNA, 23 bp, Artificial Sequence
  Description of Artificial Sequence: Synthetic primer
aatcgaactgtcaagcggctttt

SEQ ID NO: 334 - DNA, 20 bp, Artificial Sequence
  Description of Artificial Sequence: Synthetic primer
ggtatcggagctgcagcaat

SEQ ID NO: 335 - DNA, 33 bp, Artificial Sequence
  Description of Artificial Sequence: Synthetic probe
tcccacagccaatttctacccctttcacttcct

SEQ ID NO: 336 - DNA, 25 bp, Artificial Sequence
  Description of Artificial Sequence: Synthetic primer
ctgatttggtcacattctccaacga

SEQ ID NO: 337 - DNA, 26 bp, Artificial Sequence
  Description of Artificial Sequence: Synthetic primer
tggtgtaaaaagcacccctagtatga

SEQ ID NO: 338 - DNA, 30 bp, Artificial Sequence
  Description of Artificial Sequence: Synthetic primer
agacgaagcttcaaaatatgatagccgtat

SEQ ID NO: 339 - DNA, 32 bp, Artificial Sequence
  Description of Artificial Sequence: Synthetic primer
cagcaatctccatgaataaaataatgctccat

SEQ ID NO: 340 - DNA, 27 bp, Artificial Sequence
  Description of Artificial Sequence: Synthetic primer
tggtgtaaaaagcacccctagtatgat

SEQ ID NO: 341 - DNA, 27 bp, Artificial Sequence
  Description of Artificial Sequence: Synthetic primer
ttggtgtaaaaagcacccctagtatga

SEQ ID NO: 342 - DNA, 28 bp, Artificial Sequence
  Description of Artificial Sequence: Synthetic primer
ttggtgtaaaaagcacccctagtatgat

SEQ ID NO: 343 - DNA, 29 bp, Artificial Sequence
  Description of Artificial Sequence: Synthetic primer
tggtgtaaaaagcacccctagtatgatta

SEQ ID NO: 344 - DNA, 30 bp, Artificial Sequence
  Description of Artificial Sequence: Synthetic primer
ttggtgtaaaaagcacccctagtatgatta

SEQ ID NO: 345 - DNA, 32 bp, Artificial Sequence
  Description of Artificial Sequence: Synthetic primer
acccctagtatgattatagaaaagggacaaga

SEQ ID NO: 346 - DNA, 26 bp, Artificial Sequence
  Description of Artificial Sequence: Synthetic primer
agggacaagacctacaaaaagtcgat

SEQ ID NO: 347 - DNA, 28 bp, Artificial Sequence
  Description of Artificial Sequence: Synthetic primer
aaagggacaagacctacaaaaagtcgat

SEQ ID NO: 348 - DNA, 29 bp, Artificial Sequence
  Description of Artificial Sequence: Synthetic primer
ttggtgtaaaaagcacccctagtatgatt

SEQ ID NO: 349 - DNA, 30 bp, Artificial Sequence
  Description of Artificial Sequence: Synthetic primer
tgcagcaatctccatgaataaaataatgct

SEQ ID NO: 350 - DNA, 31 bp, Artificial Sequence
  Description of Artificial Sequence: Synthetic primer
tagacgaagcttcaaaatatgatagccgtat

SEQ ID NO: 351 - DNA, 28 bp, Artificial Sequence
  Description of Artificial Sequence: Synthetic primer
gagctgcagcaatctccatgaataaaat

SEQ ID NO: 352 - DNA, 30 bp, Artificial Sequence
  Description of Artificial Sequence: Synthetic primer
gagctgcagcaatctccatgaataaaataa

SEQ ID NO: 353 - DNA, 21 bp, Artificial Sequence
  Description of Artificial Sequence: Synthetic primer
tgaggcaggctcatcaaaagg

SEQ ID NO: 354 - DNA, 28 bp, Artificial Sequence
  Description of Artificial Sequence: Synthetic primer
tggtgtaaaaagcacccctagtatgatt

SEQ ID NO: 355 - DNA, 32 bp, Artificial Sequence
  Description of Artificial Sequence: Synthetic primer
gcaatctccatgaataaaataatgctccatca

SEQ ID NO: 356 - DNA, 27 bp, Artificial Sequence
  Description of Artificial Sequence: Synthetic primer
agctgcagcaatctccatgaataaaat

SEQ ID NO: 357 - DNA, 29 bp, Artificial Sequence
  Description of Artificial Sequence: Synthetic primer
aaaagggacaagacctacaaaaagtcgat

SEQ ID NO: 358 - DNA, 31 bp, Artificial Sequence
  Description of Artificial Sequence: Synthetic primer
cacccctagtatgattatagaaaagggacaa

SEQ ID NO: 359 - DNA, 32 bp, Artificial Sequence
  Description of Artificial Sequence: Synthetic primer
cccctagtatgattatagaaaagggacaagac

SEQ ID NO: 360 - DNA, 29 bp, Artificial Sequence
  Description of Artificial Sequence: Synthetic primer
gagctgcagcaatctccatgaataaaata

SEQ ID NO: 361 - DNA, 31 bp, Artificial Sequence
  Description of Artificial Sequence: Synthetic primer
cccctagtatgattatagaaaagggacaaga

SEQ ID NO: 362 - DNA, 25 bp, Artificial Sequence
  Description of Artificial Sequence: Synthetic primer
ggcaggctcatcaaaaggaattagc

SEQ ID NO: 363 - DNA, 33 bp, Artificial Sequence
  Description of Artificial Sequence: Synthetic primer
aaggaattagcaaggtagaacaaaaaagtgatt

SEQ ID NO: 364 - DNA, 30 bp, Artificial Sequence
  Description of Artificial Sequence: Synthetic primer
agctgcagcaatctccatgaataaaataat

SEQ ID NO: 365 - DNA, 31 bp, Artificial Sequence
  Description of Artificial Sequence: Synthetic primer
ccatcaatttgctgaaacaattggtgtaaaa

SEQ ID NO: 366 - DNA, 29 bp, Artificial Sequence
  Description of Artificial Sequence: Synthetic primer
agctgcagcaatctccatgaataaaataa

SEQ ID NO: 367 - DNA, 30 bp, Artificial Sequence
  Description of Artificial Sequence: Synthetic primer
ctgcagcaatctccatgaataaaataatgc

SEQ ID NO: 368 - DNA, 29 bp, Artificial Sequence
  Description of Artificial Sequence: Synthetic primer
gacgaagcttcaaaatatgatagccgtat

SEQ ID NO: 369 - DNA, 30 bp, Artificial Sequence
  Description of Artificial Sequence: Synthetic primer
ccatcaatttgctgaaacaattggtgtaaa

SEQ ID NO: 370 - DNA, 25 bp, Artificial Sequence
  Description of Artificial Sequence: Synthetic primer
agctgcagcaatctccatgaataaa

SEQ ID NO: 371 - DNA, 26 bp, Artificial Sequence
  Description of Artificial Sequence: Synthetic primer
ttggtgtaaaaagcacccctagtatg

SEQ ID NO: 372 - DNA, 30 bp, Artificial Sequence
  Description of Artificial Sequence: Synthetic primer
gaaaagggacaagacctacaaaaagtcgat

SEQ ID NO: 373 - DNA, 28 bp, Artificial Sequence
  Description of Artificial Sequence: Synthetic primer
agctgcagcaatctccatgaataaaata

SEQ ID NO: 374 - DNA, 27 bp, Artificial Sequence
  Description of Artificial Sequence: Synthetic primer
ggtgtaaaaagcacccctagtatgatt

SEQ ID NO: 375 - DNA, 31 bp, Artificial Sequence
  Description of Artificial Sequence: Synthetic primer
gctgcagcaatctccatgaataaaataatgc

SEQ ID NO: 376 - DNA, 30 bp, Artificial Sequence
  Description of Artificial Sequence: Synthetic primer
acccctagtatgattatagaaaagggacaa

SEQ ID NO: 377 - DNA, 32 bp, Artificial Sequence
  Description of Artificial Sequence: Synthetic primer
agtatgattatagaaaagggacaagacctaca

SEQ ID NO: 378 - DNA, 25 bp, Artificial Sequence
  Description of Artificial Sequence: Synthetic primer
tggtgtaaaaagcacccctagtatg

SEQ ID NO: 379 - DNA, 24 bp, Artificial Sequence
  Description of Artificial Sequence: Synthetic primer
agctgcagcaatctccatgaataa

SEQ ID NO: 380 - DNA, 29 bp, Artificial Sequence
  Description of Artificial Sequence: Synthetic primer
atttgctgaaacaattggtgtaaaaagca

SEQ ID NO: 381 - DNA, 26 bp, Artificial Sequence
  Description of Artificial Sequence: Synthetic primer
ccacaagactgatttggtcacattct

SEQ ID NO: 382 - DNA, 32 bp, Artificial Sequence
  Description of Artificial Sequence: Synthetic primer
tccatcaatttgctgaaacaattggtgtaaaa

SEQ ID NO: 383 - DNA, 21 bp, Artificial Sequence
  Description of Artificial Sequence: Synthetic primer
gagctgcagcaatctccatga

SEQ ID NO: 384 - DNA, 31 bp, Artificial Sequence
  Description of Artificial Sequence: Synthetic primer
tcaatttgctgaaacaattggtgtaaaaagc

SEQ ID NO: 385 - DNA, 31 bp, Artificial Sequence
  Description of Artificial Sequence: Synthetic primer
gacaagacctacaaaaagtcgatgaatttgc

SEQ ID NO: 386 - DNA, 23 bp, Artificial Sequence
  Description of Artificial Sequence: Synthetic primer
gagctgcagcaatctccatgaat

SEQ ID NO: 387 - DNA, 32 bp, Artificial Sequence
  Description of Artificial Sequence: Synthetic primer
atcaatttgctgaaacaattggtgtaaaaagc

SEQ ID NO: 388 - DNA, 19 bp, Artificial Sequence
  Description of Artificial Sequence: Synthetic primer
tgcgccatactgggaaacg

SEQ ID NO: 389 - DNA, 30 bp, Artificial Sequence
  Description of Artificial Sequence: Synthetic probe
tgatccacctcgccagccatgagatcattt

SEQ ID NO: 390 - DNA, 20 bp, Artificial Sequence
  Description of Artificial Sequence: Synthetic primer
agccgtgtctcagctcaatc

SEQ ID NO: 391 - DNA, 20 bp, Artificial Sequence
  Description of Artificial Sequence: Synthetic primer
ctgcgccatactgggaaacg

SEQ ID NO: 392 - DNA, 26 bp, Artificial Sequence
  Description of Artificial Sequence: Synthetic probe
tctgatccacctcgccagccatgaga

SEQ ID NO: 393 - DNA, 25 bp, Artificial Sequence
  Description of Artificial Sequence: Synthetic primer
ttaaaaaagccgtgtctcagctcaa

SEQ ID NO: 394 - DNA, 19 bp, Artificial Sequence
  Description of Artificial Sequence: Synthetic primer
ggtaggctgcgccatactg

SEQ ID NO: 395 - DNA, 21 bp, Artificial Sequence
  Description of Artificial Sequence: Synthetic primer
aaaagccgtgtctcagctcaa

SEQ ID NO: 396 - DNA, 20 bp, Artificial Sequence
  Description of Artificial Sequence: Synthetic primer
gctgcgccatactgggaaac

SEQ ID NO: 397 - DNA, 32 bp, Artificial Sequence
  Description of Artificial Sequence: Synthetic probe
tctgatccacctcgccagccatgagatcattt

SEQ ID NO: 398 - DNA, 26 bp, Artificial Sequence
  Description of Artificial Sequence: Synthetic primer
cttaaaaaagccgtgtctcagctcaa

SEQ ID NO: 399 - DNA, 26 bp, Artificial Sequence
  Description of Artificial Sequence: Synthetic primer
agattttgattgaagaggccgttacc

SEQ ID NO: 400 - DNA, 27 bp, Artificial Sequence
  Description of Artificial Sequence: Synthetic probe
ttcccagtatggcgcagcctacctcac

SEQ ID NO: 401 - DNA, 22 bp, Artificial Sequence
  Description of Artificial Sequence: Synthetic primer
ctcgccagccatgagatcattt

SEQ ID NO: 402 - DNA, 26 bp, Artificial Sequence
  Description of Artificial Sequence: Synthetic probe
cccagtatggcgcagcctacctcact

SEQ ID NO: 403 - DNA, 22 bp, Artificial Sequence
  Description of Artificial Sequence: Synthetic probe
tggcgcagcctacctcactccc

SEQ ID NO: 404 - DNA, 25 bp, Artificial Sequence
  Description of Artificial Sequence: Synthetic probe
tcccagtatggcgcagcctacctca

SEQ ID NO: 405 - DNA, 26 bp, Artificial Sequence
  Description of Artificial Sequence: Synthetic probe
tcccagtatggcgcagcctacctcac

SEQ ID NO: 406 - DNA, 26 bp, Artificial Sequence
  Description of Artificial Sequence: Synthetic probe
agtatggcgcagcctacctcactccc

SEQ ID NO: 407 - DNA, 27 bp, Artificial Sequence
  Description of Artificial Sequence: Synthetic probe
tttcccagtatggcgcagcctacctca

SEQ ID NO: 408 - DNA, 28 bp, Artificial Sequence
  Description of Artificial Sequence: Synthetic probe
tttcccagtatggcgcagcctacctcac

SEQ ID NO: 409 - DNA, 20 bp, Artificial Sequence
  Description of Artificial Sequence: Synthetic primer
aggtaggctgcgccatactg

SEQ ID NO: 410 - DNA, 31 bp, Artificial Sequence
  Description of Artificial Sequence: Synthetic probe
ctgatccacctcgccagccatgagatcattt

SEQ ID NO: 411 - DNA, 24 bp, Artificial Sequence
  Description of Artificial Sequence: Synthetic primer
aaaaaagccgtgtctcagctcaat

SEQ ID NO: 412 - DNA, 25 bp, Artificial Sequence
  Description of Artificial Sequence: Synthetic probe
cccagtatggcgcagcctacctcac

SEQ ID NO: 413 - DNA, 23 bp, Artificial Sequence
  Description of Artificial Sequence: Synthetic probe
atggcgcagcctacctcactccc

SEQ ID NO: 414 - DNA, 26 bp, Artificial Sequence
  Description of Artificial Sequence: Synthetic probe
ttcccagtatggcgcagcctacctca

SEQ ID NO: 415 - DNA, 27 bp, Artificial Sequence
  Description of Artificial Sequence: Synthetic primer
aagattttgattgaagaggccgttacc

SEQ ID NO: 416 - DNA, 28 bp, Artificial Sequence
  Description of Artificial Sequence: Synthetic primer
caagattttgattgaagaggccgttacc

SEQ ID NO: 417 - DNA, 18 bp, Artificial Sequence
  Description of Artificial Sequence: Synthetic primer
cctcgccagccatgagat

SEQ ID NO: 418 - DNA, 29 bp, Artificial Sequence
  Description of Artificial Sequence: Synthetic probe
caatctgatccacctcgccagccatgaga

SEQ ID NO: 419 - DNA, 28 bp, Artificial Sequence
  Description of Artificial Sequence: Synthetic primer
cctgatgaatcttaaaaaagccgtgtct

SEQ ID NO: 420 - DNA, 31 bp, Artificial Sequence
  Description of Artificial Sequence: Synthetic probe
ctcaatctgatccacctcgccagccatgaga

SEQ ID NO: 421 - DNA, 29 bp, Artificial Sequence
  Description of Artificial Sequence: Synthetic primer
tcctgatgaatcttaaaaaagccgtgtct

SEQ ID NO: 422 - DNA, 32 bp, Artificial Sequence
  Description of Artificial Sequence: Synthetic probe
ctcaatctgatccacctcgccagccatgagat

SEQ ID NO: 423 - DNA, 30 bp, Artificial Sequence
  Description of Artificial Sequence: Synthetic probe
ctcaatctgatccacctcgccagccatgag

SEQ ID NO: 424 - DNA, 30 bp, Artificial Sequence
  Description of Artificial Sequence: Synthetic probe
tcaatctgatccacctcgccagccatgaga

SEQ ID NO: 425 - DNA, 28 bp, Artificial Sequence
  Description of Artificial Sequence: Synthetic primer
agcaagattttgattgaagaggccgtta

SEQ ID NO: 426 - DNA, 17 bp, Artificial Sequence
  Description of Artificial Sequence: Synthetic primer
cacctcgccagccatga

SEQ ID NO: 427 - DNA, 31 bp, Artificial Sequence
  Description of Artificial Sequence: Synthetic probe
tcaatctgatccacctcgccagccatgagat

SEQ ID NO: 428 - DNA, 24 bp, Artificial Sequence
  Description of Artificial Sequence: Synthetic primer
ggcttgcttgaattgtcaggcatt

SEQ ID NO: 429 - DNA, 33 bp, Artificial Sequence
  Description of Artificial Sequence: Synthetic probe
cacggagctttgaatatcgcatcccacatacgg

SEQ ID NO: 430 - DNA, 24 bp, Artificial Sequence
  Description of Artificial Sequence: Synthetic primer
aacggtatatgcaagcgccttatc

SEQ ID NO: 431 - DNA, 27 bp, Artificial Sequence
  Description of Artificial Sequence: Synthetic probe
agctcaatctgatccacctcgccagcc

SEQ ID NO: 432 - DNA, 29 bp, Artificial Sequence
  Description of Artificial Sequence: Synthetic probe
tcaatctgatccacctcgccagccatgag

SEQ ID NO: 433 - DNA, 27 bp, Artificial Sequence
  Description of Artificial Sequence: Synthetic probe
tctgatccacctcgccagccatgagat

SEQ ID NO: 434 - DNA, 19 bp, Artificial Sequence
  Description of Artificial Sequence: Synthetic primer
cggctgtgcttcctgatga

SEQ ID NO: 435 - DNA, 26 bp, Artificial Sequence
  Description of Artificial Sequence: Synthetic primer
ccatacaaggcttgcttgaattgtca

SEQ ID NO: 436 - DNA, 34 bp, Artificial Sequence
  Description of Artificial Sequence: Synthetic probe
acggagctttgaatatcgcatcccacatacggaa

SEQ ID NO: 437 - DNA, 25 bp, Artificial Sequence
  Description of Artificial Sequence: Synthetic primer
atacccgcatttttcacaacggtat

SEQ ID NO: 438 - DNA, 18 bp, Artificial Sequence
  Description of Artificial Sequence: Synthetic primer
ggccgttaccgggagtga

SEQ ID NO: 439 - DNA, 30 bp, Artificial Sequence
  Description of Artificial Sequence: Synthetic primer
ttcctgatgaatcttaaaaaagccgtgtct

SEQ ID NO: 440 - DNA, 19 bp, Artificial Sequence
  Description of Artificial Sequence: Synthetic primer
aggaagcacagccggagaa

SEQ ID NO: 441 - DNA, 26 bp, Artificial Sequence
  Description of Artificial Sequence: Synthetic probe
cctcatccggtaaggcggctggaact

SEQ ID NO: 442 - DNA, 27 bp, Artificial Sequence
  Description of Artificial Sequence: Synthetic primer
agccaagtatccggtaaatcttcattg

SEQ ID NO: 443 - DNA, 20 bp, Artificial Sequence
  Description of Artificial Sequence: Synthetic primer
ccgttaccgggagtgaggta

SEQ ID NO: 444 - DNA, 28 bp, Artificial Sequence
  Description of Artificial Sequence: Synthetic primer
tgatgaatcttaaaaaagccgtgtctca

SEQ ID NO: 445 - DNA, 21 bp, Artificial Sequence
  Description of Artificial Sequence: Synthetic primer
cgctctcgtatccggtctttg

SEQ ID NO: 446 - DNA, 30 bp, Artificial Sequence
  Description of Artificial Sequence: Synthetic probe
ccgttccggctcctcttttggcgtgaataa

SEQ ID NO: 447 - DNA, 20 bp, Artificial Sequence
  Description of Artificial Sequence: Synthetic primer
ttccggctgtgcttcctgat

SEQ ID NO: 448 - DNA, 19 bp, Artificial Sequence
  Description of Artificial Sequence: Synthetic primer
ccggctgtgcttcctgatg

SEQ ID NO: 449 - DNA, 19 bp, Artificial Sequence
  Description of Artificial Sequence: Synthetic primer
tccggctgtgcttcctgat

SEQ ID NO: 450 - DNA, 21 bp, Artificial Sequence
  Description of Artificial Sequence: Synthetic primer
tcctacctcactcccggaaac

SEQ ID NO: 451 - DNA, 20 bp, Artificial Sequence
  Description of Artificial Sequence: Synthetic primer
cctacctcactcccggaaac

SEQ ID NO: 452 - DNA, 18 bp, Artificial Sequence
  Description of Artificial Sequence: Synthetic primer
gaccgctgcctgcagttc

SEQ ID NO: 453 - DNA, 22 bp, Artificial Sequence
  Description of Artificial Sequence: Synthetic primer
atcctacctcactcccggaaac

SEQ ID NO: 454 - DNA, 22 bp, Artificial Sequence
  Description of Artificial Sequence: Synthetic primer
catcctacctcactcccggaaa

SEQ ID NO: 455 - DNA, 18 bp, Artificial Sequence
  Description of Artificial Sequence: Synthetic primer
ccggctgtgcttcctgat

SEQ ID NO: 456 - DNA, 25 bp, Artificial Sequence
  Description of Artificial Sequence: Synthetic primer
ggttgtgaaaaatgcgggaattgag

SEQ ID NO: 457 - DNA, 25 bp, Artificial Sequence
  Description of Artificial Sequence: Synthetic probe
ctgcccgttccggctcctcttttgg

SEQ ID NO: 458 - DNA, 23 bp, Artificial Sequence
  Description of Artificial Sequence: Synthetic primer
tctgcccggcataccttattcac

SEQ ID NO: 459 - DNA, 24 bp, Artificial Sequence
  Description of Artificial Sequence: Synthetic primer
cagtatggcacatcctacctcact

SEQ ID NO: 460 - DNA, 24 bp, Artificial Sequence
  Description of Artificial Sequence: Synthetic primer
cagccaagtacccggtaaatcttc

SEQ ID NO: 461 - DNA, 27 bp, Artificial Sequence
  Description of Artificial Sequence: Synthetic primer
gctgtgcttcctgatggatcttaaaaa

SEQ ID NO: 462 - DNA, 20 bp, Artificial Sequence
  Description of Artificial Sequence: Synthetic primer
cgttccggctcctcttttgg

SEQ ID NO: 463 - DNA, 23 bp, Artificial Sequence
  Description of Artificial Sequence: Synthetic probe
accgctgcctgcagttcctctgc

SEQ ID NO: 464 - DNA, 19 bp, Artificial Sequence
  Description of Artificial Sequence: Synthetic primer
cggaaacggcctcctcaac

SEQ ID NO: 465 - DNA, 20 bp, Artificial Sequence
  Description of Artificial Sequence: Synthetic primer
ggctcctcttttggcgtgaa

SEQ ID NO: 466 - DNA, 25 bp, Artificial Sequence
  Description of Artificial Sequence: Synthetic probe
tgcagttcctctgcccggcatacct

SEQ ID NO: 467 - DNA, 20 bp, Artificial Sequence
  Description of Artificial Sequence: Synthetic primer
ctacctcactcccggaaacg

SEQ ID NO: 468 - DNA, 19 bp, Artificial Sequence
  Description of Artificial Sequence: Synthetic primer
ccgttccggctcctctttt

SEQ ID NO: 469 - DNA, 27 bp, Artificial Sequence
  Description of Artificial Sequence: Synthetic probe
ctctgcccggcataccttattcacgcc

SEQ ID NO: 470 - DNA, 16 bp, Artificial Sequence
  Description of Artificial Sequence: Synthetic primer
accgctgcctgcagtt

SEQ ID NO: 471 - DNA, 21 bp, Artificial Sequence
  Description of Artificial Sequence: Synthetic primer
ctgcttgagctgtccggcatt

SEQ ID NO: 472 - DNA, 32 bp, Artificial Sequence
  Description of Artificial Sequence: Synthetic probe
agaactcgaaacccaggtacctcaattcccgc

SEQ ID NO: 473 - DNA, 19 bp, Artificial Sequence
  Description of Artificial Sequence: Synthetic primer
tccaggctgtccccctttt

SEQ ID NO: 474 - DNA, 22 bp, Artificial Sequence
  Description of Artificial Sequence: Synthetic primer
taaggtatgccgggcagaggaa

SEQ ID NO: 475 - DNA, 26 bp, Artificial Sequence
  Description of Artificial Sequence: Synthetic probe
tcccggaaacggcctcctcaaccaat

SEQ ID NO: 476 - DNA, 22 bp, Artificial Sequence
  Description of Artificial Sequence: Synthetic primer
cccagtatggcacatcctacct

SEQ ID NO: 477 - DNA, 24 bp, Artificial Sequence
  Description of Artificial Sequence: Synthetic primer
gtcgctcgcttatatggttgtgaa

SEQ ID NO: 478 - DNA, 31 bp, Artificial Sequence
  Description of Artificial Sequence: Synthetic probe
actcgaaacccaggtacctcaattcccgcat

SEQ ID NO: 479 - DNA, 21 bp, Artificial Sequence
  Description of Artificial Sequence: Synthetic primer
aggctgtcccccttttgtaga

SEQ ID NO: 480 - DNA, 24 bp, Artificial Sequence
  Description of Artificial Sequence: Synthetic primer
ctcctcttttggcgtgaataaggt

SEQ ID NO: 481 - DNA, 24 bp, Artificial Sequence
  Description of Artificial Sequence: Synthetic probe
tctgtgaccgctgcctgcagttcc

SEQ ID NO: 482 - DNA, 20 bp, Artificial Sequence
  Description of Artificial Sequence: Synthetic primer
aaacggcctcctcaaccaat

SEQ ID NO: 483 - DNA, 25 bp, Artificial Sequence
  Description of Artificial Sequence: Synthetic primer
tggataagtcgctcgcttatatggt

SEQ ID NO: 484 - DNA, 30 bp, Artificial Sequence
  Description of Artificial Sequence: Synthetic probe
actcgaaacccaggtacctcaattcccgca

SEQ ID NO: 485 - DNA, 32 bp, Artificial Sequence
  Description of Artificial Sequence: Synthetic probe
acccaggtacctcaattcccgcatttttcaca

SEQ ID NO: 486 - DNA, 20 bp, Artificial Sequence
  Description of Artificial Sequence: Synthetic primer
caggctgtcccccttttgta

SEQ ID NO: 487 - DNA, 33 bp, Artificial Sequence
  Description of Artificial Sequence: Synthetic probe
aacccaggtacctcaattcccgcatttttcaca

SEQ ID NO: 488 - DNA, 26 bp, Artificial Sequence
  Description of Artificial Sequence: Synthetic primer
cctcttttggcgtgaataaggtatgc

SEQ ID NO: 489 - DNA, 25 bp, Artificial Sequence
  Description of Artificial Sequence: Synthetic probe
ctctgtgaccgctgcctgcagttcc

SEQ ID NO: 490 - DNA, 19 bp, Artificial Sequence
  Description of Artificial Sequence: Synthetic primer
gaaacggcctcctcaacca

SEQ ID NO: 491 - DNA, 25 bp, Artificial Sequence
  Description of Artificial Sequence: Synthetic primer
tcgctcgcttatatggttgtgaaaa

SEQ ID NO: 492 - DNA, 28 bp, Artificial Sequence
  Description of Artificial Sequence: Synthetic probe
tcgaaacccaggtacctcaattcccgca

SEQ ID NO: 493 - DNA, 19 bp, Artificial Sequence
  Description of Artificial Sequence: Synthetic primer
caggctgtcccccttttgt

SEQ ID NO: 494 - DNA, 26 bp, Artificial Sequence
  Description of Artificial Sequence: Synthetic primer
atggataagtcgctcgcttatatggt

SEQ ID NO: 495 - DNA, 26 bp, Artificial Sequence
  Description of Artificial Sequence: Synthetic primer
gataagtcgctcgcttatatggttgt

SEQ ID NO: 496 - DNA, 27 bp, Artificial Sequence
  Description of Artificial Sequence: Synthetic primer
tatggataagtcgctcgcttatatggt

SEQ ID NO: 497 - DNA, 28 bp, Artificial Sequence
  Description of Artificial Sequence: Synthetic primer
gtatggataagtcgctcgcttatatggt

SEQ ID NO: 498 - DNA, 28 bp, Artificial Sequence
  Description of Artificial Sequence: Synthetic primer
tgtatggataagtcgctcgcttatatgg

SEQ ID NO: 499 - DNA, 33 bp, Artificial Sequence
  Description of Artificial Sequence: Synthetic probe
acccaggtacctcaattcccgcatttttcacaa

SEQ ID NO: 500 - DNA, 27 bp, Artificial Sequence
  Description of Artificial Sequence: Synthetic primer
gtatggataagtcgctcgcttatatgg

SEQ ID NO: 501 - DNA, 29 bp, Artificial Sequence
  Description of Artificial Sequence: Synthetic primer
ctgtatggataagtcgctcgcttatatgg

SEQ ID NO: 502 - DNA, 30 bp, Artificial Sequence
  Description of Artificial Sequence: Synthetic primer
tgtatggataagtcgctcgcttatatggtt

SEQ ID NO: 503 - DNA, 324 bp, Artificial Sequence
  Description of Artificial Sequence: Synthetic polynucleotide
gcgaagtgagaatacgccgtgtcgcagtttccttgagcagtgtctctaaatgcctcaaaccgtcgcatttttggttatagcagtaactatatggaggtccgtaggcggcgtgcgtgggggcaccaaactcatccaacggtcgactgcgcctgtagggtcttaagaagcggcacctcagaccgatagcatagcacttaaagaggaattgaataatcaagatgggtatccgaccgacgcggagtgaccgaggaagaggaccctgcatgtatcctgagagtatagttgtcagagcagcaattgattcaccaccaagggacttagtct

SEQ ID NO: 504 - DNA, 24 bp, Artificial Sequence
  Description of Artificial Sequence: Synthetic primer
cagaccgatagcatagcacttaaa

SEQ ID NO: 505 - DNA, 32 bp, Artificial Sequence
  Description of Artificial Sequence: Synthetic probe
tgctgctctgacaactatactctcaggataca

SEQ ID NO: 506 - DNA, 20 bp, Artificial Sequence
  Description of Artificial Sequence: Synthetic primer
tcccttggtggtgaatcaat

SEQ ID NO: 507 - DNA, 298 bp, Artificial Sequence
  Description of Artificial Sequence: Synthetic polynucleotide
tagaaagattattatctgatatggacgctattccaacagatatcaagacagctgtacgtaacaatggtggcggacatgctaaccattcatttttctgggaaatcatggcaccaaatgctggtggcgaacctacaggagaaataaaagaagcgattaatgaagcttttggtgatttttcttcttttaaagaagaattcaaaaaagcagccgctggacgatttggttctggatgggcttggcatgtaatggaaattggaaaattagctattacctctactgcaaatcaagattctccatt

SEQ ID NO: 508 - DNA, 286 bp, Artificial Sequence
  Description of Artificial Sequence: Synthetic polynucleotide
tctgtagaaaacctaatttcagatatgaatgctattcctgaagatatccgcacagctgttcgtaacaatggtggcggtcacgcaaaccatacattcttctgggaaattatggcaccaaatgctggtggacaaccaactggcgctattaaagaagcaatcgatgaaacatttggtagctttgatgaaatgaaagctgctttcaaaacagctgcaactggccgctttggttcaggttgggcttggttagttgtgaataacggtaaattagaaatcacttcaacaccaa

SEQ ID NO: 509 - DNA, 220 bp, Artificial Sequence
  Description of Artificial Sequence: Synthetic polynucleotide
acgcgttcgttttcgttctcttctagcaaaaatactccccgtatcccgcttcagaggtttcgccttcttatggatgtccttatagcgatccatccgactcattgtggttcctcctcgtttcttactgataaataaaggatacgataaccaatcgacaaaagcctatcattttttgataatttaataaaaaataacgaaataaatgcttcgatacataaag

SEQ ID NO: 510 - DNA, 1510 bp, Artificial Sequence
  Description of Artificial Sequence: Synthetic polynucleotide
ctcgttccttttagcaaaagtaattgggacaagcgtcgcacatcttcaggagctaaatcaatttttttaatcgtatgatacagcgtttctaattgtttcactgtctctggattaggtactccgtcaagtactcgtacttgaaaatcatctaaaatattttcaccattttcaatatacgcatctaaaaaagaagtatccaatgactgttgcaataactggattgcttgttccattggctcgaaagcctcttcaattttttctgggaacaaattagcacctctattctattcaaaatgtaacaacttccatacctgttttattttaacgaaaaaagaaagaataatcaatgcctttactgtctttcttctaaaatctaataatttgtagaaaaacaacgctattccagtattgtttccgcttttttttgatgagaaactctctcttgatattggtattttcgaccagatttgtttaataaaacagttattcttaattgttcatttttgtatgtataatcgagggtcacttgcgcgtatacgagcctttcttctgtttttaactttcgtctattttgtttaacatctgcaagaaataaatgaaagatacttttccctgcatagtattcctgggtagcttgcgtaaatttttgtgttaactgacgttcttctagtaactgaagggcgagaaaactcatcaaataaacaatggccatggccgtgaacaataagtttcctgaatatttttgcctcattaaaattctccctttatcggaaaaaaactatcaaatgtttttccagaagtaaaagtaacttccaaagtaaacgattgtccttcatttttaaactgtccatttttgataccaattaacagcggttgatagcctttaccattgactcgcttaataattttatcttctttgatttgaattgaaatagtcttgtttgtttctgagtcgaggaaagaaatctcttgagcagagcctgtttggaaaacgagcttttgacattctttttctaattgaattaaaaaaatatgccaggatttcgaatcattgtttttcaaatattgatttccgataaaactttgttgaatcatcaactgaaacaattgacacatacaacttaaaacgactagtgctactaaacactctaacatcgtaaaacccgcatattttttattgaccaatcaaaatcgctcctcttgaatctgtgacttttagtatccttcttccatcgttctccatcaaagaaaatcggtaagtttctccttgatatatccgctctttgaaacgtaaatcacccgtctgctctaaaaataaaattgcttcgtatcctagacgagtgcgcgttaattcttgctcccgttgataattttgctgaattaattgtgtaatagtcagggagaatatccctgctatcacacatacaatactaaaactaacgagactctctaataaaataaaaccgctataaatagttaattttcttcgtatatttgccactccccatct

SEQ ID NO: 511 - DNA, 162 bp, Artificial Sequence
  Description of Artificial Sequence: Synthetic polynucleotide
cgagcgttctcgagaatactcgacgctgaaatttttgcaccatttattgctttatcgtgacgtgactttgcaataacaaatcctaaaccaagaccgacaatcaaaccgacgatagcgaggagaatatttaaaaccatatttccacctccatactatcttttt

SEQ ID NO: 512 - DNA, 139 bp, Artificial Sequence
  Description of Artificial Sequence: Synthetic polynucleotide
gctgatgattgtgcaccagctatttctttctcgtgacgtgactttgcgaccataaaccctaaaccaagaccgacaattaaaccgatgatagcgaggagaatgttgaataccataaaatccacctccatactatcttttt

SEQ ID NO: 513 - DNA, 21 bp, Artificial Sequence
  Description of Artificial Sequence: Synthetic primer
ggacatgctaaccattcattt

SEQ ID NO: 514 - DNA, 20 bp, Artificial Sequence
  Description of Artificial Sequence: Synthetic primer
cgctggacgatttggttctg

SEQ ID NO: 515 - DNA, 24 bp, Artificial Sequence
  Description of Artificial Sequence: Synthetic primer
agcgattaatgaagcttttggtga

SEQ ID NO: 516 - DNA, 20 bp, Artificial Sequence
  Description of Artificial Sequence: Synthetic primer
ggaaatcatggcaccgaatg

SEQ ID NO: 517 - DNA, 22 bp, Artificial Sequence
  Description of Artificial Sequence: Synthetic primer
tggtggacatgctaaccattca

SEQ ID NO: 518 - DNA, 25 bp, Artificial Sequence
  Description of Artificial Sequence: Synthetic primer
agcgattaatgaagcttttggtgat

SEQ ID NO: 519 - DNA, 25 bp, Artificial Sequence
  Description of Artificial Sequence: Synthetic primer
cagtagaggtaatagctaattttcc

SEQ ID NO: 520 - DNA, 19 bp, Artificial Sequence
  Description of Artificial Sequence: Synthetic primer
atttctcctgtaggttcgc

SEQ ID NO: 521 - DNA, 16 bp, Artificial Sequence
  Description of Artificial Sequence: Synthetic primer
gccaagcccatccaga

SEQ ID NO: 522 - DNA, 17 bp, Artificial Sequence
  Description of Artificial Sequence: Synthetic primer
ccattacaagccaagcc

SEQ ID NO: 523 - DNA, 22 bp, Artificial Sequence
  Description of Artificial Sequence: Synthetic primer
agcttcattaatcgcttctttt

SEQ ID NO: 524 - DNA, 18 bp, Artificial Sequence
  Description of Artificial Sequence: Synthetic primer
gccaagcccatccagaac

SEQ ID NO: 525 - DNA, 16 bp, Artificial Sequence
  Description of Artificial Sequence: Synthetic primer
gccaagcccaaccaga

SEQ ID NO: 526 - DNA, 25 bp, Artificial Sequence
  Description of Artificial Sequence: Synthetic primer
gcttcattaattgcttcttttattg

SEQ ID NO: 527 - DNA, 21 bp, Artificial Sequence
  Description of Artificial Sequence: Synthetic primer
ggcagtagaggtaatagctaa

SEQ ID NO: 528 - DNA, 22 bp, Artificial Sequence
  Description of Artificial Sequence: Synthetic primer
ggcagtagaggtaatagctaat

SEQ ID NO: 529 - DNA, 25 bp, Artificial Sequence
  Description of Artificial Sequence: Synthetic primer
ccaaaagcttcattaatcgcttctt

SEQ ID NO: 530 - DNA, 22 bp, Artificial Sequence
  Description of Artificial Sequence: Synthetic primer
catccagaaccaaatcgtccag

SEQ ID NO: 531 - DNA, 24 bp, Artificial Sequence
  Description of Artificial Sequence: Synthetic primer
tcaccaaaagcttcattaatcgct

SEQ ID NO: 532 - DNA, 25 bp, Artificial Sequence
  Description of Artificial Sequence: Synthetic primer
tcttttatttctcctgtaggttcgc

SEQ ID NO: 533 - DNA, 25 bp, Artificial Sequence
  Description of Artificial Sequence: Synthetic primer
cattaatcgcttcttttatttctcc

SEQ ID NO: 534 - DNA, 25 bp, Artificial Sequence
  Description of Artificial Sequence: Synthetic primer
taatcgcttcttttatttctcctgt

SEQ ID NO: 535 - DNA, 23 bp, Artificial Sequence
  Description of Artificial Sequence: Synthetic primer
cattttccattacaagccaagcc

SEQ ID NO: 536 - DNA, 22 bp, Artificial Sequence
  Description of Artificial Sequence: Synthetic primer
caagcccatccagaaccaaatc

SEQ ID NO: 537 - DNA, 17 bp, Artificial Sequence
  Description of Artificial Sequence: Synthetic primer
gtccagcggctgctttt

SEQ ID NO: 538 - DNA, 21 bp, Artificial Sequence
  Description of Artificial Sequence: Synthetic primer
catccagaaccaaatcgtcca

SEQ ID NO: 539 - DNA, 19 bp, Artificial Sequence
  Description of Artificial Sequence: Synthetic primer
tgccaagcccatccagaac

SEQ ID NO: 540 - DNA, 21 bp, Artificial Sequence
  Description of Artificial Sequence: Synthetic primer
caaccagaaccaaaacgtcca

SEQ ID NO: 541 - DNA, 25 bp, Artificial Sequence
  Description of Artificial Sequence: Synthetic primer
tcgccaaaagcttcattaattgctt

SEQ ID NO: 542 - DNA, 21 bp, Artificial Sequence
  Description of Artificial Sequence: Synthetic primer
aaaatcgccaaaagcttcatt

SEQ ID NO: 543 - DNA, 18 bp, Artificial Sequence
  Description of Artificial Sequence: Synthetic primer
gccaagcccaaccagaac

SEQ ID NO: 544 - DNA, 21 bp, Artificial Sequence
  Description of Artificial Sequence: Synthetic primer
attacaagccaagcccaacca

SEQ ID NO: 545 - DNA, 22 bp, Artificial Sequence
  Description of Artificial Sequence: Synthetic primer
attctccattacaagccaagcc

SEQ ID NO: 546 - DNA, 18 bp, Artificial Sequence
  Description of Artificial Sequence: Synthetic primer
cgtccagctgctgctttt

SEQ ID NO: 547 - DNA, 25 bp, Artificial Sequence
  Description of Artificial Sequence: Synthetic primer
taattgcttcttttattgccccagt

SEQ ID NO: 548 - DNA, 24 bp, Artificial Sequence
  Description of Artificial Sequence: Synthetic primer
tttcccattctccattacaagcca

SEQ ID NO: 549 - DNA, 25 bp, Artificial Sequence
  Description of Artificial Sequence: Synthetic primer
agctaatttcccattctccattaca

SEQ ID NO: 550 - DNA, 25 bp, Artificial Sequence
  Description of Artificial Sequence: Synthetic primer
tggagaatcttgattggcagtagag

SEQ ID NO: 551 - DNA, 24 bp, Artificial Sequence
  Description of Artificial Sequence: Synthetic primer
ttgattggcagtagaggtaatagc

SEQ ID NO: 552 - DNA, 25 bp, Artificial Sequence
  Description of Artificial Sequence: Synthetic primer
gattggcagtagaggtaatagctaa

SEQ ID NO: 553 - DNA, 23 bp, Artificial Sequence
  Description of Artificial Sequence: Synthetic primer
tattacaagccaagcccatccag

SEQ ID NO: 554 - DNA, 19 bp, Artificial Sequence
  Description of Artificial Sequence: Synthetic probe
catggcaccaaatgctggt

SEQ ID NO: 555 - DNA, 23 bp, Artificial Sequence
  Description of Artificial Sequence: Synthetic probe
cggtgccatgatttcccagaaaa

SEQ ID NO: 556 - DNA, 20 bp, Artificial Sequence
  Description of Artificial Sequence: Synthetic probe
ccaaaacgtccagctgctgc

SEQ ID NO: 557 - DNA, 17 bp, Artificial Sequence
  Description of Artificial Sequence: Synthetic probe
atggcaccgaatgcggg

SEQ ID NO: 558 - DNA, 27 bp, Artificial Sequence
  Description of Artificial Sequence: Synthetic probe
agaagcaattaatgaagcttttggcga

SEQ ID NO: 559 - DNA, 26 bp, Artificial Sequence
  Description of Artificial Sequence: Synthetic probe
attgcttcttttattgccccagtagg

SEQ ID NO: 560 - DNA, 35 bp, Artificial Sequence
  Description of Artificial Sequence: Synthetic probe
agctaattttccattttccattacaagccaagccc

SEQ ID NO: 561 - DNA, 35 bp, Artificial Sequence
  Description of Artificial Sequence: Synthetic probe
tgggcttggcttgtaatggaaaatggaaaattagc

SEQ ID NO: 562 - DNA, 31 bp, Artificial Sequence
  Description of Artificial Sequence: Synthetic probe
ccagcatttggtgccatgatttcccagaaaa

SEQ ID NO: 563 - DNA, 34 bp, Artificial Sequence
  Description of Artificial Sequence: Synthetic probe
ttttccattttccattacaagccaagcccatcca

SEQ ID NO: 564 - DNA, 32 bp, Artificial Sequence
  Description of Artificial Sequence: Synthetic probe
ttttccattacaagccaagcccatccagaacc

SEQ ID NO: 565 - DNA, 30 bp, Artificial Sequence
  Description of Artificial Sequence: Synthetic probe
attacaagccaagcccatccagaaccaaat

SEQ ID NO: 566 - DNA, 25 bp, Artificial Sequence
  Description of Artificial Sequence: Synthetic probe
accaaatgctggtggcgaacctaca

SEQ ID NO: 567 - DNA, 28 bp, Artificial Sequence
  Description of Artificial Sequence: Synthetic probe
atttggttctggatgggcttggcttgta

SEQ ID NO: 568 - DNA, 26 bp, Artificial Sequence
  Description of Artificial Sequence: Synthetic probe
caaccagaaccaaaacgtccagctgc

SEQ ID NO: 569 - DNA, 35 bp, Artificial Sequence
  Description of Artificial Sequence: Synthetic probe
cgccaaaagcttcattaattgcttcttttattgcc

SEQ ID NO: 570 - DNA, 27 bp, Artificial Sequence
  Description of Artificial Sequence: Synthetic probe
cgcattcggtgccatgatttcccagaa

SEQ ID NO: 571 - DNA, 28 bp, Artificial Sequence
  Description of Artificial Sequence: Synthetic probe
ttttctgggaaatcatggcaccgaatgc

SEQ ID NO: 572 - DNA, 27 bp, Artificial Sequence
  Description of Artificial Sequence: Synthetic probe
ttctgggaaatcatggcaccgaatgcg

SEQ ID NO: 573 - DNA, 35 bp, Artificial Sequence
  Description of Artificial Sequence: Synthetic probe
caataaaagaagcaattaatgaagcttttggcgat

SEQ ID NO: 574 - DNA, 35 bp, Artificial Sequence
  Description of Artificial Sequence: Synthetic probe
agcttcattaattgcttcttttattgccccagtag

SEQ ID NO: 575 - DNA, 15 bp, Artificial Sequence
  Description of Artificial Sequence: Synthetic primer
ctggccgctttggtt

SEQ ID NO: 576 - DNA, 22 bp, Artificial Sequence
  Description of Artificial Sequence: Synthetic primer
acattcttctgggaaattatgg

SEQ ID NO: 577 - DNA, 25 bp, Artificial Sequence
  Description of Artificial Sequence: Synthetic primer
tgaatgctattcctgaagatatccg

SEQ ID NO: 578 - DNA, 25 bp, Artificial Sequence
  Description of Artificial Sequence: Synthetic primer
aaagaagcaatcgatgaaacatttg

SEQ ID NO: 579 - DNA, 20 bp, Artificial Sequence
  Description of Artificial Sequence: Synthetic primer
gggaaattatggcaccaaat

SEQ ID NO: 580 - DNA, 25 bp, Artificial Sequence
  Description of Artificial Sequence: Synthetic primer
ttctgggaaattatggcaccaaatg

SEQ ID NO: 581 - DNA, 24 bp, Artificial Sequence
  Description of Artificial Sequence: Synthetic primer
tggcgctattaaagaagcaatcga

SEQ ID NO: 582 - DNA, 25 bp, Artificial Sequence
  Description of Artificial Sequence: Synthetic primer
ttcttctgggaaattatggcaccaa

SEQ ID NO: 583 - DNA, 24 bp, Artificial Sequence
  Description of Artificial Sequence: Synthetic primer
gcaatcgatgaaacatttggtagc

SEQ ID NO: 584 - DNA, 23 bp, Artificial Sequence
  Description of Artificial Sequence: Synthetic primer
caactggcgctattaaagaagca

SEQ ID NO: 585 - DNA, 22 bp, Artificial Sequence
  Description of Artificial Sequence: Synthetic primer
acagctgttcgtaacaatggtg

SEQ ID NO: 586 - DNA, 25 bp, Artificial Sequence
  Description of Artificial Sequence: Synthetic primer
tcagatatgaatgctattcctgaag

SEQ ID NO: 587 - DNA, 25 bp, Artificial Sequence
  Description of Artificial Sequence: Synthetic primer
ggtagctttgatgaaatgaaagctg

SEQ ID NO: 588 - DNA, 20 bp, Artificial Sequence
  Description of Artificial Sequence: Synthetic primer
tgctggtggacaaccaactg

SEQ ID NO: 589 - DNA, 20 bp, Artificial Sequence
  Description of Artificial Sequence: Synthetic primer
accaaatgctggtggacaac

SEQ ID NO: 590 - DNA, 24 bp, Artificial Sequence
  Description of Artificial Sequence: Synthetic primer
ggtcacgcaaaccatacattcttc

SEQ ID NO: 591 - DNA, 24 bp, Artificial Sequence
  Description of Artificial Sequence: Synthetic primer
tgaaagctgctttcaaaacagctg

SEQ ID NO: 592 - DNA, 25 bp, Artificial Sequence
  Description of Artificial Sequence: Synthetic primer
ttgatgaaatgaaagctgctttcaa

SEQ ID NO: 593 - DNA, 25 bp, Artificial Sequence
  Description of Artificial Sequence: Synthetic primer
gaaatgaaagctgctttcaaaacag

SEQ ID NO: 594 - DNA, 25 bp, Artificial Sequence
  Description of Artificial Sequence: Synthetic primer
ctattcctgaagatatccgtactgc

SEQ ID NO: 595 - DNA, 25 bp, Artificial Sequence
  Description of Artificial Sequence: Synthetic primer
aacattcttctgggaaattatggca

SEQ ID NO: 596 - DNA, 25 bp, Artificial Sequence
  Description of Artificial Sequence: Synthetic primer
gcaaaccaaacattcttctgggaaa

SEQ ID NO: 597 - DNA, 25 bp, Artificial Sequence
  Description of Artificial Sequence: Synthetic primer
caaacattcttctgggaaattatgg

SEQ ID NO: 598 - DNA, 22 bp, Artificial Sequence
  Description of Artificial Sequence: Synthetic primer
ggtcacgcaaaccaaacattct

SEQ ID NO: 599 - DNA, 25 bp, Artificial Sequence
  Description of Artificial Sequence: Synthetic primer
gatgaaacatttggcagctttgatg

SEQ ID NO: 600 - DNA, 24 bp, Artificial Sequence
  Description of Artificial Sequence: Synthetic primer
acatttggcagctttgatgaaatg

SEQ ID NO: 601 - DNA, 14 bp, Artificial Sequence
  Description of Artificial Sequence: Synthetic primer
tggcgggcacgcaa

SEQ ID NO: 602 - DNA, 23 bp, Artificial Sequence
  Description of Artificial Sequence: Synthetic primer
caaccaactggcgctattaaaga

SEQ ID NO: 603 - DNA, 19 bp, Artificial Sequence
  Description of Artificial Sequence: Synthetic primer
ggacaaccaactggcgcta

SEQ ID NO: 604 - DNA, 16 bp, Artificial Sequence
  Description of Artificial Sequence: Synthetic primer
tgcgactggccgcttt

SEQ ID NO: 605 - DNA, 21 bp, Artificial Sequence
  Description of Artificial Sequence: Synthetic primer
tttcaaaacagctgcgactgg

SEQ ID NO: 606 - DNA, 25 bp, Artificial Sequence
  Description of Artificial Sequence: Synthetic primer
gtttcatcgattgcttctttaatag

SEQ ID NO: 607 - DNA, 14 bp, Artificial Sequence
  Description of Artificial Sequence: Synthetic primer
ccaaagcggccagt

SEQ ID NO: 608 - DNA, 20 bp, Artificial Sequence
  Description of Artificial Sequence: Synthetic primer
agaagaatgtatggtttgcg

SEQ ID NO: 609 - DNA, 22 bp, Artificial Sequence
  Description of Artificial Sequence: Synthetic primer
gccataatttcccagaagaatg

SEQ ID NO: 610 - DNA, 25 bp, Artificial Sequence
  Description of Artificial Sequence: Synthetic primer
ttctaatttaccgttattcacaact

SEQ ID NO: 611 - DNA, 21 bp, Artificial Sequence
  Description of Artificial Sequence: Synthetic primer
gaaagcagctttcatttcatc

SEQ ID NO: 612 - DNA, 25 bp, Artificial Sequence
  Description of Artificial Sequence: Synthetic primer
ggtgatttctaatttaccgttattc

SEQ ID NO: 613 - DNA, 15 bp, Artificial Sequence
  Description of Artificial Sequence: Synthetic primer
agcgccagttggttg

SEQ ID NO: 614 - DNA, 24 bp, Artificial Sequence
  Description of Artificial Sequence: Synthetic primer
ttctttaatagcgccagttggttg

SEQ ID NO: 615 - DNA, 25 bp, Artificial Sequence
  Description of Artificial Sequence: Synthetic primer
ccaaatgtttcatcgattgcttctt

SEQ ID NO: 616 - DNA, 25 bp, Artificial Sequence
  Description of Artificial Sequence: Synthetic primer
tttcatcgattgcttctttaatagc

SEQ ID NO: 617 - DNA, 25 bp, Artificial Sequence
  Description of Artificial Sequence: Synthetic primer
tttggtgccataatttcccagaaga

SEQ ID NO: 618 - DNA, 25 bp, Artificial Sequence
  Description of Artificial Sequence: Synthetic primer
gattgcttctttaatagcgccagtt

SEQ ID NO: 619 - DNA, 18 bp, Artificial Sequence
  Description of Artificial Sequence: Synthetic primer
tgaaccaaagcggccagt

SEQ ID NO: 620 - DNA, 17 bp, Artificial Sequence
  Description of Artificial Sequence: Synthetic primer
caaagcggccagttgca

SEQ ID NO: 621 - DNA, 15 bp, Artificial Sequence
  Description of Artificial Sequence: Synthetic primer
agcggccagttgcag

SEQ ID NO: 622 - DNA, 25 bp, Artificial Sequence
  Description of Artificial Sequence: Synthetic primer
taccgttattcacaactaaccaagc

SEQ ID NO: 623 - DNA, 25 bp, Artificial Sequence
  Description of Artificial Sequence: Synthetic primer
atttaccgttattcacaactaacca

SEQ ID NO: 624 - DNA, 25 bp, Artificial Sequence
  Description of Artificial Sequence: Synthetic primer
aatttaccgttattcacaactaacc

SEQ ID NO: 625 - DNA, 24 bp, Artificial Sequence
  Description of Artificial Sequence: Synthetic primer
ccgttattcacaactaaccaagcc

SEQ ID NO: 626 - DNA, 24 bp, Artificial Sequence
  Description of Artificial Sequence: Synthetic primer
aagcagctttcatttcatcaaagc

SEQ ID NO: 627 - DNA, 19 bp, Artificial Sequence
  Description of Artificial Sequence: Synthetic primer
ccagttggttgtccaccag

SEQ ID NO: 628 - DNA, 20 bp, Artificial Sequence
  Description of Artificial Sequence: Synthetic primer
acaactaaccaagcccaacc

SEQ ID NO: 629 - DNA, 23 bp, Artificial Sequence
  Description of Artificial Sequence: Synthetic primer
cagttgcagctgttttgaaagca

SEQ ID NO: 630 - DNA, 21 bp, Artificial Sequence
  Description of Artificial Sequence: Synthetic primer
taaccaagcccaacctgaacc

SEQ ID NO: 631 - DNA, 24 bp, Artificial Sequence
  Description of Artificial Sequence: Synthetic primer
gctgttttgaaagcagctttcatt

SEQ ID NO: 632 - DNA, 19 bp, Artificial Sequence
  Description of Artificial Sequence: Synthetic primer
gcccaacctgaaccaaagc

SEQ ID NO: 633 - DNA, 25 bp, Artificial Sequence
  Description of Artificial Sequence: Synthetic primer
tcaaagctaccaaatgtttcatcga

SEQ ID NO: 634 - DNA, 25 bp, Artificial Sequence
  Description of Artificial Sequence: Synthetic primer
atttcccagaagaatgtttggtttg

SEQ ID NO: 635 - DNA, 25 bp, Artificial Sequence
  Description of Artificial Sequence: Synthetic primer
tttcccagaagaatgtttggtttgc

SEQ ID NO: 636 - DNA, 23 bp, Artificial Sequence
  Description of Artificial Sequence: Synthetic primer
gctgccaaatgtttcatcgattg

SEQ ID NO: 637 - DNA, 24 bp, Artificial Sequence
  Description of Artificial Sequence: Synthetic primer
tgccaaatgtttcatcgattgctt

SEQ ID NO: 638 - DNA, 25 bp, Artificial Sequence
  Description of Artificial Sequence: Synthetic primer
tttcatcaaagctgccaaatgtttc

SEQ ID NO: 639 - DNA, 25 bp, Artificial Sequence
  Description of Artificial Sequence: Synthetic primer
ttcatcaaagctgccaaatgtttca

SEQ ID NO: 640 - DNA, 23 bp, Artificial Sequence
  Description of Artificial Sequence: Synthetic primer
ccagcatttggtgccataatttc

SEQ ID NO: 641 - DNA, 25 bp, Artificial Sequence
  Description of Artificial Sequence: Synthetic probe
cttctttaatagcgccagttggttg

SEQ ID NO: 642 - DNA, 32 bp, Artificial Sequence
  Description of Artificial Sequence: Synthetic probe
cgctattaaagaagcaatcgatgaaacatttg

SEQ ID NO: 643 - DNA, 22 bp, Artificial Sequence
  Description of Artificial Sequence: Synthetic probe
caggttgggcttggttagttgt

SEQ ID NO: 644 - DNA, 25 bp, Artificial Sequence
  Description of Artificial Sequence: Synthetic probe
ctgggaaattatggcaccaaatgct

SEQ ID NO: 645 - DNA, 35 bp, Artificial Sequence
  Description of Artificial Sequence: Synthetic probe
agaagcaatcgatgaaacatttggtagctttgatg

SEQ ID NO: 646 - DNA, 35 bp, Artificial Sequence
  Description of Artificial Sequence: Synthetic probe
ccaactggcgctattaaagaagcaatcgatgaaac

SEQ ID NO: 647 - DNA, 35 bp, Artificial Sequence
  Description of Artificial Sequence: Synthetic probe
aatgtttcatcgattgcttctttaatagcgccagt

SEQ ID NO: 648 - DNA, 35 bp, Artificial Sequence
  Description of Artificial Sequence: Synthetic probe
atcgatgaaacatttggtagctttgatgaaatgaa

SEQ ID NO: 649 - DNA, 33 bp, Artificial Sequence
  Description of Artificial Sequence: Synthetic probe
atcgattgcttctttaatagcgccagttggttg

SEQ ID NO: 650 - DNA, 23 bp, Artificial Sequence
  Description of Artificial Sequence: Synthetic probe
tggccgctttggttcaggttggg

SEQ ID NO: 651 - DNA, 34 bp, Artificial Sequence
  Description of Artificial Sequence: Synthetic probe
tgccataatttcccagaagaatgtatggtttgcg

SEQ ID NO: 652 - DNA, 34 bp, Artificial Sequence
  Description of Artificial Sequence: Synthetic probe
cagcatttggtgccataatttcccagaagaatgt

SEQ ID NO: 653 - DNA, 23 bp, Artificial Sequence
  Description of Artificial Sequence: Synthetic probe
ccaacctgaaccaaagcggccag

SEQ ID NO: 654 - DNA, 25 bp, Artificial Sequence
  Description of Artificial Sequence: Synthetic probe
cagctgttcgtaacaatggtggcgg

SEQ ID NO: 655 - DNA, 32 bp, Artificial Sequence
  Description of Artificial Sequence: Synthetic probe
ttctttaatagcgccagttggttgtccaccag

SEQ ID NO: 656 - DNA, 27 bp, Artificial Sequence
  Description of Artificial Sequence: Synthetic probe
taaccaagcccaacctgaaccaaagcg

SEQ ID NO: 657 - DNA, 32 bp, Artificial Sequence
  Description of Artificial Sequence: Synthetic probe
tggtggacaaccaactggcgctattaaagaag

SEQ ID NO: 658 - DNA, 35 bp, Artificial Sequence
  Description of Artificial Sequence: Synthetic probe
agcagctttcatttcatcaaagctaccaaatgttt

SEQ ID NO: 659 - DNA, 26 bp, Artificial Sequence
  Description of Artificial Sequence: Synthetic probe
caaatgctggtggacaaccaactggc

SEQ ID NO: 660 - DNA, 35 bp, Artificial Sequence
  Description of Artificial Sequence: Synthetic probe
aaacatttggtagctttgatgaaatgaaagctgct

SEQ ID NO: 661 - DNA, 26 bp, Artificial Sequence
  Description of Artificial Sequence: Synthetic probe
cgtgaccgccaccattgttacgaaca

SEQ ID NO: 662 - DNA, 22 bp, Artificial Sequence
  Description of Artificial Sequence: Synthetic probe
cgccagttggttgtccaccagc

SEQ ID NO: 663 - DNA, 27 bp, Artificial Sequence
  Description of Artificial Sequence: Synthetic probe
ctgctttcaaaacagctgcaactggcc

SEQ ID NO: 664 - DNA, 28 bp, Artificial Sequence
  Description of Artificial Sequence: Synthetic probe
agaagaatgtatggtttgcgtgaccgcc

SEQ ID NO: 665 - DNA, 21 bp, Artificial Sequence
  Description of Artificial Sequence: Synthetic probe
aaagcggccagttgcagctgt

SEQ ID NO: 666 - DNA, 35 bp, Artificial Sequence
  Description of Artificial Sequence: Synthetic probe
aaccaaacattcttctgggaaattatggcaccaaa

SEQ ID NO: 667 - DNA, 25 bp, Artificial Sequence
  Description of Artificial Sequence: Synthetic probe
cgccaccattgttacgaacggctgt

SEQ ID NO: 668 - DNA, 35 bp, Artificial Sequence
  Description of Artificial Sequence: Synthetic probe
cacgcaaaccatacattcttctgggaaattatggc

SEQ ID NO: 669 - DNA, 35 bp, Artificial Sequence
  Description of Artificial Sequence: Synthetic probe
ttcatttcatcaaagctgccaaatgtttcatcgat

SEQ ID NO: 670 - DNA, 35 bp, Artificial Sequence
  Description of Artificial Sequence: Synthetic probe
cgctattaaagaagcaatcgatgaaacatttggca

SEQ ID NO: 671 - DNA, 35 bp, Artificial Sequence
  Description of Artificial Sequence: Synthetic probe
aagctgccaaatgtttcatcgattgcttctttaat

SEQ ID NO: 672 - DNA, 35 bp, Artificial Sequence
  Description of Artificial Sequence: Synthetic probe
tcatcaaagctgccaaatgtttcatcgattgcttc

SEQ ID NO: 673 - DNA, 26 bp, Artificial Sequence
  Description of Artificial Sequence: Synthetic probe
ccagttgcagctgttttgaaagcagc

SEQ ID NO: 674 - DNA, 34 bp, Artificial Sequence
  Description of Artificial Sequence: Synthetic probe
cattcttctgggaaattatggcaccaaatgctgg

SEQ ID NO: 675 - DNA, 28 bp, Artificial Sequence
  Description of Artificial Sequence: Synthetic probe
tgggaaattatggcaccaaatgctggcg

SEQ ID NO: 676 - DNA, 23 bp, Artificial Sequence
  Description of Artificial Sequence: Synthetic probe
ctgcgactggccgctttggttca

SEQ ID NO: 677 - DNA, 23 bp, Artificial Sequence
  Description of Artificial Sequence: Synthetic probe
tgaaccaaagcggccagtcgcag

SEQ ID NO: 678 - DNA, 20 bp, Artificial Sequence
  Description of Artificial Sequence: Synthetic primer
cctcctcgtttcttactgat

SEQ ID NO: 679 - DNA, 18 bp, Artificial Sequence
  Description of Artificial Sequence: Synthetic primer
ggttcctcctcgtttctt

SEQ ID NO: 680 - DNA, 23 bp, Artificial Sequence
  Description of Artificial Sequence: Synthetic primer
gttttcgttctcttctagcaaaa

SEQ ID NO: 681 - DNA, 19 bp, Artificial Sequence
  Description of Artificial Sequence: Synthetic primer
cgatccatccgactcattg

SEQ ID NO: 682 - DNA, 18 bp, Artificial Sequence
  Description of Artificial Sequence: Synthetic primer
cgatccatccgactcatt

SEQ ID NO: 683 - DNA, 25 bp, Artificial Sequence
  Description of Artificial Sequence: Synthetic primer
tttcgccttcttatggatgtcctta

SEQ ID NO: 684 - DNA, 22 bp, Artificial Sequence
  Description of Artificial Sequence: Synthetic primer
gatccatccgactcattgtggt

SEQ ID NO: 685 - DNA, 25 bp, Artificial Sequence
  Description of Artificial Sequence: Synthetic primer
ttatagcgatccatccgactcattg

SEQ ID NO: 686 - DNA, 24 bp, Artificial Sequence
  Description of Artificial Sequence: Synthetic primer
ttatagcgatccatccgactcatt

SEQ ID NO: 687 - DNA, 25 bp, Artificial Sequence
  Description of Artificial Sequence: Synthetic primer
tcttatggatgtccttatagcgatc

SEQ ID NO: 688 - DNA, 24 bp, Artificial Sequence
  Description of Artificial Sequence: Synthetic primer
tggatgtccttatagcgatccatc

SEQ ID NO: 689 - DNA, 24 bp, Artificial Sequence
  Description of Artificial Sequence: Synthetic primer
ttgtggttcctcctcgtttcttac

SEQ ID NO: 690 - DNA, 23 bp, Artificial Sequence
  Description of Artificial Sequence: Synthetic primer
tcattgtggttcctcctcgtttc

SEQ ID NO: 691 - DNA, 21 bp, Artificial Sequence
  Description of Artificial Sequence: Synthetic primer
tccgactcattgtggttcctc

SEQ ID NO: 692 - DNA, 24 bp, Artificial Sequence
  Description of Artificial Sequence: Synthetic primer
ttcagaggtttcgccttcttatgg

SEQ ID NO: 693 - DNA, 23 bp, Artificial Sequence
  Description of Artificial Sequence: Synthetic primer
gcttcagaggtttcgccttctta

SEQ ID NO: 694 - DNA, 22 bp, Artificial Sequence
  Description of Artificial Sequence: Synthetic primer
gcttcagaggtttcgccttctt

SEQ ID NO: 695 - DNA, 21 bp, Artificial Sequence
  Description of Artificial Sequence: Synthetic primer
gtatcccgcttcagaggtttc

SEQ ID NO: 696 - DNA, 19 bp, Artificial Sequence
  Description of Artificial Sequence: Synthetic primer
tcccgcttcagaggtttcg

SEQ ID NO: 697 - DNA, 18 bp, Artificial Sequence
  Description of Artificial Sequence: Synthetic primer
cgtatcccgcttcagagg

SEQ ID NO: 698 - DNA, 23 bp, Artificial Sequence
  Description of Artificial Sequence: Synthetic primer
ggtttcgccttcttatggatgtc

SEQ ID NO: 699 - DNA, 21 bp, Artificial Sequence
  Description of Artificial Sequence: Synthetic primer
tactccccgtatcccgcttca

SEQ ID NO: 700 - DNA, 18 bp, Artificial Sequence
  Description of Artificial Sequence: Synthetic primer
cccgtatcccgcttcaga

SEQ ID NO: 701 - DNA, 18 bp, Artificial Sequence
  Description of Artificial Sequence: Synthetic primer
aagaaacgaggaggaacc

SEQ ID NO: 702 - DNA, 22 bp, Artificial Sequence
  Description of Artificial Sequence: Synthetic primer
cgctataaggacatccataaga

SEQ ID NO: 703 - DNA, 18 bp, Artificial Sequence
  Description of Artificial Sequence: Synthetic primer
cgaggaggaaccacaatg

SEQ ID NO: 704 - DNA, 22 bp, Artificial Sequence
  Description of Artificial Sequence: Synthetic primer
cttttgtcgattggttatcgta

SEQ ID NO: 705 - DNA, 23 bp, Artificial Sequence
  Description of Artificial Sequence: Synthetic primer
cttttgtcgattggttatcgtat

SEQ ID NO: 706 - DNA, 25 bp, Artificial Sequence
  Description of Artificial Sequence: Synthetic primer
tatcagtaagaaacgaggaggaacc

SEQ ID NO: 707 - DNA, 25 bp, Artificial Sequence
  Description of Artificial Sequence: Synthetic primer
atgataggcttttgtcgattggtta

SEQ ID NO: 708 - DNA, 21 bp, Artificial Sequence
  Description of Artificial Sequence: Synthetic primer
cacaatgagtcggatggatcg

SEQ ID NO: 709 - DNA, 20 bp, Artificial Sequence
  Description of Artificial Sequence: Synthetic primer
accacaatgagtcggatgga

SEQ ID NO: 710 - DNA, 23 bp, Artificial Sequence
  Description of Artificial Sequence: Synthetic primer
caatgagtcggatggatcgctat

SEQ ID NO: 711 - DNA, 22 bp, Artificial Sequence
  Description of Artificial Sequence: Synthetic primer
caatgagtcggatggatcgcta

SEQ ID NO: 712 - DNA, 21 bp, Artificial Sequence
  Description of Artificial Sequence: Synthetic primer
gtcggatggatcgctataagg

SEQ ID NO: 713 - DNA, 25 bp, Artificial Sequence
  Description of Artificial Sequence: Synthetic primer
atttatcagtaagaaacgaggagga

SEQ ID NO: 714 - DNA, 24 bp, Artificial Sequence
  Description of Artificial Sequence: Synthetic primer
ggatggatcgctataaggacatcc

SEQ ID NO: 715 - DNA, 24 bp, Artificial Sequence
  Description of Artificial Sequence: Synthetic primer
taagaaacgaggaggaaccacaat

SEQ ID NO: 716 - DNA, 22 bp, Artificial Sequence
  Description of Artificial Sequence: Synthetic primer
gaggaggaaccacaatgagtcg

SEQ ID NO: 717 - DNA, 25 bp, Artificial Sequence
  Description of Artificial Sequence: Synthetic primer
ataaggacatccataagaaggcgaa

SEQ ID NO: 718 - DNA, 25 bp, Artificial Sequence
  Description of Artificial Sequence: Synthetic primer
atcgctataaggacatccataagaa

SEQ ID NO: 719 - DNA, 25 bp, Artificial Sequence
  Description of Artificial Sequence: Synthetic primer
tgtatcgaagcatttatttcgttat

SEQ ID NO: 720 - DNA, 25 bp, Artificial Sequence
  Description of Artificial Sequence: Synthetic primer
ggcttttgtcgattggttatcgtat

SEQ ID NO: 721 - DNA, 24 bp, Artificial Sequence
  Description of Artificial Sequence: Synthetic primer
ggcttttgtcgattggttatcgta

SEQ ID NO: 722 - DNA, 25 bp, Artificial Sequence
  Description of Artificial Sequence: Synthetic primer
tttgtcgattggttatcgtatcctt

SEQ ID NO: 723 - DNA, 25 bp, Artificial Sequence
  Description of Artificial Sequence: Synthetic primer
tgtcgattggttatcgtatccttta

SEQ ID NO: 724 - DNA, 20 bp, Artificial Sequence
  Description of Artificial Sequence: Synthetic primer
cgaggaggaaccacaatgag

SEQ ID NO: 725 - DNA, 25 bp, Artificial Sequence
  Description of Artificial Sequence: Synthetic primer
aaatgataagcttttgtcgattggt

SEQ ID NO: 726 - DNA, 27 bp, Artificial Sequence
  Description of Artificial Sequence: Synthetic probe
cggatggatcgctataaggacatccat

SEQ ID NO: 727 - DNA, 27 bp, Artificial Sequence
  Description of Artificial Sequence: Synthetic probe
aggatacgataaccaatcgacaaaagc

SEQ ID NO: 728 - DNA, 23 bp, Artificial Sequence
  Description of Artificial Sequence: Synthetic probe
cgatccatccgactcattgtggt

SEQ ID NO: 729 - DNA, 35 bp, Artificial Sequence
  Description of Artificial Sequence: Synthetic probe
actgataaataaaggatacgataaccaatcgacaa

SEQ ID NO: 730 - DNA, 20 bp, Artificial Sequence
  Description of Artificial Sequence: Synthetic probe
cccgtatcccgcttcagagg

SEQ ID NO: 731 - DNA, 19 bp, Artificial Sequence
  Description of Artificial Sequence: Synthetic probe
aggcgaaacctctgaagcg

SEQ ID NO: 732 - DNA, 35 bp, Artificial Sequence
  Description of Artificial Sequence: Synthetic probe
ttcttatggatgtccttatagcgatccatccgact

SEQ ID NO: 733 - DNA, 34 bp, Artificial Sequence
  Description of Artificial Sequence: Synthetic probe
tggatgtccttatagcgatccatccgactcattg

SEQ ID NO: 734 - DNA, 35 bp, Artificial Sequence
  Description of Artificial Sequence: Synthetic probe
tggttcctcctcgtttcttactgataaataaagga

SEQ ID NO: 735 - DNA, 34 bp, Artificial Sequence
  Description of Artificial Sequence: Synthetic probe
ttcgccttcttatggatgtccttatagcgatcca

SEQ ID NO: 736 - DNA, 35 bp, Artificial Sequence
  Description of Artificial Sequence: Synthetic probe
aatgataggcttttgtcgattggttatcgtatcct

SEQ ID NO: 737 - DNA, 35 bp, Artificial Sequence
  Description of Artificial Sequence: Synthetic probe
caatgagtcggatggatcgctataaggacatccat

SEQ ID NO: 738 - DNA, 35 bp, Artificial Sequence
  Description of Artificial Sequence: Synthetic probe
cctttatttatcagtaagaaacgaggaggaaccac

SEQ ID NO: 739 - DNA, 34 bp, Artificial Sequence
  Description of Artificial Sequence: Synthetic probe
ttatagcgatccatccgactcattgtggttcctc

SEQ ID NO: 740 - DNA, 34 bp, Artificial Sequence
  Description of Artificial Sequence: Synthetic probe
tcagtaagaaacgaggaggaaccacaatgagtcg

SEQ ID NO: 741 - DNA, 35 bp, Artificial Sequence
  Description of Artificial Sequence: Synthetic probe
tcgctataaggacatccataagaaggcgaaacctc

SEQ ID NO: 742 - DNA, 35 bp, Artificial Sequence
  Description of Artificial Sequence: Synthetic probe
ctcctcgtttcttactgataaataaaggatacgat

SEQ ID NO: 743 - DNA, 35 bp, Artificial Sequence
  Description of Artificial Sequence: Synthetic probe
tcgtatcctttatttatcagtaagaaacgaggagg

SEQ ID NO: 744 - DNA, 35 bp, Artificial Sequence
  Description of Artificial Sequence: Synthetic probe
aaataaaggatacgataaccaatcgacaaaagcct

SEQ ID NO: 745 - DNA, 35 bp, Artificial Sequence
  Description of Artificial Sequence: Synthetic probe
ataaataaaggatacgataaccaatcgacaaaagc

SEQ ID NO: 746 - DNA, 35 bp, Artificial Sequence
  Description of Artificial Sequence: Synthetic probe
cttcagaggtttcgccttcttatggatgtccttat

SEQ ID NO: 747 - DNA, 35 bp, Artificial Sequence
  Description of Artificial Sequence: Synthetic probe
taaggacatccataagaaggcgaaacctctgaagc

SEQ ID NO: 748 - DNA, 35 bp, Artificial Sequence
  Description of Artificial Sequence: Synthetic probe
ttgtcgattggttatcgtatcctttatttatcagt

SEQ ID NO: 749 - DNA, 30 bp, Artificial Sequence
  Description of Artificial Sequence: Synthetic probe
catccataagaaggcgaaacctctgaagcg

SEQ ID NO: 750 - DNA, 27 bp, Artificial Sequence
  Description of Artificial Sequence: Synthetic probe
cgaggaggaaccacaatgagtcggatg

SEQ ID NO: 751 - DNA, 33 bp, Artificial Sequence
  Description of Artificial Sequence: Synthetic probe
cgactcattgtggttcctcctcgtttcttactg

SEQ ID NO: 752 - DNA, 35 bp, Artificial Sequence
  Description of Artificial Sequence: Synthetic probe
aggatacgataaccaatcgacaaaagcttatcatt

SEQ ID NO: 753 - DNA, 35 bp, Artificial Sequence
  Description of Artificial Sequence: Synthetic probe
aagcttttgtcgattggttatcgtatcctttattt

SEQ ID NO: 754 - DNA, 22 bp, Artificial Sequence
  Description of Artificial Sequence: Synthetic primer
catcttcaggagctaaatcaat

SEQ ID NO: 755 - DNA, 25 bp, Artificial Sequence
  Description of Artificial Sequence: Synthetic primer
aagtactcgtacttgaaaatcatct

SEQ ID NO: 756 - DNA, 23 bp, Artificial Sequence
  Description of Artificial Sequence: Synthetic primer
gtcgcacatcttcaggagctaaa

SEQ ID NO: 757 - DNA, 25 bp, Artificial Sequence
  Description of Artificial Sequence: Synthetic primer
ctaattgtttcactgtctctggatt

SEQ ID NO: 758 - DNA, 25 bp, Artificial Sequence
  Description of Artificial Sequence: Synthetic primer
aagaagtatccaatgactgttgcaa

SEQ ID NO: 759 - DNA, 25 bp, Artificial Sequence
  Description of Artificial Sequence: Synthetic primer
tccaatgactgttgcaataactgga

SEQ ID NO: 760 - DNA, 25 bp, Artificial Sequence
  Description of Artificial Sequence: Synthetic primer
ttcactgtctctggattaggtactc

SEQ ID NO: 761 - DNA, 25 bp, Artificial Sequence
  Description of Artificial Sequence: Synthetic primer
aattgtttcactgtctctggattag

SEQ ID NO: 762 - DNA, 25 bp, Artificial Sequence
  Description of Artificial Sequence: Synthetic primer
tcaccattttcaatatacgcatcta

SEQ ID NO: 763 - DNA, 25 bp, Artificial Sequence
  Description of Artificial Sequence: Synthetic primer
ttcaccattttcaatatacgcatct

SEQ ID NO: 764 - DNA, 25 bp, Artificial Sequence
  Description of Artificial Sequence: Synthetic primer
cgtatgatacagcgtttctaattgt

SEQ ID NO: 765 - DNA, 25 bp, Artificial Sequence
  Description of Artificial Sequence: Synthetic primer
agcgtttctaattgtttcactgtct

SEQ ID NO: 766 - DNA, 25 bp, Artificial Sequence
  Description of Artificial Sequence: Synthetic primer
cagcgtttctaattgtttcactgtc

SEQ ID NO: 767 - DNA, 24 bp, Artificial Sequence
  Description of Artificial Sequence: Synthetic primer
ttaggtactccgtcaagtactcgt

SEQ ID NO: 768 - DNA, 20 bp, Artificial Sequence
  Description of Artificial Sequence: Synthetic primer
gacaagcgtcgcacatcttc

SEQ ID NO: 769 - DNA, 25 bp, Artificial Sequence
  Description of Artificial Sequence: Synthetic primer
aaaagaagtatccaatgactgttgc

SEQ ID NO: 770 - DNA, 25 bp, Artificial Sequence
  Description of Artificial Sequence: Synthetic primer
ttttaatcgtatgatacagcgtttc

SEQ ID NO: 771 - DNA, 25 bp, Artificial Sequence
  Description of Artificial Sequence: Synthetic primer
ggaacaaattagcacctctattcta

SEQ ID NO: 772 - DNA, 20 bp, Artificial Sequence
  Description of Artificial Sequence: Synthetic primer
gattgcttgttccattggct

SEQ ID NO: 773 - DNA, 25 bp, Artificial Sequence
  Description of Artificial Sequence: Synthetic primer
tgttgcaataactggattgcttgtt

SEQ ID NO: 774 - DNA, 24 bp, Artificial Sequence
  Description of Artificial Sequence: Synthetic primer
tctggattaggtactccgtcaagt

SEQ ID NO: 775 - DNA, 24 bp, Artificial Sequence
  Description of Artificial Sequence: Synthetic primer
taactggattgcttgttccattgg

SEQ ID NO: 776 - DNA, 20 bp, Artificial Sequence
  Description of Artificial Sequence: Synthetic primer
taattgggacaagcgtcgca

SEQ ID NO: 777 - DNA, 25 bp, Artificial Sequence
  Description of Artificial Sequence: Synthetic primer
tttctgggaacaaattagcacctct

SEQ ID NO: 778 - DNA, 25 bp, Artificial Sequence
  Description of Artificial Sequence: Synthetic primer
tccgtcaagtactcgtacttgaaaa

SEQ ID NO: 779 - DNA, 25 bp, Artificial Sequence
  Description of Artificial Sequence: Synthetic primer
attgtttcactgtctctggattagg

SEQ ID NO: 780 - DNA, 20 bp, Artificial Sequence
  Description of Artificial Sequence: Synthetic primer
gctttcgagccaatggaaca

SEQ ID NO: 781 - DNA, 19 bp, Artificial Sequence
  Description of Artificial Sequence: Synthetic primer
cggagtacctaatccagag

SEQ ID NO: 782 - DNA, 25 bp, Artificial Sequence
  Description of Artificial Sequence: Synthetic primer
ttcgttaaaataaaacaggtatgga

SEQ ID NO: 783 - DNA, 25 bp, Artificial Sequence
  Description of Artificial Sequence: Synthetic primer
agtaaaggcattgattattctttct

SEQ ID NO: 784 - DNA, 25 bp, Artificial Sequence
  Description of Artificial Sequence: Synthetic primer
acagtaaaggcattgattattcttt

SEQ ID NO: 785 - DNA, 25 bp, Artificial Sequence
  Description of Artificial Sequence: Synthetic primer
atggaagttgttacattttgaatag

SEQ ID NO: 786 - DNA, 25 bp, Artificial Sequence
  Description of Artificial Sequence: Synthetic primer
ttgcaacagtcattggatacttctt

SEQ ID NO: 787 - DNA, 25 bp, Artificial Sequence
  Description of Artificial Sequence: Synthetic primer
ttagatgattttcaagtacgagtac

SEQ ID NO: 788 - DNA, 23 bp, Artificial Sequence
  Description of Artificial Sequence: Synthetic primer
gagccaatggaacaagcaatcca

SEQ ID NO: 789 - DNA, 25 bp, Artificial Sequence
  Description of Artificial Sequence: Synthetic primer
gaggtgctaatttgttcccagaaaa

SEQ ID NO: 790 - DNA, 25 bp, Artificial Sequence
  Description of Artificial Sequence: Synthetic primer
gtacgagtacttgacggagtaccta

SEQ ID NO: 791 - DNA, 24 bp, Artificial Sequence
  Description of Artificial Sequence: Synthetic primer
aaaataaaacaggtatggaagttg

SEQ ID NO: 792 - DNA, 25 bp, Artificial Sequence
  Description of Artificial Sequence: Synthetic primer
tccagttattgcaacagtcattgga

SEQ ID NO: 793 - DNA, 25 bp, Artificial Sequence
  Description of Artificial Sequence: Synthetic primer
agaagaaagacagtaaaggcattga

SEQ ID NO: 794 - DNA, 25 bp, Artificial Sequence
  Description of Artificial Sequence: Synthetic primer
taaaacaggtatggaagttgttaca

SEQ ID NO: 795 - DNA, 22 bp, Artificial Sequence
  Description of Artificial Sequence: Synthetic primer
tttcgagccaatggaacaagca

SEQ ID NO: 796 - DNA, 25 bp, Artificial Sequence
  Description of Artificial Sequence: Synthetic primer
tggaacaagcaatccagttattgca

SEQ ID NO: 797 - DNA, 25 bp, Artificial Sequence
  Description of Artificial Sequence: Synthetic primer
tgaatagaatagaggtgctaatttg

SEQ ID NO: 798 - DNA, 20 bp, Artificial Sequence
  Description of Artificial Sequence: Synthetic primer
gagccaatggaacaagcaat

SEQ ID NO: 799 - DNA, 25 bp, Artificial Sequence
  Description of Artificial Sequence: Synthetic primer
aacaggtatggaagttgttacattt

SEQ ID NO: 800 - DNA, 24 bp, Artificial Sequence
  Description of Artificial Sequence: Synthetic primer
caagcaatccagttattgcaacag

SEQ ID NO: 801 - DNA, 25 bp, Artificial Sequence
  Description of Artificial Sequence: Synthetic primer
agacagtaaaggcattgattattct

SEQ ID NO: 802 - DNA, 25 bp, Artificial Sequence
  Description of Artificial Sequence: Synthetic primer
gcaacagtcattggatacttctttt

SEQ ID NO: 803 - DNA, 25 bp, Artificial Sequence
  Description of Artificial Sequence: Synthetic primer
tagaggtgctaatttgttcccagaa

SEQ ID NO: 804 - DNA, 25 bp, Artificial Sequence
  Description of Artificial Sequence: Synthetic primer
aaaggcattgattattctttctttt

SEQ ID NO: 805 - DNA, 25 bp, Artificial Sequence
  Description of Artificial Sequence: Synthetic primer
gaatagaatagaggtgctaatttgt

SEQ ID NO: 806 - DNA, 25 bp, Artificial Sequence
  Description of Artificial Sequence: Synthetic primer
atagaatagaggtgctaatttgttc

SEQ ID NO: 807 - DNA, 25 bp, Artificial Sequence
  Description of Artificial Sequence: Synthetic primer
gtacttgacggagtacctaatccag

SEQ ID NO: 808 - DNA, 25 bp, Artificial Sequence
  Description of Artificial Sequence: Synthetic primer
ttttcaagtacgagtacttgacgga

SEQ ID NO: 809 - DNA, 25 bp, Artificial Sequence
  Description of Artificial Sequence: Synthetic primer
agatgcgtatattgaaaatggtgaa

SEQ ID NO: 810 - DNA, 30 bp, Artificial Sequence
  Description of Artificial Sequence: Synthetic probe
ccagttattgcaacagtcattggatacttc

SEQ ID NO: 811 - DNA, 35 bp, Artificial Sequence
  Description of Artificial Sequence: Synthetic probe
cgttaaaataaaacaggtatggaagttgttacatt

SEQ ID NO: 812 - DNA, 35 bp, Artificial Sequence
  Description of Artificial Sequence: Synthetic probe
ccgtcaagtactcgtacttgaaaatcatctaaaat

SEQ ID NO: 813 - DNA, 35 bp, Artificial Sequence
  Description of Artificial Sequence: Synthetic probe
atgtaacaacttccatacctgttttattttaacga

SEQ ID NO: 814 - DNA, 35 bp, Artificial Sequence
  Description of Artificial Sequence: Synthetic probe
acattttgaatagaatagaggtgctaatttgttcc

SEQ ID NO: 815 - DNA, 29 bp, Artificial Sequence
  Description of Artificial Sequence: Synthetic probe
aatagaggtgctaatttgttcccagaaaa

SEQ ID NO: 816 - DNA, 35 bp, Artificial Sequence
  Description of Artificial Sequence: Synthetic probe
ccagagacagtgaaacaattagaaacgctgtatca

SEQ ID NO: 817 - DNA, 35 bp, Artificial Sequence
  Description of Artificial Sequence: Synthetic probe
cgtatgatacagcgtttctaattgtttcactgtct

SEQ ID NO: 818 - DNA, 35 bp, Artificial Sequence
  Description of Artificial Sequence: Synthetic probe
agaagtatccaatgactgttgcaataactggattg

SEQ ID NO: 819 - DNA, 35 bp, Artificial Sequence
  Description of Artificial Sequence: Synthetic probe
acagtgaaacaattagaaacgctgtatcatacgat

SEQ ID NO: 820 - DNA, 35 bp, Artificial Sequence
  Description of Artificial Sequence: Synthetic probe
ctgtctctggattaggtactccgtcaagtactcgt

SEQ ID NO: 821 - DNA, 35 bp, Artificial Sequence
  Description of Artificial Sequence: Synthetic probe
ctggattaggtactccgtcaagtactcgtacttga

SEQ ID NO: 822 - DNA, 35 bp, Artificial Sequence
  Description of Artificial Sequence: Synthetic probe
aggtactccgtcaagtactcgtacttgaaaatcat

SEQ ID NO: 823 - DNA, 35 bp, Artificial Sequence
  Description of Artificial Sequence: Synthetic probe
agcgtttctaattgtttcactgtctctggattagg

SEQ ID NO: 824 - DNA, 35 bp, Artificial Sequence
  Description of Artificial Sequence: Synthetic probe
atccaatgactgttgcaataactggattgcttgtt

SEQ ID NO: 825 - DNA, 35 bp, Artificial Sequence
  Description of Artificial Sequence: Synthetic probe
aaacaggtatggaagttgttacattttgaatagaa

SEQ ID NO: 826 - DNA, 35 bp, Artificial Sequence
  Description of Artificial Sequence: Synthetic probe
aatggaacaagcaatccagttattgcaacagtcat

SEQ ID NO: 827 - DNA, 34 bp, Artificial Sequence
  Description of Artificial Sequence: Synthetic probe
ctttcgagccaatggaacaagcaatccagttatt

SEQ ID NO: 828 - DNA, 35 bp, Artificial Sequence
  Description of Artificial Sequence: Synthetic probe
acctaatccagagacagtgaaacaattagaaacgc

SEQ ID NO: 829 - DNA, 35 bp, Artificial Sequence
  Description of Artificial Sequence: Synthetic probe
agcacctctattctattcaaaatgtaacaacttcc

SEQ ID NO: 830 - DNA, 35 bp, Artificial Sequence
  Description of Artificial Sequence: Synthetic probe
attgtttcactgtctctggattaggtactccgtca

SEQ ID NO: 831 - DNA, 35 bp, Artificial Sequence
  Description of Artificial Sequence: Synthetic probe
tgttgcaataactggattgcttgttccattggctc

SEQ ID NO: 832 - DNA, 33 bp, Artificial Sequence
  Description of Artificial Sequence: Synthetic probe
taactggattgcttgttccattggctcgaaagc

SEQ ID NO: 833 - DNA, 35 bp, Artificial Sequence
  Description of Artificial Sequence: Synthetic probe
attctattcaaaatgtaacaacttccatacctgtt

SEQ ID NO: 834 - DNA, 35 bp, Artificial Sequence
  Description of Artificial Sequence: Synthetic probe
atgattttcaagtacgagtacttgacggagtacct

SEQ ID NO: 835 - DNA, 35 bp, Artificial Sequence
  Description of Artificial Sequence: Synthetic probe
ctgggaacaaattagcacctctattctattcaaaa

SEQ ID NO: 836 - DNA, 35 bp, Artificial Sequence
  Description of Artificial Sequence: Synthetic probe
caagtacgagtacttgacggagtacctaatccaga

SEQ ID NO: 837 - DNA, 35 bp, Artificial Sequence
  Description of Artificial Sequence: Synthetic probe
tgacggagtacctaatccagagacagtgaaacaat

SEQ ID NO: 838 - DNA, 35 bp, Artificial Sequence
  Description of Artificial Sequence: Synthetic probe
tggaagttgttacattttgaatagaatagaggtgc

SEQ ID NO: 839 - DNA, 35 bp, Artificial Sequence
  Description of Artificial Sequence: Synthetic probe
tgaatagaatagaggtgctaatttgttcccagaaa

SEQ ID NO: 840 - DNA, 34 bp, Artificial Sequence
  Description of Artificial Sequence: Synthetic probe
cgagtacttgacggagtacctaatccagagacag

SEQ ID NO: 841 - DNA, 35 bp, Artificial Sequence
  Description of Artificial Sequence: Synthetic probe
attttagatgattttcaagtacgagtacttgacgg

SEQ ID NO: 842 - DNA, 35 bp, Artificial Sequence
  Description of Artificial Sequence: Synthetic probe
agcaatccagttattgcaacagtcattggatactt

SEQ ID NO: 843 - DNA, 24 bp, Artificial Sequence
  Description of Artificial Sequence: Synthetic primer
ttgctttatcgtgacgtgactttg

SEQ ID NO: 844 - DNA, 33 bp, Artificial Sequence
  Description of Artificial Sequence: Synthetic probe
cattctcctcgctatcatcggtttaattgtcgg

SEQ ID NO: 845 - DNA, 25 bp, Artificial Sequence
  Description of Artificial Sequence: Synthetic primer
gatagtatggaggtggaaatatggt

SEQ ID NO: 846 - DNA, 25 bp, Artificial Sequence
  Description of Artificial Sequence: Synthetic primer
tatggaggtggattttatggtattc

SEQ ID NO: 847 - DNA, 10 bp, Artificial Sequence
  Description of Artificial Sequence: Synthetic probe
aaacacgtgc

SEQ ID NO: 848 - DNA, 10 bp, Artificial Sequence
  Description of Artificial Sequence: Synthetic probe
ccttgttcca

SEQ ID NO: 849 - DNA, 10 bp, Artificial Sequence
  Description of Artificial Sequence: Synthetic probe
cagggacgat